(19) United States
(12) Patent Application Publication
(10) Pub. No.: US 2002/0048759 A1
(43) Pub. Date: Apr. 25, 2002

(54) Compositions and methods for the therapy and diagnosis of ovarian and endometrial cancer

(76) Inventors: Jiangchun Xu, Bellevue, WA (US); Ruth A. Pyle, Seattle, WA (US); John A. Stolk, Bothell, WA (US)

Correspondence Address: SEED INTELLECTUAL PROPERTY LAW GROUP PLLC, 701 FIFTH AVE, SUITE 6300, SEATTLE, WA, 98104-7092, US

(21) Appl. No.: 09/813,358

(22) Filed: Mar. 21, 2001

Related U.S. Application Data
(60) Provisional application No. 60/190,710, filed on Mar. 21, 2000.
(60) Provisional application No. 60/213,748, filed on Jun. 22, 2000.
(60) Provisional application No. 60/257,276, filed on Dec. 19, 2000.

(51) Int. Cl. C12Q 1/68; G01N 33/574; C07H 21/04; A61K 39/395; A61K 48/00

(52) U.S. Cl. 435/6; 435/7.23; 435/69.1; 435/325; 424/93.21; 514/44; 514/12; 424/155.1; 536/23.5

(57) ABSTRACT

Compositions and methods for the therapy and diagnosis of cancer, such as ovarian or endometrial cancer, are disclosed. Compositions may comprise one or more ovarian carcinoma proteins, immunogenic portions thereof, or polynucleotides that encode such portions. Alternatively, a therapeutic composition may comprise an antigen presenting cell that expresses such an antigen, or a T cell that is specific for cells expressing such an antigen. Such compositions may be used, for example, for the prevention and treatment of diseases such as ovarian and endometrial cancer. Diagnostic methods based on detecting an ovarian carcinoma protein, or mRNA encoding such an antigen, in a sample are also provided.

### CROSS REFERENCE TO RELATED APPLICATIONS

This application is related to U.S. Provisional Applications 60/190,710, filed Mar. 21, 2000; 60/213,748, filed Jun. 22, 2000; and 60/257,276, filed Dec. 19, 2000, all incorporated in their entirety herein by reference.

### TECHNICAL FIELD OF THE INVENTION

The present invention relates generally to therapy and diagnosis of cancer, such as ovarian or endometrial cancer. The invention is more specifically related to polypeptides comprising at least a portion of an ovarian carcinoma protein, and to polynucleotides encoding such polypeptides. Such polypeptides and polynucleotides may be used in vaccines and pharmaceutical compositions for prevention and treatment of cancers such as ovarian and endometrial cancer, and for the diagnosis and monitoring of such cancers.

### BACKGROUND OF THE INVENTION

Ovarian cancer is a significant health problem for women in the United States and throughout the world. Although advances have been made in detection and therapy of this cancer, no vaccine or other universally successful method for prevention or treatment is currently available. Management of the disease currently relies on a combination of early diagnosis and aggressive treatment, which may include one or more of a variety of treatments such as surgery, radiotherapy, chemotherapy and hormone therapy. The course of treatment for a particular cancer is often selected based on a variety of prognostic parameters, including an analysis of specific tumor markers. However, the use of established markers often leads to a result that is difficult to interpret, and high mortality continues to be observed in many cancer patients.

Inmunotherapies have the potential to substantially improve cancer treatment and survival. Such therapies may involve the generation or enhancement of an immune response to an ovarian carcinoma protein. However, to date, relatively few ovarian carcinoma proteins are known and the generation of an immune response against such antigens has not been shown to be therapeutically beneficial.

Accordingly, there is a need in the art for improved methods for detecting and treating cancers such as ovarian cancer. The present invention fulfills these needs and further provides other related advantages.

### SUMMARY OF THE INVENTION

Briefly stated, the present invention provides compositions and methods for the diagnosis and therapy of cancer, such as ovarian and endometrial cancer. In one aspect, the present invention provides polypeptides comprising at least a portion of an ovarian carcinoma protein, or a variant thereof. Certain portions and other variants are immunogenic, such that the ability of the variant to react with antigen-specific antisera is not substantially diminished. Within certain embodiments, the polypeptide comprises a sequence that is encoded by a polynucleotide sequence selected from the group consisting of: (a) sequences recited in SEQ ID NO:1-222; (b) variants of a sequence recited in SEQ ID NO:1-222 and (c) complements of a sequence of (a) or (b).

The present invention further provides polynucleotides that encode a polypeptide as described above, or a portion thereof (such as a portion encoding at least 15 amino acid residues of an ovarian carcinoma protein), expression vectors comprising such polynucleotides and host cells transformed or transfected with such expression vectors.

Within other aspects, the present invention provides pharmaceutical compositions comprising a polypeptide or polynucleotide as described above and a physiologically acceptable carrier.

Within a related aspect of the present invention, vaccines for prophylactic or therapeutic use are provided. Such vaccines comprise a polypeptide or polynucleotide as described above and an immunostimulant.

The present invention further provides pharmaceutical compositions that comprise: (a) an antibody or antigen-binding fragment thereof that specifically binds to an ovarian carcinoma protein; and (b) a physiologically acceptable carrier.

Within further aspects, the present invention provides pharmaceutical compositions comprising: (a) an antigen presenting cell that expresses a polypeptide as described above and (b) a pharmaceutically acceptable carrier or excipient. Antigen presenting cells include dendritic cells, macrophages, monocytes, fibroblasts and B cells.

Within related aspects, vaccines are provided that comprise: (a) an antigen presenting cell that expresses a polypeptide as described above and (b) an immunostimulant.

The present invention further provides, in other aspects, fusion proteins that comprise at least one polypeptide as described above, as well as polynucleotides encoding such fusion proteins.

Within related aspects, pharmaceutical compositions comprising a fusion protein, or a polynucleotide encoding a fusion protein, in combination with a physiologically acceptable carrier are provided.

Vaccines are further provided, within other aspects, that comprise a fusion protein, or a polynucleotide encoding a fusion protein, in combination with an immunostimulant.

Within further aspects, the present invention provides methods for inhibiting the development of a cancer in a patient, comprising administering to a patient a pharmaceutical composition or vaccine as recited above. The patient may be afflicted with ovarian or endometrial cancer, in which case the methods provide treatment for the disease, or patient considered at risk for such a disease may be treated prophylactically.

The present invention further provides, within other aspects, methods for removing tumor cells from a biological sample, comprising contacting a biological sample with T cells that specifically react with an ovarian carcinoma protein, wherein the step of contacting is performed under conditions and for a time sufficient to permit the removal of cells expressing the protein from the sample.

Within related aspects, methods are provided for inhibiting the development of a cancer in a patient, comprising administering to a patient a biological sample treated as described above.

Methods are further provided, within other aspects, for stimulating and/or expanding T cells specific for an ovarian carcinoma protein, comprising contacting T cells with one or more of: (i) a polypeptide as described above; (ii) a polynucleotide encoding such a polypeptide; and/or (iii) an antigen presenting cell that expresses such a polypeptide; under conditions and for a time sufficient to permit the stimulation and/or expansion of T cells. Isolated T cell populations comprising T cells prepared as described above are also provided.

Within further aspects, the present invention provides methods for inhibiting the development of a cancer in a patient, comprising administering to a patient an effective amount of a T cell population as described above.

The present invention further provides methods for inhibiting the development of a cancer in a patient, comprising the steps of: (a) incubating CD4⁺ and/or CD8⁺ T cells isolated from a patient with one or more of: (i) a polypeptide comprising at least an immunogenic portion of an ovarian carcinoma protein; (ii) a polynucleotide encoding such a polypeptide; and (iii) an antigen-presenting cell that expresses such a polypeptide; and (b) administering to the patient an effective amount of the proliferated T cells, and thereby inhibiting the development of a cancer in the patient. Proliferated cells may, but need not, be cloned prior to administration to the patient.

Within further aspects, the present invention provides methods for determining the presence or absence of a cancer in a patient, comprising: (a) contacting a biological sample obtained from a patient with a binding agent that binds to a polypeptide as recited above; (b) detecting in the sample an amount of polypeptide that binds to the binding agent; and (c) comparing the amount of polypeptide with a predetermined cut-off value, and therefrom determining the presence or absence of a cancer in the patient. Within preferred embodiments, the binding agent is an antibody, more preferably a monoclonal antibody. The cancer may be ovarian or endometrial cancer.

The present invention also provides, within other aspects, methods for monitoring the progression of a cancer in a patient. Such methods comprise the steps of: (a) contacting a biological sample obtained from a patient at a first point in time with a binding agent that binds to a polypeptide as recited above; (b) detecting in the sample an amount of polypeptide that binds to the binding agent; (c) repeating steps (a) and (b) using a biological sample obtained from the patient at a subsequent point in time; and (d) comparing the amount of polypeptide detected in step (c) with the amount detected in step (b) and therefrom monitoring the progression of the cancer in the patient.

The present invention further provides, within other aspects, methods for determining the presence or absence of a cancer in a patient, comprising the steps of: (a) contacting a biological sample obtained from a patient with an oligonucleotide that hybridizes to a polynucleotide that encodes an ovarian carcinoma protein; (b) detecting in the sample a level of a polynucleotide, preferably mRNA, that hybridizes to the oligonucleotide; and (c) comparing the level of polynucleotide that hybridizes to the oligonucleotide with a predetermined cut-off value, and therefrom determining the presence or absence of a cancer in the patient. Within certain embodiments, the amount of mRNA is detected via polymerase chain reaction using, for example, at least one oligonucleotide primer that hybridizes to a polynucleotide encoding a polypeptide as recited above, or a complement of such a polynucleotide. Within other embodiments, the amount of mRNA is detected using a hybridization technique, employing an oligonucleotide probe that hybridizes to a polynucleotide that encodes a polypeptide as recited above, or a complement of such a polynucleotide.

In related aspects, methods are provided for monitoring the progression of a cancer in a patient, comprising the steps of: (a) contacting a biological sample obtained from a patient with an oligonucleotide that hybridizes to a polynucleotide that encodes an ovarian carcinoma protein; (b) detecting in the sample an amount of a polynucleotide that hybridizes to the oligonucleotide; (c) repeating steps (a) and (b) using a biological sample obtained from the patient at a subsequent point in time; and (d) comparing the amount of polynucleotide detected in step (c) with the amount detected in step (b) and therefrom monitoring the progression of the cancer in the patient.

Within further aspects, the present invention provides antibodies, such as monoclonal antibodies, that bind to a polypeptide as described above, as well as diagnostic kits comprising such antibodies. Diagnostic kits comprising one or more oligonucleotide probes or primers as described above are also provided.

These and other aspects of the present invention will become apparent upon reference to the following detailed description. All references disclosed herein are hereby incorporated by reference in their entirety as if each was incorporated individually.

### BRIEF DESCRIPTION OF THE SEQUENCE IDENTIFIERS

SEQ ID NOs: 1-41 are identified in Example 1.

SEQ ID NO:42 is the determined cDNA sequence for clone R0198:A03

SEQ ID NO:43 is the determined cDNA sequence for clone R0198:A07

SEQ ID NO:44 is the determined cDNA sequence for clone R0198:A08

SEQ ID NO:45 is the determined cDNA sequence for clone R0198:A09

SEQ ID NO:46 is the determined cDNA sequence for clone R0198:B01

SEQ ID NO:47 is the determined cDNA sequence for clone R0198:B02

SEQ ID NO:48 is the determined cDNA sequence for clone R0198:B04

SEQ ID NO:49 is the determined cDNA sequence for clone R0198:B08

SEQ ID NO:50 is the determined cDNA sequence for clone R0198:B11

SEQ ID NO:51 is the determined cDNA sequence for clone R0198:C01

SEQ ID NO:52 is the determined cDNA sequence for clone R0198:C02

SEQ ID NO:53 is the determined cDNA sequence for clone R0198:C03

SEQ ID NO:54 is the determined cDNA sequence for clone R0198:C04

SEQ ID NO:55 is the determined cDNA sequence for clone R0198:C05

SEQ ID NO:56 is the determined cDNA sequence for clone R0198:C06

SEQ ID NO:57 is the determined cDNA sequence for clone R0198:C08

SEQ ID NO:58 is the determined cDNA sequence for clone R0198:C09

SEQ ID NO:59 is the determined cDNA sequence for clone R0198:C10

SEQ ID NO:60 is the determined cDNA sequence for clone R0198:C12

SEQ ID NO:61 is the determined cDNA sequence for clone R0198:D01

SEQ ID NO:62 is the determined cDNA sequence for clone R0198:D02

SEQ ID NO:63 is the determined cDNA sequence for clone R0198:D03

SEQ ID NO:64 is the determined cDNA sequence for clone R0198:D04

SEQ ID NO:65 is the determined cDNA sequence for clone R0198:D05

SEQ ID NO:66 is the determined cDNA sequence for clone R0198:D06

SEQ ID NO:67 is the determined cDNA sequence for clone R0198:D07

SEQ ID NO:68 is the determined cDNA sequence for clone R0198:D08

SEQ ID NO:69 is the determined cDNA sequence for clone R0198:D09

SEQ ID NO:70 is the determined cDNA sequence for clone R0198:D11

SEQ ID NO:71 is the determined cDNA sequence for clone R0198:E01

SEQ ID NO:72 is the determined cDNA sequence for clone R0198:E03

SEQ ID NO:73 is the determined cDNA sequence for clone R0198:E05

SEQ ID NO:74 is the determined cDNA sequence for clone R0198:E06

SEQ ID NO:75 is the determined cDNA sequence for clone R0198:E09

SEQ ID NO:76 is the determined cDNA sequence for clone R0198:E10

SEQ ID NO:77 is the determined cDNA sequence for clone R0198:E11

SEQ ID NO:78 is the determined cDNA sequence for clone R0198:E12

SEQ ID NO:79 is the determined cDNA sequence for clone R0198:F01

SEQ ID NO:80 is the determined cDNA sequence for clone R0198:F02

SEQ ID NO:81 is the determined cDNA sequence for clone R0198:F03

SEQ ID NO:82 is the determined cDNA sequence for clone R0198:F04

SEQ ID NO:83 is the determined cDNA sequence for clone R0198:F06

SEQ ID NO:84 is the determined cDNA sequence for clone R0198:F07

SEQ ID NO:85 is the determined cDNA sequence for clone R0198:F09

SEQ ID NO:86 is the determined cDNA sequence for clone R0198:F10

SEQ ID NO:87 is the determined cDNA sequence for clone R0198:F11

SEQ ID NO:88 is the determined cDNA sequence for clone R0198:F12

SEQ ID NO:89 is the determined cDNA sequence for clone R0198:G01

SEQ ID NO:90 is the determined cDNA sequence for clone R0198:G02

SEQ ID NO:91 is the determined cDNA sequence for clone R0198:G03

SEQ ID NO:92 is the determined cDNA sequence for clone R0198:G04

SEQ ID NO:93 is the determined cDNA sequence for clone R0198:G05

SEQ ID NO:94 is the determined cDNA sequence for clone R0198:G06

SEQ ID NO:95 is the determined cDNA sequence for clone R0198:G09

SEQ ID NO:96 is the determined cDNA sequence for clone R0198:G11

SEQ ID NO:97 is the determined cDNA sequence for clone R0198:G12

SEQ ID NO:98 is the determined cDNA sequence for clone R0198:H01

SEQ If NO:99 is the determined cDNA sequence for clone R0198:H03

SEQ ID NO:100 is the determined cDNA sequence for clone R0198:H04

SEQ ID NO:101 is the determined cDNA sequence for clone R0198:H06

SEQ ID NO:102 is the determined cDNA sequence for clone R0198:H09

SEQ ID NO:103 is the determined cDNA sequence for clone R0198:H10

SEQ ID NO:104 is the determined cDNA sequence for clone R0199:A03

SEQ ID NO:105 is the determined cDNA sequence for clone R0199:A05

SEQ ID NO:106 is the determined cDNA sequence for clone R0199:A06

SEQ ID NO:107 is the determined cDNA sequence for clone R0199:A07

SEQ If NO:108 is the determined cDNA sequence for clone R0199:A08

SEQ ID NO:109 is the determined cDNA sequence for clone R0199:A11

SEQ ID NO:110 is the determined cDNA sequence for clone R0199:B01

SEQ ID NO:111 is the determined cDNA sequence for clone R0199:B03

SEQ ID NO:112 is the determined cDNA sequence for clone R0199:B06

SEQ ID NO:113 is the determined cDNA sequence for clone R0199:B07

SEQ ID NO:114 is the determined cDNA sequence for clone R0199:B08

SEQ ID NO:115 is the determined cDNA sequence for clone R0199:B09

SEQ ID NO:116 is the determined cDNA sequence for clone R0199:B11

SEQ ID NO:117 is the determined cDNA sequence for clone R0199:C01

SEQ ID NO:118 is the determined cDNA sequence for clone R0199:C02

SEQ ID NO:119 is the determined cDNA sequence for clone R0199:C06

SEQ ID NO:120 is the determined cDNA sequence for clone R0199:C07

SEQ ID NO:121 is the determined cDNA sequence for clone R0199:C08

SEQ ID NO:122 is the determined cDNA sequence for clone R0199:C09

SEQ ID NO:123 is the determined cDNA sequence for clone R0199:C10

SEQ ID NO:124 is the determined cDNA sequence for clone R0199:C11

SEQ ID NO:125 is the determined cDNA sequence for clone R0199:C12

SEQ ID NO:126 is the determined cDNA sequence for clone R0199:D01

SEQ ID NO:127 is the determined cDNA sequence for clone R0199:D02

SEQ ID NO:128 is the determined cDNA sequence for clone R0199:D04

SEQ ID NO:129 is the determined cDNA sequence for clone R0199:D06

SEQ ID NO:130 is the determined cDNA sequence for clone R0199:D07

SEQ ID NO:131 is the determined cDNA sequence for clone R0199:D08

SEQ ID NO:132 is the determined cDNA sequence for clone R0199:D11

SEQ ID NO:133 is the determined cDNA sequence for clone R0199:E02

SEQ ID NO:134 is the determined cDNA sequence for clone R0199:E03

SEQ ID NO:135 is the determined cDNA sequence for clone R0199:E05

SEQ ID NO:136 is the determined cDNA sequence for clone R0199:E06

SEQ ID NO:137 is the determined cDNA sequence for clone R0199:E08

SEQ ID NO:138 is the determined cDNA sequence for clone R0199:E09

SEQ ID NO:139 is the determined cDNA sequence for clone R0199:E10

SEQ ID NO:140 is the determined cDNA sequence for clone R0199:E12

SEQ ID NO:141 is the determined cDNA sequence for clone R0199:F01

SEQ ID NO:142 is the determined cDNA sequence for clone R0199:F03

SEQ ID NO:143 is the determined cDNA sequence for clone R0199:F04

SEQ ID NO:144 is the determined cDNA sequence for clone R0199:F06

SEQ ID NO:145 is the determined cDNA sequence for clone R0199:F09

SEQ ID NO:146 is the determined cDNA sequence for clone R0199:F10

SEQ ID NO:147 is the determined cDNA sequence for clone R0199:G01

SEQ ID NO:148 is the determined cDNA sequence for clone R0199:G05

SEQ ID NO:149 is the determined cDNA sequence for clone R0199:G06

SEQ ID NO:150 is the determined cDNA sequence for clone R0199:G08

SEQ ID NO:151 is the determined cDNA sequence for clone R0199:G11

SEQ ID NO:152 is the determined cDNA sequence for clone R0199:G12

SEQ ID NO:153 is the determined cDNA sequence for clone R0199:H02

SEQ ID NO:154 is the determined cDNA sequence for clone R0199:H03

SEQ ID NO:155 is the determined cDNA sequence for clone R0200:A05

SEQ ID NO:156 is the determined cDNA sequence for clone R0200:A06

SEQ ID NO:157 is the determined cDNA sequence for clone R0200:A10

SEQ ID NO:158 is the determined cDNA sequence for clone R0200:A12

SEQ ID NO:159 is the determined cDNA sequence for clone R0200:B03

SEQ ID NO:160 is the determined cDNA sequence for clone R0200:B04

SEQ ID NO:161 is the determined cDNA sequence for clone R0200:B07

SEQ ID NO:162 is the determined cDNA sequence for clone R0200:B10

SEQ ID NO:163 is the determined cDNA sequence for clone R0200:B12

SEQ ID NO:164 is the determined cDNA sequence for clone R0200:C02

SEQ ID NO:165 is the determined cDNA sequence for clone R0200:C07

SEQ ID NO:166 is the determined cDNA sequence for clone R0200:C09

SEQ ID NO:167 is the determined cDNA sequence for clone R0200:C10

SEQ ID NO:168 is the determined cDNA sequence for clone R0200:D01

SEQ ID NO:169 is the determined cDNA sequence for clone R0200:D03

SEQ ID NO:170 is the determined cDNA sequence for clone R0200:D05

SEQ ID NO:171 is the determined cDNA sequence for clone R0200:D06

SEQ ID NO:172 is the determined cDNA sequence for clone R0200:D07

SEQ ID NO:173 is the determined cDNA sequence for clone R0200:D08

SEQ ID NO:174 is the determined cDNA sequence for clone R0200:D09

SEQ ID NO:175 is the determined cDNA sequence for clone R0200:D11

SEQ ID NO:176 is the determined cDNA sequence for clone R0200:D12

SEQ ID NO:177 is the determined cDNA sequence for clone R0200:E03

SEQ ID NO:178 is the determined cDNA sequence for clone R0200:E04

SEQ ID NO:179 is the determined cDNA sequence for clone R0200:E06

SEQ ID NO:180 is the determined cDNA sequence for clone R0200:E07

SEQ ID NO:181 is the determined cDNA sequence for clone R0200:E08

SEQ ID NO:182 is the determined cDNA sequence for clone R0200:E09

SEQ ID NO:183 is the determined cDNA sequence for clone R0200:E12

SEQ ID NO:184 is the determined cDNA sequence for clone R0200:F01

SEQ ID NO:185 is the determined cDNA sequence for clone R0200:F04

SEQ ID NO:186 is the determined cDNA sequence for clone R0200:F05

SEQ ID NO:187 is the determined cDNA sequence for clone R0200:F07

SEQ ID NO:188 is the determined cDNA sequence for clone R0200:F08

SEQ ID NO:189 is the determined cDNA sequence for clone R0200:F09

SEQ ID NO:190 is the determined cDNA sequence for clone R0200:F10

SEQ ID NO:191 is the determined cDNA sequence for clone R0200:F11

SEQ ID NO:192 is the determined cDNA sequence for clone R0200:F12

SEQ ID NO:193 is the determined cDNA sequence for clone R0200:G02

SEQ ID NO:194 is the determined cDNA sequence for clone R0200:G07

SEQ ID NO:195 is the determined cDNA sequence for clone R0200:G08

SEQ ID NO:196 is the determined cDNA sequence for clone R0200:G09

SEQ If NO:197 is the determined cDNA sequence for clone R0200:G10

SEQ ID NO:198 is the determined cDNA sequence for clone R0200:G12

SEQ ID NO:199 is the determined cDNA sequence for clone R0200:H03

SEQ ID NO:200 is the determined cDNA sequence for clone R0200:H05

SEQ ID NO:201 is the determined cDNA sequence for clone R0200:H07

SEQ ID NO:202 is the determined cDNA sequence for clone R0200:H09

SEQ ID NO:203 is the determined cDNA sequence for clone R0200:H11

SEQ ID NO:204 is the determined cDNA sequence for clone57877.2

SEQ ID NO:205 is the determined cDNA sequence for clone57879.3

SEQ ID NO:206 is the determined cDNA sequence for clone57881.2

SEQ ID NO:207 is the determined cDNA sequence for clone57882.1

SEQ ID NO:208 is the determined cDNA sequence for clone57884.2

SEQ ID NO:209 is the determined cDNA sequence for clone57888.2

SEQ ID NO:210 is an extended cDNA sequence for clone R0198 C12 (SEQ ID NO: 60), also referred to as O593S

SEQ ID NO:211 is an extended cDNA sequence for clone R0198 F2 (SEQ ID NO: 80), also referred to as O594S

SEQ ID NO:212 is an extended cDNA sequence for clone R0199 A7 (SEQ ID NO: 107), also referred to as O595S

SEQ ID NO:213 is an extended cDNA sequence for clone R0199 C12 (SEQ ID NO: 125), also referred to as O596S

SEQ ID NO:214 is a full length cDNA sequence for HSPCO67, a sequence having homology with O596S

SEQ ID NO:215 is an extended cDNA sequence for clone RO200 A10 (SEQ ID NO: 157), also referred to as O597S

SEQ ID NO:216 is an extended cDNA sequence for clone R0200 A12 (SEQ ID NO: 158), also referred to as O598S

SEQ ID NO:217 is a full length cDNA sequence for monocarboxylate transporter (MCT3), a sequence having homology with O598S

SEQ ID NO:218 is an extended cDNA sequence for clone RO200 E10 (57881.2; SEQ ID NO: 206), also referred to as O599S

SEQ ID NO:219 is an extended cDNA sequence for clone RO200 G2 (SEQ ID NO: 193), also referred to as O600S

SEQ ID NO:220 is an extended cDNA sequence for clone RO200 B4 (57882.1; SEQ ID NO: 207), also referred to as O601S

SEQ ID NO:221 is a full length cDNA sequence for lysophospholipase I (LYPLA1), a sequence having homology with O601S

SEQ ID NO:222 is an extended cDNA sequence for clone RO201 D1 (57884.2; SEQ ID NO: 208), also referred to as O602S

### DETAILED DESCRIPTION OF THE INVENTION

As noted above, the present invention is generally directed to compositions and methods for using the compositions, for example in the therapy and diagnosis of cancer, such as ovarian and endometrial cancer. Certain illustrative compositions described herein include ovarian tumor polypeptides, polynucleotides encoding such polypeptides, binding agents such as antibodies, antigen presenting cells (APCs) and/or immune system cells (e.g., T cells). An “ovarian tumor protein,” as the term is used herein, refers generally to a protein that is expressed in ovarian tumor cells at a level that is at least two fold, and preferably at least five fold, greater than the level of expression in a normal tissue, as determined using a representative assay provided herein. Certain ovarian tumor proteins are tumor proteins that react detectably (within an immunoassay, such as an ELISA or Western blot) with antisera of a patient afflicted with ovarian cancer.

Therefore, in accordance with the above, and as described further below, the present invention provides illustrative polynucleotide compositions having sequences set forth in SEQ ID NO:1-222, antibody compositions capable of binding polypeptides encoded by the polynucleotides, and numerous additional embodiments employing such compositions, for example in the detection, diagnosis and/or therapy of human ovarian cancer.

Polynucleotide Compositions

As used herein, the terms “DNA segment” and “polynucleotide” refer to a DNA molecule that has been isolated free of total genomic DNA of a particular species. Therefore, a DNA segment encoding a polypeptide refers to a DNA segment that contains one or more coding sequences yet is substantially isolated away from, or purified free from, total genomic DNA of the species from which the DNA segment is obtained. Included within the terms “DNA segment” and “polynucleotide” are DNA segments and smaller fragments of such segments, and also recombinant vectors, including, for example, plasmids, cosmids, phagemids, phage, viruses, and the like.

As will be understood by those skilled in the art, the DNA segments of this invention can include genomic sequences, extra-genomic and plasmid-encoded sequences and smaller engineered gene segments that express, or may be adapted to express, proteins, polypeptides, peptides and the like. Such segments may be naturally isolated, or modified synthetically by the hand of man.

“Isolated,” as used herein, means that a polynucleotide is substantially away from other coding sequences, and that the DNA segment does not contain large portions of unrelated coding DNA, such as large chromosomal fragments or other functional genes or polypeptide coding regions. Of course, this refers to the DNA segment as originally isolated, and does not exclude genes or coding regions later added to the segment by the hand of man.

As will be recognized by the skilled artisan, polynucleotides may be single-stranded (coding or antisense) or double-stranded, and may be DNA (genomic, cDNA or synthetic) or RNA molecules. RNA molecules include HnRNA molecules, which contain introns and correspond to a DNA molecule in a one-to-one manner, and mRNA molecules, which do not contain introns. Additional coding or non-coding sequences may, but need not, be present within a polynucleotide of the present invention, and a polynucleotide may, but need not, be linked to other molecules and/or support materials.

Polynucleotides may comprise a native sequence (i.e., an endogenous sequence that encodes an ovarian tumor protein or a portion thereof) or may comprise a variant, or a biological or antigenic functional equivalent of such a sequence. Polynucleotide variants may contain one or more substitutions, additions, deletions and/or insertions, as further described below, preferably such that the immunogenicity of the encoded polypeptide is not diminished, relative to a native tumor protein. The effect on the immunogenicity of the encoded polypeptide may generally be assessed as described herein. The term “variants” also encompasses homologous genes of xenogenic origin.

When comparing polynucleotide or polypeptide sequences, two sequences are said to be “identical” if the sequence of nucleotides or amino acids in the two sequences is the same when aligned for maximum correspondence, as described below. Comparisons between two sequences are typically performed by comparing the sequences over a comparison window to identify and compare local regions of sequence similarity. A “comparison window” as used herein, refers to a segment of at least about 20 contiguous positions, usually 30 to about 75, 40 to about 50, in which a sequence may be compared to a reference sequence of the same number of contiguous positions after the two sequences are optimally aligned.

Optimal alignment of sequences for comparison may be conducted using the Megalign program in the Lasergene suite of bioinformatics software (DNASTAR, Inc., Madison, Wis.), using default parameters. This program embodies several alignment schemes described in the following references: Dayhoff, M. O. (1978) A model of evolutionary change in proteins—Matrices for detecting distant relationships. In Dayhoff, M. O. (ed.) Atlas of Protein Sequence and Structure, National Biomedical Research Foundation, Washington D.C. Vol. 5, Suppl. 3, pp. 345-358; Hein J. (1990) Unified Approach to Alignment and Phylogenes pp. 626-645 Methods in Enzymology vol. 183, Academic Press, Inc., San Diego, Calif.; Higgins, D. G. and Sharp, P. M. (1989) CABIOS 5:151-153; Myers, E. W. and Muller W. (1988) CABIOS 4:11-17; Robinson, E. D. (1971) Comb. Theor 11:105; Santou, N. Nes, M. (1987) Mol. Biol. Evol. 4:406-425; Sneath, P. H. A. and Sokal, R. R. (1973) Numerical Taxonomy—the Principles and Practice of Numerical Taxonomy, Freeman Press, San Francisco, Calif.; Wilbur, W. J. and Lipman, D. J. (1983) Proc. Natl. Acad., Sci. USA 80:726-730.

Alternatively, optimal alignment of sequences for comparison may be conducted by the local identity algorithm of Smith and Waterman (1981) Add. APL. Math 2:482, by the identity alignment algorithm of Needleman and Wunsch (1970) J. Mol. Biol. 48:443, by the search for similarity methods of Pearson and Lipman (1988) Proc. Natl. Acad. Sci. USA 85: 2444, by computerized implementations of these algorithms (GAP, BESTFIT, BLAST, FASTA, and TFASTA in the Wisconsin Genetics Software Package, Genetics Computer Group (GCG), 575 Science Dr., Madison, Wis.), or by inspection.

One preferred example of algorithms that are suitable for determining percent sequence identity and sequence similarity are the BLAST and BLAST 2.0 algorithms, which are described in Altschul et al (1977) Nucl. Acids Res. 25:3389-3402 and Altschul et al. (1990) J. Mol. Biol. 215:403-410, respectively. BLAST and BLAST 2.0 can be used, for example with the parameters described herein, to determine percent sequence identity for the polynucleotides and polypeptides of the invention. Software for performing BLAST analyses is publicly available through the National Center for Biotechnology Information. In one illustrative example, cumulative scores can be calculated using, for nucleotide sequences, the parameters M (reward score for a pair of matching residues; always >0) and N (penalty score for mismatching residues; always <0). For amino acid sequences, a scoring matrix can be used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLASTN program (for nucleotide sequences) uses as defaults a wordlength (W) of 11, and expectation (E) of 10, and the BLOSUM62 scoring matrix (see Henikoff and Henikoff (1989) Proc. Natl. Acad. Sci. USA 89:10915) alignments, (B) of 50, expectation (E) of 10, M−5, N−-4 and a comparison of both strands.

Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity.

Therefore, the present invention encompasses polynucleotide and polypeptide sequences having substantial identity to the sequences disclosed herein, for example those comprising at least 50% sequence identity, preferably at least 55%, 60%, 65%, 70%, 75%, 80%, 85%, 90%, 95%, 96%, 97%, 98%, or 99% or higher, sequence identity compared to a polynucleotide or polypeptide sequence of this invention using the methods described herein, (e.g., BLAST analysis using standard parameters, as described below). One skilled in this art will recognize that these values can be appropriately adjusted to determine corresponding identity of proteins encoded by two nucleotide sequences by taking into account codon degeneracy, amino acid similarity, reading frame positioning and the like.

In additional embodiments, the present invention provides isolated polynucleotides and polypeptides comprising various lengths of contiguous stretches of sequence identical to or complementary to one or more of the sequences disclosed herein. For example, polynucleotides are provided by this invention that comprise at least about 15, 20, 30, 40, 50, 75, 100, 150, 200, 300, 400, 500 or 1000 or more contiguous nucleotides of one or more of the sequences disclosed herein as well as all intermediate lengths there between. It will be readily understood that “intermediate lengths”, in this context, means any length between the quoted values, such as 16, 17, 18, 19, etc.; 21, 22, 23, etc.; 30, 31, 32, etc.; 50, 51, 52, 53, etc.; 100, 101, 102, 103, etc.; 150, 151, 152, 153, etc.; including all integers through 200-500; 500-1,000, and the like.

The polynucleotides of the present invention, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative DNA segments with total lengths of about 10,000, about 5000, about 3000, about 2,000, about 1,000, about 500, about 200, about 100, about 50 base pairs in length, and the like, (including all intermediate lengths) are contemplated to be useful in many implementations of this invention.

In other embodiments, the present invention is directed to polynucleotides that are capable of hybridizing under moderately stringent conditions to a polynucleotide sequence provided herein, or a fragment thereof, or a complementary sequence thereof. Hybridization techniques are well known in the art of molecular biology. For purposes of illustration, suitable moderately stringent conditions for testing the hybridization of a polynucleotide of this invention with other polynucleotides include prewashing in a solution of 5×SSC, 0.5% SDS, 1.0 mM EDTA (pH 8.0); hybridizing at 50° C.-65° C., 5×SSC, overnight; followed by washing twice at 65° C. for 20 minutes with each of 2×, 0.5× and 0.2×SSC containing 0.1% SDS.

Moreover, it will be appreciated by those of ordinary skill in the art that, as a result of the degeneracy of the genetic code, there are many nucleotide sequences that encode a polypeptide as described herein. Some of these polynucleotides bear minimal homology to the nucleotide sequence of any native gene. Nonetheless, polynucleotides that vary due to differences in codon usage are specifically contemplated by the present invention. Further, alleles of the genes comprising the polynucleotide sequences provided herein are within the scope of the present invention. Alleles are endogenous genes that are altered as a result of one or more mutations, such as deletions, additions and/or substitutions of nucleotides. The resulting mRNA and protein may, but need not, have an altered structure or function. Alleles may be identified using standard techniques (such as hybridization, amplification and/or database sequence comparison).

Probes and Primers

In other embodiments of the present invention, the polynucleotide sequences provided herein can be advantageously used as probes or primers for nucleic acid hybridization. As such, it is contemplated that nucleic acid segments that comprise a sequence region of at least about 15 nucleotide long contiguous sequence that has the same sequence as, or is complementary to, a 15 nucleotide long contiguous sequence disclosed herein will find particular utility. Longer contiguous identical or complementary sequences, e.g., those of about 20, 30, 40, 50, 100, 200, 500, 1000 (including all intermediate lengths) and even up to full length sequences will also be of use in certain embodiments.

The ability of such nucleic acid probes to specifically hybridize to a sequence of interest will enable them to be of use in detecting the presence of complementary sequences in a given sample. However, other uses are also envisioned, such as the use of the sequence information for the preparation of mutant species primers, or primers for use in preparing other genetic constructions.

Polynucleotide molecules having sequence regions consisting of contiguous nucleotide stretches of 10-14, 15-20, 30, 50, or even of 100-200 nucleotides or so (including intermediate lengths as well), identical or complementary to a polynucleotide sequence disclosed herein, are particularly contemplated as hybridization probes for use in, e.g., Southern and Northern blotting. This would allow a gene product, or fragment thereof, to be analyzed, both in diverse cell types and also in various bacterial cells. The total size of fragment, as well as the size of the complementary stretch(es), will ultimately depend on the intended use or application of the particular nucleic acid segment. Smaller fragments will generally find use in hybridization embodiments, wherein the length of the contiguous complementary region may be varied, such as between about 15 and about 100 nucleotides, but larger contiguous complementarity stretches may be used, according to the length complementary sequences one wishes to detect.

The use of a hybridization probe of about 15-25 nucleotides in length allows the formation of a duplex molecule that is both stable and selective. Molecules having contiguous complementary sequences over stretches greater than 15 bases in length are generally preferred, though, in order to increase stability and selectivity of the hybrid, and thereby improve the quality and degree of specific hybrid molecules obtained. One will generally prefer to design nucleic acid molecules having gene-complementary stretches of 15 to 25 contiguous nucleotides, or even longer where desired.

Hybridization probes may be selected from any portion of any of the sequences disclosed herein. All that is required is to review the sequence set forth in SEQ ID NO:1-222, or to any continuous portion of the sequence, from about 15-25 nucleotides in length up to and including the full length sequence, that one wishes to utilize as a probe or primer. The choice of probe and primer sequences may be governed by various factors. For example, one may wish to employ primers from towards the termini of the total sequence.

Small polynucleotide segments or fragments may be readily prepared by, for example, directly synthesizing the fragment by chemical means, as is commonly practiced using an automated oligonucleotide synthesizer. Also, fragments may be obtained by application of nucleic acid reproduction technology, such as the PCR™ technology of U.S. Pat. No. 4,683,202 (incorporated herein by reference), by introducing selected sequences into recombinant vectors for recombinant production, and by other recombinant DNA techniques generally known to those of skill in the art of molecular biology.

The nucleotide sequences of the invention may be used for their ability to selectively form duplex molecules with complementary stretches of the entire gene or gene fragments of interest. Depending on the application envisioned, one will typically desire to employ varying conditions of hybridization to achieve varying degrees of selectivity of probe towards target sequence. For applications requiring high selectivity, one will typically desire to employ relatively stringent conditions to form the hybrids, e.g., one will select relatively low salt and/or high temperature conditions, such as provided by a salt concentration of from about 0.02 M to about 0.15 M salt at temperatures of from about 50° C. to about 70° C. Such selective conditions tolerate little, if any, mismatch between the probe and the template or target strand, and would be particularly suitable for isolating related sequences.

Of course, for some applications, for example, where one desires to prepare mutants employing a mutant primer strand hybridized to an underlying template, less stringent (reduced stringency) hybridization conditions will typically be needed in order to allow formation of the heteroduplex. In these circumstances, one may desire to employ salt conditions such as those of from about 0.15 M to about 0.9 M salt, at temperatures ranging from about 20° C. to about 55° C. Cross-hybridizing species can thereby be readily identified as positively hybridizing signals with respect to control hybridizations. In any case, it is generally appreciated that conditions can be rendered more stringent by the addition of increasing amounts of formamide, which serves to destabilize the hybrid duplex in the same manner as increased temperature. Thus, hybridization conditions can be readily manipulated, and thus will generally be a method of choice depending on the desired results.

Polynucleotide Identification and Characterization

Polynucleotides may be identified, prepared and/or manipulated using any of a variety of well established techniques. For example, a polynucleotide may be identified, as described in more detail below, by screening a microarray of cDNAs for tumor-associated expression (i.e., expression that is at least two fold greater in a tumor than in normal tissue, as determined using a representative assay provided herein). Such screens may be performed, for example, using a Synteni microarray (Palo Alto, Calif.) according to the manufacturer's instructions (and essentially as described by Schena et al, Proc. Natl. Acad. Sci. USA 93:10614-10619, 1996 and Heller et al., Proc. Natl. Acad. Sci. USA 94:2150-2155, 1997). Alternatively, polynucleotides may be amplified from cDNA prepared from cells expressing the proteins described herein, such as ovarian tumor cells. Such polynucleotides may be amplified via polymerase chain reaction (PCR). For this approach, sequence-specific primers may be designed based on the sequences provided herein, and may be purchased or synthesized.

An amplified portion of a polynucleotide of the present invention may be used to isolate a full length gene from a suitable library (e.g., an ovarian tumor cDNA library) using well known techniques. Within such techniques, a library (cDNA or genomic) is screened using one or more polynucleotide probes or primers suitable for amplification. Preferably, a library is size-selected to include larger molecules. Random primed libraries may also be preferred for identifying 5′ and upstream regions of genes. Genomic libraries are preferred for obtaining introns and extending 5′ sequences.

For hybridization techniques, a partial sequence may be labeled (e.g., by nick-translation or end-labeling with ³²P) using well known techniques. A bacterial or bacteriophage library is then generally screened by hybridizing filters containing denatured bacterial colonies (or lawns containing phage plaques) with the labeled probe (see Sambrook et al., Molecular Cloning: A Laboratory Manual, Cold Spring Harbor Laboratories, Cold Spring Harbor, N.Y., 1989). Hybridizing colonies or plaques are selected and expanded, and the DNA is isolated for further analysis. cDNA clones may be analyzed to determine the amount of additional sequence by, for example, PCR using a primer from the partial sequence and a primer from the vector. Restriction maps and partial sequences may be generated to identify one or more overlapping clones. The complete sequence may then be determined using standard techniques, which may involve generating a series of deletion clones. The resulting overlapping sequences can then assembled into a single contiguous sequence. A full length cDNA molecule can be generated by ligating suitable fragments, using well known techniques.

Alternatively, there are numerous amplification techniques for obtaining a full length coding sequence from a partial cDNA sequence. Within such techniques, amplification is generally performed via PCR. Any of a variety of commercially available kits may be used to perform the amplification step. Primers may be designed using, for example, software well known in the art. Primers are preferably 22-30 nucleotides in length, have a GC content of at least 50% and anneal to the target sequence at temperatures of about 68° C. to 72° C. The amplified region may be sequenced as described above, and overlapping sequences assembled into a contiguous sequence.

One such amplification technique is inverse PCR (see Triglia et al., Nucl. Acids Res. 16:8186, 1988), which uses restriction enzymes to generate a fragment in the known region of the gene. The fragment is then circularized by intramolecular ligation and used as a template for PCR with divergent primers derived from the known region. Within an alternative approach, sequences adjacent to a partial sequence may be retrieved by amplification with a primer to a linker sequence and a primer specific to a known region. The amplified sequences are typically subjected to a second round of amplification with the same linker primer and a second primer specific to the known region. A variation on this procedure, which employs two primers that initiate extension in opposite directions from the known sequence, is described in WO 96/38591. Another such technique is known as “rapid amplification of cDNA ends” or RACE. This technique involves the use of an internal primer and an external primer, which hybridizes to a polyA region or vector sequence, to identify sequences that are 5′ and 3′ of a known sequence. Additional techniques include capture PCR (Lagerstrom et al, PCR Methods Applic. 1:111 -19, 1991) and walking PCR (Parker et al., Nucl. Acids. Res. 19:3055-60, 1991). Other methods employing amplification may also be employed to obtain a full length cDNA sequence.

In certain instances, it is possible to obtain a full length cDNA sequence by analysis of sequences provided in an expressed sequence tag (EST) database, such as that available from GenBank. Searches for overlapping ESTs may generally be performed using well known programs (e.g., NCBI BLAST searches), and such ESTs may be used to generate a contiguous full length sequence. Full length DNA sequences may also be obtained by analysis of genomic fragments.

Polynucleotide Expression in Host Cells

In other embodiments of the invention, polynucleotide sequences or fragments thereof which encode polypeptides of the invention, or fusion proteins or functional equivalents thereof, may be used in recombinant DNA molecules to direct expression of a polypeptide in appropriate host cells. Due to the inherent degeneracy of the genetic code, other DNA sequences that encode substantially the same or a functionally equivalent amino acid sequence may be produced and these sequences may be used to clone and express a given polypeptide.

As will be understood by those of skill in the art, it may be advantageous in some instances to produce polypeptide-encoding nucleotide sequences possessing non-naturally occurring codons. For example, codons preferred by a particular prokaryotic or eukaryotic host can be selected to increase the rate of protein expression or to produce a recombinant RNA transcript having desirable properties, such as a half-life which is longer than that of a transcript generated from the naturally occurring sequence.

Moreover, the polynucleotide sequences of the present invention can be engineered using methods generally known in the art in order to alter polypeptide encoding sequences for a variety of reasons, including but not limited to, alterations which modify the cloning, processing, and/or expression of the gene product. For example, DNA shuffling by random fragmentation and PCR reassembly of gene fragments and synthetic oligonucleotides may be used to engineer the nucleotide sequences. In addition, site-directed mutagenesis may be used to insert new restriction sites, alter glycosylation patterns, change codon preference, produce splice variants, or introduce mutations, and so forth.

In another embodiment of the invention, natural, modified, or recombinant nucleic acid sequences may be ligated to a heterologous sequence to encode a fusion protein. For example, to screen peptide libraries for inhibitors of polypeptide activity, it may be useful to encode a chimeric protein that can be recognized by a commercially available antibody. A fusion protein may also be engineered to contain a cleavage site located between the polypeptide-encoding sequence and the heterologous protein sequence, so that the polypeptide may be cleaved and purified away from the heterologous moiety.

Sequences encoding a desired polypeptide may be synthesized, in whole or in part, using chemical methods well known in the art (see Caruthers, M. H. et al (1980) Nucl. Acids Res. Symp. Ser. 215-223, Horn, T. et al. (1980) Nucl. Acids Res. Symp. Ser. 225-232). Alternatively, the protein itself may be produced using chemical methods to synthesize the amino acid sequence of a polypeptide, or a portion thereof. For example, peptide synthesis can be performed using various solid-phase techniques (Roberge, J. Y. et al. (1995) Science 269:202-204) and automated synthesis may be achieved, for example, using the ABI 431A Peptide Synthesizer (Perkin Elmer, Palo Alto, Calif.).

A newly synthesized peptide may be substantially purified by preparative high performance liquid chromatography (e.g., Creighton, T. (1983) Proteins, Structures and Molecular Principles, WH Freeman and Co., New York, N.Y.) or other comparable techniques available in the art. The composition of the synthetic peptides may be confirmed by amino acid analysis or sequencing (e.g., the Edman degradation procedure). Additionally, the amino acid sequence of a polypeptide, or any part thereof, may be altered during direct synthesis and/or combined using chemical methods with sequences from other proteins, or any part thereof, to produce a variant polypeptide.

In order to express a desired polypeptide, the nucleotide sequences encoding the polypeptide, or functional equivalents, may be inserted into appropriate expression vector, i.e., a vector which contains the necessary elements for the transcription and translation of the inserted coding sequence. Methods which are well known to those skilled in the art may be used to construct expression vectors containing sequences encoding a polypeptide of interest and appropriate transcriptional and translational control elements. These methods include in vitro recombinant DNA techniques, synthetic techniques, and in vivo genetic recombination. Such techniques are described in Sambrook, J. et al. (1989) Molecular Cloning, A Laboratory Manual, Cold Spring Harbor Press, Plainview, N.Y., and Ausubel, F. M. et al. (1989) Current Protocols in Molecular Biology, John Wiley & Sons, New York. N.Y.

A variety of expression vector/host systems may be utilized to contain and express polynucleotide sequences. These include, but are not limited to, microorganisms such as bacteria transformed with recombinant bacteriophage, plasmid, or cosmid DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV) or with bacterial expression vectors (e.g., Ti or pBR322 plasmids); or animal cell systems.

The “control elements” or “regulatory sequences” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the PBLUESCRIPT phagemid (Stratagene, La Jolla, Calif.) or PSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

In bacterial systems, a number of expression vectors may be selected depending upon the use intended for the expressed polypeptide. For example, when large quantities are needed, for example for the induction of antibodies, vectors which direct high level expression of fusion proteins that are readily purified may be used. Such vectors include, but are not limited to, the multifunctional E. coli cloning and expression vectors such as BLUESCRIPT (Stratagene), in which the sequence encoding the polypeptide of interest may be ligated into the vector in frame with sequences for the amino-terminal Met and the subsequent 7 residues of .beta.-galactosidase so that a hybrid protein is produced; pIN vectors (Van Heeke, G. and S. M. Schuster (1989) J. Biol. Chem. 264:5503-5509); and the like. pGEX Vectors (Promega, Madison, Wis.) may also be used to express foreign polypeptides as fusion proteins with glutathione S-transferase (GST). In general, such fusion proteins are soluble and can easily be purified from lysed cells by adsorption to glutathione-agarose beads followed by elution in the presence of free glutathione. Proteins made in such systems may be designed to include heparin, thrombin, or factor XA protease cleavage sites so that the cloned polypeptide of interest can be released from the GST moiety at will.

In the yeast, Saccharomyces cerevisiae, a number of vectors containing constitutive or inducible promoters such as alpha factor, alcohol oxidase, and PGH may be used. For reviews, see Ausubel et al. (supra) and Grant et al. (1987) Methods Enzymol. 153:516-544.

In cases where plant expression vectors are used, the expression of sequences encoding polypeptides may be driven by any of a number of promoters. For example, viral promoters such as the 35S and 19S promoters of CaMV may be used alone or in combination with the omega leader sequence from TMV (Takamatsu, N. (1987) EMBO J. 6:307-311. Alternatively, plant promoters such as the small subunit of RUBISCO or heat shock promoters may be used (Coruzzi, G. et al. (1984) EMBO J. 3:1671-1680; Broglie, R. et al. (1984) Science 224:838-843; and Winter, J. et al. (1991) Results Probl. Cell Differ. 17:85-105). These constructs can be introduced into plant cells by direct DNA transformation or pathogen-mediated transfection. Such techniques are described in a number of generally available reviews (see, for example, Hobbs, S. or Murry, L. E. in McGraw Hill Yearbook of Science and Technology (1992) McGraw Hill, New York, N.Y.; pp. 191-196).

An insect system may also be used to express a polypeptide of interest. For example, in one such system, Autographa californica nuclear polyhedrosis virus (AcNPV) is used as a vector to express foreign genes in Spodoptera frugiperda cells or in Trichoplusia larvae. The sequences encoding the polypeptide may be cloned into a non-essential region of the virus, such as the polyhedrin gene, and placed under control of the polyhedrin promoter. Successful insertion of the polypeptide-encoding sequence will render the polyhedrin gene inactive and produce recombinant virus lacking coat protein. The recombinant viruses may then be used to infect, for example, S. frugiperda cells or Trichoplusia larvae in which the polypeptide of interest may be expressed (Engelhard, E. K. et al. (1994) Proc. Natl. Acad. Sci. 91 :3224-3227).

In mammalian host cells, a number of viral-based expression systems are generally available. For example, in cases where an adenovirus is used as an expression vector, sequences encoding a polypeptide of interest may be ligated into an adenovirus transcription/translation complex consisting of the late promoter and tripartite leader sequence. Insertion in a non-essential E1 or E3 region of the viral genome may be used to obtain a viable virus which is capable of expressing the polypeptide in infected host cells (Logan, J. and Shenk, T. (1984) Proc. Natl. Acad. Sci. 81:3655-3659). In addition, transcription enhancers, such as the Rous sarcoma virus (RSV) enhancer, may be used to increase expression in mammalian host cells.

Specific initiation signals may also be used to achieve more efficient translation of sequences encoding a polypeptide of interest. Such signals include the ATG initiation codon and adjacent sequences. In cases where sequences encoding the polypeptide, its initiation codon, and upstream sequences are inserted into the appropriate expression vector, no additional transcriptional or translational control signals may be needed. However, in cases where only coding sequence, or a portion thereof, is inserted, exogenous translational control signals including the ATG initiation codon should be provided. Furthermore, the initiation codon should be in the correct reading frame to ensure translation of the entire insert. Exogenous translational elements and initiation codons may be of various origins, both natural and synthetic. The efficiency of expression may be enhanced by the inclusion of enhancers which are appropriate for the particular cell system which is used, such as those described in the literature (Scharf, D. et al. (1994) Results Probl. Cell Differ. 20:125-162).

In addition, a host cell strain may be chosen for its ability to modulate the expression of the inserted sequences or to process the expressed protein in the desired fashion. Such modifications of the polypeptide include, but are not limited to, acetylation, carboxylation, glycosylation, phosphorylation, lipidation, and acylation. Post-translational processing which cleaves a “prepro” form of the protein may also be used to facilitate correct insertion, folding and/or function. Different host cells such as CHO, HeLa, MDCK, HEK293, and WI38, which have specific cellular machinery and characteristic mechanisms for such post-translational activities, may be chosen to ensure the correct modification and processing of the foreign protein.

For long-term, high-yield production of recombinant proteins, stable expression is generally preferred. For example, cell lines which stably express a polynucleotide of interest may be transformed using expression vectors which may contain viral origins of replication and/or endogenous expression elements and a selectable marker gene on the same or on a separate vector. Following the introduction of the vector, cells may be allowed to grow for 1-2 days in an enriched media before they are switched to selective media. The purpose of the selectable marker is to confer resistance to selection, and its presence allows growth and recovery of cells which successfully express the introduced sequences. Resistant clones of stably transformed cells may be proliferated using tissue culture techniques appropriate to the cell type.

Any number of selection systems may be used to recover transformed cell lines. These include, but are not limited to, the herpes simplex virus thymidine kinase (Wigler, M. et al. (1977) Cell 11:223-32) and adenine phosphoribosyltransferase (Lowy, I. et al. (1990) Cell 22:817-23) genes which can be employed in tk.sup.- or aprt.sup.-cells, respectively. Also, antimetabolite, antibiotic or herbicide resistance can be used as the basis for selection; for example, dhfr which confers resistance to methotrexate (Wigler, M. et al (1980) Proc. Natl. Acad. Sci. 77:3567-70); npt, which confers resistance to the aminoglycosides, neomycin and G-418 (Colbere-Garapin, F. et al (1981) J. Mol. Biol. 150:1-14); and als or pat, which confer resistance to chlorsulfuron and phosphinotricin acetyltransferase, respectively (Murry, supra). Additional selectable genes have been described, for example, trpB, which allows cells to utilize indole in place of tryptophan, or hisD, which allows cells to utilize histinol in place of histidine (Hartman, S. C. and R. C. Mulligan (1988) Proc. Natl. Acad. Sci. 85:8047-51). Recently, the use of visible markers has gained popularity with such markers as anthocyanins, beta-glucuronidase and its substrate GUS, and luciferase and its substrate luciferin, being widely used not only to identify transformants, but also to quantify the amount of transient or stable protein expression attributable to a specific vector system (Rhodes, C. A. et al. (1995) Methods Mol. Biol. 55:121-131).

Although the presence/absence of marker gene expression suggests that the gene of interest is also present, its presence and expression may need to be confirmed. For example, if the sequence encoding a polypeptide is inserted within a marker gene sequence, recombinant cells containing sequences can be identified by the absence of marker gene function. Alternatively, a marker gene can be placed in tandem with a polypeptide-encoding sequence under the control of a single promoter. Expression of the marker gene in response to induction or selection usually indicates expression of the tandem gene as well.

Alternatively, host cells which contain and express a desired polynucleotide sequence may be identified by a variety of procedures known to those of skill in the art. These procedures include, but are not limited to, DNA-DNA or DNA-RNA hybridizations and protein bioassay or immunoassay techniques which include membrane, solution, or chip based technologies for the detection and/or quantification of nucleic acid or protein.

A variety of protocols for detecting and measuring the expression of polynucleotide-encoded products, using either polyclonal or monoclonal antibodies specific for the product are known in the art. Examples include enzyme-linked immunosorbent assay (ELISA), radioimmunoassay (RIA), and fluorescence activated cell sorting (FACS). A two-site, monoclonal-based immunoassay utilizing monoclonal antibodies reactive to two non-interfering epitopes on a given polypeptide may be preferred for some applications, but a competitive binding assay may also be employed. These and other assays are described, among other places, in Hampton, R. et al. (1990; Serological Methods, a Laboratory Manual, APS Press, St Paul. Minn.) and Maddox, D. E. et al. (1983; J. Exp. Med. 158:1211-1216).

A wide variety of labels and conjugation techniques are known by those skilled in the art and may be used in various nucleic acid and amino acid assays. Means for producing labeled hybridization or PCR probes for detecting sequences related to polynucleotides include oligolabeling, nick translation, end-labeling or PCR amplification using a labeled nucleotide. Alternatively, the sequences, or any portions thereof may be cloned into a vector for the production of an mRNA probe. Such vectors are known in the art, are commercially available, and may be used to synthesize RNA probes in vitro by addition of an appropriate RNA polymerase such as T7, T3, or SP6 and labeled nucleotides. These procedures may be conducted using a variety of commercially available kits. Suitable reporter molecules or labels, which may be used include radionuclides, enzymes, fluorescent, chemiluminescent, or chromogenic agents as well as substrates, cofactors, inhibitors, magnetic particles, and the like.

Host cells transformed with a polynucleotide sequence of interest may be cultured under conditions suitable for the expression and recovery of the protein from cell culture. The protein produced by a recombinant cell may be secreted or contained intracellularly depending on the sequence and/or the vector used. As will be understood by those of skill in the art, expression vectors containing polynucleotides of the invention may be designed to contain signal sequences which direct secretion of the encoded polypeptide through a prokaryotic or eukaryotic cell membrane. Other recombinant constructions may be used to join sequences encoding a polypeptide of interest to nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). The inclusion of cleavable linker sequences such as those specific for Factor XA or enterokinase (Invitrogen. San Diego, Calif.) between the purification domain and the encoded polypeptide may be used to facilitate purification. One such expression vector provides for expression of a fusion protein containing a polypeptide of interest and a nucleic acid encoding 6 histidine residues preceding a thioredoxin or an enterokinase cleavage site. The histidine residues facilitate purification on IMIAC (immobilized metal ion affinity chromatography) as described in Porath, J. et al. (1992, Prot. Exp. Purif. 3:263-281) while the enterokinase cleavage site provides a means for purifyng the desired polypeptide from the fusion protein. A discussion of vectors which contain fusion proteins is provided in Kroll, D. J. et al. (1993; DNA Cell Biol. 12:441-453).

In addition to recombinant production methods, polypeptides of the invention, and fragments thereof, may be produced by direct peptide synthesis using solid-phase techniques (Merrifield J. (1963) J. Am. Chem. Soc. 85:2149-2154). Protein synthesis may be performed using manual techniques or by automation. Automated synthesis may be achieved, for example, using Applied Biosystems 431A Peptide Synthesizer (Perkin Elmer). Alternatively, various fragments may be chemically synthesized separately and combined using chemical methods to produce the full length molecule.

Site-Specific Mutagenesis

Site-specific mutagenesis is a technique useful in the preparation of individual peptides, or biologically functional equivalent polypeptides, through specific mutagenesis of the underlying polynucleotides that encode them. The technique, well-known to those of skill in the art, further provides a ready ability to prepare and test sequence variants, for example, incorporating one or more of the foregoing considerations, by introducing one or more nucleotide sequence changes into the DNA. Site-specific mutagenesis allows the production of mutants through the use of specific oligonucleotide sequences which encode the DNA sequence of the desired mutation, as well as a sufficient number of adjacent nucleotides, to provide a primer sequence of sufficient size and sequence complexity to form a stable duplex on both sides of the deletion junction being traversed. Mutations may be employed in a selected polynucleotide sequence to improve, alter, decrease, modify, or otherwise change the properties of the polynucleotide itself, and/or alter the properties, activity, composition, stability, or primary sequence of the encoded polypeptide.

In certain embodiments of the present invention, the inventors contemplate the mutagenesis of the disclosed polynucleotide sequences to alter one or more properties of the encoded polypeptide, such as the antigenicity of a polypeptide vaccine. The techniques of site-specific mutagenesis are well-known in the art, and are widely used to create variants of both polypeptides and polynucleotides. For example, site-specific mutagenesis is often used to alter a specific portion of a DNA molecule. In such embodiments, a primer comprising typically about 14 to about 25 nucleotides or so in length is employed, with about 5 to about 10 residues on both sides of the junction of the sequence being altered.

As will be appreciated by those of skill in the art, site-specific mutagenesis techniques have often employed a phage vector that exists in both a single stranded and double stranded form. Typical vectors useful in site-directed mutagenesis include vectors such as the M13 phage. These phage are readily commercially-available and their use is generally well-known to those skilled in the art. Double-stranded plasmids are also routinely employed in site directed mutagenesis that eliminates the step of transferring the gene of interest from a plasmid to a phage.

In general, site-directed mutagenesis in accordance herewith is performed by first obtaining a single-stranded vector or melting apart of two strands of a double-stranded vector that includes within its sequence a DNA sequence that encodes the desired peptide. An oligonucleotide primer bearing the desired mutated sequence is prepared, generally synthetically. This primer is then annealed with the single-stranded vector, and subjected to DNA polymerizing enzymes such as E. coli polymerase I Klenow fragment, in order to complete the synthesis of the mutation-bearing strand. Thus, a heteroduplex is formed wherein one strand encodes the original non-mutated sequence and the second strand bears the desired mutation. This heteroduplex vector is then used to transform appropriate cells, such as E. coli cells, and clones are selected which include recombinant vectors bearing the mutated sequence arrangement.

The preparation of sequence variants of the selected peptide-encoding DNA segments using site-directed mutagenesis provides a means of producing potentially useful species and is not meant to be limiting as there are other ways in which sequence variants of peptides and the DNA sequences encoding them may be obtained. For example, recombinant vectors encoding the desired peptide sequence may be treated with mutagenic agents, such as hydroxylamine, to obtain sequence variants. Specific details regarding these methods and protocols are found in the teachings of Maloy et al., 1994; Segal, 1976; Prokop and Bajpai, 1991; Kuby, 1994; and Maniatis et al., 1982, each incorporated herein by reference, for that purpose.

As used herein, the term “oligonucleotide directed mutagenesis procedure” refers to template-dependent processes and vector-mediated propagation which result in an increase in the concentration of a specific nucleic acid molecule relative to its initial concentration, or in an increase in the concentration of a detectable signal, such as amplification. As used herein, the term “oligonucleotide directed mutagenesis procedure” is intended to refer to a process that involves the template-dependent extension of a primer molecule. The term template dependent process refers to nucleic acid synthesis of an RNA or a DNA molecule wherein the sequence of the newly synthesized strand of nucleic acid is dictated by the well-known rules of complementary base pairing (see, for example, Watson, 1987). Typically, vector mediated methodologies involve the introduction of the nucleic acid fragment into a DNA or RNA vector, the clonal amplification of the vector, and the recovery of the amplified nucleic acid fragment. Examples of such methodologies are provided by U.S. Pat. No. 4,237,224, specifically incorporated herein by reference in its entirety.

Polynucleotides Amplification Techniques

A number of template dependent processes are available to amplify the target sequences of interest present in a sample. One of the best known amplification methods is the polymerase chain reaction (PCR™) which is described in detail in U.S. Pat. Nos. 4,683,195, 4,683,202 and 4,800,159, each of which is incorporated herein by reference in its entirety. Briefly, in PCR™, two primer sequences are prepared which are complementary to regions on opposite complementary strands of the target sequence. An excess of deoxynucleoside triphosphates is added to a reaction mixture along with a DNA polymerase (e.g., Taq polymerase). If the target sequence is present in a sample, the primers will bind to the target and the polymerase will cause the primers to be extended along the target sequence by adding on nucleotides. By raising and lowering the temperature of the reaction mixture, the extended primers will dissociate from the target to form reaction products, excess primers will bind to the target and to the reaction product and the process is repeated. Preferably reverse transcription and PCR™ amplification procedure may be performed in order to quantify the amount of mRNA amplified. Polymerase chain reaction methodologies are well known in the art.

Another method for amplification is the ligase chain reaction (referred to as LCR), disclosed in Eur. Pat. Appl. Publ. No. 320,308 (specifically incorporated herein by reference in its entirety). In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750, incorporated herein by reference in its entirety, describes an alternative method of amplification similar to LCR for binding probe pairs to a target sequence.

Qbeta Replicase, described in PCT Intl. Pat. Appl. Publ. No. PCT/US87/00880, incorporated herein by reference in its entirety, may also be used as still another amplification method in the present invention. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[α-thio]triphosphates in one strand of a restriction site (Walker et al., 1992, incorporated herein by reference in its entirety), may also be useful in the amplification of nucleic acids in the present invention.

Strand Displacement Amplification (SDA) is another method of carrying out isothermal amplification of nucleic acids which involves multiple rounds of strand displacement and synthesis, i.e. nick translation. A similar method, called Repair Chain Reaction (RCR) is another method of amplification which may be useful in the present invention and is involves annealing several probes throughout a region targeted for amplification, followed by a repair reaction in which only two of the four bases are present. The other two bases can be added as biotinylated derivatives for easy detection. A similar approach is used in SDA.

Sequences can also be detected using a cyclic probe reaction (CPR). In CPR, a probe having a 3′ and 5′ sequences of non-target DNA and an internal or “middle” sequence of the target protein specific RNA is hybridized to DNA which is present in a sample. Upon hybridization, the reaction is treated with RNaseH, and the products of the probe are identified as distinctive products by generating a signal that is released after digestion. The original template is annealed to another cycling probe and the reaction is repeated. Thus, CPR involves amplifying a signal generated by hybridization of a probe to a target gene specific expressed nucleic acid.

Still other amplification methods described in Great Britain Pat. Appl. No. 2 202 328, and in PCT Intl. Pat. Appl. Publ. No. PCT/US89/01025, each of which is incorporated herein by reference in its entirety, may be used in accordance with the present invention. In the former application, “modified” primers are used in a PCR-like, template and enzyme dependent synthesis. The primers may be modified by labeling with a capture moiety (e.g., biotin) and/or a detector moiety (e.g., enzyme). In the latter application, an excess of labeled probes is added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS) (Kwoh et al., 1989; PCT Intl. Pat. Appl. Publ. No. WO 88/10315, incorporated herein by reference in its entirety), including nucleic acid sequence based amplification (NASBA) and 3SR. In NASBA, the nucleic acids can be prepared for amplification by standard phenol/chloroform extraction, heat denaturation of a sample, treatment with lysis buffer and minispin columns for isolation of DNA and RNA or guanidinium chloride extraction of RNA. These amplification techniques involve annealing a primer that has sequences specific to the target sequence. Following polymerization, DNA/RNA hybrids are digested with RNase H while double stranded DNA molecules are heat-denatured again. In either case the single stranded DNA is made fully double stranded by addition of second target-specific primer, followed by polymerization. The double stranded DNA molecules are then multiply transcribed by a polymerase such as T7 or SP6. In an isothermal cyclic reaction, the RNAs are reverse transcribed into DNA, and transcribed once again with a polymerase such as T7 or SP6. The resulting products, whether truncated or complete, indicate target-specific sequences.

Eur. Pat. Appl. Publ. No. 329,822, incorporated herein by reference in its entirety, disclose a nucleic acid amplification process involving cyclically synthesizing single-stranded RNA (“ssRNA”), ssDNA, and double-stranded DNA (dsDNA), which may be used in accordance with the present invention. The ssRNA is a first template for a first primer oligonucleotide, which is elongated by reverse transcriptase (RNA-dependent DNA polymerase). The RNA is then removed from resulting DNA:RNA duplex by the action of ribonuclease II (RNase II, an RNase specific for RNA in a duplex with either DNA or RNA). The resultant ssDNA is a second template for a second primer, which also includes the sequences of an RNA polymerase promoter (exemplified by T7 RNA polymerase) 5′ to its homology to its template. This primer is then extended by DNA polymerase (exemplified by the large “Klenow” fragment of E. coli DNA polymerase I), resulting as a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

PCT Intl. Pat. Appl. Publ. No. WO 89/06700, incorporated herein by reference in its entirety, disclose a nucleic acid sequence amplification scheme based on the hybridization of a promoter/primer sequence to a target single-stranded DNA (“ssDNA”) followed by transcription of many RNA copies of the sequence. This scheme is not cyclic; i.e. new templates are not produced from the resultant RNA transcripts. Other amplification methods include “RACE” (Frohman, 1990), and “one-sided PCR” (Ohara, 1989) which are well-known to those of skill in the art.

Methods based on ligation of two (or more) oligonucleotides in the presence of nucleic acid having the sequence of the resulting “di-oligonucleotide”, thereby amplifying the di-oligonucleotide (Wu and Dean, 1996, incorporated herein by reference in its entirety), may also be used in the amplification of DNA sequences of the present invention.

Biological Functional Equivalents

Modification and changes may be made in the structure of the polynucleotides and polypeptides of the present invention and still obtain a functional molecule that encodes a polypeptide with desirable characteristics. As mentioned above, it is often desirable to introduce one or more mutations into a specific polynucleotide sequence. In certain circumstances, the resulting encoded polypeptide sequence is altered by this mutation, or in other cases, the sequence of the polypeptide is unchanged by one or more mutations in the encoding polynucleotide.

When it is desirable to alter the amino acid sequence of a polypeptide to create an equivalent, or even an improved, second-generation molecule, the amino acid changes may be achieved by changing one or more of the codons of the encoding DNA sequence, according to Table 1.

For example, certain amino acids may be substituted for other amino acids in a protein structure without appreciable loss of interactive binding capacity with structures such as, for example, antigen-binding regions of antibodies or binding sites on substrate molecules. Since it is the interactive capacity and nature of a protein that defines that protein's biological functional activity, certain amino acid sequence substitutions can be made in a protein sequence, and, of course, its underlying DNA coding sequence, and nevertheless obtain a protein with like properties. It is thus contemplated by the inventors that various changes may be made in the peptide sequences of the disclosed compositions, or corresponding DNA sequences which encode said peptides without appreciable loss of their biological utility or activity.

TABLE 1

| Amino Acids | | | Codons | | | | | |
|---|---|---|---|---|---|---|---|---|
| Alanine | Ala | A | GCA | GCC | GCG | GCU | | |
| Cysteine | Cys | C | UGC | UGU | | | | |
| Aspartic acid | Asp | D | GAC | GAU | | | | |
| Glutamic acid | Glu | E | GAA | GAG | | | | |
| Phenylalanine | Phe | F | UUC | UUU | | | | |
| Glycine | Gly | G | GGA | GGC | GGG | GGU | | |
| Histidine | His | H | CAC | CAU | | | | |
| Isoleucine | Ile | I | AUA | AUC | AUU | | | |
| Lysine | Lys | K | AAA | AAG | | | | |
| Leucine | Leu | L | UUA | UUG | CUA | CUC | CUG | CUU |
| Methionine | Met | M | AUG | | | | | |
| Asparagine | Asn | N | AAC | AAU | | | | |
| Proline | Pro | P | CCA | CCC | CCG | CCU | | |
| Glutamine | Gln | Q | CAA | CAG | | | | |
| Arginine | Arg | R | AGA | AGG | CGA | CGC | CGG | CGU |
| Serine | Ser | S | AGC | AGU | UCA | UCC | UCG | UCU |
| Threonine | Thr | T | ACA | ACC | ACG | ACU | | |
| Valine | Val | V | GUA | GUC | GUG | GUU | | |
| Tryptophan | Trp | W | UGG | | | | | |
| Tyrosine | Tyr | Y | UAC | UAU | | | | |

In making such changes, the hydropathic index of amino acids may be considered. The importance of the hydropathic amino acid index in conferring interactive biologic function on a protein is generally understood in the art (Kyte and Doolittle, 1982, incorporated herein by reference). It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of its hydrophobicity and charge characteristics (Kyte and Doolittle, 1982). These values are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is known in the art that certain amino acids may be substituted by other amino acids having a similar hydropathic index or score and still result in a protein with similar biological activity, i.e. still obtain a biological functionally equivalent protein. In making such changes, the substitution of amino acids whose hydropathic indices are within ±2 is preferred, those within ±1 are particularly preferred, and those within ±0.5 are even more particularly preferred. It is also understood in the art that the substitution of like amino acids can be made effectively on the basis of hydrophilicity. U.S. Pat. No. 4,554,101 (specifically incorporated herein by reference in its entirety), states that the greatest local average hydrophilicity of a protein, as governed by the hydrophilicity of its adjacent amino acids, correlates with a biological property of the protein.

As detailed in U.S. Pat. No. 4,554,101, the following hydrophilicity values have been assigned to amino acid residues: arginine (+3.0); lysine (+3.0); aspartate (+3.0±1); glutamate (+3.0±1); serine (+0.3); asparagine (+0.2); glutamine (+0.2); glycine (0) threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent protein. In such changes, the substitution of amino acids whose hydrophilicity values are within ±2 is preferred, those within ±1 are particularly preferred, and those within ±0.5 are even more particularly preferred.

As outlined above, amino acid substitutions are generally therefore based on the relative similarity of the amino acid side-chain substituents, for example, their hydrophobicity, hydrophilicity, charge, size, and the like. Exemplary substitutions that take various of the foregoing characteristics into consideration are well known to those of skill in the art and include: arginine and lysine; glutamate and aspartate; serine and threonine; glutamine and asparagine; and valine, leucine and isoleucine.

In addition, any polynucleotide may be further modified to increase stability in vivo. Possible modifications include, but are not limited to, the addition of flanking sequences at the 5′ and/or 3′ ends; the use of phosphorothioate or 2′O-methyl rather than phosphodiesterase linkages in the backbone; and/or the inclusion of nontraditional bases such as inosine, queosine and wybutosine, as well as acetyl- methyl-, thio- and other modified forms of adenine, cytidine, guanine, thymine and uridine.

In Vivo Polynucleotide Delivery Techniques

In additional embodiments, genetic constructs comprising one or more of the polynucleotides of the invention are introduced into cells in vivo. This may be achieved using any of a variety or well known approaches, several of which are outlined below for the purpose of illustration.

1. Adenovirus

One of the preferred methods for in vivo delivery of one or more nucleic acid sequences involves the use of an adenovirus expression vector. “Adenovirus expression vector” is meant to include those constructs containing adenovirus sequences sufficient to (a) support packaging of the construct and (b) to express a polynucleotide that has been cloned therein in a sense or antisense orientation. Of course, in the context of an antisense construct, expression does not require that the gene product be synthesized.

The expression vector comprises a genetically engineered form of an adenovirus. Knowledge of the genetic organization of adenovirus, a 36 kb, linear, double-stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and no genome rearrangement has been detected after extensive amplification. Adenovirus can infect virtually all epithelial cells regardless of their cell cycle stage. So far, adenoviral infection appears to be linked only to mild disease such as acute respiratory disease in humans.

Adenovirus is particularly suitable for use as a gene transfer vector because of its mid-sized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, (located at 16.8 m.u.) is particularly efficient during the late phase of infection, and all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure.

Generation and propagation of the current adenovirus vectors, which are replication deficient, depend on a unique helper cell line, designated 293, which was transformed from human embryonic kidney cells by Ad5 DNA fragments and constitutively expresses E1 proteins (Graham et al., 1977). Since the E3 region is dispensable from the adenovirus genome (Jones and Shenk, 1978), the current adenovirus vectors, with the help of 293 cells, carry foreign DNA in either the E1, the D3 or both regions (Graham and Prevec, 1991). In nature, adenovirus can package approximately 105% of the wild-type genome (Ghosh-Choudhury et al., 1987), providing capacity for about 2 extra kB of DNA. Combined with the approximately 5.5 kB of DNA that is replaceable in the E1 and E3 regions, the maximum capacity of the current adenovirus vector is under 7.5 kB, or about 15% of the total length of the vector. More than 80% of the adenovirus viral genome remains in the vector backbone and is the source of vector-borne cytotoxicity. Also, the replication deficiency of the E1-deleted virus is incomplete. For example, leakage of viral gene expression has been observed with the currently available vectors at high multiplicities of infection (MOI) (Mulligan, 1993).

Helper cell lines may be derived from human cells such as human embryonic kidney cells, muscle cells, hematopoietic cells or other human embryonic mesenchymal or epithelial cells. Alternatively, the helper cells may be derived from the cells of other mammalian species that are permissive for human adenovirus. Such cells include, e.g., Vero cells or other monkey embryonic mesenchymal or epithelial cells. As stated above, the currently preferred helper cell line is 293.

Recently, Racher et al. (1995) disclosed improved methods for culturing 293 cells and propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlemneyer flask and left stationary, with occasional agitation, for 1 to 4 h. The medium is then replaced with 50 ml of fresh medium and shaking initiated. For virus production, cells are allowed to grow to about 80% confluence, after which time the medium is replaced (to 25% of the final volume) and adenovirus added at an MOI of 0.05. Cultures are left stationary overnight, following which the volume is increased to 100% and shaking commenced for another 72 h.

Other than the requirement that the adenovirus vector be replication defective, or at least conditionally defective, the nature of the adenovirus vector is not believed to be crucial to the successful practice of the invention. The adenovirus may be of any of the 42 different known serotypes or subgroups A-F. Adenovirus type 5 of subgroup C is the preferred starting material in order to obtain a conditional replication-defective adenovirus vector for use in the present invention, since Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the polynucleotide encoding the gene of interest at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) or in the E4 region where a helper cell line or helper virus complements the E4 defect.

Adenovirus is easy to grow and manipulate and exhibits broad host range in vitro and in vivo. This group of viruses can be obtained in high titers, e.g., 10⁹-10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus vectors have been used in eukaryotic gene expression (Levrero et al., 1991; Gomez-Foix et al, 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotactic inoculation into the brain (Le Gal La Salle et al., 1993).

2. Retroviruses

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding one or more oligonucleotide or polynucleotide sequences of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was recently developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al, 1989). Using antibodies against major histocompatibility complex class I and class II antigens, they demonstrated the infection of a variety of human cells that bore those surface antigens with an ecotropic virus in vitro (Roux et al., 1989).

3. Adeno-Associated Viruses

AAV (Ridgeway, 1988; Hermonat and Muzycska, 1984) is a parovirus, discovered as a contamination of adenoviral stocks. It is a ubiquitous virus (antibodies are present in 85% of the US human population) that has not been linked to any disease. It is also classified as a dependovirus, because its replications is dependent on the presence of a helper virus, such as adenovirus. Five serotypes have been isolated, of which AAV-2 is the best characterized. AAV has a single-stranded linear DNA that is encapsidated into capsid proteins VP1, VP2 and VP3 to form an icosahedral virion of 20 to 24 nm in diameter (Muzyczka and McLaughlin, 1988).

The AAV DNA is approximately 4.7 kilobases long. It contains two open reading frames and is flanked by two ITRs (FIG. 2). There are two major genes in the AAV genome: rep and cap. The rep gene codes for proteins responsible for viral replications, whereas cap codes for capsid protein VP1-3. Each ITR forms a T-shaped hairpin structure. These terminal repeats are the only essential cis components of the AAV for chromosomal integration. Therefore, the AAV can be used as a vector with all viral coding sequences removed and replaced by the cassette of genes for delivery. Three viral promoters have been identified and named p5, p19, and p40, according to their map position. Transcription from p5 and p19 results in production of rep proteins, and transcription from p40 produces the capsid proteins (Hermonat and Muzyczka, 1984).

There are several factors that prompted researchers to study the possibility of using rAAV as an expression vector One is that the requirements for delivering a gene to integrate into the host chromosome are surprisingly few. It is necessary to have the 145-bp ITRs, which are only 6% of the AAV genome. This leaves room in the vector to assemble a 4.5-kb DNA insertion. While this carrying capacity may prevent the AAV from delivering large genes, it is amply suited for delivering the antisense constructs of the present invention.

AAV is also a good choice of delivery vehicles due to its safety. There is a relatively complicated rescue mechanism: not only wild type adenovirus but also AAV genes are required to mobilize rAAV. Likewise, AAV is not pathogenic and not associated with any disease. The removal of viral coding sequences minimizes immune reactions to viral gene expression, and therefore, rAAV does not evoke an inflammatory response.

4. Other Viral Vectors as Expression Contstructs

Other viral vectors may be employed as expression constructs in the present invention for the delivery of oligonucleotide or polynucleotide sequences to a host cell. Vectors derived from viruses such as vaccinia virus (Ridgeway, 1988; Coupar et al., 1988), lentiviruses, polio viruses and herpes viruses may be employed. They offer several attractive features for various mammalian cells (Friedmann, 1989; Ridgeway, 1988; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al. (1991) introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

5. Non-Viral Vectors

In order to effect expression of the oligonucleotide or polynucleotide sequences of the present invention, the expression construct must be delivered into a cell. This delivery may be accomplished in vitro, as in laboratory procedures for transforming cells lines, or in vivo or ex vivo, as in the treatment of certain disease states. As described above, one preferred mechanism for delivery is via viral infection where the expression construct is encapsulated in an infectious viral particle.

Once the expression construct has been delivered into the cell the nucleic acid encoding the desired oligonucleotide or polynucleotide sequences may be positioned and expressed at different sites. In certain embodiments, the nucleic acid encoding the construct may be stably integrated into the genome of the cell. This integration may be in the specific location and orientation via homologous recombination (gene replacement) or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In certain embodiments of the invention, the expression construct comprising one or more oligonucleotide or polynucleotide sequences may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. Dubensky et al. (1984) successfully injected polyomavirus DNA in the form of calcium phosphate precipitates into liver and spleen of adult and newborn mice demonstrating active viral replication and acute infection. Benvenisty and Reshef (1986) also demonstrated that direct intraperitoneal injection of calcium phosphate-precipitated plasmids results in expression of the transfected genes. It is envisioned that DNA encoding a gene of interest may also be transferred in a similar manner in vivo and express the gene product.

Another embodiment of the invention for transferring a naked DNA expression construct into cells may involve particle bombardment. This method depends on the ability to accelerate DNA-coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al., 1990; Zelenin et al., 1991). This may require surgical exposure of the tissue or cells, to eliminate any intervening tissue between the gun and the target organ, i.e. ex vivo treatment. Again, DNA encoding a particular gene may be delivered via this method and still be incorporated by the present invention.

Antisense Oligonucleotides

The end result of the flow of genetic information is the synthesis of protein. DNA is transcribed by polymerases into messenger RNA and translated on the ribosome to yield a folded, functional protein. Thus there are several steps along the route where protein synthesis can be inhibited. The native DNA segment coding for a polypeptide described herein, as all such mammalian DNA strands, has two strands: a sense strand and an antisense strand held together by hydrogen bonding. The messenger RNA coding for polypeptide has the same nucleotide sequence as the sense DNA strand except that the DNA thymidine is replaced by uridine. Thus, synthetic antisense nucleotide sequences will bind to a mRNA and inhibit expression of the protein encoded by that mRNA.

The targeting of antisense oligonucleotides to mRNA is thus one mechanism to shut down protein synthesis, and, consequently, represents a powerful and targeted therapeutic approach. For example, the synthesis of polygalactauronase and the muscarine type 2 acetylcholine receptor are inhibited by antisense oligonucleotides directed to their respective mRNA sequences (U.S. Pat. Nos. 5,739,119 and 5,759,829, each specifically incorporated herein by reference in its entirety). Further, examples of antisense inhibition have been demonstrated with the nuclear protein cyclin, the multiple drug resistance gene (MDG1), ICAM-1, E-selectin, STK-1, striatal GABA_{A} receptor and human EGF (Jaskulski et al., 1988; Vasanthakumar and Ahmed, 1989; Peris et al., 1998; U.S. Pat. Nos. 5,801,154; 5,789,573; 5,718,709 and 5,610,288, each specifically incorporated herein by reference in its entirety). Antisense constructs have also been described that inhibit and can be used to treat a variety of abnormal cellular proliferations, e.g. cancer (U.S. Pat. Nos. 5,747,470; 5,591,317 and 5,783,683, each specifically incorporated herein by reference in its entirety).

Therefore, in exemplary embodiments, the invention provides oligonucleotide sequences that comprise all, or a portion of, any sequence that is capable of specifically binding to polynucleotide sequence described herein, or a complement thereof. In one embodiment, the antisense oligonucleotides comprise DNA or derivatives thereof. In another embodiment, the oligonucleotides comprise RNA or derivatives thereof In a third embodiment, the oligonucleotides are modified DNAs comprising a phosphorothioated modified backbone. In a fourth embodiment, the oligonucleotide sequences comprise peptide nucleic acids or derivatives thereof. In each case, preferred compositions comprise a sequence region that is complementary, and more preferably substantially-complementary, and even more preferably, completely complementary to one or more portions of polynucleotides disclosed herein.

Selection of antisense compositions specific for a given gene sequence is based upon analysis of the chosen target sequence (i.e. in these illustrative examples the rat and human sequences) and determination of secondary structure, Tₘ, binding energy, relative stability, and antisense compositions were selected based upon their relative inability to form dimers, hairpins, or other secondary structures that would reduce or prohibit specific binding to the target mRNA in a host cell.

Highly preferred target regions of the mRNA, are those which are at or near the AUG translation initiation codon, and those sequences which were substantially complementary to 5′ regions of the mRNA. These secondary structure analyses and target site selection considerations were performed using v.4 of the OLIGO primer analysis software (Rychlik, 1997) and the BLASTN 2.0.5 algorithm software (Altschul et al., 1997).

The use of an antisense delivery method employing a short peptide vector, termed MPG (27 residues), is also contemplated. The MPG peptide contains a hydrophobic domain derived from the fusion sequence of HIV gp41 and a hydrophilic domain from the nuclear localization sequence of SV40 T-antigen (Morris et al., 1997). It has been demonstrated that several molecules of the MPG peptide coat the antisense oligonucleotides and can be delivered into cultured mammalian cells in less than 1 hour with relatively high efficiency (90%). Further, the interaction with MPG strongly increases both the stability of the oligonucleotide to nuclease and the ability to cross the plasma membrane (Morris et al., 1997).

Ribozymes

Although proteins traditionally have been used for catalysis of nucleic acids, another class of macromolecules has emerged as useful in this endeavor. Ribozymes are RNA-protein complexes that cleave nucleic acids in a site-specific fashion. Ribozymes have specific catalytic domains that possess endonuclease activity (Kim and Cech, 1987; Gerlach et al., 1987; Forster and Symons, 1987). For example, a large number of ribozymes accelerate phosphoester transfer reactions with a high degree of specificity, often cleaving only one of several phosphoesters in an oligonucleotide substrate (Cech et al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence-specific cleavage/ligation reactions involving nucleic acids (Joyce, 1989; Cech et al., 1981). For example, U.S. Pat. No. 5,354,855 (specifically incorporated herein by reference) reports that certain ribozymes can act as endonucleases with a sequence specificity greater than that of known ribonucleases and approaching that of the DNA restriction enzymes. Thus, sequence-specific ribozyme-mediated inhibition of gene expression may be particularly suited to therapeutic applications (Scanlon et al., 1991; Sarver et al., 1990). Recently, it was reported that ribozymes elicited genetic changes in some cells lines to which they were applied; the altered genes included the oncogenes H-ras, c-fos and genes of HIV. Most of this work involved the modification of a target mRNA, based on a specific mutant codon that is cleaved by a specific ribozyme.

Six basic varieties of naturally-occurring enzymatic RNAs are known presently. Each can catalyze the hydrolysis of RNA phosphodiester bonds in trans (and thus can cleave other RNA molecules) under physiological conditions. In general, enzymatic nucleic acids act by first binding to a target RNA. Such binding occurs through the target binding portion of a enzymatic nucleic acid which is held in close proximity to an enzymatic portion of the molecule that acts to cleave the target RNA. Thus, the enzymatic nucleic acid first recognizes and then binds a target RNA through complementary base-pairing, and once bound to the correct site, acts enzymatically to cut the target RNA. Strategic cleavage of such a target RNA will destroy its ability to direct synthesis of an encoded protein. After an enzymatic nucleic acid has bound and cleaved its RNA target, it is released from that RNA to search for another target and can repeatedly bind and cleave new targets.

The enzymatic nature of a ribozyme is advantageous over many technologies, such as antisense technology (where a nucleic acid molecule simply binds to a nucleic acid target to block its translation) since the concentration of ribozyme necessary to affect a therapeutic treatment is lower than that of an antisense oligonucleotide. This advantage reflects the ability of the ribozyme to act enzymatically. Thus, a single ribozyme molecule is able to cleave many molecules of target RNA. In addition, the ribozyme is a highly specific inhibitor, with the specificity of inhibition depending not only on the base pairing mechanism of binding to the target RNA, but also on the mechanism of target RNA cleavage. Single mismatches, or base-substitutions, near the site of cleavage can completely eliminate catalytic activity of a ribozyme. Similar mismatches in antisense molecules do not prevent their action (Woolf et al, 1992). Thus, the specificity of action of a ribozyme is greater than that of an antisense oligonucleotide binding the same RNA site.

The enzymatic nucleic acid molecule may be formed in a hammerhead, hairpin, a hepatitis δ virus, group I intron or RNaseP RNA (in association with an RNA guide sequence) or Neurospora VS RNA motif. Examples of hammerhead motifs are described by Rossi et al. (1992). Examples of hairpin motifs are described by Hampel et al. (Eur. Pat. Appl. Publ. No. EP 0360257), Hampel and Tritz (1989), Hampel et al (1990) and U.S. Pat. No. 5,631,359 (specifically incorporated herein by reference). An example of the hepatitis δ virus motif is described by Perrotta and Been (1992); an example of the RNaseP motif is described by Guerrier-Takada et al. (1983); Neurospora VS RNA ribozyme motif is described by Collins (Saville and Collins, 1990; Saville and Collins, 1991; Collins and Olive, 1993); and an example of the Group I intron is described in (U.S. Pat. No. 4,987,071, specifically incorporated herein by reference). All that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule. Thus the ribozyme constructs need not be limited to specific motifs mentioned herein.

In certain embodiments, it may be important to produce enzymatic cleaving agents which exhibit a high degree of specificity for the RNA of a desired target, such as one of the sequences disclosed herein. The enzymatic nucleic acid molecule is preferably targeted to a highly conserved sequence region of a target mRNA. Such enzymatic nucleic acid molecules can be delivered exogenously to specific cells as required. Alternatively, the ribozymes can be expressed from DNA or RNA vectors that are delivered to specific cells.

Small enzymatic nucleic acid motifs (e.g., of the hammerhead or the hairpin structure) may also be used for exogenous delivery. The simple structure of these molecules increases the ability of the enzymatic nucleic acid to invade targeted regions of the mRNA structure. Alternatively, catalytic RNA molecules can be expressed within cells from eukaryotic promoters (e.g., Scanlon et al., 1991; Kashani-Sabet et al., 1992; Dropulic et al., 1992; Weerasinghe et al., 1991; Ojwang et al., 1992; Chen et al., 1992; Sarver et al., 1990). Those skilled in the art realize that any ribozyme can be expressed in eukaryotic cells from the appropriate DNA vector. The activity of such ribozymes can be augmented by their release from the primary transcript by a second ribozyme (Int. Pat. Appl. Publ. No. WO 93/23569, and Int. Pat. Appl. Publ. No. WO 94/02595, both hereby incorporated by reference; Ohkawa et al., 1992; Taira et al., 1991; and Ventura et al., 1993).

Ribozymes may be added directly, or can be complexed with cationic lipids, lipid complexes, packaged within liposomes, or otherwise delivered to target cells. The RNA or RNA complexes can be locally administered to relevant tissues ex vivo, or in vivo through injection, aerosol inhalation, infusion pump or stent, with or without their incorporation in biopolymers.

Ribozymes may be designed as described in Int. Pat. Appl. Publ. No. WO 93/23569 and hit. Pat. Appl. Publ. No. WO 94/02595, each specifically incorporated herein by reference) and synthesized to be tested in vitro and in vivo, as described. Such ribozymes can also be optimized for delivery. While specific examples are provided, those in the art will recognize that equivalent RNA targets in other species can be utilized when necessary.

Hammerhead or hairpin ribozymes may be individually analyzed by computer folding (Jaeger et al., 1989) to assess whether the ribozyme sequences fold into the appropriate secondary structure. Those ribozymes with unfavorable intramolecular interactions between the binding arms and the catalytic core are eliminated from consideration. Varying binding arm lengths can be chosen to optimize activity. Generally, at least 5 or so bases on each arm are able to bind to, or otherwise interact with, the target RNA.

Ribozymes of the hammerhead or hairpin motif may be designed to anneal to various sites in the mRNA message, and can be chemically synthesized. The method of synthesis used follows the procedure for normal RNA synthesis as described in Usman et al. (1987) and in Scaringe et al. (1990) and makes use of common nucleic acid protecting and coupling groups, such as dimethoxytrityl at the 5′-end, and phosphoramidites at the 3′-end. Average stepwise coupling yields are typically >98%. Hairpin ribozymes may be synthesized in two parts and annealed to reconstruct an active ribozyme (Chowrira and Burke, 1992). Ribozymes may be modified extensively to enhance stability by modification with nuclease resistant groups, for example, 2′-amino, 2′-C-allyl, 2′-flouro, 2′-o-methyl, 2′-H (for a review see e.g., Usman and Cedergren, 1992). Ribozymes may be purified by gel electrophoresis using general methods or by high pressure liquid chromatography and resuspended in water.

Ribozyme activity can be optimized by altering the length of the ribozyme binding arms, or chemically synthesizing ribozymes with modifications that prevent their degradation by serum ribonucleases (see e.g., Int. Pat. Appl. Publ. No. WO 92/07065; Perrault et al, 1990; Pieken et al., 1991; Usman and Cedergren, 1992; Int. Pat. Appl. Publ. No. WO 93/15187; Int. Pat. Appl. Publ. No. WO 91/03162; Eur. Pat. Appl. Publ. No. 92110298.4; U.S. Pat. No. 5,334,711; and Int. Pat. Appl. Publ. No. WO 94/13688, which describe various chemical modifications that can be made to the sugar moieties of enzymatic RNA molecules), modifications which enhance their efficacy in cells, and removal of stem II bases to shorten RNA synthesis times and reduce chemical requirements.

Sullivan et al. (Int. Pat. Appl. Publ. No. WO 94/02595) describes the general methods for delivery of enzymatic RNA molecules. Ribozymes may be administered to cells by a variety of methods known to those familiar to the art, including, but not restricted to, encapsulation in liposomes, by iontophoresis, or by incorporation into other vehicles, such as hydrogels, cyclodextrins, biodegradable nanocapsules, and bioadhesive microspheres. For some indications, ribozymes may be directly delivered ex vivo to cells or tissues with or without the aforementioned vehicles. Alternatively, the RNA/vehicle combination may be locally delivered by direct inhalation, by direct injection or by use of a catheter, infusion pump or stent. Other routes of delivery include, but are not limited to, intravascular, intramuscular, subcutaneous or joint injection, aerosol inhalation, oral (tablet or pill form), topical, systemic, ocular, intraperitoneal and/or intrathecal delivery. More detailed descriptions of ribozyme delivery and administration are provided in Int. Pat. Appl. Publ. No. WO 94/02595 and Int. Pat. Appl. Publ. No. WO 93/23569, each specifically incorporated herein by reference.

Another means of accumulating high concentrations of a ribozyme(s) within cells is to incorporate the ribozyme-encoding sequences into a DNA expression vector. Transcription of the ribozyme sequences are driven from a promoter for eukaryotic RNA polymerase I (pol I), RNA polymerase II (pol II), or RNA polymerase II (pol III). Transcripts from pol II or pol III promoters will be expressed at high levels in all cells; the levels of a given pol II promoter in a given cell type will depend on the nature of the gene regulatory sequences (enhancers, silencers, etc.) present nearby. Prokaryotic RNA polymerase promoters may also be used, providing that the prokaryotic RNA polymerase enzyme is expressed in the appropriate cells (Elroy-Stein and Moss, 1990; Gao and Huang, 1993; Lieber et al., 1993; Zhou et al., 1990). Ribozymes expressed from such promoters can function in mammalian cells (e.g. Kashani-Saber et al., 1992; Ojwang et al., 1992; Chen et al., 1992; Yu et al., 1993; L'Huillier et al., 1992; Lisziewicz et al., 1993). Such transcription units can be incorporated into a variety of vectors for introduction into mammalian cells, including but not restricted to, plasmid DNA vectors, viral DNA vectors (such as adenovirus or adeno-associated vectors), or viral RNA vectors (such as retroviral, semliki forest virus, sindbis virus vectors).

Ribozymes may be used as diagnostic tools to examine genetic drift and mutations within diseased cells. They can also be used to assess levels of the target RNA molecule. The close relationship between ribozyme activity and the structure of the target RNA allows the detection of mutations in any region of the molecule which alters the base-pairing and three-dimensional structure of the target RNA. By using multiple ribozymes, one may map nucleotide changes which are important to RNA structure and function in vitro, as well as in cells and tissues. Cleavage of target RNAs with ribozymes may be used to inhibit gene expression and define the role (essentially) of specified gene products in the progression of disease. In this manner, other genetic targets may be defined as important mediators of the disease. These studies will lead to better treatment of the disease progression by affording the possibility of combinational therapies (e.g., multiple ribozymes targeted to different genes, ribozymes coupled with known small molecule inhibitors, or intermittent treatment with combinations of ribozymes and/or other chemical or biological molecules). Other in vitro uses of ribozymes are well known in the art, and include detection of the presence of mRNA associated with an IL-5 related condition. Such RNA is detected by determining the presence of a cleavage product after treatment with a ribozyme using standard methodology.

Peptide Nucleic Acids

In certain embodiments, the inventors contemplate the use of peptide nucleic acids (PNAs) in the practice of the methods of the invention. PNA is a DNA mimic in which the nucleobases are attached to a pseudopeptide backbone (Good and Nielsen, 1997). PNA is able to be utilized in a number methods that traditionally have used RNA or DNA. Often PNA sequences perform better in techniques than the corresponding RNA or DNA sequences and have utilities that are not inherent to RNA or DNA. A review of PNA including methods of making, characteristics of, and methods of using, is provided by Corey (1997) and is incorporated herein by reference. As such, in certain embodiments, one may prepare PNA sequences that are complementary to one or more portions of the ACE mRNA sequence, and such PNA compositions may be used to regulate, alter, decrease, or reduce the translation of ACE-specific mRNA, and thereby alter the level of ACE activity in a host cell to which such PNA compositions have been administered.

PNAs have 2-aminoethyl-glycine linkages replacing the normal phosphodiester backbone of DNA (Nielsen et al., 1991; Hanvey et al, 1992; Hyrup and Nielsen, 1996; Neilsen, 1996). This chemistry has three important consequences: firstly, in contrast to DNA or phosphorothioate oligonucleotides, PNAs are neutral molecules; secondly, PNAs are achiral, which avoids the need to develop a stereoselective synthesis; and thirdly, PNA synthesis uses standard Boc (Dueholm et al., 1994) or Fmoc (Thomson et al., 1995) protocols for solid-phase peptide synthesis, although other methods, including a modified Merrifield method, have been used (Christensen et al., 1995).

PNA monomers or ready-made oligomers are commercially available from PerSeptive Biosystems (Framingham, Mass.). PNA syntheses by either Boc or Fmoc protocols are straightforward using manual or automated protocols (Norton et al., 1995). The manual protocol lends itself to the production of chemically modified PNAs or the simultaneous synthesis of families of closely related PNAs.

As with peptide synthesis, the success of a particular PNA synthesis will depend on the properties of the chosen sequence. For example, while in theory PNAs can incorporate any combination of nucleotide bases, the presence of adjacent purines can lead to deletions of one or more residues in the product. In expectation of this difficulty, it is suggested that, in producing PNAs with adjacent purines, one should repeat the coupling of residues likely to be added inefficiently. This should be followed by the purification of PNAs by reverse-phase high-pressure liquid chromatography (Norton et al., 1995) providing yields and purity of product similar to those observed during the synthesis of peptides.

Modifications of PNAs for a given application may be accomplished by coupling amino acids during solid-phase synthesis or by attaching compounds that contain a carboxylic acid group to the exposed N-terminal amine. Alternatively, PNAs can be modified after synthesis by coupling to an introduced lysine or cysteine. The ease with which PNAs can be modified facilitates optimization for better solubility or for specific functional requirements. Once synthesized, the identity of PNAs and their derivatives can be confirmed by mass spectrometry. Several studies have made and utilized modifications of PNAs (Norton et al., 1995; Haaima et al., 1996; Stetsenko et al., 1996; Petersen et al., 1995; Ulmann et al., 1996; Koch et al., 1995; Orum et al., 1995; Footer et al., 1996; Griffith et al., 1995; Kremsky et al., 1996; Pardridge et al., 1995; Boffa et al., 1995; Landsdorp et al., 1996; Gambacorti-Passerini et al., 1996; Armitage et al., 1997; Seeger et al., 1997; Ruskowski et al., 1997). U.S. Pat. No. 5,700,922 discusses PNA-DNA-PNA chimeric molecules and their uses in diagnostics, modulating protein in organisms, and treatment of conditions susceptible to therapeutics.

In contrast to DNA and RNA, which contain negatively charged linkages, the PNA backbone is neutral. In spite of this dramatic alteration, PNAs recognize complementary DNA and RNA by Watson-Crick pairing (Egholm et al., 1993), validating the initial modeling by Nielsen et al. (1991). PNAs lack 3′ to 5′ polarity and can bind in either parallel or antiparallel fashion, with the antiparallel mode being preferred (Egholm et al., 1993).

Hybridization of DNA oligonucleotides to DNA and RNA is destabilized by electrostatic repulsion between the negatively charged phosphate backbones of the complementary strands. By contrast, the absence of charge repulsion in PNA-DNA or PNA-RNA duplexes increases the melting temperature (Tₘ) and reduces the dependence of Tₘ on the concentration of mono- or divalent cations (Nielsen et al., 1991). The enhanced rate and affinity of hybridization are significant because they are responsible for the surprising ability of PNAs to perform strand invasion of complementary sequences within relaxed double-stranded DNA. In addition, the efficient hybridization at inverted repeats suggests that PNAs can recognize secondary structure effectively within double-stranded DNA. Enhanced recognition also occurs with PNAs immobilized on surfaces, and Wang et al. have shown that support-bound PNAs can be used to detect hybridization events (Wang et al., 1996).

One might expect that tight binding of PNAs to complementary sequences would also increase binding to similar (but not identical) sequences, reducing the sequence specificity of PNA recognition. As with DNA hybridization, however, selective recognition can be achieved by balancing oligomer length and incubation temperature. Moreover, selective hybridization of PNAs is encouraged by PNA-DNA hybridization being less tolerant of base mismatches than DNA-DNA hybridization. For example, a single mismatch within a 16 bp PNA-DNA duplex can reduce the Tₘ by up to 15° C. (Egholm et al, 1993). This high level of discrimination has allowed the development of several PNA-based strategies for the analysis of point mutations (Wang et al., 1996; Carlsson et al., 1996; Thiede et al., 1996; Webb and Hurskainen, 1996; Perry-O'Keefe et al., 1996).

High-affinity binding provides clear advantages for molecular recognition and the development of new applications for PNAs. For example, 11-13 nucleotide PNAs inhibit the activity of telomerase, a ribonucleo-protein that extends telomere ends using an essential RNA template, while the analogous DNA oligomers do not (Norton et al., 1996).

Neutral PNAs are more hydrophobic than analogous DNA oligomers, and this can lead to difficulty solubilizing them at neutral pH, especially if the PNAs have a high purine content or if they have the potential to form secondary structures. Their solubility can be enhanced by attaching one or more positive charges to the PNA termini (Nielsen et al., 1991).

Findings by Allfrey and colleagues suggest that strand invasion will occur spontaneously at sequences within chromosomal DNA (Boffa et al., 1995; Boffa et al., 1996). These studies targeted PNAs to triplet repeats of the nucleotides CAG and used this recognition to purify transcriptionally active DNA (Boffa et al., 1995) and to inhibit transcription (Boffa et al, 1996). This result suggests that if PNAs can be delivered within cells then they will have the potential to be general sequence-specific regulators of gene expression. Studies and reviews concerning the use of PNAs as antisense and anti-gene agents include Nielsen et al. (1993b), Hanvey et al. (1992), and Good and Nielsen (1997). Koppelhus et al. (1997) have used PNAs to inhibit HIV-1 inverse transcription, showing that PNAs may be used for antiviral therapies.

Methods of characterizing the antisense binding properties of PNAs are discussed in Rose (1993) and Jensen et al. (1997). Rose uses capillary gel electrophoresis to determine binding of PNAs to their complementary oligonucleotide, measuring the relative binding kinetics and stoichiometry. Similar types of measurements were made by Jensen et al. using BIAcore™ technology.

Other applications of PNAs include use in DNA strand invasion (Nielsen et al., 1991), antisense inhibition (Hanvey et al., 1992), mutational analysis (Orum et al., 1993), enhancers of transcription (Mollegaard et al., 1994), nucleic acid purification (Orum et al., 1995), isolation of transcriptionally active genes (Boffa et al., 1995), blocking of transcription factor binding (Vickers et al., 1995), genome cleavage (Veselkov et al., 1996), biosensors (Wang et al., 1996), in situ hybridization (Thisted et al., 1996), and in a alternative to Southern blotting (Perry-O'Keefe, 1996).

Polypeptide Compositions

The present invention, in other aspects, provides polypeptide compositions. Generally, a polypeptide of the invention will be an isolated polypeptide (or an epitope, variant, or active fragment thereof) derived from a mammalian species. Preferably, the polypeptide is encoded by a polynucleotide sequence disclosed herein or a sequence which hybridizes under moderately stringent conditions to a polynucleotide sequence disclosed herein. Alternatively, the polypeptide may be defined as a polypeptide which comprises a contiguous amino acid sequence from an amino acid sequence disclosed herein, or which polypeptide comprises an entire amino acid sequence disclosed herein.

In the present invention, a polypeptide composition is also understood to comprise one or more polypeptides that are immunologically reactive with antibodies generated against a polypeptide of the invention, particularly a polypeptide having the amino acid sequence encoded by SEQ ID NOs:1-222, or to active fragments, or to variants or biological functional equivalents thereof.

Likewise, a polypeptide composition of the present invention is understood to comprise one or more polypeptides that are capable of eliciting antibodies that are immunologically reactive with one or more polypeptides encoded by one or more contiguous nucleic acid sequences contained in SEQ ID NOs:1-222, or to active fragments, or to variants thereof, or to one or more nucleic acid sequences which hybridize to one or more of these sequences under conditions of moderate to high stringency.

As used herein, an active fragment of a polypeptide includes a whole or a portion of a polypeptide which is modified by conventional techniques, e.g., mutagenesis, or by addition, deletion, or substitution, but which active fragment exhibits substantially the same structure function, antigenicity, etc., as a polypeptide as described herein.

In certain illustrative embodiments, the polypeptides of the invention will comprise at least an immunogenic portion of an ovarian tumor protein or a variant thereof, as described herein. As noted above, an “ovarian tumor protein” is a protein that is expressed by ovarian tumor cells. Proteins that are ovarian tumor proteins also react detectably within an immunoassay (such as an ELISA) with antisera from a patient with ovarian cancer. Polypeptides as described herein may be of any length. Additional sequences derived from the native protein and/or heterologous sequences may be present, and such sequences may (but need not) possess further immunogenic or antigenic properties.

An “immunogenic portion,” as used herein is a portion of a protein that is recognized (i.e., specifically bound) by a B-cell and/or T-cell surface antigen receptor. Such immunogenic portions generally comprise at least 5 amino acid residues, more preferably at least 10, and still more preferably at least 20 amino acid residues of an ovarian tumor protein or a variant thereof. Certain preferred immunogenic portions include peptides in which an N-terminal leader sequence and/or transmembrane domain have been deleted. Other preferred immunogenic portions may contain a small N- and/or C-terminal deletion (e.g., 1-30 amino acids, preferably 5-15 amino acids), relative to the mature protein.

Immunogenic portions may generally be identified using well known techniques, such as those summarized in Paul, Fundamental Immunology, 3rd ed., 243-247 (Raven Press, 1993) and references cited therein. Such techniques include screening polypeptides for the ability to react with antigen-specific antibodies, antisera and/or T-cell lines or clones. As used herein, antisera and antibodies are “antigen-specific” if they specifically bind to an antigen (i.e., they react with the protein in an ELISA or other immunoassay, and do not react detectably with unrelated proteins). Such antisera and antibodies may be prepared as described herein, and using well known techniques. An immunogenic portion of a native ovarian tumor protein is a portion that reacts with such antisera and/or T-cells at a level that is not substantially less than the reactivity of the full length polypeptide (e.g., in an ELISA and/or T-cell reactivity assay). Such immunogenic portions may react within such assays at a level that is similar to or greater than the reactivity of the full length polypeptide. Such screens may generally be performed using methods well known to those of ordinary skill in the art, such as those described in Harlow and Lane, Antibodies: A Laboratory Manual, Cold Spring Harbor Laboratory, 1988. For example, a polypeptide may be immobilized on a solid support and contacted with patient sera to allow binding of antibodies within the sera to the immobilized polypeptide. Unbound sera may then be removed and bound antibodies detected using, for example, ¹²⁵I-labeled Protein A.

As noted above, a composition may comprise a variant of a native ovarian tumor protein. A polypeptide “variant,” as used herein, is a polypeptide that differs from a native ovarian tumor protein in one or more substitutions, deletions, additions and/or insertions, such that the immunogenicity of the polypeptide is not substantially diminished. In other words, the ability of a variant to react with antigen-specific antisera may be enhanced or unchanged, relative to the native protein, or may be diminished by less than 50%, and preferably less than 20%, relative to the native protein. Such variants may generally be identified by modifying one of the above polypeptide sequences and evaluating the reactivity of the modified polypeptide with antigen-specific antibodies or antisera as described herein. Preferred variants include those in which one or more portions, such as an N-terminal leader sequence or transmembrane domain, have been removed. Other preferred variants include variants in which a small portion (e.g., 1-30 amino acids, preferably 5-15 amino acids) has been removed from the N- and/or C-terminal of the mature protein.

Polypeptide variants encompassed by the present invention include those exhibiting at least about 70%, 75%, 80%, 85%, 90%, 91%, 92%, 93%, 94%, 95%, 96%, 97%, 98%, or 99% or more identity (determined as described above) to the polypeptides disclosed herein.

Preferably, a variant contains conservative substitutions. A “conservative substitution” is one in which an amino acid is substituted for another amino acid that has similar properties, such that one skilled in the art of peptide chemistry would expect the secondary structure and hydropathic nature of the polypeptide to be substantially unchanged. Amino acid substitutions may generally be made on the basis of similarity in polarity, charge, solubility, hydrophobicity, hydrophilicity and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine and valine; glycine and alanine; asparagine and glutamine; and serine, threonine, phenylalanine and tyrosine. Other groups of amino acids that may represent conservative changes include: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. A variant may also, or alternatively, contain nonconservative changes. In a preferred embodiment, variant polypeptides differ from a native sequence by substitution, deletion or addition of five amino acids or fewer. Variants may also (or alternatively) be modified by, for example, the deletion or addition of amino acids that have minimal influence on the immunogenicity, secondary structure and hydropathic nature of the polypeptide.

As noted above, polypeptides may comprise a signal (or leader) sequence at the N-terminal end of the protein, which co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region.

Polypeptides may be prepared using any of a variety of well known techniques. Recombinant polypeptides encoded by DNA sequences as described above may be readily prepared from the DNA sequences using any of a variety of expression vectors known to those of ordinary skill in the art. Expression may be achieved in any appropriate host cell that has been transformed or transfected with an expression vector containing a DNA molecule that encodes a recombinant polypeptide. Suitable host cells include prokaryotes, yeast, and higher eukaryotic cells, such as mammalian cells and plant cells. Preferably, the host cells employed are E. coli, yeast or a mammalian cell line such as COS or CHO. Supernatants from suitable host/vector systems which secrete recombinant protein or polypeptide into culture media may be first concentrated using a commercially available filter. Following concentration, the concentrate may be applied to a suitable purification matrix such as an affinity matrix or an ion exchange resin. Finally, one or more reverse phase HPLC steps can be employed to further purify a recombinant polypeptide.

Portions and other variants having less than about 100 amino acids, and generally less than about 50 amino acids, may also be generated by synthetic means, using techniques well known to those of ordinary skill in the art. For example, such polypeptides may be synthesized using any of the commercially available solid-phase techniques, such as the Merrifield solid-phase synthesis method, where amino acids are sequentially added to a growing amino acid chain. See Merrifield, J. Am. Chem. Soc. 85:2149-2146, 1963. Equipment for automated synthesis of polypeptides is commercially available from suppliers such as Perkin Elmer/Applied BioSystems Division (Foster City, Calif.), and may be operated according to the manufacturer's instructions.

Within certain specific embodiments, a polypeptide may be a fusion protein that comprises multiple polypeptides as described herein, or that comprises at least one polypeptide as described herein and an unrelated sequence, such as a known tumor protein. A fusion partner may, for example, assist in providing T helper epitopes (an immunological fusion partner), preferably T helper epitopes recognized by humans, or may assist in expressing the protein (an expression enhancer) at higher yields than the native recombinant protein. Certain preferred fusion partners are both immunological and expression enhancing fusion partners. Other fusion partners may be selected so as to increase the solubility of the protein or to enable the protein to be targeted to desired intracellular compartments. Still further fusion partners include affinity tags, which facilitate purification of the protein.

Fusion proteins may generally be prepared using standard techniques, including chemical conjugation. Preferably, a fusion protein is expressed as a recombinant protein, allowing the production of increased levels, relative to a non-fused protein, in an expression system. Briefly, DNA sequences encoding the polypeptide components may be assembled separately, and ligated into an appropriate expression vector. The 3′ end of the DNA sequence encoding one polypeptide component is ligated, with or without a peptide linker, to the 5′ end of a DNA sequence encoding the second polypeptide component so that the reading frames of the sequences are in phase. This permits translation into a single fusion protein that retains the biological activity of both component polypeptides.

A peptide linker sequence may be employed to separate the first and second polypeptide components by a distance sufficient to ensure that each polypeptide folds into its secondary and tertiary structures. Such a peptide linker sequence is incorporated into the fusion protein using standard techniques well known in the art. Suitable peptide linker sequences may be chosen based on the following factors: (1) their ability to adopt a flexible extended conformation; (2) their inability to adopt a secondary structure that could interact with functional epitopes on the first and second polypeptides; and (3) the lack of hydrophobic or charged residues that might react with the polypeptide functional epitopes. Preferred peptide linker sequences contain Gly, Asn and Ser residues. Other near neutral amino acids, such as Thr and Ala may also be used in the linker sequence. Amino acid sequences which may be usefully employed as linkers include those disclosed in Maratea et al., Gene 40:39-46, 1985; Murphy et al., Proc. Natl. Acad. Sci. USA 83:8258-8262, 1986; U.S. Pat. Nos. 4,935,233 and 4,751,180. The linker sequence may generally be from 1 to about 50 amino acids in length. Linker sequences are not required when the first and second polypeptides have non-essential N-terminal amino acid regions that can be used to separate the functional domains and prevent steric interference.

The ligated DNA sequences are operably linked to suitable transcriptional or translational regulatory elements. The regulatory elements responsible for expression of DNA are located only 5′ to the DNA sequence encoding the first polypeptides. Similarly, stop codons required to end translation and transcription termination signals are only present 3′ to the DNA sequence encoding the second polypeptide.

Fusion proteins are also provided. Such proteins comprise a polypeptide as described herein together with an unrelated immunogenic protein. Preferably the immunogenic protein is capable of eliciting a recall response. Examples of such proteins include tetanus, tuberculosis and hepatitis proteins (see, for example, Stoute et al. New Engl. J. Med., 336:86-91, 1997).

Within preferred embodiments, an immunological fusion partner is derived from protein D, a surface protein of the gram-negative bacterium Haemophilus influenza B (WO 91/18926). Preferably, a protein D derivative comprises approximately the first third of the protein (e.g., the first N-terminal 100-110 amino acids), and a protein D derivative may be lipidated. Within certain preferred embodiments, the first 109 residues of a Lipoprotein D fusion partner is included on the N-terminus to provide the polypeptide with additional exogenous T-cell epitopes and to increase the expression level in E. coli (thus functioning as an expression enhancer). The lipid tail ensures optimal presentation of the antigen to antigen presenting cells. Other fusion partners include the non-structural protein from influenzae virus, NS1 (hemaglutinin). Typically, the N-terminal 81 amino acids are used, although different fragments that include T-helper epitopes may be used.

In another embodiment, the immunological fusion partner is the protein known as LYTA, or a portion thereof (preferably a C-terminal portion). LYTA is derived from Streptococcus pneumoniae, which synthesizes an N-acetyl-L-alanine amidase known as amidase LYTA (encoded by the LytA gene; Gene 43:265-292, 1986). LYTA is an autolysin that specifically degrades certain bonds in the peptidoglycan backbone. The C-terminal domain of the LYTA protein is responsible for the affinity to the choline or to some choline analogues such as DEAE. This property has been exploited for the development of E. coli C-LYTA expressing plasmids useful for expression of fusion proteins. Purification of hybrid proteins containing the C-LYTA fragment at the amino terminus has been described (see Biotechnology 10:795-798, 1992). Within a preferred embodiment, a repeat portion of LYTA may be incorporated into a fusion protein. A repeat portion is found in the C-terminal region starting at residue 178. A particularly preferred repeat portion incorporates residues 188-305.

In general, polypeptides (including fusion proteins) and polynucleotides as described herein are isolated. An “isolated” polypeptide or polynucleotide is one that is removed from its original environment. For example, a naturally-occurring protein is isolated if it is separated from some or all of the coexisting materials in the natural system. Preferably, such polypeptides are at least about 90% pure, more preferably at least about 95% pure and most preferably at least about 99% pure. A polynucleotide is considered to be isolated if, for example, it is cloned into a vector that is not a part of the natural environment.

Binding Agents

The present invention further provides agents, such as antibodies and antigen-binding fragments thereof, that specifically bind to an ovarian tumor protein. As used herein, an antibody, or antigen-binding fragment thereof, is said to “specifically bind” to an ovarian tumor protein if it reacts at a detectable level (within, for example, an ELISA) with an ovarian tumor protein, and does not react detectably with unrelated proteins under similar conditions. As used herein, “binding” refers to a noncovalent association between two separate molecules such that a complex is formed. The ability to bind may be evaluated by, for example, determining a binding constant for the formation of the complex. The binding constant is the value obtained when the concentration of the complex is divided by the product of the component concentrations. In general, two compounds are said to “bind,” in the context of the present invention, when the binding constant for complex formation exceeds about 10³ L/mol. The binding constant may be determined using methods well known in the art.

Binding agents may be further capable of differentiating between patients with and without a cancer, such as ovarian cancer, using the representative assays provided herein. In other words, antibodies or other binding agents that bind to an ovarian tumor protein will generate a signal indicating the presence of a cancer in at least about 20% of patients with the disease, and will generate a negative signal indicating the absence of the disease in at least about 90% of individuals without the cancer. To determine whether a binding agent satisfies this requirement, biological samples (e.g., blood, sera, sputum, urine and/or tumor biopsies) from patients with and without a cancer (as determined using standard clinical tests) may be assayed as described herein for the presence of polypeptides that bind to the binding agent. It will be apparent that a statistically significant number of samples with and without the disease should be assayed. Each binding agent should satisfy the above criteria; however, those of ordinary skill in the art will recognize that binding agents may be used in combination to improve sensitivity.

Any agent that satisfies the above requirements may be a binding agent. For example, a binding agent may be a ribosome, with or without a peptide component, an RNA molecule or a polypeptide. In a preferred embodiment, a binding agent is an antibody or an antigen-binding fragment thereof. Antibodies may be prepared by any of a variety of techniques known to those of ordinary skill in the art. See, e.g., Harlow and Lane, Antibodies: A Laboratory Manual, Cold Spring Harbor Laboratory, 1988. In general, antibodies can be produced by cell culture techniques, including the generation of monoclonal antibodies as described herein, or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheep or goats). In this step, the polypeptides of this invention may serve as the immunogen without modification. Alternatively, particularly for relatively short polypeptides, a superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for an antigenic polypeptide of interest may be prepared, for example, using the technique of Kohler and Milstein, Eur. J. Immunol. 6:511-519, 1976, and improvements thereto. Briefly, these methods involve the preparation of immortal cell lines capable of producing antibodies having the desired specificity (i.e., reactivity with the polypeptide of interest). Such cell lines may be produced, for example, from spleen cells obtained from an animal immunized as described above. The spleen cells are then immortalized by, for example, fusion with a myeloma cell fusion partner, preferably one that is syngeneic with the immunized animal. A variety of fusion techniques may be employed. For example, the spleen cells and myeloma cells may be combined with a nonionic detergent for a few minutes and then plated at low density on a selective medium that supports the growth of hybrid cells, but not myeloma cells. A preferred selection technique uses HAT (hypoxanthine, aminopterin, thymidine) selection. After a sufficient time, usually about 1 to 2 weeks, colonies of hybrids are observed. Single colonies are selected and their culture supernatants tested for binding activity against the polypeptide. Hybridomas having high reactivity and specificity are preferred.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the ascites fluid or the blood. Contaminants may be removed from the antibodies by conventional techniques, such as chromatography, gel filtration, precipitation, and extraction. The polypeptides of this invention may be used in the purification process in, for example, an affinity chromatography step.

Within certain embodiments, the use of antigen-binding fragments of antibodies may be preferred. Such fragments include Fab fragments, which may be prepared using standard techniques. Briefly, immunoglobulins may be purified from rabbit serum by affinity chromatography on Protein A bead columns (Harlow and Lane, Antibodies: A Laboratory Manual, Cold Spring Harbor Laboratory, 1988) and digested by papain to yield Fab and Fc fragments. The Fab and Fc fragments may be separated by affinity chromatography on protein A bead columns.

Monoclonal antibodies of the present invention may be coupled to one or more therapeutic agents. Suitable agents in this regard include radionuclides, differentiation inducers, drugs, toxins, and derivatives thereof. Preferred radionuclides include ⁹⁰Y, ¹²³I, ¹²⁵I, ¹³¹I, ¹⁸⁶Re, ¹⁸⁸Re, ²¹¹At, ²¹²Bi. Preferred drugs include methotrexate, and pyrimidine and purine analogs. Preferred differentiation inducers include phorbol esters and butyric acid. Preferred toxins include ricin, abrin, diptheria toxin, cholera toxin, gelonin, Pseudomonas exotoxin, Shigella toxin, and pokeweed antiviral protein.

A therapeutic agent may be coupled (e.g., covalently bonded) to a suitable monoclonal antibody either directly or indirectly (e.g., via a linker group). A direct reaction between an agent and an antibody is possible when each possesses a substituent capable of reacting with the other. For example, a nucleophilic group, such as an amino or sulfhydryl group, on one may be capable of reacting with a carbonyl-containing group, such as an anhydride or an acid halide, or with an alkyl group containing a good leaving group (e.g., a halide) on the other.

Alternatively, it may be desirable to couple a therapeutic agent and an antibody via a linker group. A linker group can function as a spacer to distance an antibody from an agent in order to avoid interference with binding capabilities. A linker group can also serve to increase the chemical reactivity of a substituent on an agent or an antibody, and thus increase the coupling efficiency. An increase in chemical reactivity may also facilitate the use of agents, or functional groups on agents, which otherwise would not be possible.

It will be evident to those skilled in the art that a variety of bifunctional or polyfunctional reagents, both homo- and hetero-functional (such as those described in the catalog of the Pierce Chemical Co., Rockford, Ill.), may be employed as the linker group. Coupling may be effected, for example, through amino groups, carboxyl groups, sulfhydryl groups or oxidized carbohydrate residues. There are numerous references describing such methodology, e.g., U.S. Pat. No. 4,671,958, to Rodwell et al.

Where a therapeutic agent is more potent when free from the antibody portion of the immunoconjugates of the present invention, it may be desirable to use a linker group which is cleavable during or upon internalization into a cell. A number of different cleavable linker groups have been described. The mechanisms for the intracellular release of an agent from these linker groups include cleavage by reduction of a disulfide bond (e.g., U.S. Pat. No. 4,489,710, to Spitler), by irradiation of a photolabile bond (e.g., U.S. Pat. No. 4,625,014, to Senter et al.), by hydrolysis of derivatized amino acid side chains (e.g., U.S. Pat. No. 4,638,045, to Kohn et al.), by serum complement-mediated hydrolysis (e.g., U.S. Pat. No. 4,671,958, to Rodwell et al.), and acid-catalyzed hydrolysis (e.g., U.S. Pat. No. 4,569,789, to Blattler et al.).

It may be desirable to couple more than one agent to an antibody. In one embodiment, multiple molecules of an agent are coupled to one antibody molecule. In another embodiment, more than one type of agent may be coupled to one antibody. Regardless of the particular embodiment, immunoconjugates with more than one agent may be prepared in a variety of ways. For example, more than one agent may be coupled directly to an antibody molecule, or linkers that provide multiple sites for attachment can be used. Alternatively, a carrier can be used.

A carrier may bear the agents in a variety of ways, including covalent bonding either directly or via a linker group. Suitable carriers include proteins such as albumins (e.g., U.S. Pat. No. 4,507,234, to Kato et al.), peptides and polysaccharides such as aminodextran (e.g., U.S. Pat. No. 4,699,784, to Shih et al.). A carrier may also bear an agent by noncovalent bonding or by encapsulation, such as within a liposome vesicle (e.g., U.S. Pat. Nos. 4,429,008 and 4,873,088). Carriers specific for radionuclide agents include radiohalogenated small molecules and chelating compounds. For example, U.S. Pat. No. 4,735,792 discloses representative radiohalogenated small molecules and their synthesis. A radionuclide chelate may be formed from chelating compounds that include those containing nitrogen and sulfur atoms as the donor atoms for binding the metal, or metal oxide, radionuclide. For example, U.S. Pat. No. 4,673,562, to Davison et al. discloses representative chelating compounds and their synthesis.

A variety of routes of administration for the antibodies and immunoconjugates may be used. Typically, administration will be intravenous, intramuscular, subcutaneous or in the bed of a resected tumor. It will be evident that the precise dose of the antibody/immunoconjugate will vary depending upon the antibody used, the antigen density on the tumor, and the rate of clearance of the antibody.

T Cells

Immunotherapeutic compositions may also, or alternatively, comprise T cells specific for an ovarian tumor protein. Such cells may generally be prepared in vitro or ex vivo, using standard procedures. For example, T cells may be isolated from bone marrow, peripheral blood, or a fraction of bone marrow or peripheral blood of a patient, using a commercially available cell separation system, such as the Isolex™ System, available from Nexell Therapeutics, Inc. (Irvine, Calif.; see also U.S. Pat. Nos. 5,240,856; 5,215,926; WO 89/06280; WO 91/16116 and WO 92/07243). Alternatively, T cells may be derived from related or unrelated humans, non-human mammals, cell lines or cultures.

T cells may be stimulated with an ovarian tumor polypeptide, polynucleotide encoding an ovarian tumor polypeptide and/or an antigen presenting cell (APC) that expresses such a polypeptide. Such stimulation is performed under conditions and for a time sufficient to permit the generation of T cells that are specific for the polypeptide. Preferably, an ovarian tumor polypeptide or polynucleotide is present within a delivery vehicle, such as a microsphere, to facilitate the generation of specific T cells.

T cells are considered to be specific for an ovarian tumor polypeptide if the T cells specifically proliferate, secrete cytokines or kill target cells coated with the polypeptide or expressing a gene encoding the polypeptide. T cell specificity may be evaluated using any of a variety of standard techniques. For example, within a chromium release assay or proliferation assay, a stimulation index of more than two fold increase in lysis and/or proliferation, compared to negative controls, indicates T cell specificity. Such assays may be performed, for example, as described in Chen et al., Cancer Res. 54:1065-1070, 1994. Alternatively, detection of the proliferation of T cells may be accomplished by a variety of known techniques. For example, T cell proliferation can be detected by measuring an increased rate of DNA synthesis (e.g., by pulse-labeling cultures of T cells with tritiated thymidine and measuring the amount of tritiated thymidine incorporated into DNA). Contact with an ovarian tumor polypeptide (100 ng/ml -100 μg/ml, preferably 200 ng/ml -25 μg/ml) for 3 -7 days should result in at least a two fold increase in proliferation of the T cells. Contact as described above for 2-3 hours should result in activation of the T cells, as measured using standard cytokine assays in which a two fold increase in the level of cytokine release (e.g., TNF or IFN-γ) is indicative of T cell activation (see Coligan et al., Current Protocols in Immunology, vol. 1, Wiley Interscience (Greene 1998)). T cells that have been activated in response to an ovarian tumor polypeptide, polynucleotide or polypeptide-expressing APC may be CD4⁺ and/or CD8⁺. Ovarian tumor protein-specific T cells may be expanded using standard techniques. Within preferred embodiments, the T cells are derived from a patient, a related donor or an unrelated donor, and are administered to the patient following stimulation and expansion.

For therapeutic purposes, CD4⁺ or CD8⁺ T cells that proliferate in response to an ovarian tumor polypeptide, polynucleotide or APC can be expanded in number either in vitro or in vivo. Proliferation of such T cells in vitro may be accomplished in a variety of ways. For example, the T cells can be re-exposed to an ovarian tumor polypeptide, or a short peptide corresponding to an immunogenic portion of such a polypeptide, with or without the addition of T cell growth factors, such as interleukin-2, and/or stimulator cells that synthesize an ovarian tumor polypeptide. Alternatively, one or more T cells that proliferate in the presence of an ovarian tumor protein can be expanded in number by cloning. Methods for cloning cells are well known in the art, and include limiting dilution.

Pharmaceutical Compositions

In additional embodiments, the present invention concerns formulation of one or more of the polynucleotide, polypeptide, T-cell and/or antibody compositions disclosed herein in pharmaceutically-acceptable solutions for administration to a cell or an animal, either alone, or in combination with one or more other modalities of therapy.

It will also be understood that, if desired, the nucleic acid segment, RNA, DNA or PNA compositions that express a polypeptide as disclosed herein may be administered in combination with other agents as well, such as, e.g., other proteins or polypeptides or various pharmaceutically-active agents. In fact, there is virtually no limit to other components that may also be included, given that the additional agents do not cause a significant adverse effect upon contact with the target cells or host tissues. The compositions may thus be delivered along with various other agents as required in the particular instance. Such compositions may be purified from host cells or other biological sources, or alternatively may be chemically synthesized as described herein. Likewise, such compositions may further comprise substituted or derivatized RNA or DNA compositions.

Formulation of pharmaceutically-acceptable excipients and carrier solutions is well-known to those of skill in the art, as is the development of suitable dosing and treatment regimens for using the particular compositions described herein in a variety of treatment regimens, including e.g., oral, parenteral, intravenous, intranasal, and intramuscular administration and formulation.

1. Oral Delivery

In certain applications, the pharmaceutical compositions disclosed herein may be delivered via oral administration to an animal. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

The active compounds may even be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792,451, each specifically incorporated herein by reference in its entirety). The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

Typically, these formulations may contain at least about 0.1% of the active compound or more, although the percentage of the active ingredient(s) may, of course, be varied and may conveniently be between about 1 or 2% and about 60% or 70% or more of the weight or volume of the total formulation. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

For oral administration the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively the compositions may be fashioned into a tablet or solution form that may be placed under the tongue or otherwise dissolved in the mouth.

2. Injectable Delivery

In certain circumstances it will be desirable to deliver the pharmaceutical compositions disclosed herein parenterally, intravenously, intramuscularly, or even intraperitoneally as described in U.S. Pat. No. 5,543,158; 5,641,515 and 5,399,363 (each specifically incorporated herein by reference in its entirety). Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be facilitated by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art in light of the present disclosure. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The compositions disclosed herein may be formulated in a neutral or salt form. Pharmaceutically-acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

The phrase “pharmaceutically-acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

3. Nasal Delivery

In certain embodiments, the pharmaceutical compositions may be delivered by intranasal sprays, inhalation, and/or other aerosol delivery vehicles. Methods for delivering genes, nucleic acids, and peptide compositions directly to the lungs via nasal aerosol sprays has been described e.g., in U.S. Pat. No. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety).

4. Liposome-, Nanocapsule-, and Microparticle-Medicated Delivery

In certain embodiments, the inventors contemplate the use of liposomes, nanocapsules, microparticles, microspheres, lipid particles, vesicles, and the like, for the introduction of the compositions of the present invention into suitable host cells. In particular, the compositions of the present invention may be formulated for delivery either encapsulated in a lipid particle, a liposome, a vesicle, a nanosphere, or a nanoparticle or the like.

Such formulations may be preferred for the introduction of pharmaceutically-acceptable formulations of the nucleic acids or constructs disclosed herein. The formation and use of liposomes is generally known to those of skill in the art (see for example, Couvreur et al., 1977; Couvreur, 1988; Lasic, 1998; which describes the use of liposomes and nanocapsules in the targeted antibiotic therapy for intracellular bacterial infections and diseases). Recently, liposomes were developed with improved serum stability and circulation half-times (Gabizon and Papahadjopoulos, 1988; Allen and Choun, 1987; U.S. Pat. No. 5,741,516, specifically incorporated herein by reference in its entirety). Further, various methods of liposome and liposome like preparations as potential drug carriers have been reviewed (Takakura, 1998; Chandran et al., 1997; Margalit, 1995; U.S. Pat. Nos. 5,567,434; 5,552,157; 5,565,213; 5,738,868 and 5,795,587, each specifically incorporated herein by reference in its entirety).

Liposomes have been used successfully with a number of cell types that are normally resistant to transfection by other procedures including T cell suspensions, primary hepatocyte cultures and PC 12 cells (Renneisen et al., 1990; Muller et al., 1990). In addition, liposomes are free of the DNA length constraints that are typical of viral-based delivery systems. Liposomes have been used effectively to introduce genes, drugs (Heath and Martin, 1986; Heath et al., 1986; Balazsovits et al., 1989; Fresta and Puglisi, 1996), radiotherapeutic agents (Pikul et al., 1987), enzymes (Imaizumi et al., 1990a; Imaizumi et al., 1990b), viruses (Faller and Baltimore, 1984), transcription factors and allosteric effectors (Nicolau and Gersonde, 1979) into a variety of cultured cell lines and animals. In addition, several successful clinical trails examining the effectiveness of liposome-mediated drug delivery have been completed (Lopez-Berestein et al., 1985a; 1985b; Coune, 1988; Sculier et al., 1988). Furthermore, several studies suggest that the use of liposomes is not associated with autoimmune responses, toxicity or gonadal localization after systemic delivery (Mori and Fukatsu, 1992).

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Å, containing an aqueous solution in the core.

Liposomes bear resemblance to cellular membranes and are contemplated for use in connection with the present invention as carriers for the peptide compositions. They are widely suitable as both water- and lipid-soluble substances can be entrapped, i.e. in the aqueous spaces and within the bilayer itself, respectively. It is possible that the drug-bearing liposomes may even be employed for site-specific delivery of active agents by selectively modifying the liposomal formulation.

In addition to the teachings of Couvreur et al. (1977; 1988), the following information may be utilized in generating liposomal formulations. Phospholipids can form a variety of structures other than liposomes when dispersed in water, depending on the molar ratio of lipid to water. At low ratios the liposome is the preferred structure. The physical characteristics of liposomes depend on pH, ionic strength and the presence of divalent cations. Liposomes can show low permeability to ionic and polar substances, but at elevated temperatures undergo a phase transition which markedly alters their permeability. The phase transition involves a change from a closely packed, ordered structure, known as the gel state, to a loosely packed, less-ordered structure, known as the fluid state. This occurs at a characteristic phase-transition temperature and results in an increase in permeability to ions, sugars and drugs.

In addition to temperature, exposure to proteins can alter the permeability of liposomes. Certain soluble proteins, such as cytochrome c, bind, deform and penetrate the bilayer, thereby causing changes in permeability. Cholesterol inhibits this penetration of proteins, apparently by packing the phospholipids more tightly. It is contemplated that the most useful liposome formations for antibiotic and inhibitor delivery will contain cholesterol.

The ability to trap solutes varies between different types of liposomes. For example, MLVs are moderately efficient at trapping solutes, but SUVs are extremely inefficient. SUVs offer the advantage of homogeneity and reproducibility in size distribution, however, and a compromise between size and trapping efficiency is offered by large unilamellar vesicles (LUVs). These are prepared by ether evaporation and are three to four times more efficient at solute entrapment than MLVs.

In addition to liposome characteristics, an important determinant in entrapping compounds is the physicochemical properties of the compound itself. Polar compounds are trapped in the aqueous spaces and nonpolar compounds bind to the lipid bilayer of the vesicle. Polar compounds are released through permeation or when the bilayer is broken, but nonpolar compounds remain affiliated with the bilayer unless it is disrupted by temperature or exposure to lipoproteins. Both types show maximum efflux rates at the phase transition temperature.

Liposomes interact with cells via four different mechanisms: endocytosis by phagocytic cells of the reticuloendothelial system such as macrophages and neutrophils; adsorption to the cell surface, either by nonspecific weak hydrophobic or electrostatic forces, or by specific interactions with cell-surface components; fusion with the plasma cell membrane by insertion of the lipid bilayer of the liposome into the plasma membrane, with simultaneous release of liposomal contents into the cytoplasm; and by transfer of liposomal lipids to cellular or subcellular membranes, or vice versa, without any association of the liposome contents. It often is difficult to determine which mechanism is operative and more than one may operate at the same time.

The fate and disposition of intravenously injected liposomes depend on their physical properties, such as size, fluidity, and surface charge. They may persist in tissues for h or days, depending on their composition, and half lives in the blood range from min to several h. Larger liposomes, such as MLVs and LUVs, are taken up rapidly by phagocytic cells of the reticuloendothelial system, but physiology of the circulatory system restrains the exit of such large species at most sites. They can exit only in places where large openings or pores exist in the capillary endothelium, such as the sinusoids of the liver or spleen. Thus, these organs are the predominate site of uptake. On the other hand, SUVs show a broader tissue distribution but still are sequestered highly in the liver and spleen. In general, this in vivo behavior limits the potential targeting of liposomes to only those organs and tissues accessible to their large size. These include the blood, liver, spleen, bone marrow, and lymphoid organs.

Targeting is generally not a limitation in terms of the present invention. However, should specific targeting be desired, methods are available for this to be accomplished. Antibodies may be used to bind to the liposome surface and to direct the antibody and its drug contents to specific antigenic receptors located on a particular cell-type surface. Carbohydrate determinants (glycoprotein or glycolipid cell-surface components that play a role in cell-cell recognition, interaction and adhesion) may also be used as recognition sites as they have potential in directing liposomes to particular cell types. Mostly, it is contemplated that intravenous injection of liposomal preparations would be used, but other routes of administration are also conceivable.

Alternatively, the invention provides for pharmaceutically-acceptable nanocapsule formulations of the compositions of the present invention. Nanocapsules can generally entrap compounds in a stable and reproducible way (Henry-Michelland et al., 1987; Quintanar-Guerrero et al., 1998; Douglas et al., 1987). To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use in the present invention. Such particles may be are easily made, as described (Couvreur et al., 1980; 1988; zur Muhlen et al., 1998; Zambaux et al. 1998; Pinto-Alphandry et al., 1995 and U.S. Pat. No. 5,145,684, specifically incorporated herein by reference in its entirety).

Vaccines

In certain preferred embodiments of the present invention, vaccines are provided. The vaccines will generally comprise one or more pharmaceutical compositions, such as those discussed above, in combination with an immunostimulant. An immunostimulant may be any substance that enhances or potentiates an immune response (antibody and/or cell-mediated) to an exogenous antigen. Examples of immunostimulants include adjuvants, biodegradable microspheres (e.g., polylactic galactide) and liposomes (into which the compound is incorporated; see e.g., Fullerton, U.S. Pat. No. 4,235,877). Vaccine preparation is generally described in, for example, M. F. Powell and M. J. Newman, eds., “Vaccine Design (the subunit and adjuvant approach),” Plenum Press (NY, 1995). Pharmaceutical compositions and vaccines within the scope of the present invention may also contain other compounds, which may be biologically active or inactive. For example, one or more immunogenic portions of other tumor antigens may be present, either incorporated into a fusion polypeptide or as a separate compound, within the composition or vaccine.

Illustrative vaccines may contain DNA encoding one or more of the polypeptides as described above, such that the polypeptide is generated in situ. As noted above, the DNA may be present within any of a variety of delivery systems known to those of ordinary skill in the art, including nucleic acid expression systems, bacteria and viral expression systems. Numerous gene delivery techniques are well known in the art, such as those described by Rolland, Crit. Rev. Therap. Drug Carrier Systems 15:143-198, 1998, and references cited therein. Appropriate nucleic acid expression systems contain the necessary DNA sequences for expression in the patient (such as a suitable promoter and terminating signal). Bacterial delivery systems involve the administration of a bacterium (such as Bacillus-Calmette-Guerrin) that expresses an immunogenic portion of the polypeptide on its cell surface or secretes such an epitope. In a preferred embodiment, the DNA may be introduced using a viral expression system (e.g., vaccinia or other pox virus, retrovirus, or adenovirus), which may involve the use of a non-pathogenic (defective), replication competent virus. Suitable systems are disclosed, for example, in Fisher-Hoch et al., Proc. Natl. Acad. Sci. USA 86:317-321, 1989; Flexner et al., Ann. N.Y. Acad. Sci. 569:86-103, 1989; Flexner et al., Vaccine 8:17-21, 1990; U.S. Pat. Nos. 4,603,112, 4,769,330, and 5,017,487; WO 89/01973; U.S. Pat. No. 4,777,127; GB 2,200,651; EP 0,345,242; WO 91/02805; Berkner, Biotechniques 6:616-627, 1988; Rosenfeld et al., Science 252:431-434, 1991; Kolls et al., Proc. Natl. Acad. Sci. USA 91:215-219, 1994; Kass-Eisler et al., Proc. Natl. Acad. Sci. USA 90:11498-11502, 1993; Guzman et al., Circulation 88:2838-2848, 1993; and Guzman et al., Cir. Res. 73:1202-1207, 1993. Techniques for incorporating DNA into such expression systems are well known to those of ordinary skill in the art. The DNA may also be “naked,” as described, for example, in Ulmer et al., Science 259:1745-1749, 1993 and reviewed by Cohen, Science 259:1691-1692, 1993. The uptake of naked DNA may be increased by coating the DNA onto biodegradable beads, which are efficiently transported into the cells. It will be apparent that a vaccine may comprise both a polynucleotide and a polypeptide component. Such vaccines may provide for an enhanced immune response.

It will be apparent that a vaccine may contain pharmaceutically acceptable salts of the polynucleotides and polypeptides provided herein. Such salts may be prepared from pharmaceutically acceptable non-toxic bases, including organic bases (e.g., salts of primary, secondary and tertiary amines and basic amino acids) and inorganic bases (e.g., sodium, potassium, lithium, ammonium, calcium and magnesium salts).

While any suitable carrier known to those of ordinary skill in the art may be employed in the vaccine compositions of this invention, the type of carrier will vary depending on the mode of administration. Compositions of the present invention may be formulated for any appropriate manner of administration, including for example, topical, oral, nasal, intravenous, intracranial, intraperitoneal, subcutaneous or intramuscular administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactate polyglycolate) may also be employed as carriers for the pharmaceutical compositions of this invention. Suitable biodegradable microspheres are disclosed, for example, in U.S. Pat. Nos. 4,897,268; 5,075,109; 5,928,647; 5,811,128; 5,820,883; 5,853,763; 5,814,344 and 5,942,252. One may also employ a carrier comprising the particulate-protein complexes described in U.S. Pat. No. 5,928,647, which are capable of inducing a class I-restricted cytotoxic T lymphocyte responses in a host.

Such compositions may also comprise buffers (e.g., neutral buffered saline or phosphate buffered saline), carbohydrates (e.g., glucose, mannose, sucrose or dextrans), mannitol, proteins, polypeptides or amino acids such as glycine, antioxidants, bacteriostats, chelating agents such as EDTA or glutathione, adjuvants (e.g., aluminum hydroxide), solutes that render the formulation isotonic, hypotonic or weakly hypertonic with the blood of a recipient, suspending agents, thickening agents and/or preservatives. Alternatively, compositions of the present invention may be formulated as a lyophilizate. Compounds may also be encapsulated within liposomes using well known technology.

Any of a variety of immunostimulants may be employed in the vaccines of this invention. For example, an adjuvant may be included. Most adjuvants contain a substance designed to protect the antigen from rapid catabolism, such as aluminum hydroxide or mineral oil, and a stimulator of immune responses, such as lipid A, Bortadella pertussis or Mycobacterium tuberculosis derived proteins. Suitable adjuvants are commercially available as, for example, Freund's Incomplete Adjuvant and Complete Adjuvant (Difco Laboratories, Detroit, Mich.); Merck Adjuvant 65 (Merck and Company, Inc., Rahway, N.J.); AS-2 (SmithKline Beecham, Philadelphia, Pa.); aluminum salts such as aluminum hydroxide gel (alum) or aluminum phosphate; salts of calcium, iron or zinc; an insoluble suspension of acylated tyrosine; acylated sugars; cationically or anionically derivatized polysaccharides; polyphosphazenes; biodegradable microspheres; monophosphoryl lipid A and quil A. Cytokines, such as GM-CSF or interleukin-2, -7, or -12, may also be used as adjuvants.

Within the vaccines provided herein, the adjuvant composition is preferably designed to induce an immune response predominantly of the Th1 type. High levels of Th1-type cytokines (e.g., IFN-γ, TNFα, IL-2 and IL-12) tend to favor the induction of cell mediated immune responses to an administered antigen. In contrast, high levels of Th2-type cytokines (e.g., IL-4, IL-5, IL-6 and IL-10) tend to favor the induction of humoral immune responses. Following application of a vaccine as provided herein, a patient will support an immune response that includes Th1- and Th2-type responses. Within a preferred embodiment, in which a response is predominantly Th1-type, the level of Th1-type cytokines will increase to a greater extent than the level of Th2-type cytokines. The levels of these cytokines may be readily assessed using standard assays. For a review of the families of cytokines, see Mosmann and Coffman, Ann. Rev. Immunol. 7:145-173, 1989.

Preferred adjuvants for use in eliciting a predominantly Th1-type response include, for example, a combination of monophosphoryl lipid A, preferably 3-de-O-acylated monophosphoryl lipid A (3D-MPL), together with an aluminum salt. MPL adjuvants are available from Corixa Corporation (Seattle, Wash.; see U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant is a saponin, preferably QS21 (Aquila Biopharmaceuticals Inc., Framingham, Mass.), which may be used alone or in combination with other adjuvants. For example, an enhanced system involves the combination of a monophosphoryl lipid A and saponin derivative, such as the combination of QS21 and 3D-MPL as described in WO 94/00153, or a less reactogenic composition where the QS21 is quenched with cholesterol, as described in WO 96/33739. Other preferred formulations comprise an oil-in-water emulsion and tocopherol. A particularly potent adjuvant formulation involving QS21, 3D-MPL and tocopherol in an oil-in-water emulsion is described in WO 95/17210.

Other preferred adjuvants include Montanide ISA 720 (Seppic, France), SAF (Chiron, Calif., United States), ISCOMS (CSL), MF-59 (Chiron), the SBAS series of adjuvants (e.g., SBAS-2 or SBAS-4, available from SmithKline Beecham, Rixensart, Belgium), Detox (Corixa, Hamilton, Mont.), RC-529 (Corixa, Hamilton, Mont.) and other aminoalkyl glucosaminide 4-phosphates (AGPs), such as those described in pending U.S. patent application Ser. Nos. 08/853,826 and 09/074,720, the disclosures of which are incorporated herein by reference in their entireties.

Any vaccine provided herein may be prepared using well known methods that result in a combination of antigen, immune response enhancer and a suitable carrier or excipient. The compositions described herein may be administered as part of a sustained release formulation (i.e., a formulation such as a capsule, sponge or gel (composed of polysaccharides, for example) that effects a slow release of compound following administration). Such formulations may generally be prepared using well known technology (see, e.g., Coombes et al., Vaccine 14:1429-1438, 1996) and administered by, for example, oral, rectal or subcutaneous implantation, or by implantation at the desired target site. Sustained-release formulations may contain a polypeptide, polynucleotide or antibody dispersed in a carrier matrix and/or contained within a reservoir surrounded by a rate controlling membrane.

Carriers for use within such formulations are biocompatible, and may also be biodegradable; preferably the formulation provides a relatively constant level of active component release. Such carriers include microparticles of poly(lactide-co-glycolide), polyacrylate, latex, starch, cellulose, dextran and the like. Other delayed-release carriers include supramolecular biovectors, which comprise a non-liquid hydrophilic core (e.g., a cross-linked polysaccharide or oligosaccharide) and, optionally, an external layer comprising an amphiphilic compound, such as a phospholipid (see e.g., U.S. Pat. No. 5,151,254 and PCT applications WO 94/20078, WO/94/23701 and WO 96/06638). The amount of active compound contained within a sustained release formulation depends upon the site of implantation, the rate and expected duration of release and the nature of the condition to be treated or prevented.

Any of a variety of delivery vehicles may be employed within pharmaceutical compositions and vaccines to facilitate production of an antigen-specific immune response that targets tumor cells. Delivery vehicles include antigen presenting cells (APCs), such as dendritic cells, macrophages, B cells, monocytes and other cells that may be engineered to be efficient APCs. Such cells may, but need not, be genetically modified to increase the capacity for presenting the antigen, to improve activation and/or maintenance of the T cell response, to have anti-tumor effects per se and/or to be immunologically compatible with the receiver (i.e., matched HLA haplotype). APCs may generally be isolated from any of a variety of biological fluids and organs, including tumor and peritumoral tissues, and may be autologous, allogeneic, syngeneic or xenogeneic cells.

Certain preferred embodiments of the present invention use dendritic cells or progenitors thereof as antigen-presenting cells. Dendritic cells are highly potent APCs (Banchereau and Steinman, Nature 392:245-251, 1998) and have been shown to be effective as a physiological adjuvant for eliciting prophylactic or therapeutic antitumor immunity (see Timmerman and Levy, Ann. Rev. Med. 50:507-529, 1999). In general, dendritic cells may be identified based on their typical shape (stellate in situ, with marked cytoplasmic processes (dendrites) visible in vitro), their ability to take up, process and present antigens with high efficiency and their ability to activate naive T cell responses. Dendritic cells may, of course, be engineered to express specific cell-surface receptors or ligands that are not commonly found on dendritic cells in vivo or ex vivo, and such modified dendritic cells are contemplated by the present invention. As an alternative to dendritic cells, secreted vesicles antigen-loaded dendritic cells (called exosomes) may be used within a vaccine (see Zitvogel et al., Nature Med. 4:594-600, 1998).

Dendritic cells and progenitors may be obtained from peripheral blood, bone marrow, tumor-infiltrating cells, peritumoral tissues-infiltrating cells, lymph nodes, spleen, skin, umbilical cord blood or any other suitable tissue or fluid. For example, dendritic cells may be differentiated ex vivo by adding a combination of cytokines such as GM-CSF, IL-4, IL-13 and/or TNFα to cultures of monocytes harvested from peripheral blood. Alternatively, CD34 positive cells harvested from peripheral blood, umbilical cord blood or bone marrow may be differentiated into dendritic cells by adding to the culture medium combinations of GM-CSF, IL-3, TNFα, CD40 ligand, LPS, flt3 ligand and/or other compound(s) that induce differentiation, maturation and proliferation of dendritic cells.

Dendritic cells are conveniently categorized as “immature” and “mature” cells, which allows a simple way to discriminate between two well characterized phenotypes. However, this nomenclature should not be construed to exclude all possible intermediate stages of differentiation. Immature dendritic cells are characterized as APC with a high capacity for antigen uptake and processing, which correlates with the high expression of Fcγ receptor and mannose receptor. The mature phenotype is typically characterized by a lower expression of these markers, but a high expression of cell surface molecules responsible for T cell activation such as class I and class II MHC, adhesion molecules (e.g., CD54 and CD11) and costimulatory molecules (e.g., CD40, CD80, CD86 and 4-1BB).

APCs may generally be transfected with a polynucleotide encoding an ovarian tumor protein (or portion or other variant thereof) such that the ovarian tumor polypeptide, or an immunogenic portion thereof, is expressed on the cell surface. Such transfection may take place ex vivo, and a composition or vaccine comprising such transfected cells may then be used for therapeutic purposes, as described herein. Alternatively, a gene delivery vehicle that targets a dendritic or other antigen presenting cell may be administered to a patient, resulting in transfection that occurs in vivo. In vivo and ex vivo transfection of dendritic cells, for example, may generally be performed using any methods known in the art, such as those described in WO 97/24447, or the gene gun approach described by Mahvi et al., Immunology and cell Biology 75:456-460, 1997. Antigen loading of dendritic cells may be achieved by incubating dendritic cells or progenitor cells with the ovarian tumor polypeptide, DNA (naked or within a plasmid vector) or RNA; or with antigen-expressing recombinant bacterium or viruses (e.g., vaccinia, fowlpox, adenovirus or lentivirus vectors). Prior to loading, the polypeptide may be covalently conjugated to an immunological partner that provides T cell help (e.g., a carrier molecule). Alternatively, a dendritic cell may be pulsed with a non-conjugated immunological partner, separately or in the presence of the polypeptide.

Vaccines and pharmaceutical compositions may be presented in unit-dose or multi-dose containers, such as sealed ampoules or vials. Such containers are preferably hermetically sealed to preserve sterility of the formulation until use. In general, formulations may be stored as suspensions, solutions or emulsions in oily or aqueous vehicles. Alternatively, a vaccine or pharmaceutical composition may be stored in a freeze-dried condition requiring only the addition of a sterile liquid carrier immediately prior to use.

Cancer Therapy

In further aspects of the present invention, the compositions described herein may be used for immunotherapy of cancer, such as ovarian cancer. Within such methods, pharmaceutical compositions and vaccines are typically administered to a patient. As used herein, a “patient” refers to any warm-blooded animal, preferably a human. A patient may or may not be afflicted with cancer. Accordingly, the above pharmaceutical compositions and vaccines may be used to prevent the development of a cancer or to treat a patient afflicted with a cancer. A cancer may be diagnosed using criteria generally accepted in the art, including the presence of a malignant tumor. Pharmaceutical compositions and vaccines may be administered either prior to or following surgical removal of primary tumors and/or treatment such as administration of radiotherapy or conventional chemotherapeutic drugs. Administration may be by any suitable method, including administration by intravenous, intraperitoneal, intramuscular, subcutaneous, intranasal, intradermal, anal, vaginal, topical and oral routes.

Within certain embodiments, immunotherapy may be active immunotherapy, in which treatment relies on the in vivo stimulation of the endogenous host immune system to react against tumors with the administration of immune response-modifying agents (such as polypeptides and polynucleotides as provided herein).

Within other embodiments, immunotherapy may be passive immunotherapy, in which treatment involves the delivery of agents with established tumor-immune reactivity (such as effector cells or antibodies) that can directly or indirectly mediate antitumor effects and does not necessarily depend on an intact host immune system. Examples of effector cells include T cells as discussed above, T lymphocytes (such as CD8⁺ cytotoxic T lymphocytes and CD4⁺ T-helper tumor-infiltrating lymphocytes), killer cells (such as Natural Killer cells and lymphokine-activated killer cells), B cells and antigen-presenting cells (such as dendritic cells and macrophages) expressing a polypeptide provided herein. T cell receptors and antibody receptors specific for the polypeptides recited herein may be cloned, expressed and transferred into other vectors or effector cells for adoptive immunotherapy. The polypeptides provided herein may also be used to generate antibodies or anti-idiotypic antibodies (as described above and in U.S. Pat. No. 4,918,164) for passive immunotherapy.

Effector cells may generally be obtained in sufficient quantities for adoptive immunotherapy by growth in vitro, as described herein. Culture conditions for expanding single antigen-specific effector cells to several billion in number with retention of antigen recognition in vivo are well known in the art. Such in vitro culture conditions typically use intermittent stimulation with antigen, often in the presence of cytokines (such as IL-2) and non-dividing feeder cells. As noted above, immunoreactive polypeptides as provided herein may be used to rapidly expand antigen-specific T cell cultures in order to generate a sufficient number of cells for immunotherapy. In particular, antigen-presenting cells, such as dendritic, macrophage, monocyte, fibroblast and/or B cells, may be pulsed with immunoreactive polypeptides or transfected with one or more polynucleotides using standard techniques well known in the art. For example, antigen-presenting cells can be transfected with a polynucleotide having a promoter appropriate for increasing expression in a recombinant virus or other expression system. Cultured effector cells for use in therapy must be able to grow and distribute widely, and to survive long term in vivo. Studies have shown that cultured effector cells can be induced to grow in vivo and to survive long term in substantial numbers by repeated stimulation with antigen supplemented with IL-2 (see, for example, Cheever et al., Immunological Reviews 157:177, 1997).

Alternatively, a vector expressing a polypeptide recited herein may be introduced into antigen presenting cells taken from a patient and clonally propagated ex vivo for transplant back into the same patient. Transfected cells may be reintroduced into the patient using any means known in the art, preferably in sterile form by intravenous, intracavitary, intraperitoneal or intratumor administration.

Routes and frequency of administration of the therapeutic compositions described herein, as well as dosage, will vary from individual to individual, and may be readily established using standard techniques. In general, the pharmaceutical compositions and vaccines may be administered by injection (e.g., intracutaneous, intramuscular, intravenous or subcutaneous), intranasally (e.g., by aspiration) or orally. Preferably, between 1 and 10 doses may be administered over a 52 week period. Preferably, 6 doses are administered, at intervals of 1 month, and booster vaccinations may be given periodically thereafter. Alternate protocols may be appropriate for individual patients. A suitable dose is an amount of a compound that, when administered as described above, is capable of promoting an anti-tumor immune response, and is at least 10-50% above the basal (i.e., untreated) level. Such response can be monitored by measuring the anti-tumor antibodies in a patient or by vaccine-dependent generation of cytolytic effector cells capable of killing the patient's tumor cells in vitro. Such vaccines should also be capable of causing an immune response that leads to an improved clinical outcome (e.g., more frequent remissions, complete or partial or longer disease-free survival) in vaccinated patients as compared to non-vaccinated patients. In general, for pharmaceutical compositions and vaccines comprising one or more polypeptides, the amount of each polypeptide present in a dose ranges from about 25 μg to 5 mg per kg of host. Suitable dose sizes will vary with the size of the patient, but will typically range from about 0.1 mL to about 5 mL.

In general, an appropriate dosage and treatment regimen provides the active compound(s) in an amount sufficient to provide therapeutic and/or prophylactic benefit. Such a response can be monitored by establishing an improved clinical outcome (e.g., more frequent remissions, complete or partial, or longer disease-free survival) in treated patients as compared to non-treated patients. Increases in preexisting immune responses to an ovarian tumor protein generally correlate with an improved clinical outcome. Such immune responses may generally be evaluated using standard proliferation, cytotoxicity or cytokine assays, which may be performed using samples obtained from a patient before and after treatment.

Cancer Detection and Diagnosis

In general, a cancer may be detected in a patient based on the presence of one or more ovarian tumor proteins and/or polynucleotides encoding such proteins in a biological sample (for example, blood, sera, sputum urine and/or tumor biopsies) obtained from the patient. In other words, such proteins may be used as markers to indicate the presence or absence of a cancer such as ovarian cancer. In addition, such proteins may be useful for the detection of other cancers. The binding agents provided herein generally permit detection of the level of antigen that binds to the agent in the biological sample. Polynucleotide primers and probes may be used to detect the level of mRNA encoding a tumor protein, which is also indicative of the presence or absence of a cancer. In general, an ovarian tumor sequence should be present at a level that is at least three fold higher in tumor tissue than in normal tissue

There are a variety of assay formats known to those of ordinary skill in the art for using a binding agent to detect polypeptide markers in a sample. See, e.g., Harlow and Lane, Antibodies: A Laboratory Manual, Cold Spring Harbor Laboratory, 1988. In general, the presence or absence of a cancer in a patient may be determined by (a) contacting a biological sample obtained from a patient with a binding agent; (b) detecting in the sample a level of polypeptide that binds to the binding agent; and (c) comparing the level of polypeptide with a predetermined cut-off value.

In a preferred embodiment, the assay involves the use of binding agent immobilized on a solid support to bind to and remove the polypeptide from the remainder of the sample. The bound polypeptide may then be detected using a detection reagent that contains a reporter group and specifically binds to the binding agent/polypeptide complex. Such detection reagents may comprise, for example, a binding agent that specifically binds to the polypeptide or an antibody or other agent that specifically binds to the binding agent, such as an anti-immunoglobulin, protein G, protein A or a lectin. Alternatively, a competitive assay may be utilized, in which a polypeptide is labeled with a reporter group and allowed to bind to the immobilized binding agent after incubation of the binding agent with the sample. The extent to which components of the sample inhibit the binding of the labeled polypeptide to the binding agent is indicative of the reactivity of the sample with the immobilized binding agent. Suitable polypeptides for use within such assays include full length ovarian tumor proteins and portions thereof to which the binding agent binds, as described above.

The solid support may be any material known to those of ordinary skill in the art to which the tumor protein may be attached. For example, the solid support may be a test well in a microtiter plate or a nitrocellulose or other suitable membrane. Alternatively, the support may be a bead or disc, such as glass, fiberglass, latex or a plastic material such as polystyrene or polyvinylchloride. The support may also be a magnetic particle or a fiber optic sensor, such as those disclosed, for example, in U.S. Pat. No. 5,359,681. The binding agent may be immobilized on the solid support using a variety of techniques known to those of skill in the art, which are amply described in the patent and scientific literature. In the context of the present invention, the term “immobilization” refers to both noncovalent association, such as adsorption, and covalent attachment (which may be a direct linkage between the agent and functional groups on the support or may be a linkage by way of a cross-linking agent). Immobilization by adsorption to a well in a microtiter plate or to a membrane is preferred. In such cases, adsorption may be achieved by contacting the binding agent, in a suitable buffer, with the solid support for a suitable amount of time. The contact time varies with temperature, but is typically between about 1 hour and about 1 day. In general, contacting a well of a plastic microtiter plate (such as polystyrene or polyvinylchloride) with an amount of binding agent ranging from about 10 ng to about 10 μg, and preferably about 100 ng to about 1 μg, is sufficient to immobilize an adequate amount of binding agent.

Covalent attachment of binding agent to a solid support may generally be achieved by first reacting the support with a bifunctional reagent that will react with both the support and a functional group, such as a hydroxyl or amino group, on the binding agent. For example, the binding agent may be covalently attached to supports having an appropriate polymer coating using benzoquinone or by condensation of an aldehyde group on the support with an amine and an active hydrogen on the binding partner (see, e.g., Pierce Immunotechnology Catalog and Handbook, 1991, at A12-A13).

In certain embodiments, the assay is a two-antibody sandwich assay. This assay may be performed by first contacting an antibody that has been immobilized on a solid support, commonly the well of a microtiter plate, with the sample, such that polypeptides within the sample are allowed to bind to the immobilized antibody. Unbound sample is then removed from the immobilized polypeptide-antibody complexes and a detection reagent (preferably a second antibody capable of binding to a different site on the polypeptide) containing a reporter group is added. The amount of detection reagent that remains bound to the solid support is then determined using a method appropriate for the specific reporter group.

More specifically, once the antibody is immobilized on the support as described above, the remaining protein binding sites on the support are typically blocked. Any suitable blocking agent known to those of ordinary skill in the art, such as bovine serum albumin or Tween 20™ (Sigma Chemical Co., St. Louis, Mo.). The immobilized antibody is then incubated with the sample, and polypeptide is allowed to bind to the antibody. The sample may be diluted with a suitable diluent, such as phosphate-buffered saline (PBS) prior to incubation. In general, an appropriate contact time (i.e., incubation time) is a period of time that is sufficient to detect the presence of polypeptide within a sample obtained from an individual with ovarian cancer. Preferably, the contact time is sufficient to achieve a level of binding that is at least about 95% of that achieved at equilibrium between bound and unbound polypeptide. Those of ordinary skill in the art will recognize that the time necessary to achieve equilibrium may be readily determined by assaying the level of binding that occurs over a period of time. At room temperature, an incubation time of about 30 minutes is generally sufficient.

Unbound sample may then be removed by washing the solid support with an appropriate buffer, such as PBS containing 0.1% Tween20™ TM. The second antibody, which contains a reporter group, may then be added to the solid support. Preferred reporter groups include those groups recited above.

The detection reagent is then incubated with the immobilized antibody-polypeptide complex for an amount of time sufficient to detect the bound polypeptide. An appropriate amount of time may generally be determined by assaying the level of binding that occurs over a period of time. Unbound detection reagent is then removed and bound detection reagent is detected using the reporter group. The method employed for detecting the reporter group depends upon the nature of the reporter group. For radioactive groups, scintillation counting or autoradiographic methods are generally appropriate. Spectroscopic methods may be used to detect dyes, luminescent groups and fluorescent groups. Biotin may be detected using avidin, coupled to a different reporter group (commonly a radioactive or fluorescent group or an enzyme). Enzyme reporter groups may generally be detected by the addition of substrate (generally for a specific period of time), followed by spectroscopic or other analysis of the reaction products.

To determine the presence or absence of a cancer, such as ovarian cancer, the signal detected from the reporter group that remains bound to the solid support is generally compared to a signal that corresponds to a predetermined cut-off value. In one preferred embodiment, the cut-off value for the detection of a cancer is the average mean signal obtained when the immobilized antibody is incubated with samples from patients without the cancer. In general, a sample generating a signal that is three standard deviations above the predetermined cut-off value is considered positive for the cancer. In an alternate preferred embodiment, the cut-off value is determined using a Receiver Operator Curve, according to the method of Sackett et al., Clinical Epidemiology: A Basic Science for Clinical Medicine, Little Brown and Co., 1985, p. 106-7. Briefly, in this embodiment, the cut-off value may be determined from a plot of pairs of true positive rates (i.e., sensitivity) and false positive rates (100%-specificity) that correspond to each possible cut-off value for the diagnostic test result. The cut-off value on the plot that is the closest to the upper left-hand corner (i.e., the value that encloses the largest area) is the most accurate cut-off value, and a sample generating a signal that is higher than the cut-off value determined by this method may be considered positive. Alternatively, the cut-off value may be shifted to the left along the plot, to minimize the false positive rate, or to the right, to minimize the false negative rate. In general, a sample generating a signal that is higher than the cut-off value determined by this method is considered positive for a cancer.

In a related embodiment, the assay is performed in a flow-through or strip test format, wherein the binding agent is immobilized on a membrane, such as nitrocellulose. In the flow-through test, polypeptides within the sample bind to the immobilized binding agent as the sample passes through the membrane. A second, labeled binding agent then binds to the binding agent-polypeptide complex as a solution containing the second binding agent flows through the membrane. The detection of bound second binding agent may then be performed as described above. In the strip test format, one end of the membrane to which binding agent is bound is immersed in a solution containing the sample. The sample migrates along the membrane through a region containing second binding agent and to the area of immobilized binding agent. Concentration of second binding agent at the area of immobilized antibody indicates the presence of a cancer. Typically, the concentration of second binding agent at that site generates a pattern, such as a line, that can be read visually. The absence of such a pattern indicates a negative result. In general, the amount of binding agent immobilized on the membrane is selected to generate a visually discernible pattern when the biological sample contains a level of polypeptide that would be sufficient to generate a positive signal in the two-antibody sandwich assay, in the format discussed above. Preferred binding agents for use in such assays are antibodies and antigen-binding fragments thereof. Preferably, the amount of antibody immobilized on the membrane ranges from about 25 ng to about 1 μg, and more preferably from about 50 ng to about 500 ng. Such tests can typically be performed with a very small amount of biological sample.

Of course, numerous other assay protocols exist that are suitable for use with the tumor proteins or binding agents of the present invention. The above descriptions are intended to be exemplary only. For example, it will be apparent to those of ordinary skill in the art that the above protocols may be readily modified to use ovarian tumor polypeptides to detect antibodies that bind to such polypeptides in a biological sample. The detection of such ovarian tumor protein specific antibodies may correlate with the presence of a cancer.

A cancer may also, or alternatively, be detected based on the presence of T cells that specifically react with an ovarian tumor protein in a biological sample. Within certain methods, a biological sample comprising CD4⁺ and/or CD8⁺ T cells isolated from a patient is incubated with an ovarian tumor polypeptide, a polynucleotide encoding such a polypeptide and/or an APC that expresses at least an immunogenic portion of such a polypeptide, and the presence or absence of specific activation of the T cells is detected. Suitable biological samples include, but are not limited to, isolated T cells. For example, T cells may be isolated from a patient by routine techniques (such as by Ficoll/Hypaque density gradient centrifugation of peripheral blood lymphocytes). T cells may be incubated in vitro for 2-9 days (typically 4 days) at 37° C. with polypeptide (e.g., 5-25 μg/ml). It may be desirable to incubate another aliquot of a T cell sample in the absence of ovarian tumor polypeptide to serve as a control. For CD4⁺ T cells, activation is preferably detected by evaluating proliferation of the T cells. For CD8⁺ T cells, activation is preferably detected by evaluating cytolytic activity. A level of proliferation that is at least two fold greater and/or a level of cytolytic activity that is at least 20% greater than in disease-free patients indicates the presence of a cancer in the patient.

As noted above, a cancer may also, or alternatively, be detected based on the level of mRNA encoding an ovarian tumor protein in a biological sample. For example, at least two oligonucleotide primers may be employed in a polymerase chain reaction (PCR) based assay to amplify a portion of an ovarian tumor cDNA derived from a biological sample, wherein at least one of the oligonucleotide primers is specific for (i.e., hybridizes to) a polynucleotide encoding the ovarian tumor protein. The amplified cDNA is then separated and detected using techniques well known in the art, such as gel electrophoresis. Similarly, oligonucleotide probes that specifically hybridize to a polynucleotide encoding an ovarian tumor protein may be used in a hybridization assay to detect the presence of polynucleotide encoding the tumor protein in a biological sample.

To permit hybridization under assay conditions, oligonucleotide primers and probes should comprise an oligonucleotide sequence that has at least about 60%, preferably at least about 75% and more preferably at least about 90%, identity to a portion of a polynucleotide encoding an ovarian tumor protein that is at least 10 nucleotides, and preferably at least 20 nucleotides, in length. Preferably, oligonucleotide primers and/or probes hybridize to a polynucleotide encoding a polypeptide described herein under moderately stringent conditions, as defined above. Oligonucleotide primers and/or probes which may be usefully employed in the diagnostic methods described herein preferably are at least 10-40 nucleotides in length. In a preferred embodiment, the oligonucleotide primers comprise at least 10 contiguous nucleotides, more preferably at least 15 contiguous nucleotides, of a DNA molecule having a sequence recited in SEQ ID NOs:1-222. Techniques for both PCR based assays and hybridization assays are well known in the art (see, for example, Mullis et al., Cold Spring Harbor Symp. Quant. Biol., 51:263, 1987; Erlich ed., PCR Technology, Stockton Press, NY, 1989).

One preferred assay employs RT-PCR, in which PCR is applied in conjunction with reverse transcription. Typically, RNA is extracted from a biological sample, such as biopsy tissue, and is reverse transcribed to produce cDNA molecules. PCR amplification using at least one specific primer generates a cDNA molecule, which may be separated and visualized using, for example, gel electrophoresis. Amplification may be performed on biological samples taken from a test patient and from an individual who is not afflicted with a cancer. The amplification reaction may be performed on several dilutions of cDNA spanning two orders of magnitude. A two-fold or greater increase in expression in several dilutions of the test patient sample as compared to the same dilutions of the non-cancerous sample is typically considered positive.

In another embodiment, the compositions described herein may be used as markers for the progression of cancer. In this embodiment, assays as described above for the diagnosis of a cancer may be performed over time, and the change in the level of reactive polypeptide(s) or polynucleotide(s) evaluated. For example, the assays may be performed every 24-72 hours for a period of 6 months to 1 year, and thereafter performed as needed. In general, a cancer is progressing in those patients in whom the level of polypeptide or polynucleotide detected increases over time. In contrast, the cancer is not progressing when the level of reactive polypeptide or polynucleotide either remains constant or decreases with time.

Certain in vivo diagnostic assays may be performed directly on a tumor. One such assay involves contacting tumor cells with a binding agent. The bound binding agent may then be detected directly or indirectly via a reporter group. Such binding agents may also be used in histological applications. Alternatively, polynucleotide probes may be used within such applications.

As noted above, to improve sensitivity, multiple ovarian tumor protein markers may be assayed within a given sample. It will be apparent that binding agents specific for different proteins provided herein may be combined within a single assay. Further, multiple primers or probes may be used concurrently. The selection of tumor protein markers may be based on routine experiments to determine combinations that results in optimal sensitivity. In addition, or alternatively, assays for tumor proteins provided herein may be combined with assays for other known tumor antigens.

Diagnostic Kits

The present invention further provides kits for use within any of the above diagnostic methods. Such kits typically comprise two or more components necessary for performing a diagnostic assay. Components may be compounds, reagents, containers and/or equipment. For example, one container within a kit may contain a monoclonal antibody or fragment thereof that specifically binds to an ovarian tumor protein. Such antibodies or fragments may be provided attached to a support material, as described above. One or more additional containers may enclose elements, such as reagents or buffers, to be used in the assay. Such kits may also, or alternatively, contain a detection reagent as described above that contains a reporter group suitable for direct or indirect detection of antibody binding.

Alternatively, a kit may be designed to detect the level of mRNA encoding an ovarian tumor protein in a biological sample. Such kits generally comprise at least one oligonucleotide probe or primer, as described above, that hybridizes to a polynucleotide encoding an ovarian tumor protein. Such an oligonucleotide may be used, for example, within a PCR or hybridization assay. Additional components that may be present within such kits include a second oligonucleotide and/or a diagnostic reagent or container to facilitate the detection of a polynucleotide encoding an ovarian tumor protein.

The following Examples are offered by way of illustration and not by way of limitation.

### EXAMPLE 1

### Identification of cDNAs Encoding Ovarian and Endometrial Tumor Proteins

An ovarian/endometrial tumor cell line subtracted library was constructed. A library was prepared from endometrial and ovarian tumor cell lines: EndoTL 391-73 (100% undifferentiated endometrial carcinoma), OTL 298-95 (100% moderately differentiated papillary serous ovarian adenocarcinoma) and OTL 522-24 (30% mesothelial cells/70% poorly differentiated metastatic ovarian adenocarcinoma). This library was subtracted with liver, pancreas, skin, bone marrow, resting PBMC, stomach, and brain cDNA and spiked with eukaryotic elongation factor 1α. Resulting cDNA was cloned into the pcDNA3.1 (+) (Invitrogen) vector to generate the ovarian tumor cell line subtraction 4 library (OTCLS4). The OTCLS4 library contained 117,200 clones (background 58,400), with a 1333 bp average insert size (inserts ranged from 200 to 5650 bp).

Thirty clones were sequenced. Of these 12 were full length. The clones maybe grouped as follows (SEQ ID NOs are provided in Table 2):

7 Novel

4 Homo sapiens aldehyde dehydrogenase 6 (ALDH6) mRNA

3 Human ferritin heavy chain mRNA, complete cds

2 Human lysyl oxidase gene, partial cds

2 Human mitochondrion, complete genome

1 Homo sapiens aldehyde reductase 1 (low Km aldose reductase) ALDR1) mRNA

1 Homo sapiens chromosome 11q12.2 PAC clone pDJ519o13

1 Homo sapiens chromosome-associated polypeptide C (CAP-C) mRNA

1 Homo sapiens clone 24452 mRNA sequence

1 Homo sapiens dipeptidylpeptidase IV (CD26, adenosine deaminase complexing protein 2) (DPP4 mRNA)

1 Homo sapiens guanine nucleotide binding protein (G protein), beta polypeptide 2-like 1 (GNB2L1), mRNA

1 Homo sapiens heat shock 27kD protein 1 (HSPB1) mRNA

1 Homo sapiens homeo box B2 (HOXB2) mRNA

1 Homo sapiens mRNA for KIAA0865 protein, partial cds

1 Homo sapiens mRNA; cDNA DKFZp564A2416 (from clone DKFZp564A2416)

1 Homo sapiens NADH-ubiquinone oxidoreductase 39kDA subunit mRNA, nuclear gene encoding mitochondrial protein, complete cds

1 Homo sapiens Sk/Dkk-1 protein precursor, mRNA, complete cds

1 Homo sapiens sodium channel, nonvoltage-gated 1 alpha (SCNN1A) mRNA

1 Homo sapiens SRP1 mRNA, partial sequence

1 Homo sapiens zinc finger protein SLUG (SLUG) gene, complete cds

1 Human 28S ribosomal RNA gene

1 Human cofilin mRNA, partial cds

1 Human DNA sequence from clone 967N21 on chromosome 20p12.3-13

1 Human fibroblast collagenase inhibitor mRNA, complete eds

1 Human fibroblast mRNA for aldolase A

1 Human HepG2 3′ region MboI cDNA, clone hmd6a06m3

1 Human MAP kinase kinase MEK5c mRNA, complete cds

1 Human mRNA for coupling protein G(s) alpha-subunit (alpha-S1)

1 Human mRNA for KIAA0026 gene, completecds|gi|4808630|gb|AF100620.1| AF100620 Homo sapiens transcription factor-like protein MRGX (MRGX) mRNA, complete cds

1 Human mRNA for KIAA0064 gene, complete cds

1 Human mRNA for KIAA0204 gene, complete cds

1 Human plasminogen activator inhibitor-1 (PAI-1) mRNA, complete cds

1 Human protocadherin 43 mRNA, 3′ end of cds for alternative splicing PC43-12

1 Human putative RNA binding protein Koc1 mRNA, complete cds

1 Human TCB gene encoding cytosolic thyroid hormone-binding protein, complete cds

1 Human ubiquitin-homology domain protein PIC1 mRNA, complete cds

TABLE 2
Ovarian/Endometrial Carcinoma Associated cDNA Sequences

| | SEQ ID | |
|---|---|---|
| Sequence | NO | Comments |
| 32609 | 36 | Homo sapiens aldehyde dehydrogenase 6 (ALDH6) |
| | | mRNA |
| 32515 | 4 | Homo sapiens aldehyde reductase 1 (low Km aldose |
| | | reductase) (ALDR1) mRNA |
| 32562 | 29 | Homo sapiens Chromosome 11q12.2 PAC clone |
| | | pDJ519o13 |
| 32523 | 9 | Homo sapiens chromosome-associated polypeptide |
| | | C (CAP-C) mRNA |
| 32551 | 24 | Homo sapiens clone 24452 mRNA sequence |
| 32518 | 6 | Homo sapiens dipeptidylpeptidase IV (CD26, |
| | | adenosine deaminase complexing protein 2) (DPP4) |
| | | mRNA |
| 32534 | 13 | Homo sapiens guanine nucleotide binding protein (G |
| | | protein), beta polypeptide 2-like 1 (GNB2L1), |
| | | mRNA |
| 32507 | 2 | Homo sapiens heat shock 27 kD protein 1 (HSPB1) |
| | | mRNA |
| 32533 | 12 | Homo sapiens homeo box B2 (HOXB2) mRNA |
| 32565 | 20 | Homo sapiens mRNA for KIAA0865 protein, |
| | | partial cds |
| 32553 | 19 | Homo sapiens mRNA; cDNA DKFZp564A2416 |
| | | (from clone DKFZp564A2416) |
| 32561 | 28 | Homo sapiens NADH-ubiquinone oxidoreductase |
| | | 39 kDa subunit mRNA, nuclear gene encoding |
| | | mitochondrial protein, complete cds |
| 32510 | 3 | Homo sapiens Sk/Dkk-1 protein precursor, mRNA, |
| | | complete cds |
| 32546 | 16 | Homo sapiens sodium channel, nonvoltage-gated 1 |
| | | alpha (SCNN1A) mRNA |
| 32559 | 27 | Homo sapiens SRP1 mRNA, partial sequence |
| 32506 | 1 | Homo sapiens zinc finger protein SLUG gene, |
| | | complete cds |
| 32519 | 7 | Human 28S ribosomal RNA gene |
| 32602 | 22 | Human cofilin mRNA, partial cds |
| 32569 | 31 | Human DNA sequence from clone 967N21 on |
| | | chromosome 20p12.3-13 |
| 32525 | 10 | Human ferritin heavy chain mRNA, complete cds |
| 32557 | 26 | Human fibroblast collagenase inhibitor mRNA, |
| | | complete cds |
| 32517 | 5 | Human fibroblast mRNA for aldolase A |
| 32568 | 30 | Human HepG2 3′ region MboI cDNA, clone |
| | | hmd6a06m3 |
| 32548 | 17 | Human lysyl oxidase gene, partial cds |
| 32520 | 8 | Human mitochondrion, complete genome |
| 32617 | 23 | Human mRNA for coupling protein G(s) alpha- |
| | | subunit (alpha-S1) |
| 32572 | 32 | Human mRNA for KIAA0026 gene, complete |
| | | cds\|gi\|4808630\|gb\|AF100620.1\|AF100620 |
| | | Homo sapiens transcription factor-like protein |
| | | MRGX (MRGX) mRNA, complete cds |
| 32600 | 21 | Human mRNA for KIAA0064 gene, complete cds |
| 32537 | 14 | Human mRNA for KIAA0204 gene, complete cds |
| 32552 | 25 | Human plasminogen activator inhibitor-1 (PAI-1) |
| | | mRNA, complete cds |
| 32615 | 39 | Human protocadherin 43 mRNA, 3′ end of cds for |
| | | alternative splicing PC43-12 |
| 32613 | 38 | Human putative RNA binding protein Koc 1 |
| | | mRNA, complete cds |
| 32610 | 37 | Human TCB gene encoding cytosolic thyroid |
| | | hormone-binding protein, complete cds |
| 32539 | 15 | Human ubiquitin-homology domain protein PIC1 |
| | | mRNA, complete cds |
| 32619 | 40 | Novel |
| 32576 | 33 | Novel |
| 32608 | 35 | Novel |
| 32607 | 34 | Novel |
| 32620 | 41 | Novel |
| 32550 | 18 | Novel |
| 32529 | 11 | Novel |

Using the methods outlined above, an additional 162 clones were isolated and sequenced. The cDNA sequences are shown in SEQ ID NO:42-203.

SEQ ID NO:204-209 represent additional clones from the OTCL S4 library. SEQ ID NO:206 (clone 57881), 208 (clone 57884), 107 (clone R0199:A07) and 80 (clone U0198:F02) represent novel sequences. The remaining sequences are shown in Table 3, which includes additional results from homology searches.

TABLE 3

| | SEQ ID | |
|---|---|---|
| Sequence | NO | Comments |
| 57877 | 204 | H. Sapiens novel gene from PAC 117P20, |
| | | chromosome 1 |
| 57879 | 205 | Urokinase plasmingen activator surface receptor |
| | | (uPAR) |
| 57882 | 207 | Lysophospholipase 1 (LYPA1) |
| 57888 | 209 | IGF-II mRNA binding protein 3 (IMP-3) mRNA |
| R0198:H03 | 99 | Homo sapiens laminin |
| R0199:B03 | 111 | Human cyclin protein gene, complete cds |
| R0200:A12 | 158 | Homo sapiens monocarboxylate transporter |
| | | (MCT3) mRNA |
| R0199:C12 | 125 | Unigene: Hs93379 |
| R0200:A10 | 157 | Human mRNA for KIAA0101 gene, complete |
| | | cds |
| R0198:D01 | 61 | Unigene: Hs42116 |
| R0200:C02 | 164 | Human proliferating cell nuclear antigen (PCNA) |
| | | gene |
| R0200:G02 | 193 | Homo sapiens Xq28 BAC RP5-1014016 |

### EXAMPLE 2

### Analysis of cDNA Expression using Microarray Technology

In additional studies, sequences disclosed herein were found to be overexpressed in specific tumor tissues as determined by microarray analysis. Using this approach, cDNA sequences are PCR amplified and their mRNA expression profiles in tumor and normal tissues are examined using cDNA microarray technology essentially as described (Shena et al., 1995). In brief, the clones are arrayed onto glass slides as multiple replicas, with each location corresponding to a unique cDNA clone (as many as 5500 clones can be arrayed on a single slide, or chip). Each chip is hybridized with a pair of cDNA probes that are fluorescence-labeled with Cy3 and Cy5, respectively. Typically, 1 μg of polyA⁺ RNA is used to generate each cDNA probe. After hybridization, the chips are scanned and the fluorescence intensity recorded for both Cy3 and Cy5 channels. There are multiple built-in quality control steps. First, the probe quality is monitored using a panel of ubiquitously expressed genes. Secondly, the control plate also can include yeast DNA fragments of which complementary RNA may be spiked into the probe synthesis for measuring the quality of the probe and the sensitivity of the analysis. Currently, the technology offers a sensitivity of 1 in 100,000 copies of mRNA. Finally, the reproducibility of this technology can be ensured by including duplicated control cDNA elements at different locations.

A total of 428 clones from the OCTLS4 library were analyzed on Ovarian Chip-3. The following table, Table 4, provides a list of probes used to interrogate these clones. A total of 16 clones were identified which showed at least 2-fold overexpression in ovarian tumors when compared to non-ovarian essential normal tissues and had a mean non-ovarian essential normal tissue expression of less than 0.2. These clones are represented by SEQ ID NO:204-209 and by SEQ ID NO:61, 99, 111, 125, 157, 158, 164 and 193.

TABLE 4

| | | | Tumor |
|---|---|---|---|
| Tissue | Clone ID | Microarray ID | information |
| Ovarian tumor | 261A | 391cy3 | Stage IIIC |
| Adrenal gland | SPACT37 | 391cy5 | |
| normal | | | |
| Ovary tumor | 264A | 392cy3 | Stage IIIC |
| Skin normal | 396A | 392cy5 | |
| Ovary tumor | 265A | 393cy3 | Stage IIIC |
| Thymus normal | SPACT56 | 393cy5 | |
| Ovary tumor | 288A | 394cy3 | Stage IIIC |
| Bronchus normal | 600C | 394cy5 | |
| Ovary tumor | 854A | 395cy3 | |
| | 785B | 395cy5 | |
| Ovary tumor | 855A | 396cy3 | Grade III, Stage IA |
| Bone normal | 407B | 396cy5 | |
| Ovary tumor | 856A | 397cy3 | Serous papillary |
| Peritoneum | 484A | 397cy5 | |
| epithelium normal | | | |
| Ovary tumor | 603A | 398cy3 | Metastatic |
| Pituitary gland | SPACT52 | 398cy5 | adenocarcinoma, |
| | | | Grade III, Stage III |
| Ovary tumor | 857A | 399cy3 | Papillary serous |
| Skeletal muscle | SPACT40 | 399cy5 | cystadenocarcinoma |
| normal | | | Grade III, Stage IB |
| Ovary tumor | 385A | 400cy3 | Papillary serous |
| Stomach normal | SPACT55 | 400cy5 | adenocarcinoma |
| Ovary tumor | 392A | 401cy3 | Papillary serous |
| Spleen normal | SPACT54 | 401cy5 | neoplasm, Stage 1C |
| Ovary tumor | 858A | 402cy3 | Papillary serous |
| Pancreas normal | 862A | 402cy5 | cystadenocarcinoma |
| | | | Grade II-III, Stage |
| | | | IA |
| Ovary tumor | 859A | 403cy3 | Papillary serous |
| Ovary normal | S27 | 403cy5 | adenocarcinoma |
| | | | Grade II-III, Stage |
| | | | IIB |
| Ovary tumor | 605A | 404cy3 | Serous borderline |
| Spinal cord normal | SPACT45 | 404cy5 | tumor, stage IIIC |
| Ovary tumor | 495A | 405cy3 | Papillary serous |
| Heart normal | SPAAm1 | 405cy5 | carcinoma, Grade II, |
| | | | Stage III |
| Ovary tumor | 381C | 414cy3 | Mucinous |
| Ovary normal | S7 | 414cy5 | adenocarcinoma, |
| | | | Grade I, Sage IB |
| Ovary tumor | 382A | 416cy3 | Mucinous |
| Ovary normal | S449A | 416cy5 | adenocarcinoma |
| Ovary tumor | 428B | 417cy3 | Mucinous |
| metastases | SPACT53 | 417cy5 | adenocarcinoma |
| Small intestine | | | |
| normal | | | |
| Ovary tumor | 491A | 418cy3 | Endometriod |
| Esophagus normal | 502B | 418cy5 | adenocarcinoma |
| Ovary tumor | 335A | 419cy3 | Endometriod |
| Colon normal | 199A | 419cy5 | adenocarcinoma |
| | | | Grade II, Stage II |
| Ovary tumor | 494A | 421cy3 | Adenocarcinoma |
| Thyroid gland | SPACT46 | 421cy5 | Grade III, Stage II- |
| normal | | | III |
| Ovary tumor | 860A | 42cy3 | Endometriod |
| PBMC (resting) | 783A | 422cy5 | adenocarcinoma |
| | | | Grade II-III, Stage |
| | | | IIIC |
| Ovary tumor | 604A | 423cy3 | Clear cell carcinoma |
| Aorta normal | 415A | 423cy5 | |
| Ovary tumor | 607A | 424cy3 | Clear cell, Stage IA |
| Trachea normal | 776A | 424cy5 | |
| Ovary tumor | S25 | 425cy3 | Granulosa cell |
| Trachea normal | CT25 | 425cy5 | tumor, Stage IA |
| Ovary tumor | S22 | 426cy3 | Granulosa cell |
| Pancreas normal | PAN2000 | 426cy5 | tumor, Stage IA |
| pool | | | |
| Ovary tumor | 386A | 427cy3 | Germ cell tumor, |
| Breast (HMEC) | S92 | 427cy5 | Stage I |
| normal | | | |
| Ovary tumor | 602A | 429cy3 | Papillary serous |
| Bladder normal | 328B/C | 429cy5 | carcinoma, Grade |
| | | | III, Stage IIIB |
| Ovary tumor | S23 | 430cy3 | Papillary serous |
| Bone marrow | SPACT49 | 430cy5 | adenocarcinoma |
| normal | | | Grade III, Stage |
| | | | IIIC |
| Ovary tumor | 606A | 428cy3 | Papillary serous |
| Lung normal | SPAAm2 | 428cy5 | cystadenocarcinoma |
| | | | Grade II, Stage IIIB |
| Ovary tumor | 383A | 431cy3 | Metastatic papillary |
| metastases | 302B | 431cy5 | adenocarcinoma, |
| Kidney normal | | | Grade III, Stage |
| | | | IIIA |
| Ovary tumor | 384A | 423cy3 | Papillary serous |
| metastases | S40.782A | 423cy5 | adenocarcinoma |
| PBMC (activated) | | | Grade II, Stage IIIB |
| Ovary tumor | 426A | 433cy3 | Papillary serous |
| metastases | 603A | 433cy5 | adenocarcinoma |
| Ovary tumor match | | | Grade III, Stage |
| with CY3 | | | IIIB |
| Ovary tumor | 429A | 434cy3 | Papillary |
| metastases | 270B | 434cy5 | adenocarcinoma |
| Liver normal | | | Grade III, Stage III |
| Ovary tumor | 427A | 435cy3 | Papillary serous |
| Brain normal | SPACT50 | 435cy5 | adenocarcinoma |
| | | | Grade III, Stage |
| | | | IIIC |
| Ovary tumor | 855A | 436cy3 | Grade III, Stage IA |
| Bone normal | 407B | 436cy5 | |
| Ovary tumor | 605A | 437cy3 | Serous borderline |
| Spinal cord normal | SPACT45 | 437cy5 | tumor, Stage IIIC |
| Ovary tumor | 495A | 438cy3 | Papillary serous |
| Heart normal | SPAAm1 | 438cy5 | carcinoma, Grade II, |
| | | | Stage III |
| Ovary tumor | 381C | 439cy3 | Mucnous |
| Ovary normal | S7 | 439cy5 | adenocarcinoma, |
| | | | Grade I, Stage IB |

### EXAMPLE 3

### Synthesis of Polypeptides

Polypeptides may be synthesized on a Perkin Elmer/Applied Biosystems Division 430A peptide synthesizer using FMOC chemistry with HPTU (O-Benzotriazole-N,N,N′,N′-tetramethyluronium hexafluorophosphate) activation. A Gly-Cys-Gly sequence may be attached to the amino terminus of the peptide to provide a method of conjugation, binding to an immobilized surface, or labeling of the peptide. Cleavage of the peptides from the solid support may be carried out using the following cleavage mixture: trifluoroacetic acid:ethanedithiol:thioanisole:water:phenol (40:1:2:2:3). After cleaving for 2 hours, the peptides may be precipitated in cold methyl-t-butyl-ether. The peptide pellets may then be dissolved in water containing 0.1% trifluoroacetic acid (TFA) and lyophilized prior to purification by C18 reverse phase HPLC. A gradient of 0%-60% acetonitrile (containing 0.1% TFA) in water (containing 0.1% TFA) may be used to elute the peptides. Following lyophilization of the pure fractions, the peptides may be characterized using electrospray or other types of mass spectrometry and by amino acid analysis.

From the foregoing it will be appreciated that, although specific embodiments of the invention have been described herein for purposes of illustration, various modifications may be made without deviating from the spirit and scope of the invention. Accordingly, the invention is not limited except as by the appended claims.

What is claimed:

1. An isolated polynucleotide comprising a sequence selected from the group consisting of:
(a) sequences provided in SEQ ID NO: 1-222;
(b) complements of the sequences provided in SEQ ID NO: 1-222;
(c) sequences consisting of at least 20 contiguous residues of a sequence provided in SEQ ID NO: 1-222;
(d) sequences that hybridize to a sequence provided in SEQ ID NO: 1-222, under moderately stringent conditions;
(e) sequences having at least 75% identity to a sequence of SEQ ID) NO: 1-222;
(f) sequences having at least 90% identity to a sequence of SEQ ID) NO: 1-222; and
(g) degenerate variants of a sequence provided in SEQ ID NO: 1-222.

2. An isolated polypeptide comprising an amino acid sequence selected from the group consisting of:
(a) sequences encoded by a polynucleotide of claim 1; and
(b) sequences having at least 70% identity to a sequence encoded by a polynucleotide of claim 1; and
(c) sequences having at least 90% identity to a sequence encoded by a polynucleotide of claim 1.

3. An expression vector comprising a polynucleotide of claim 1 operably linked to an expression control sequence.

4. A host cell transformed or transfected with an expression vector according to claim 3.

5. An isolated antibody, or antigen-binding fragment thereof, that specifically binds to a polypeptide of claim 2.

6. A method for detecting the presence of a cancer in a patient, comprising the steps of:
(a) obtaining a biological sample from the patient;
(b) contacting the biological sample with a binding agent that binds to a polypeptide of claim 2;
(c) detecting in the sample an amount of polypeptide that binds to the binding agent; and
(d) comparing the amount of polypeptide to a predetermined cut-off value and therefrom determining the presence of a cancer in the patient.

7. A fusion protein comprising at least one polypeptide according to claim 2.

8. An oligonucleotide that hybridizes to a sequence recited in SEQ ID NO: 1-222 under moderately stringent conditions.

9. A method for stimulating and/or expanding T cells specific for a tumor protein, comprising contacting T cells with at least one component selected from the group consisting of:
(a) polypeptides according to claim 2;
(b) polynucleotides according to claim 1; and
(c) antigen-presenting cells that express a polypeptide according to claim 2, under conditions and for a time sufficient to permit the stimulation and/or expansion of T cells.

10. An isolated T cell population, comprising T cells prepared according to the method of claim 9.

11. A composition comprising a first component selected from the group consisting of physiologically acceptable carriers and immunostimulants, and a second component selected from the group consisting of:
(a) polypeptides according to claim 2;
(b) polynucleotides according to claim 1;
(c) antibodies according to claim 5;
(d) fusion proteins according to claim 7;
(e) T cell populations according to claim 10; and
(f) antigen presenting cells that express a polypeptide according to claim 2.

12. A method for stimulating an immune response in a patient, comprising administering to the patient a composition of claim 11.

13. A method for the treatment of a cancer in a patient, comprising administering to the patient a composition of claim 11.

14. A method for determining the presence of a cancer in a patient, comprising the steps of:
(a) obtaining a biological sample from the patient;
(b) contacting the biological sample with an oligonucleotide according to claim 8;
(c) detecting in the sample an amount of a polynucleotide that hybridizes to the oligonucleotide; and
(d) compare the amount of polynucleotide that hybridizes to the oligonucleotide to a predetermined cut-off value, and therefrom determining the presence of the cancer in the patient.

15. A diagnostic kit comprising at least one oligonucleotide according to claim 8.

16. A diagnostic kit comprising at least one antibody according to claim 5 and a detection reagent, wherein the detection reagent comprises a reporter group.

17. A method for inhibiting the development of a cancer in a patient, comprising the steps of:
(a) incubating CD4+ and/or CD8+ T cells isolated from a patient with at least one component selected from the group consisting of: (i) polypeptides according to claim 2; (ii) polynucleotides according to claim 1; and (iii) antigen presenting cells that express a polypeptide of claim 2, such that T cell proliferate;
(b) administering to the patient an effective amount of the proliferated T cells, and thereby inhibiting the development of a cancer in the patient.


SEQUENCE LISTING (222 sequences)

SEQ ID NO: 1 - DNA, 595 bp, Homo sapien
  misc_feature (1)...(595): n = A,T,C or G
cctttttttttttttttaaatcaaaactgtttattgtaaaaaaaacttgaaaattgttttttaaaaaagaaacattgatttcacaagtcttcaggttgtttatagacatagctatagacaacatctcagtttcatacagaactcattcaatcatataaaaataaacacaaatttacattgactcatcaactatacaatttaaaaaggcacttggaaggggtattgtattattgcatttgtggtatgcatttgaaatagtttaagtacattaatgaatttgtaagaatcctcttttgcacttattcccatctttaattaattttcaaaaattattaaaatgttttaaaatagtaagacaatggagcatgcgccaggaatgtttcaaagctaatctttccctcctcccccaaggcacatactgttaattgggcaaaaacaaaaacaaacaaaaatacttttaatacattctcctgggggttggnncttggnaatttttttttcccctttaaaaatatacctttaangcnctcaggtaatcaaaaaaaaggctttagtcacaantggcnacccgnccaacccactngcacnggnntan

SEQ ID NO: 2 - DNA, 1700 bp, Homo sapien
  misc_feature (1)...(1700): n = A,T,C or G
aaaagcgcagccgagcccagcgccccgcacttttctgagcagacgtccagagcagagtcagccagcatgaccgagcgccgcgtccccttctcgctcctgcggggccccagctgggaccccttccgcgactggtacccgcatagccgcctcttcgaccaggccttcgggctgccccggctgccggaggagtggtcgcagtggttaggcggcagcagctggccaggctacgtgcgccccctgccccccgccgccatcgagagccccgcagtggccgcgcccgcctacagccgcgcgctcagccggcaactcagcagcggggtctcggagatccggcacactgcggaccgctggcgcgtgtccctggatgtcaaccacttcgccccggacgagctgacggtcaagaccaaggatggcgtggtggagatcaccggcaagcacgangagcggcaggacgagcatggctacatctcccggtgcttcacgcggaaatacacgctgccccccggtgtggaccccacccaagtttcttctccctgtcccctgagggcacactgaccgnggaggnccccatgcccaagctagccacgcagtccaacgagatcaccatnccagtnaccttcnantngcgggcccagcttgggggnccanaanctnnnaaaatccnataagantggccgccaanaaannccttannnccgggatgcccaccccttgntgcngccnntgggtngggccttcccccnccnccnggggggnnntttnnananannnanntnnggnnnnnnnnnaaaaggnnnnnangnnnccccnnnnnnnnnnnnnnnnnnnnnnnnnnnnnnnnnnnnnnnnnnnnnnnnnnnnnnnnnnnnnnnnnnnnnnnnnnnnngnggngnnnnnnnnnnnnnntnnnnnnnnnnccncnngnnnnnnnnngnnnnnnnnnnnnnnnnnnnnnnnnnnnnnnnnnnnnnnnnnnnnnnnnnnnnnnnnnnnnnnnnnnnnnnnnnnnnnngggnntnntnntnnnnnnnnnnnnnnnnnnnnnnnnnnnnnnnnnngnnncncnnnnnnnnnnnnnnnnnnnnnggnnnnnnnnnnnnnnnnnnnnnnnnnnnnnnnnnnnnnnnnnnnnnnnnnnnnnnnnngnngnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncncnnnnnnnnnnnnnnnnnnnnnnnnnnnnnnnnnnnnnnnnnnnnnnnnnnnnnnnnncccccccccccccccccccccccccccccccccccnnnnnnnnnnnnnncccccccccccccccnnttttttnccccccccccccccccccccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnngn

SEQ ID NO: 3 - DNA, 583 bp, Homo sapien
  misc_feature (1)...(583): n = A,T,C or G
ccttttttttttttttttgatattaaatgttaaattttatttcaaaaactatcacagcctaaagggaaatataatttaagcattaagatagtacatttcagaaaataagctagtattttcatgttacattttaggtacctatcatttgtcattccaagagatccttgcgttctagactctanaattaaatggggtaaagggttatgcttttaagaactataagctgaaatgatttacttcagttcaatatagaatatttgtcagtcaagataacaatcaatgtgtcaaaaatttacataacaagaggaaaaataggcagtgcagcacctttagaaaaataattaaaagtttcattgcatttacangnaagtgccacactgagaatttacaatacagtaatttactgcaatcacaggggagttccataaagaaacaaagctcttcactccaggtttttggaanggggtattggaagcttaactgaaaccccaaaacntggttantcctnngaatgagttgatgaaaggcataaaaagggttcttagccctnttntntaaaagggggccccgctttgggaaacng

SEQ ID NO: 4 - DNA, 448 bp, Homo sapien
  misc_feature (1)...(448): n = A,T,C or G
ccttttttttttttttttcacaaaagcactttttatttgaggcaaagagaagtcttgctgaaaggattccagttccaagcagtcaaaactcaaccgttagtggcactattttgacctggtagattttgcttctctttggtcanaaaagggtattcaggttgtactttccccagcagggtagaaagaagggcaaagcaaactggaagagacttctactctactgacagggctcttgagatccaacatcaagctagacacgccctcgctggccactctacaggttgctgtcccactgctgagtgacacaggccatactacatttgcaaggaaaaaaatgaggcaagaaacacaggtataggtcacttggggacgagcaggcaaccacagcttcaaaactcttcatggaaggggtaatccttgnggggaggnacagctcaagtcgaccggc

SEQ ID NO: 5 - DNA, 2067 bp, Homo sapien
  misc_feature (1)...(2067): n = A,T,C or G
ccgaggctaaatcggctgcgttcctctcggaacgcgccgcananggggtcctggtgacgagtcccgcgttctctccttgaatccactcgccagcccgccgccctctgccgccgcaccctgcacacccgcccctctcctgtgccaggaacttgctactaccagcaccatgccctaccaatatccagcactgaccccggagcagaagaangagctgtctgacatcgctcaccgcatcgtggacctggcaagggcatcctgnctgcagatgagtccactgggagcattgncaagcggctgcattccattggcaccgagaacaccgaggagaaccggcgcttctaccgccagctgctgctgacagctgacgaccgcgtgaacccctgcattgggggtgtcatnctcttccatgagacactctnccagaaggcggatgatgggcgtcccttcccccaagttatcaaatccaagggcggtgttggggggcatcaaggtagacaagggcgnggtccccctggcaggggacaaatggngagactaccacccaaagggttggatgggctgtctgaancnctgngcccagnacnaanaangacggagctgacttccccaantggngtttgngtgctnaaaaattggggaaaacaacccccctnaaacccttcngcattnatggaaaaatncccaatgttntgggnccntnangccnngntntnccannnnnaangggatttnngcnccntnnnngganccnnnnananccnccccttgnggggggnaacatnnaannttnnaanngnnnngnncnnnnnnngnnnnancnnnannanaanggnnnnnnngnnnntgnnnnnnnncnnannanggnncnnnnnnnnngngngancgcnnnccnnnnnnnngnnnancnngnnaaagnganaccnngnatnntnnnnangnnncnanannnngngtnnnnnnnnannnnnnnnnnnngnggggcgnnngcngnnnccnnngnngnnngnnnnnnnnnnnnncnggnnaaaaannnnccnccccnncnnnnnncncnnnnnnaannnnntnnnnnnnncnnncccnngnanncnnngnnnnnngnannnnnnngngnacgnnnnnngnnnngnngnnncnnnnntnnnnnncgnnnnnnngnnnnannnnnnnnnnnanannnnnannnnnnnagngngnnngnggggnngntnntngnatgnncnnnnnnnnnnnnnnnncnnnntnntnnnnnnnnnnnnnnnnnnannngnnnnngnncnnnnangnnnnnnnnnnnnngnnnnnnnnnnnnngnnnnnnnnnnnnnnnngnnnnnnncncgnnnnnnnnnggngnaaaaaaaatnncgnnctnnnnnngngngnnnnnnnnnnangngaaanannnnnnnnnnnnnnnnnnnnnnnnnnnnnnngnagnnanannnnnngnngnnnnnnnnngnnnnnnnnnnnngnnnnnnncnncgnngnncnnncnnnnnnnnnnnnncncaannnnnnncncnccnannnnnnnnnnnannnncgncngnnaannnnannnannnannccnnnncnnannnncnnnnannnngnnnnnngnnngnnnnnnnannnnnnnnnnnnnncnncgnnngganngnnnnnnnnnnnnnnnnnnntnnnnanggnggnnnnnngnnggnannnnngnnnnnnnncnnaannnnnngnnnngcgngngnnnnnnggngnnnnnnncnnncnnnnnnngnnnnannnnnnnnnnnnnnnnnnnnnnnnnnntnnngntnnnnnnnnnnnnnnnnncnnnncnnnnnnnnnngnnnnnnnngnnnnnnnnngnngngnnancngtngannananaanngncgaanaagtnngngnnnnnnngnngnngnncnncccnanncnnantnnncgnannngntgagannnangnggnnaantcnnnggccnncngncnngnnngnncannacncggnnngnnncnggnnngaanananggggggannnnnnncnggggnccncnnnnnnnnnannanangaaaaanaaanagcgn

SEQ ID NO: 6 - DNA, 643 bp, Homo sapien
  misc_feature (1)...(643): n = A,T,C or G
cctttttttttttttttttttctgaaaaaatgaaggcacatttattaaatgactgggagaaattccatagtatgtagaatgggaataataatacataacattgtattttatgttccattttttaaaatgagtccaaggaagttaaaatattcttttaattaagacactcaaagaaatgaaataagaaaaattgatgcaaggactccttcaagttaanatttgtgatacaaatattttcatcttttaacagggcaagctgatgtgttcacatctcagtttcaagctgcctctttcactaggaacatcagtatttttttttaaaagcacatttacaatgctttcccatcacccttgctgtgtttttgtagcacctatagccataactggcacctgggggcctgcgttgctggcantttcccttacatttctttggagtcttttcaactgctgggggtttacttaaaagtcagtgctttgcatatttgatttcctganantgnttgaatagnntttttaaaaaaatgngcaggctgggtgggacannttttttncaagggaatganannancntgctnnggttggntngcttggaatgggtccaaccnnncctnnttttnttcccnancccttnccngcccngcct

SEQ ID NO: 7 - DNA, 123 bp, Homo sapien
cctcgcccgtcacgcaccgcacgttcgtggggaacctggcgctaaaccattcgtagacgacctgcttctgggtcggggtttcgtacgtagcagagcagctccctcgctgcgatctattgaaag

SEQ ID NO: 8 - DNA, 655 bp, Homo sapien
  misc_feature (1)...(655): n = A,T,C or G
gtaaaacccagcccatgacccctaacaggggccctctcagccctcctaatgacctccggcctagccatgtgatttcacttccactccataacgctcctcatactaggcctactaaccaacacactaaccatataccaatgatgggcgcgatgtaacacgagaaagcacataccaaggccaccacacaccacctgtccaaaaaggccttcgatacgggataatcctatttattacctcagaagtttttttcttcgcaggatttttctgagccttttaccactccagcctagcccctaccccccaactaggagggcactggcccccaacaggcatcaccccgctaaatcccctagaagtccccgtnctaaacacatncgtattactggnatgaggagtatcaatcacctgagctcaccatagtctaatagaaaacaaccgaaaccaaataattcaagcactgcttattacaattttactgggtctctattttacccttctacangcctcanaatactttcgagtcttccttaacatttccgacggcatctaccggttaacattttttgtagccacaaggtttcacggacnttccctatcattggctnacttttcttactattggttattcgccaataaaattcacttttnntccnag

SEQ ID NO: 9 - DNA, 663 bp, Homo sapien
  misc_feature (1)...(663): n = A,T,C or G
ccggagccgaaacaccggtaggagcggggaggtgggtactacacaaccgtctccagccttggtctgagtggactgtcctgcagcgaccatgccccgtaaaggcacccagccctccactgcccggcgcagagaggaagggccgccgccgccgtcccctgacggcgccagcagcgacgcggagcctgagccgccgtccggccgcacggagagcccagccaccgccgcagagactgcaagtgaggaacttgataatagaagtttagaagagattttgaacagcattcctcctcccccgcctccagcaatgaccaatgaagctggagctcctcggcttatgataactcatattgtaaaccagaacttcaaatcctatgctggggagaaaattctgggacctttccataagcgcttttcctgtattatcgggccaaatggcagtggcaaatccaatgttattgattctatgctttttgtgtttggctatcgagcacaaaaaataagatctaaaaaactctcagtattaatacataattcttgatgaacnccaaggacnttcagaattgnacagtanaaagttctttttcaaaaaataattggttaagggaaggggtngattttgaaancntttcttaacnnaantttttngntttcccaaacggctnt

SEQ ID NO: 10 - DNA, 654 bp, Homo sapien
  misc_feature (1)...(654): n = A,T,C or G
gtcggggtttcctgcttcaacagtgcttggacggaacccggcgctcgttccccaccccggccggccgcccatagccagccctccgtcacctcttcaccgcaccctcggactgccccaaggcccccgccgccgctccagcggccgcgcagccaccgccgccgccgccgcctctccttagtcgccgccatgacgaccgcgtccacctcgcaggtgcgccagaactaccaccaggactcagaggccgccatcaaccgccagatcaacctggagctctacgcctcctacgtttacctgtccatgtcttactactttgaccgcgatgatgtggctttgaagaactttgccaaatactttcttcaccaatctcatgaggagagggaacatgctgagaaactgatgaagctgcagaaccaacgaggtggccgaatcttccttcaggatatcaagaaaccagactgtgatgactgggagagngggntgaatgccnnggagggggcattacatttggaaaaaaatgtgaatcaagcactactggaactgcaccaactggccctgacaaaatgacccccatttgngtgactttnttgaaaccatttacttgatgagcaggggaaanccttcnnnaatggggngacacgngaccaacttgcgnnt

SEQ ID NO: 11 - DNA, 653 bp, Homo sapien
  misc_feature (1)...(653): n = A,T,C or G
tttttttttttttttttttttatgggaaactgctctttatttagacctttgggacaaaattaactttggtcacatattacttaaaaaaaaatccagttttacatatttctaaatagatagaactaaatgatcagagaatttcttctgtaaaaattggccaaattttatcaaaaatctaacatacgatacaatccaaattataaaaagactacttgggatcataatattccaaatgtatgacagttataactccatcttaacaagtgtgaaaagtacttgctctcatgttgctttggtccaaaagagtagagctaactcagtaacaggaaactaagtacccaatcttttgccaaaattaatttagattgtgactggcagcanaaatatccataatgaacagctctactataacaaagaataattaaagaatacttttcgtgaacatatcacaggtcaaatacatttttataagagaaaaatatgaaggaaatgatnaaatagctntcncaaacaaaaaggaagcatttnccccntaaggggaattaanagggtggatgatgcttatatgaaangaagtngaanncngntttatttcttatttttccactcttanctttcaaaatnggtttgncatgccctaaagngaanccngg

SEQ ID NO: 12 - DNA, 375 bp, Homo sapien
  misc_feature (1)...(375): n = A,T,C or G
ttttttttttttttttttttttttttggnattataaanacatttatttaatctatgaaaataatgnacaataaatactttccccttttcctattattaaanaattttaataaataatntacagtctaaaacataaaaaagaggaaaataggnccctctagttatttttaanaaagnccccctanagtttaattattcctganatttcattggaaggagtctaccaaacggaatttttctgngngaattttaaaanataaccgagtgcccaatattttagaagaagaagaaaggaagnggattaaacgctaattcagtaatacctgaattttagcaaaacacataagtctatgcgactgagggngggagangntcg

SEQ ID NO: 13 - DNA, 658 bp, Homo sapien
  misc_feature (1)...(658): n = A,T,C or G
ctctctctttcactgcaaggcggcggcaggagaggttgtggtgctagtttctctaagccatccagtgccatcctcgtcgctgcagcgacacacgctctcgccgccgccatgactgagcagatgacccttcgtggcaccctcaagggccacaacggctgggtaacccagatcgctactaccccgcagttcccggacatgatcctctccgcctctcgagataagaccatcatcatgtggaaactgaccagggatgagaccaactatggaattccacagcgtgctctgcggggtcactcccactttgttagtgatgtggttatctcctcagatggccagtttgccctctcangctcctgggatggaaccctgcgcctctgggatctcacaacgggcaccaccacgaggcgatttgtgggccataccaaggatgtgcttgagtgtggccttctctttgacaacccggcagattgncttttggatctcnanaataaaaccatcaanctattgaataccctgggngtggtgcaaatcccntgtccangaaganaaccncttcanaangggggtctttgtgnnccntttttnncccaacncaacaaccctnttattnnntncctngggttggaaaanctggcnnggntnganccggntnactggg

SEQ ID NO: 14 - DNA, 686 bp, Homo sapien
  misc_feature (1)...(686): n = A,T,C or G
ccttttttttttttttttttttttttttttaacattatactgncatttttatcataacaatataaacaatttttatcatcatcctgaatattactttataaanatatatattttaaaaggntttcaaaacatttttcaacccagcatttgagaataaagcattaagagttttgnatacagtaacacattcatgngataagngnatgaatttacaaccatacataatatggatatatggatatatatttatataaaaaacaaacttggccanaagttaaggntacctacnaagttgtccaagtaaattatgcttggcaaaacaattataaaattcaaatcacacatgcatttttaaatcatctaaatcactgcaaacaanggtcaagcattccaaangttttaaaatnangggggangangggaancnggccctccaannttaaagggcccgtttaaaacccccttgaccccccccccacaggngntttttaactnccncccatttntgttgtttgnncntttcnccggggccttctttggcccttgganggggccncccccccctgggccttccnaaataaaagggaggaaaanngnnttcccacgnccccccccgnatgnatnctctcctntaaaaaaangggngggncgngannctaannggagnggtttggcnaancacttct

SEQ ID NO: 15 - DNA, 725 bp, Homo sapien
  misc_feature (1)...(725): n = A,T,C or G
cctttttttttttttttgatttttacaaatattgnttattttaatgaagctggtacagacaatgtccatttaaaacccatatcccaggccaaaaagtacaaataaaatcaaaaagagcagtgttctgntgtattcatttctgnatgtatagctttattaattngctaatgaaaattanaacttttctgggatcttctgacaagatttttaaaaaatcttaaaatgccttttcttcagtgaaggcacttttggagttnccaataaaggggnccccccctnccatcttnacttnaacctgatattnntnttgtgnnggggggggngggngaaattttaaaaatatnttaatttaaggaaaggncattttttcacagtctaagttctntgnaaaacttncattttcccacngaaagnganagttnangaanncccccnngggcncnccccaccntgnggggcaanttgnaaantnattatngaacncttggtattgnttgaattntttntgntaacgnnnaattgcgtgnaagaangctatcgttnctgtaaaaaaaaggggaaacttttnctatantntccnntannttctttttanaaaccccnaccccccctaaatgtganccnccgatnttttnccggggntggatnttnntcngcccttcncncnccgccctttttttanacgccnatttatattttntaantttatntaanttctcatntct

SEQ ID NO: 16 - DNA, 196 bp, Homo sapien
  misc_feature (1)...(196): n = A,T,C or G
cngaaggtngcctncaccctggcatcctcccctccttccnacttntgcccccaccccatgtctctgtccttgtcccagccaggccctgctccctctccagccttgacagcccctccccctgcctatgccaccctgggcccccgcccatctccagggggctctgcaggggccagttcctccgcctgtcctctggggg

SEQ ID NO: 17 - DNA, 667 bp, Homo sapien
  misc_feature (1)...(667): n = A,T,C or G
cagccgtgaaactggaaagtcattttgatgactgatgtgatacatccagaggtaaaatgcatttaaacatattaaagtatttgccaaagatacaattttcttgctgacataaaaatcacacaaacaagtcccccccaaaccacaactgtctctcaaatagcttaaaaaaattgaaaaacattttaggatttttcaagttttctagattttaaaaagatgttcagctattagaggaatgttaaaaattttatattatctagaacacaggaacatcatcctgggttattcaggaatcagtcacacatgtgtgtgtgtctgagatatagtctaaattagcaaagcacatagtattacatacttgaggggttggtgaacaaaggaaaaatatactttctgcaaaaccaangactgtgctgcgtaatgagacagctgtgatttcatttgaaactgtgaaaccatgtgccataatagaattttgagaattttgcttttacctaaattcaagaaaatgaaattacacttttnagttagnggnggcttaacataattttttctatnttaacccgtattnaaatctcaagtaagaatttnccgtggcccgaaacttgttangggggaattttaaaagggcctcgcattccgggttacatggcntanaantggaagg

SEQ ID NO: 18 - DNA, 1493 bp, Homo sapien
  misc_feature (1)...(1493): n = A,T,C or G
ccccatttctccattttgtggaccaagccatcctgagggcatggacattgtctctgaggaaattggggccacccttaagataccaagaaaagctcctgcccatggtcccactggaaatggactctgctgagcaaagccaccagttgaagagaacagaatccacacctgcattgaatacctgtttctccatgtgtatcgtctctgagattaccttcttgccctttccaacaccttagtgattcctcaatttctcccccattgggaaggccatagggcattaactgaaggaactgacctctctccttttcctgtacctttaacctttagtctgtcaaggaaaacccttaggacctctgaatcaagaggactgagtttgtgggtgaaccttgaaggtgctctttctgctacaagggccctgggagatagcatgggacgtgcattgagaagccagcctcagaccttagcttgaagcancttgaggccagacctactgtacctcagcatcttgctaggaggcatggaagtgatctatcctgccaggaggcctcagagtgatctgtcctgccaggaggggtgagagtgatctgtcctgtgaggcatttaggggctttaggaattantaaaagggggagtatgcctttccagaatcttccatcttcctttgganacctggccttcctcccatttcctccctttggccccaggtangaaggatggagggaggnttgttactnttncccttctgggggccctttctgggggcctaaccctgncaattttanttccnccctcccttacctngggatgnngggnccctttnccgggatttaanccttggggctgggccctaanttttttcccttttttttcccnaaaaaaaaaaaaaggggggggccccccctgnnnnngnntttttnnaaaatnccccccnngncntnnggncccnnccnnnccccnntttnnttnanccncccctggggggtcccntttngggggnnnttnnntttnnanccnnnnnnnggggntttttttttnnnnnaaaanttttttttnnncnnncnnnnncnnnnnncnntttnnnnnngggggggnggntnnnnnntttnnannnccccntttttnngnnnaaaannccnnnnnnnnnnggggggggnnnnnnnnnnnnnnnnnnnnncncccccnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncngnnncnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnc

SEQ ID NO: 19 - DNA, 1602 bp, Homo sapien
  misc_feature (1)...(1602): n = A,T,C or G
ggaaaatcaagatgtggctgaagatcagaggctcagttagcaacctgtgttgtagcagtgatgtcagtccattgattgtctttagagagttaatgttacaaaaaagaattcttaataatcagacaaacatgatctgctgaggacacatgcgcttttgtagaatttaacatctggtgtttttctgaaaaaatatatatacatatattgctttatttgaaacaaattaaaatatgctgcatttgaaaaaaaaaaaaaaaaaaaaaaaaaaaaaaaaaaaaaaaaaaaaaaaaaaaaaaaaaaaaaanaaaaaaaaaaaaaaaangggggggnccccccccnggnnngnntttttgnaaantcccccccccnnganntngggnncccnacnncggccccanntttantttaancccnnccccccttggggcccccctnnngggggggntttnatttccaaaancccccaanngngggggttnttnntttcnccaaaaancnntttttttnnaaaccnccccccggaacccccnccccccctttttcntttaagggggggnggggntttnttcccccctttttggaaaancccccttttttttttggggggcccaaaaaaaaccccccctttngnaccnnnnanggggggggggggggnaancctttgggaaaacccccccncggggagngaaaanccccttttttccccccccccctttttgttttcctnngccccaaaaaccccntcccccnntggggganntnggcnggnganncnannancccnnaaaangncccccccccccnnnnggngaaaaanncccccnnaanggggnttntntcccngggganaaaaancccnggggggggncnttttcccccgtttngnccccnaaanggggggggccccccttgggcnnnnaaaaaccccctttnntncccncccccgngggggggnnntttccccccnaaantccccccccctngccccnaangggaaaacccccnnngnggggtcccttngggnnccccccnnttttttcccccccnggggcggggggngnngggggggannccccgnnggggcctttccnnnngtttttccncccnncccctntnnngggggtgaaannaacccccccnngnnttnntnanccccccnanannnngnccccnntttttgtnccccccnccngaanncnnacccccccccccnanntttttttgnnnngggncncccccngngnntnnttnnccccccccccccccccccccgggggngnggntttttttgnnnnnnnnnncccccngggggggngccccncccccncncggnntttgggngnnncccccctnntttntttnnnnccncccccccccccccgccntttnngnnggnggngnnnnncngcncccccctnnngntcnnttntcnccccnccnnnnnnnnnnnnnnnnnnnnnnnnnnnnnnnnnnnnnnnnncnnnnnnnnnnnnnnntnncncncnngcnntcnnnnnnnnnnnnnnnnnnnnnnnnnnnnnnngnnngngnnnnnnncnnnnnncnnncnnnnngcnnnnngnnnngcnccgcccncnnncc

SEQ ID NO: 20 - DNA, 1633 bp, Homo sapien
  misc_feature (1)...(1633): n = A,T,C or G
agcacgccagccatcagcccctgaatccacctcacccactcgccagacctttttgtcgaagttcatgtccttccttagccttccaatgaagcctctacctgcctgagatgtccaaggtaatccatcagctgaggctctcagagaatgaaagtgtggccctgcaggaactcttggactggaggagaaagctctgtgaggaaggacaagactggcagcagatcctgcaccacgctgagcccagggtgcctcccccaccaccttgcaagaagcccagccttctgaagaagccggaaggggcctcctgcaacaggctgccgtctgagctctgggacaccaccatttgatgtggcctgaactgcagacttacaaaatagaactgcctactgattccgggctgcaacaacagaaggctgccttctgacatgcgctggggcttctctccacgcatttagacaaaaaaagcacaggacacagacactaaatatatgagatcccgtgtgtgtgtgtgtgtgtttgtgtgtgtgtgtgtgggttctttcttatccatctcgnggngatacactctgattttcaagctcctcatttacgggtcttgtgctacccctaggtancaagaaaanaggctgggaaaaagtgtggncgtggncnanagcgananaagtancggnnggaaaggagcnantccatgcacacttctgtaccngtngttttttntacnggntcaaacaggnntgnntnattggncnttnccaangggggttttntttantaannaccnngnnntnncnggggannaanannannnnnnnannnnnnntttnggnnnnccncccttgggggggnnnnanttggggcncnctcnctcccccccctcncncccccctcccccttcacnncgncncnccntnnnccncggcgcnnctccncntcnncnnccnnnntcgncccnnnngngggggggcggggnngnnccccnctctnctccncnnnccccccccnncnccnncncnncnncncccccncccncnccnnnncnccccccncnnccccnccccccnnnnnnngnnnnnnnnnnnnnnnncnnncccccccccccncccccccccnncnccnnnnnnnnnnnnnnnnnnnnnnnnnnnnnnnnnnnnnnnnnnnnnnnnnnnnnnnnnnnngggggccnngnnnnnnnnnnnnnnnnnnnnnnnnnncnnccccccccccnnnnnnnnnnnnnnnnnnnnccccccnnnnnngnnncnnnnnnnngnnnngngggggnngnnnnnnnnnnnnnnnnnnnnnngnnnnngnnnnnnnnnnnnnnnnnnngggnnnnnnnnnnnnnnnnnnnnnnnnnnngnnnnnnngnnnnnnnnnnnnngnnnnnnnnnnnnnnnnnnnnnnnnnnnnnngnnnnnnnnnnnnnnnnnnnnnnnngnnnnnnnnnnnnnccgnccccccgnncnnnnnnnnnngnnnnnnnnnnnnnnnnnnnnnnnnnnnnnnnnnnnnnnnnnnnggggnnngcggngnngngggnnnnnggnnnnnnnnnnnnnccnncccccnnnnnnnnnnnnnnnnnnnnnnnnnnngnnnnnngnnnnnngnnnnnnnccnnnccccccngnnn

SEQ ID NO: 21 - DNA, 1462 bp, Homo sapien
  misc_feature (1)...(1462): n = A,T,C or G
gggctcccaaaatggcgaagtgaggctgcggggactcgctgagcagcggagggggagcgtgcagagccgctgcggccctcacagtccggagcccggccgtgccgtgccgtagggaacatgcacttttccattcccgaaaccgagtcccgcagcggggacagcggcggctccgcctacgtggcctataacattcacgtgaatggagtcctgcactgtcgggtgcgctacagccagctcctggggctgcacgagcagcttcggaaggagtatggggccaatgtgcttcctgcattccccccaaagaagcttttctctctgactcctgctgaggtagaacagaggagagagcagttagagaagtacatgcaagctgttcggcaagacccattgcttgggagcagcgagactttcaacagtttcctgcgtcgggcacaacaggagacacagcaggtccccacagaggaagtgtccttggaagtgctgctcagcaacgggcagaaagttctggtcaacgtgctaacttcagatcagactgaggatgtcctggaggctgtagctgcaaagctggatcttccagatgacttgattggatactttagtctattcttagttcgagaaaaagaggatggagccttttcttttgtacngaagttgcaanaatttganctgccttatgtgtctgtcaccagccttcgagtcaanantataanatgtgctaaggaaganttattgggactctcctatgatnacnattnatgganaacccggttggccttnaaccttctttttgctcanacggtnttaaaatatttagncgnggggngggatctttggtcacccaaggaaaaannacccggnaantttaaaattttttgnnaaaaaaaaaannnttccnaaaaagggaatttctttnaaanttggccccaaanaccttgnggnnctttnggnnnntttgnncttttnanncccnnngggggnngnnttnccnnaaaaaaaatttnntttnnngggnnnnncnnnnncannnnnaannnnnnnnnnnnnncccnccngngnnnnnnnntnnaaagnnttttnnnggnncccnnaaaatngggggnncnnttttttnttttnccnnnnnnnnnnnnnnnnnnggggggggggggncccnnnnntttttnnnnnannnnnnnnnnnnnnnncnnnccccnnntnnaaannnnnnnnnnnnnnnnnnnaannnnnnnnnnnnnnnnnnnngggggggnnnnnnnnnnnnnnnnnnnncccnnnnnnnnncnnnnnnnnnnnnnnnnnnnnnnnnnnnnnnnnnnnnnnnnnnnnnnntntntngnnnnnnnnnnnnnnnnnnnnnnnnnnnnnnnnnngnnnnnnnnntnnnnnnnnnnnnnnnnnnnnnnnnnnnnnnnnnnnnnnnnnnnnnnnnnnnnnnnnnnnnnnnnnnnnnnnnnnnaaaaan

SEQ ID NO: 22 - DNA, 1601 bp, Homo sapien
  misc_feature (1)...(1601): n = A,T,C or G
cccgaagcacgacgcagagcctccggtgtggctgtctctgatggtgtcatcaaggtgttcaacgacatgaaggtgcgtaagtcttcaacgccagaggaggtgaagaagcgcaagaaggcggtgctcttctgcctgagtgaggacaagaagaacatcatcctggaggagggcaaggagatcctggtgggcgatgtgggccagactgtcgacgacccctacgccacctttgtcaagatgctgccagataaggactgccgctatgccctctatgatgcaacctatgagaccaaggagagcaagaaggaggatctggtgtttatcttctgggcccccgagtctgcgccccttaagagcaaaatgatttatgccagctccaaggacgccatcaagaagaagctgacagggatcaagcatgaattgcaagcaaactgctacgaagaggtcaaggaccgctgcaccctgcagaaaanctggggggcagtgcccgtcatctccttgaaggcaaagccttttgtgaacccccttctggccccctgcctggaagcatcttggcaagcccccccncctgccccttggggggttgcnaggcttgccccccttccttcccanaaccggaaggggcttgggggggatcccccancagggggggaaggggcnantccctttnccccccannttggccnaaaccngnnccccccccncccccttggnantttttccnttnttncccttcccatncccntttngcnggggtnnttnggnccctttccccnaaanntggggntttttngnaancntttttnnaaannnncccntntttgggggnctnnnnaaannccnnaanccccnanngtntnncccccccccccnngggncccccccccccnnntnttntnnnngggggggggnnaaancccccnnnnnnnnnnnnnnnnnnnnnnnaaaaanaaaannantncnccccccnntttttcccccccnccccncngggggnnccnnntccccccccnttttttccccnannnnnnntgggnnncnnaannttttttttnnanccccncnnnntnnnnnnnnnnctcnnngnnnnnntttnncnntntnttnnnnnnnnnnnnnnnnnnnnnnnantnnnnaannnnnnnnngnnaaaacnatnccccctcnctttnnccccnnggnnncnnnnncctttnnccccnnnnnnnnnnnnttttnccngnnnnncnnnaanggcnccttnnnntnaannnnccccttcccnngnnnngnnnccccaanggnganaantggggnnccccccccccnnngcnnnnnaantttnnnttngggggnnnnnnccccccgccgcgcctcccnctccccttcgcgccgccccgcgccgccgtccgccccgcccccccnctcccnctccccgccgtcnctcncttcnctctccnccgcgccccgcccgcgcgcccgctcgncgtcncgncncncnncnccnnnnnnnnnnncgnnnnnananaagnncnccnaccnatcccccccgccnnccccccntnccgnnnnngnnnnnnnnngnncgcccnccncccccnncccccnttcgtncccccccnttn

SEQ ID NO: 23 - DNA, 1566 bp, Homo sapien
  misc_feature (1)...(1566): n = A,T,C or G
tttttttttttttttgattttttttaatgctgcacaacacaatatttatttcatttgtttcttttatttcattttatttgtttgctgctgctgttttatttatttttactgaaagtgagagggaacttttgtggccttttttcctttttctgtaggccgccttaagctttctaaatttggaacatctaagcaagctgaagggaaaagggggtttcgcaaaatcactcgggggaagggaaaggttgctttgttaatcatgccctatggtgggtgattaactgcttgtacaattaccgtttcacttttaattaattgtgcttaaggctttaattaaatttgggggttcccttcttagagcagctcgtactgacgaaggtgcatgcgctgaatgatgtcacggcagtcgttgaacacacggcggatgttctcagtgtcccagcgcangtgaaatgagggtagcagtagtgacgcccatctccactggcagtgctgatcctcagaaactcatctcgaatgaagtacttggcccnggtcacgcgtgggtnctcttcnggctcnggagtancatnctcangagtagggtagcgagcaaattctggaaagaagcctcaatcttcnatttcccnncaaggactttctcancganccanatcttgcttgtttganggaaccaggaatcccngnnnaatggngcncaaccccttcttgttggttncccaaaangcccnttgaaaaaagggttcaaaaanccctccctgccanggccggggttngggncctgggnttgnccccccccccggnaaaaaancnctnntttnnnnaaancttgnnttggnttggggncccccccccccnaaaaaaaaanaaaaggggnnnnnnnccnccccnntnntttnnaaaaanaccccnggggnanncccccccttttggggggggggnntnnntttnnnnnncnnngggggcccccccccccnnnnnaaaaanaattntggggaaannnnnnannttttttnnccccccccnnngnnaaaantnngnnntnncnnaaaatnncccnaaannnnnngcccccnnnnnnnnaaaannnnnnnntnnnnnnnnnnnnaanaannnnncccnntntannncnnnnnnntnncnnaaaanngnggcncnnnannnnnnnnnncntngnnnnnnnnnnnnnnnnncnntttttnnccnnaanntnnnnnntnnnnnngnggggnnaannngncnncncccnccnaannnncccncnnnnggggnnnccccnnngggcccnnnnnnnnnnccnngnnnnnnnnnnnnnnnnnnnnnnnnnnnnnnnnncccngnnnnnnnnnnnnnnnnncnnnnncnnnnnnnnnnnnnnnnnnnnnnnccnnnnnnnnnnnnnnnnnnnnnnnnnnnnnnnnnnnnnnnnnnnnnnnngnnncnnngncnccncnnnnnnnannnnnnnnnnnnnnnnnnnnnnnnnnnnnnnnnnnnnnnnnnnnnnnnnnnnnnnnnnnnnnnnnnnnnnnnnnnnnnnnnnnnnnnnnncnnnnnnnnncnnncccc

SEQ ID NO: 24 - DNA, 651 bp, Homo sapien
  misc_feature (1)...(651): n = A,T,C or G
cgtcggttggcgactcccggacgtaggtagtttgttgggccgggttctgaggccttgcttctctttacttttccactctaggccacgatgccgcagtaccagacctgggaggagttcagccgcgctgccgagaagctttacctcgctgaccctatgaaggcacgtgtggttctcaaatataggcattctgatgggaacttgtgtgttaaagtaacagatgatttagtttgtttggtgtataaaacagaccaagctcaagatgtaaagaagattgagaaattccacagtcaactaatgcgacttatggtagccaaggaagcccgcaatgttaccatggaaactgagtgaatggtttgaaatgaagactttgtcgtgtacttaggaagtaaatatcttttattagagaaagtgttgggacagaaagtactttatgtaactaagtgggctgttcagaacttanaggcattttttgtaatttctttttaattactttananagctagggatgcaaatgttttcagttagaaagcctttatttacttttggaaattgaacaanaaatgctttgtcttanaactggagaatatttgatggtagggaaacatgtaatggttctctggcaaaattgnntcannatttgaaatgaaannn

SEQ ID NO: 25 - DNA, 676 bp, Homo sapien
  misc_feature (1)...(676): n = A,T,C or G
gggggacagagactcagatgaggacagagtggtttccaatgtgttcaatagatttaggagcagaaatgcaaggggctgcatgacctaccaggacagaactttccccaattacagggtgactcacagccgcattggtgactcacttcaatgtgtcatttccggctgctgtgtgtgagcagttggacacgtgaggggggggtgggtgagagagacaggcagcttgnanntnnttgcttnganntttcncntanaacccgcnagcgcttnggtagggtnngcnanggatgncnnncnttnttcnnaagncnccngttcngngtcanttgcttgnctcntctaactcnnnnnncccccnnttnngtctcctnngngntcnacccnntctgnttcttngntcnngnttgncctcgnnnttnnttcnnngctcngcncgtntggtgnnntgngnatnannctnancgngtttntnnattntnnctnncgtngancncatntganccttntnnngntnttcgnctnnntcgancgtnttcngggncncnccncgnntctnnctnncctcncccttttntcntcttgnttgtggcntnacctnnctcnttctntgtntncnngccttnnngtgnnncngatagtcnnccctntttgnnaatatctntntnntcncccccctccc

SEQ ID NO: 26 - DNA, 657 bp, Homo sapien
  misc_feature (1)...(657): n = A,T,C or G
tttttttttttttttgctgggtggtaactctttatttcattgtccggaagaaagatgggagtgggaacagggtggacactgtgcaggcttcagcttccactccgggcaggattcaggctatctgggaccgcagggactgccaggtgcacagccctggctcccgaggcaggcaggcaaggtgacgggactggaagcccttttcanagccttggaggagctggtccgtccacaagcaatgagtgccactctgcagtttgcaggggatggataaacagggaaacactgtgcattcctcacagccaacagtgtaggtcttggtgaagccccggcgctgagctaagctcaggctgttccagggagccacaaaactgcaggtagtgatgtgcaagantccatcctgcagttttccagcaatganaaactcctcctgcggttgtgggacctggggaagtatccgcanacctctcctggcgggggtgtagacnaaccggatgtcaccggcatcccctaaagnttggaaccctttatacatcttgggcatcttganctcataacgctggtataaggnggntnggtngacttttggnngtccccccaantgcccttganaccaaggccgnaattncnaaaggcccctgngggggggggggacccagn

SEQ ID NO: 27 - DNA, 646 bp, Homo sapien
  misc_feature (1)...(646): n = A,T,C or G
ggaangctgaagaattaacantttgactnctaaatgtgatactggntngtanattcccttagagcagaaaggagaggggcacatattaatttgtatcgcttttgcttctctttggtcttttgtgtcttagaatttggaagtggttcatttctgttgctggtatgaggatttcgaatacttagtaatcgaaaaccatatcctgtaatttaataaaaaaaactaaggaagaaaaaaccctccaattttcccaaatgcaatcagtgtaactaggggctgtgtttctgcattaaaataaatgtttcangctttgtggtcctgatcaaggtcctcattaaaaaattggagttcaccctagngcttttcccctctgtgactgggctcntcccccaccnctcttaggtatcgcagttattatgggntncaaatnaagnaatangnttnncaaatttnaccaaanaaagcatttttttcactgcnttntnattggggggttggcccaaccncntcaatggntcttancatggntggntacccgcnacctttncntnaacttggngnaancnngggcnntacnnttcctgggggnaaatngtntccnnccantccccncncntncnanccgaancnnaaagggnaancnnggggg

SEQ ID NO: 28 - DNA, 407 bp, Homo sapien
  misc_feature (1)...(407): n = A,T,C or G
caagagtctttgaaataagcccatttgagccctggataacaagggataaagtggagcggatgcacatcacagacatgaaattgcctcacctgcctggcttagaagaccttggtattcaggcaacaccactggaactcaaggccattgaggtgctgcggcgtcatcgcacttaccgctggctgtctgctgaaattgaggatgtgaagccggccaagaccgtcaacatttagtgcctcctgagcagctcttggttttggcgtcttttgggtcggcccatgtggtttgagcacccagccaggcggtctctttagaggatcctgtacacagttccactattaaaacatttcaggttgaaaaanannnannnnnnnnnnnnnnnnnanannannnnnnnnnnnnnnnnnnng

SEQ ID NO: 29 - DNA, 625 bp, Homo sapien
  misc_feature (1)...(625): n = A,T,C or G
tttttttttttttttttttttttttttttggggaccaaatttctttntttgaaggaatggnacaaatcaaacgaacttaagnggatgttttggnacaacttattgaaaaggnaaaggaaaccccaacatgcatgcactgncttggggaccanggaagtcaccccacgggtntggggaaattancccnaggnttanctttcattatcactgnntcccanggngngcttgnaaaanaaanattccncccagccacattnnggcnctcccatnttgcncaagttggncacgtggncacccaattctttgaaggctttcaccngctnattnaagnaangggtctcaatgaaancacaccantggggggnatttttgntnnnngcccattgggcaattcccaanatggctgaatcaaatttttttnccaaagncangcccctccaatggattnaaanccccntnccaatanaacannnggnttttttatcctccaagaaaaattnggcccntntngggntggaaggtttnantattacaagcnccttcctttaaatggggaaaaanttttgnnaaannttaaaaccncntcgccaagntttnaaaagggnaggnangcngngggttacnn

SEQ ID NO: 30 - DNA, 643 bp, Homo sapien
  misc_feature (1)...(643): n = A,T,C or G
cttaagaattggcccagcctcagatcctgtctttagcaaccagctaatatttacccagaggtactgcaatagagtatttcaaaatggaatcaggatctggtgggcctcagaaattgtctcttttctgagtttcaatttggttctcctggatgttttgctctgttttggtacctgtaatatagggaaacacaactttttttgggaaagccctttgaccccagcttgctagttgcataataataaattttctgttcctaaaaaaaaaaaaaaaaaaaaaaaaaaaanaaaaaaaaaaaaaaaaaaaaaaaaaaaggnngnaanaaaaaaataanangggnccgntaaaacnnggggggggcccntcaantttaaagggccctttaaancccctnnnnaanccnccntggnccnttttnnttcccaccttttggnggnnggncccncccccggnctttttttgncctggggggnccccccccctggtcnttncttanaaaaannangaanttgcctcccttntcngaaaanggntctttttttttngggggggggggggggggggaannngggggggggtgggggaaaaattnnggggntttgggaaccnggggcccttgcccttnngaaaagaacccntgggttt

SEQ ID NO: 31 - DNA, 645 bp, Homo sapien
  misc_feature (1)...(645): n = A,T,C or G
gtgaaagctgtaaaacaccttttatggaagaaaagaaataaaatgtagttgtcaagtctaaaaaatagtagcaacgggaatcataatgaatacatgcaatgaatttaaaatgtaaaaatgaatttaaaaagtaaaaagggctctgtggtgtaatttttcttaactacaagagtctaaatacactgcttttctttaagagttcattttaattagtaacgtcaaacaaaattattctagataatgagccctacaaattactactactagcaactgtcattttttactcgggcatcctctaggtgtcttacattctcattttattcttacaacgaactcatcctccagaaggacttcatcctccagaaggactcatcctccagaangactcatcctccaaaggacttctccagaagggggaaatggaagacccgggtaacttgctcagggcttatcacagaactatgtttgagcctgacttcgtttgaactctaaagcccacatgctctttctactgccccatgcttctcaaggnaccagactcttatttnctgcacttttgagaatctnaagatcctgantcattttaaataaatttagttttttggggagnagccnnaaaaaaaaaaaaagggcgccctccncnnt

SEQ ID NO: 32 - DNA, 668 bp, Homo sapien
  misc_feature (1)...(668): n = A,T,C or G
tcccgttctgttttaaacagaaaataaaaggagtgtaagctccttttctcatttcaaagttgctaccagtgtatgcagtaattagaacaaagaanaaacattcagtagaacattttattgcctagttgacaacattgcttgaatgctggtggttcctatccctttgacactacacaattttctaatatgngttaatgctatgtgacaaaacgccctgattcctagtgccaaaggttnaacttaatgtatatacctgaaaacccatgcatttgtgctcttttttttttttatggngcttgaagtaaaacagcccatnctntgcaagtccatgtatgcngcncttaagcnttctatctttgctcaaatngntgaangatggggaccttggctcatggcttgcgnatttgatcntaangnncntttctanctatgntatgaggcacnngccctattggaggncgccccnggtttccggaaaagngcnntnntgtngngaattgcnnctcgganttcaanaatatncggcnnttgntttgnangccnngnnnancaatcaggngngcccctcnaantcatgnaagccccgnntnaanncnctncnctnttctcgnnntgggnnttccattgccngcctcgacgnggttngcctctcnccggcnncncgcncg

SEQ ID NO: 33 - DNA, 682 bp, Homo sapien
  misc_feature (1)...(682): n = A,T,C or G
ggcttgtccgagttgatatgcgtatgctttgcctaaaaagccttaggaaattagacttgagtcacaaccatataaaaaagcttccagctacaattggagacctcatacaccttcaagaacttaacctgaatgacaatcacttggagtcatttagtgtagccttgtgtcattctacactccagaagtcacttcggagtttggacctcagcaagaacaaaatcaaggcactccctgtgcagttttgccagctccaggaacttaagaatttaaaacttgacgataatgaattgattcaatttccttgcaagataggacaactaataaaccttcgctttttgtcagcagctcgaaataagcttccatttttgcctagtgaatttagaaatttatcccttgaatacttggatctttttggaaatacttttgaacaaccaaaagtccttccagtaataaagctgcaagcaccattaactttattggaatcttctgcacgaaccatattacataataaggattccatatggctcttcatattcatttccattccatctctgcccagnatttggggatacccgcanaaaatttggggtttggggggaaaaatntggnctggaactttttttanttnaagggaaataatnaggggnggaaggggggggtttntggntgccccccccccggn

SEQ ID NO: 34 - DNA, 1549 bp, Homo sapien
  misc_feature (1)...(1549): n = A,T,C or G
ttgagagatacctccctccttctgctcagctgccttgcagtaattaaactctttctctgctgcaacacccctactgttctccgtgtattggcttttctgggcagcaggaaggaaaagctgatgcgatgctctcagtgccgcgtcgccaaatactgtagtgctaagtgtcagaaaaaagcttggccagaccacaagcgggaatgcaaatgccttaaaagctgcaaacccagatatcctccagactccgttcgacttcttggcagagttgtcttcaaacttatggatggagcaccttcagaatcagagaagctttactcattttatgatctggagtcaaatattaacaaactgactgaagataagaaagagggcctcaggcaactcgtaatgacatttcaacatttcatgagagaagaaatacaggatgcctctcagctgccacctgcctttgacctttttgaagcctttgcaaaagtgatctgcaactctttcaccatctgtaatgcggagatgcaggaagttggtgttggcctatatcccagtatctctttgctcaatcacagctgtgaccccaactgttcgattgtgttcaatgggccccacctcttactgcgagcagtccgagacatcgaggtgggagaggagctcccatctgctcctggatatgctgatgaccagtgagggagcgcccggaagcagctgagggaccagtactgcttttgaatgtgactggtttcccgttgccaaaacccaggacaanggatgctggatatggcttaacctggggggatgaaccaangtttttgggaangggaaagnttnaaanaaaatcccctggnaaaaaaaantttnnaaanaaaaccttggaanggggccccccttgggaaaaangggggggannnngggttnttnggnccnntttnncccccnnnnnannnctttaannnngnnnanttttttnnaangggggnntnnccccnntttnnaannntntntccccnnnnnangggggggtnncncnnncccccngggggnncnnnntnaacnccnnnctntnggnggaaancntttttttncttcnnccnnggnccccnanannttttcccagaancccccccngggggngnnnggaaangnnnnnnnccctcnngggggttnccccnnnaaaaaaaannnggntttttttttnanganccggggacnccccnnnnaaanntttttnnaaagcgccccccnnnntnnggnnnnnnnggnannnnnnnnttngnnnnttngcccnccnttnnnngnnccnctcnnnnnnnnnnnnnnnnnnnnnnnnnnnnnnnnnnnnnnnnnnncntntanntnntgnaaaaaanggnnnnngnnnnnnnnnnnnnnnnnnngncccccnngngnnnnnnnnnnnnnnnnnnnnggggggnngnggnnngcnnnnnnnnnnnnnnnncnnnnnnnnnnnnnnnnnnnnnnnnnnncgnnnnnnnnnnnnnnnnnnnnnnnnnnnnnnnnnnnnnnnnnnnnnnnnnnncngnngnnggnnanc

SEQ ID NO: 35 - DNA, 1440 bp, Homo sapien
  misc_feature (1)...(1440): n = A,T,C or G
ctaatctaagcctcaaactcgttattggggctataaagaaaacgtttacttacccagctgaaacaggttaagaatattcttaatctcattatagataattgcccccatgggacttgaaatacaacaccttgtgctgaaaacttcaggttggcaatatttgaaggtttcgttgtagaagagtttaacattaactcctattttgacttacaaatcttgtttctcatcactaaaatgcttttgaattaataatccaacccacatgagctgagagtttttcttttgttagaaaagaaacagacatctttctgtatgaaagtataaattgtatggttttagatacataagaattgacaaaagcgagcgaaatctttgtacttctgagttcttgctgtatgtatgttttgttttaaatctgattagggacacccagcagctggccgggattcttggattgctccttgggagttaagattgtcaatactcctgtgaagcaagggatttcagccatagaacaaagatttattgttgccacctgaaaagtttacaagtatttattgtgtatttgatacattgcttgaaaaagatgaaatctgttaaagattcttttccgatgtccaggttaagaagaaacctccttgtattgagtgaaattatatgttaaatggtattagagaatgtaggtggnatagaaattggatttttcttggnggtngaacaacctcaagttcggcaaagtttaaaatttggattaaacaagaaaaannggttcanggttgnaaaangggacttgnttagggangggacaanggcctttaaannaccngcgtcccttctccnggcnggcnngncgggcccnnccccaanctnntccangcnttcgnccncnaccnnccnccttttcctnntnncacnaanntctttnnccntttttacngggggnggggnnnccnncnccggcnncngnntncgccncccanaaannccnncnttttccnncnncccntttncnnnnnctttnnccnnnnccccccccgnnnnnnnnnnnnnnnnnnnnnnnnnnnggnnnnnnncccnnnnnnnnnnnnnnnnnnnnnnnnnnnnnnnnnggncnngggnnnnnttnntnnnnngggggncnnnnnnnnnngcgnnnnnnnnnnngnnnnnnnnnnnnncgnncnnnnnnnnnnnnnnnnnnnnnnnnnnnnnnncccnngnnancnaannncnnnncnnnnnnnnnnnnnnnnnnnnnnnncncnncnnnnnnnnngnnngnnnngnnncnnnnnnnnnnnnnnnnnnnnnnnnnnnnnngnnnnnnnncgnnnnnnnnnnnnnnnnnnnnnnnnnnnnnnngnnnnnnnnnnnnnnnnnnnnnnnnnnncnnnnnnnnnnnnnnnnnnnnnnnnnnnnnnnngnnncgaaganggccnaccg

SEQ ID NO: 36 - DNA, 1496 bp, Homo sapien
  misc_feature (1)...(1496): n = A,T,C or G
tgcataccgtggaagggcgccagggtctttgtggattgcatgttgacattgaccgtgagattcggcttcaaaccaatactgcctttggaatatgacagaatcaatagcccagagagcttagtcaaagacgatatcacggtctaccttaaccaaggcactttcttaagcagaaaatattgttgaggttacctttgctgctaaagatccaatcttctaacgccacaacagcatagcaaatcctaggataattcacctcctcatttgacaaatcagagctgtaattcactttaacaaattacgcatttctatcacgttcactaacagcttatgataagtctgtgtagtcttccttttctccagttctgttacccaatttagattaagtaaagcgtacacaactggaaagactgctgtaataacacagccttgttatttttaagtcctattttgatattaatttctgattaagttagtaaataacacctggattctatggaggacctcggtcttcatccaagtggcctgagtatttcactggcaggttgngaatttttcttttcctctttggggatccaaatgatgatgtgcaattcatgtttaacttggggaaacttgaaaggggttcccatatancttcaaaacaaaaaccaaatggtgttatccngacggatctttttatgggtnctaactagtactttnctaattggggaaaagnaanngctttnagttttgcnnaattaagtttgggggaagggcnataattaaaaattgagggccccgtnacnaaaaccaactgggggngtntaacgaaaaaccctgttttnaaaagggggccttttnccccttnnnnngnnatntnanttnccccntttgccntttccnttttnnnnnaaactttttnnnttttctccccnancnnnnaaangngnanngggtntccccccnangttnnnnttnttcnnnnnannnancccccccttngnggnnccnnnngggcnttttctcntngnnaanngttntnnnanncccttttgncnnnngggnnttgngnttcggnngnccnngggggnnnnnccnnnngnnngnnnnngannangannnnnggnggncgtntnnnnggccgcgggnnnnngngnnncgngnnnnnngnnnnnnngnnncnnngnnnnnnngnnnnnnnnnnnnangnnnnnnnnnnnnnnngngnnngngnnnnnncnnnnnnnnnnnnnnnnnnnnnnnnnnnnnnnnnnnnnnncnnncnnntntnaancnnnnnnnnnnnnnnnnnggnnnnnngnnnnnngngnnnnnngnnnnnnnnnnnnnnnnggnnnnnnnncnnnnnngnngnnngcggnnnnnngnnnnnngnnnnnnnnnnnngnnggnnnnnnggngnnnncnnnnnnnccgcnnnnnngnncncncnnnnnnnnngncnncnnnncnnnngnnnnnncnnnnnnnnngnnntnngnnnnnccgnngnnntc

SEQ ID NO: 37 - DNA, 1604 bp, Homo sapien
  misc_feature (1)...(1604): n = A,T,C or G
atgcagtcctggatggagccgactgcatcatgctgtctggagaaacagccaaaggggactatcctctggaggctgtgcgcatgcagcacctgattgcccgtgaggcagaggctgccatctaccacttgcaattatttgaggaactccgccgcctggcgcccattaccagcgaccccacagaagccaccgccgtgggtgccgtggaggcctccttcaagtgctgcagtggggccataatcgtcctcaccaagtctggcaggtctgctcaccaggtggccagataccgcccacgtgcccccatcattgctgtgacccggaatccccagacagctcgtcaggcccacctgtaccgtggcatcttccctgtgctgtgcaaggacccagtccaggaggcctgggctgaggacgtggacctccgggtgaactttgccatgaatgttggcaaggcccgaggcttcttcaagaagggagatgtggtcattgtgctgacccggatggcgccctgctccggnttcaccaacaccatgcgtgttgttcctgtgccgngatggaccccanagcccctccttcagccnctgtgccaccccctttcccanccaatccattaagncannaangcttgtanaacttcactctggnctgtaaacntggncacntgttnggtngggacaccttgggaaggaaaaatcaacncctcanttgnaaaattggggtaangnttgccantcntgtttttaaangggacnagncgcgaggaagggctnanttnnttanantnnnagggggcccccnnccccnatnnananggggcaaanaacggnanggnaaatngnttnnnnccttngnnngcncccccnnnggannncccnnnncgnggnnnnnnnagnggggntcancnncntncccttntnctnnntgnggtnnnccnnnnnnccnnnnncacgttnaaaannnaaatnnngncccnnnngnnngcctcacncnnttngnggnnngaccnanccaccnngcnnatnggngntggnagggnctctncnncaaancantnnggncttcgtnangngtgnnnnnnnnnnnnnancnngntnnnnncncnnngcnannnngtnncnngnntccncccacttgtntnncnannngngtnnnngnntngannntcnnngnttgnatcccggnaanacnannnncggncncnggcnnnccnncnncngnncnntcccnnncccnatnnnggnggnnnnctgcnanctnnnnngancncnnnnnnnnngncncanncgantngngnngnnnntnncnnnnnnnnnnnnnnnnnnnnnnnntnnnnnnnnccgnttntgctngcagtactntcgngnntntcnnnnnnnngnnnnnnnnncnnnnnnnnnctngnacnttngnacgcnnnagtcgacntnctnggacntnntnnncantcnngccnngtnnngnntngnngcnnacnnnnnnacnnnggcgnnnnnnncncatnncnncnctnanannnggtnngngngnnnccttccnnnnnagnnnnnatanngncnnnanncnccnnnnnnnnnncngnnnnncnnnntcnncgaananntgncacnacg

SEQ ID NO: 38 - DNA, 280 bp, Homo sapien
  misc_feature (1)...(280): n = A,T,C or G
tttttttttttttttaatttatcagngcttaaaaatcttcaaaatagcttagtgaggctcatgacagtgctggccccatggaaatgtagccttttgttgcgtttaaacactgtcacaccatctatgactgtcccattggtctgaagtgtagtggcaaactaagcatcctataagacaagctaaagcttgctttttgccagtcagttgaaagtcttgcatctcttcactgatgcactttctttaggtattgatagtcagaagcacaaagcatttattatgc

SEQ ID NO: 39 - DNA, 378 bp, Homo sapien
cgagtttataatcctataatgaagaatactggcacaggcaatgctcactcgaaaacttcaagtaatttctagttggttttggaatgcttgataaagttcctttacagctttattttcctgatttgttttggtttagatcaaagttcaaattaattttaacttagctaatgaactcatcaccaggacagttggagggggtaggccgaggttaaatggtccacgtttcaaaaatgttaatggctaatccataattaaagaaggtttaactgttactgaagtttacaagttttattgtcatgaacatgaaatacaaacacgatggcttcgaaatgtctttcaataaatgtttctgcatttatatggaaaaaaaaaaaaaaa

SEQ ID NO: 40 - DNA, 2039 bp, Homo sapien
  misc_feature (1)...(2039): n = A,T,C or G
caacttttgtagaagtatttttttctctgtaatatttttattggctcataaagatgttttcatatctgaactcctaaataagtgaaattacagtagattatattaacaaaatactttttaggtagccatgcttgagactttttaaaaatataactttttccttaaagttttcagctatagcaaaaggtagttatgtatgccagacctaatatgagctgccaccaacacccctagaactttcagccatggtgtcttcagaattgtagcgcatttctgaatctagcaaatcctccttttacccgttgaatgttttgaatgccctgactctaccagcgcccataaatgatctctagaaggactgttagtaccaacctgtttttcaactttgaagctaaaaaccctgatatggtaatattatggtgcatagcagaggtctcggaaaaaaaatatttctgttcactttactttcaggttaaaaatgtttctaacacgcttgcaacttcccttatggcattaatcttgttgagggagagagacagaatcctggactctccaaagtatttaactgaaagtagggcctgctctgacagggcccatgtcccacaaggctggcttnggcctcaggggggggctttggctggtgcttgggatgaaaattgntgganncnggtntttggggataaanggaccaaanggaccagccaaaagcnaaaaaatngggntttttaaangccttggggggnttacctttttcntttaaangnnggtttnaaagnattagggctaaangccantttncaaaaaaangctcccnananaaaatggtggaaaagggncccttttggncgacaggncctttgnggaaaattgcccccancngggcccttttttgncccccccnncccaaaaaaaagntgggnngaagnnttnttaaaaccctnnnggngcccntttttttngnnaaanccnccnccnngggggncgccccnctttntttnttntnttcccngggngnccnntttttttncggcngacccnncggggntcaannnctgnanaagnngntatctggcngggnnngcgcnngaaagnnnnnggnnncngngggggnnnncgcncgnnannnttntgnggggnaaaaaaaaaganccctctnttnctctcttntntnaanntnnnnngnnnnnnanncnngcnnnngnngngnngnnnnnnnngncnnncnnannnggggggngggcncncncnncnnnnantnnggggcgnctcntnnnnnnccccnctncgggnnccnnnncnnggngngngcnnntntngnngtccngnntgtgtntgnnnngncnnncncnccncgnnnnnctnnnnntntgnntnngnnngggggngnncnnncccncncgtgnnnnntntnnnnnnnnnnnnganggnnannncnnncnnnnnnnnnnnnggggngcnnnnncnnncnnntnnnnnnngggnggnggggngnnnnnnnnnnnnggnnnngnnnnnnnnnnnnncncncnnnnnnnntgngcgnnnnnncnnncnnngnnnnnnnntnnnnnnnnnnnnnnnnnncnnnnnnnnnnnnnnnnnnnnnnnngnnnnnnnnnnnnnncnnnnnnnnnnnngnggnnnnancgntgngcngnngtnnnnnnnnnnnnnnnnnnnnnnnnnnnnnnnnnnnngnnnnnnnnnnnnnnangnnnnnnnnnngnnnnnncnnnnnnngnnnnnnnnncnntgcgagcnnnngncnnnnncnnntgnnnnnnnnngnntcgcncnnnnnnnnncgnggggcgntnnnnnccncccgcngntgncnnnnnngncnnnnnncnnnnnnnnngnnntnncncnnnnnnncgnnnnnnnnncnnnagngnnnngngnncnncnncnnnatnngannnnnnnnnccnncnnnnnnnnncgnnnnngcnnngnnngnnnnnnnnnnnnntcncnncncnnngnnnnngnnnnnnnnncncncgngngnnnngnncccgtccgcgcgngcgcgg

SEQ ID NO: 41 - DNA, 319 bp, Homo sapien
ttttttttttaaaaaaaaagagtttatttagaaagtatcatagtgtaaacaaacaaattgtaccactttgattttcttggaatacaagactcgtgatgcaaagctgaagttgtgtgtacaagactcttgacagttgtgcttctctaggaggttgggtttttttaaaaaaagaattatctgtgaaccatacgtgattaataaagatttcctttaaggcagaggctggtcgagatgctgctgttatcttctgcctcagacagacagtataagtggtcttgtttctaagattcctaccaccagttactttgggccaagtatc

SEQ ID NO: 42 - DNA, 524 bp, Homo sapien
  misc_feature (1)...(524): n = A,T,C or G
ccttttttttttttttttttttttctgatttcaagtcaagatttattgctttacaaacaaacattatacttggtcttaatagaaaaatgacaccagatacatccaaaatacatttcacattgggatagctgccagttcagcacaaaacatacattactaggagcagggaggcatgaaaataaactatatcttactttttggtacgtcaggaacacttttgcctgaagtaagccctttagtactattttttattttatttatttttttaatccacccatctgcacactggncctttagtactctttaagtataaaactttacttgtcctgggctttgacccttgtgtttgatctaaatgacatttcaaacataaatgtcttttgactagtgcgcttactgntatgtacanaatttaaaatgtgatcgttngaatntaaaatctggtttgatacatgatataaaagttgtatatttaaaatncaagaaatgtttttggggaatatttctactaaagaattttaaat

SEQ ID NO: 43 - DNA, 103 bp, Homo sapien
  misc_feature (1)...(103): n = A,T,C or G
ccttttttttttttttttgcnngaaataaggaatctataaatctgaaataaagaaatcccattttaaattaaattgttaaagagacacataagaaaaaacact

SEQ ID NO: 44 - DNA, 425 bp, Homo sapien
  misc_feature (1)...(425): n = A,T,C or G
gtcgacaagataatgtactgacatctctagcaatcttttttgccagtggctttaaattgccaataagttaaagaatattgttcctatgggttaaatttttattcttattttcacatttaaatttatttttcttaatttttgtggatacataatatgtgtatatatgtatgccatatatggtatattttgatgcaggcatactctatataataatcacattagaggaaatgagatatccattacctctagcatttattctttttattacaagncaattcaattgtacactttttagttatttttaaatttacaatgttattgattacagggtcatttttatggtcataataaaaaattttatacaaaacgtgtaaaatctatacatttctgagttctgaataaatattttttaaaaattttaaaaa

SEQ ID NO: 45 - DNA, 492 bp, Homo sapien
  misc_feature (1)...(492): n = A,T,C or G
gtcgactgcccccaccgctgggcggcgctgcggggcacccaggctctgcagtcagcgccgcgccgggaatcctgtacccgggcgggaataagtaccagaccattgacaactaccagccgtacccgtgcgcagaggacgaggagtgcggcactgatgagtactgcgctagtcccacccgcggaggggacgcaggcgtgcaaatctgtctcgcctgcaggaagcgccgaaaacgctgcatgcgtcacgctatgtgctgccccgggaattactgcaaaaatggaatatgtgtgtcttctgatcaaaatcatttccgaggagaaattgaggaaaccatcactgaaagctttggtaatgatcatagcaccttggatgggtattccagaagaaccaccttgtcttcaaaaatgtatcacaccaaaggacaagaaggttctgtttgtctccggtcatcagactgtgcctcangattgtgttgtgctagacacttctggt

SEQ ID NO: 46 - DNA, 499 bp, Homo sapien
  misc_feature (1)...(499): n = A,T,C or G
ccttttttttttttttttataacatttatataatgtgctaacaatgaatccatccatgatttattgtttgtaatgaacttaaaataaccctttacaaattaaaatcattttttcaaacatgacttcatattgaaatggttctgttaaaaaagtaaaagttgaattttccagccaatttagcatctaggacctgaatcttgccaatatcctacccactatcttcattcctacctcctaccccttcaaatcagctcctccagactttcctatttctgtcaccccagttcaaaatggttttcaccatgcatttgatgtaaaatgtgcaagtgcgatatgacttcacaaagtatcaattgtgtggacaatgataactactgtgacactgctagcacccctggctaaaagtaagaagcaacaaaattacacagggttcctttctgatgaatgcagnanggattcaagaaatcccaganctggaaaaagattttcaatagatctg

SEQ ID NO: 47 - DNA, 537 bp, Homo sapien
  misc_feature (1)...(537): n = A,T,C or G
gtcgacatttttctgaggaatagtttgtgattccaatgcaggtgtcttcattaccattacctctacactgcagaagaagcaaaactcctttattagaattactgcacatgtgtatggggaaaatagttctgaaaggctagaatgatacaagtgagcaaaagttggtcagcttggctatggagtggtggcaataatctctaaacattccaaaagaccatgagctgaacctaaactcccttggaatctgaacaaaggaatataaaattgccatttgaaaactgaccagctaatctggacctcagagatagatcagccagtggcccaaagccatttcaagtacagaaattatagagactacagctaaataaatttgaacattaaatataattttaccactttttgtctttataagcatatttgtaaactcagaactgagcagaagtgactttactttctcaagtttgatactgagttgactgnttcccttatccctcaccctttccccttccctttcctaaggcaatagtgcacaactta

SEQ ID NO: 48 - DNA, 556 bp, Homo sapien
  misc_feature (1)...(556): n = A,T,C or G
gtcgacttttttttttttttttagnnntataaaatattttatttacagtagagctttacaaaaatagtcttaaattaatacaaatcccttttgcaatataacttatatgactatcttctcaaaaacgtgacattcgattataacacataaactacatttatagttgttaagtcaccttgtagtataaatatgttttcatcttttttttgtaataaggtacataccaataacaatgaacaatggacaacaaatcttattttgttattcttccaatgtaaaattcatctctggccaaaacaaaattaaccaaagaaaagtaaaacaattgtccctctgttcaacaatacagtcctttttaattatttgagagtttatctgacagagacacagcattaaactgaaagcaccatggcataaagtctagtaacattatcctcaaaagctttttccaatgnctttcctncaactgnttattcagtatttggccagtacaaaataaagattgggtctcaactctctctttcattagtctcaagngttcctattatgcactgag

SEQ ID NO: 49 - DNA, 355 bp, Homo sapien
gtcgaccgagcctctcccaccctcagtcgcatagacttatgtgttttgctaaaattcaggtattactgaattagcgtttaatccacttcctttcttcttcttctaaaatattgggcactcggttatcttttaaaattcacacagaaaaattccgtttggtagactccttccaatgaaatctcaggaataattaaactctagggggactttcttaaaaataactagagggacctattttcctcttttttatgttttagactgtagattatttattaaaattctttaataataggaaaaggggaaagtatttattgtacattattttcatagattaaataaatgtctttataatacc

SEQ ID NO: 50 - DNA, 507 bp, Homo sapien
  misc_feature (1)...(507): n = A,T,C or G
ccttttttttttttttttaaaaaaaaaaaattctgtttattgtaataattaaataagagtaaacattttaaaacatataaaaataactttaaaatatagtaacactttacaaaatatgtatctaattaaaaatacattaacatagcatccctcaaactatacaaatatagaatatatattcatgaaattctttanaaatataacatctattctttgaataaagcttaaaatttgtttataattttcaaactaanaaaagaagtagngaataatagctccatccaatttataattgtcttaaagagaatgattatgtatcatttcttgcttgtcttttctaatacccagtcaatcacctgtacagcattgttgtttgctgttttcttcatttcttcaaatagaccccttgaaagtttttaagatcctttagatagaacttagagatttcaaagagacgctggctgcatgcagtgaaacattcatgagtctcggtaatactgngtttct

SEQ ID NO: 51 - DNA, 538 bp, Homo sapien
gtcgacgcaaaagtttgactaaactttacctttttatagtttcactttttaagttatatttagaatatattgatagattataaattgattgtgaaacttttttctgaattttttcaacatgttttactcagttacatgagttaaaggatattttcagtcctgttatcttcaattgcagtctttaaaaaaacccaccctattgttctacttgttatatgtctattcatacagtaaattcatttcaaggtttatgccagtgggtattattggtgctttttgaagttgaggtgaaccatccaggaaggtcttgttaatgttatgttcatctataatggcataggggaaatatatatatttttaatattgtaaacatttgtactgaataacctttttttccccccctccgcaagcaaaactggttgaacagcggatgaagatatggaattcaaagctctaatggacctttttgaagagaagttgtggcttatgtggagtttacatgggcctctgatggaagaaagctaatctgtttagtatt

SEQ ID NO: 52 - DNA, 504 bp, Homo sapien
  misc_feature (1)...(504): n = A,T,C or G
cctttttttttttttttttaaagtacaaattcagtttattcatctgtttatgacacagtacacaggaggcaaagtgtttcacatcatagacttcacttccaactccttggaatgttcatttctttggcttacaggagagactagacaggaaggccaggcaatgcttaggcaactaaaatgaggttgggggtaatgctaacgtcaccctcacagggatggccacggggactgttattcgcaagctggttttctagacctgttagctggaagcatggtgagcaccatttctggacgctcaggccgtntcgggcttcagtcatntccaccacacaggtacagcagcgctttctggtagtcgcccttagtgtcttgctggatataatagtacagggacttgccgtactttctcttgaattcagacctaattttcaacatgtccacttcactgngggagaccatgattctgatcaggacccttatctcgcgtccccttgcccttcatgg

SEQ ID NO: 53 - DNA, 489 bp, Homo sapien
  misc_feature (1)...(489): n = A,T,C or G
gtcgactttagatgtacaggctgacananaagattcccgagagtaaatcatctttccaatccagaggaacaagcatgtctctctgccaagatccatctaaactggagtgatgttagcagacccagcttagagttcttctttctttcttaagccctttgctctggaggaagttctccagcttcagctcaactcacagcttctccaagcatcaccctgggagtttcctgagggttttctcataaatgagggctgcacattgcctgttctgcttcgaagtattcaataccgctcagtattttaaatgaagtgattctannatttggtttgggatcaatnggaaagcatatgcagccaaccaagatgcaaatgttttgaaatgatatgaccaaaattttaagtaggaaagtcacccaaacacttctgctttcacttaagtgtctggcccgnaatactgtaggaacaagcatgatcttgntactgtgatatttt

SEQ ID NO: 54 - DNA, 577 bp, Homo sapien
  misc_feature (1)...(577): n = A,T,C or G
ccttttttttttttttttttaagaactcaatacatggcttttaattattgtctataatttaaggaaataatcacctacaaataggatgtttctcaagttggcttacaaatttgttacttggcagactgaaaacatttcccacagaacaaatattatacacaatggttgggttcctttggttaatgcataatgtttactccataatttatttacccacaaacatgaattgaacatttctttgtgccanaaactattctaacactagaaatacaatagtaatgaacaaatagaaaaaaatcctattgtcattggtattacatccatagttttttctccaagagaataaaagtaagtaaaatatatagaattatagataatgatatatgctatggtgaaaaacaaagctgggtaaagggatagagaatgggggaaggataattttaactgattattagtagaatgtactagtatctctgttctaaaaggatttaagataggtattacttaccgaacctaagtattacaaataaaatagcaatgcttacactaggaaagactttcaactgagaagcattat

SEQ ID NO: 55 - DNA, 483 bp, Homo sapien
  misc_feature (1)...(483): n = A,T,C or G
cctttttttttttttttcaccaataattattttattcagggagtaaatgttattaattgccaaaatacgaattttaaatttgagaagtacagatttgtaagtatatatttgtttgaatagtatcanattggccttttattggcttattggtatttagngccagcacttacaatgtgaactcagcaacagaagataattcttatgaaatcaacattcaacttacatgaaataacttaaaaacttaccaacaatagtctaatgattatatacctttaccaaacaatgtctaatgaaagtccaaatgtaaaaatttaaaaattaaaattatagaatataatttttacacatcaattgttttgtagcaccatctcgcaaagnaaatatcatgtttattctgtagctaaaatttctccccacaagcagaaattgtttggaatatacaaaaagacaacccattaacaagtaactttaagtaatgtagtt

SEQ ID NO: 56 - DNA, 521 bp, Homo sapien
gtcgaccagacttaagcatcgagtttttaccatcttccactttaagctaagttatgatacctattccattcacaattggtgttctttttaaggtttgcaaatttcagccaattttgtagctaagattgttctgatcagctcaaaaagatttggcttagtgttttcattgcaaattataattgctgtagagccacacacaacttttgaacttttaattataagtgttatggctaaagttatttactgaaaatttcagtaaaatgtgtgaatgtttctttatgtattaacctcatagcagtaaatgacttgctgttgtttaatttttctaaggcatcttaatagacttctgttgaaaacttcagtgttaacatttttatagtttgtactaaatttaaccgtgatataaaaatgaattttatgcatagatcaggaattttaaattaaaggttttttctttaaaaaaaaaaaaaaaaaagggcggccgctcgagtctagagggcccgtttaaacccgctgatcag

SEQ ID NO: 57 - DNA, 542 bp, Homo sapien
  misc_feature (1)...(542): n = A,T,C or G
cctttttttttttttttacaacttcacattctttaatgttcattcagaatattaaatgccattaattgaccatcattattataaaatttactatttagataagtgagttttagtacagtgctatttaaagtatggaactgttactggtgtgtgatcagtacagaaattgagactaagcatttagaaacctagagcaatttgacgtagcaatcttctgtctgttgaatctaataacaaaaaaaattttttcaattttgcatatctttttaaaatttaatttgtcaaggaattcatttttagcatattttacaaaaacatcattctcctatggagactatttggaaatacaaataagaaaactggttcttaccacagatagtttttagaaacctgttttagngtaaagccatcatttagtataaagncatctattattactgttactctgaagtggttactgagcattacaacagtnggtnggattataagtttgtttactaaanatgctaggatttattaactcatgtatatatttattgaga

SEQ ID NO: 58 - DNA, 261 bp, Homo sapien
gtcgacagagaaggtctatgtcaacagagttgttatctcatagagccagttttcaaagctccttctgcattgtcactcactgatcaggtgatgaattcttcctagatagtcgcccactccacctcctacttaacctgagactcattatttagctatttctgcttttgtaaaaataattcagatattaaactccaattttaatctatcatccaagggtagatgtagttgcttagtagcattttggaaaaaaaaaaaaaaaag

SEQ ID NO: 59 - DNA, 480 bp, Homo sapien
  misc_feature (1)...(480): n = A,T,C or G
cctttttttttttttttaaaatatagaagttctgagttagacctgtttagctcanaatagtgggctaaactaccataaaattctctgtatatcttaaatggtaatgggtcaaaaactccagaaaatcatcagttgataacacacctacagataagtgcatgggtaggaggggatagccaagtgcccatgataatttgacctcagtaaattaaactgggcaatacacatatttgctattctgatactgcattagacttataaaattccatctaataagcattcataaaactggacctctctgtatatatctagcttagacagggatagggaaaagaataactgaagaaactagcttacaatagctaggtttcgtcaggcttattctatccagccagaaaccaccaccagagagaagctgagccattcagctgnctgtctcctctccctctgtttgaatagtcatgcctaggccttgctgca

SEQ ID NO: 60 - DNA, 493 bp, Homo sapien
  misc_feature (1)...(493): n = A,T,C or G
cctttttttttttttttggtccttctgtttatttcattttggatactcagtgaatgttaattaaccaggaaacttaaaagttatttcaattatgaacctcttcaatccttcatcaattattttgagtattctggtcttaaaaacatctctttcttctacaaacttctgaaagagatgaacacctccacctacaccaaaataatgtgctttgctggccaaaagtacacgtccatttttacttaacagtctaaggaaagtctggtgcaaattactataataatctgggttgtaaatggtttctgaggtgagaatgagatcatattttacaaaaagtttttcactacttagtacaagcttacaaaactcagaccactcaccagaaaaaaatcggcatttatatagttgngttacttttggtttcctgcatcttttcacatctggctcatttacatcattttcttcatcttccaaagtggagttagctactacatta

SEQ ID NO: 61 - DNA, 532 bp, Homo sapiens
  misc_feature (1)...(532): n = A,T,C or G
tttttttttttttttttgaaaaatataaaattttaataaaggctacatctcttaattacaataattattgtaccaagtaattttccttaaatgaactctttataatgcataatttacagtataagtagaacaaaatgtcatgacaaaagtcattgagtacaagacttgtaataaaaaggcataaaatatatttatacataaacccctttcaaaaaacaagggaaagcttgagccctcaatatagggcgacacacggagcgggtgaccgtgcaggtacaggtactgtactgatttaaagtcaagcactagagatagnggattaatactcttttgccgtacactatatacagatgtatagtacaagtaacaatggcaaacagaatgtacagattaacttaacacaaaaacccgaacatcaaaatgaaggtgtgtggaggaaaggtgctgctgggtctccctacaactgttcatttctttgnggggcagggggtagttcctgaatggctgnggtccaatgactaatgtaaaacaa

SEQ ID NO: 62 - DNA, 567 bp, Homo sapien
  misc_feature (1)...(567): n = A,T,C or G
gtcgactttttttttttttttaagtattttaggcatatttaataaataacttcagtaaatagcactgtaaaaagtgaactgttaaaactaaaggcacttaaaacaagaatgtgactagtgtgaaacaagatgggcaactcaaatggtgagaagtaaacatacagtggtctgttatggcactaactcaaagtaagactcgcgtaggtgagagctgttgcatagccacagtataacttcacatgttcattaaaaaggcaaattgaccgctaaaacttcaaagaaaaagtactcataaaaaaagtcttaccccaaaattgcaaacaaatacattaaaagattagaagaggtgatagaaagcaccagacattaaacaaaataaaaataataaaataaattcaactcaaaaggtccccattcagcaaatactttgtaaaagtatggcctgtatgtaaatagttgctaaatcaaggactttttagcagaaaattgctcggttcttttatctaaggcttgaatttgtaaagngaaggcataaaagttnccaaacattaagtaactcttaaaatg

SEQ ID NO: 63 - DNA, 247 bp, Homo sapien
gtcgacaaacaaacttggcttgataatcatttgggcagcttgggtaagtacgcaacttacttttccaccaaagaactgtcagcagctgcctgcttttctgtgatgtatgtatcctgttgacttttccagaaattttttaagagtttgagttactattgaatttaatcagactttctgattaaagggttttctttcttttttaataaaacacatctgtctggtgtggtatgaaaaaaaaaaaaaaaag

SEQ ID NO: 64 - DNA, 330 bp, Homo sapien
  misc_feature (1)...(330): n = A,T,C or G
cctttttttttttttttttttttttgacatggagtcttactctgtcacccaggctggagtgcagtagtgcaagctcggctcactgcaacctcaggcaggactatttttaattatttttaatacctgcaaaagggaatctgcacatgcacatccgtgtttctacanaaatctgcgatcgatggcagatctgtttgcctttgngtgtccacatgaaccatttggcaaaggcatccaatgctaacggggcccaccaactacaacggaggcaacaactctgnggattttntttcacagaaagagtaaaatttcattcaaccgttccatgtcgac

SEQ ID NO: 65 - DNA, 486 bp, Homo sapien
cctttttttttttttttactaggcaaagaactttattaatctttgtttcaaacttgattcccaggcttcttcggcttaattagctgcaaagaatgaattgtgtataagcaaaaactgaaaagagctgcagtgtccaaggggcttgggcttaaaaatattagagatctagattttatcagatccataaacaaaaatttcttaaaaagcagtcataatataaaatagcagctcccagtaacttcttcaggttttatcttcagaagttgactcaattcagtttgcctcattcttggaagcctcatcaaaattctccacaagatctggaacttcatcatcatcatcctctccagtagcaagtggtgcttttccatccacagattgtttgggcagagcttcggccagtctccttaaactagtcagactatccgcaccaagctggtttaagatgctgggtagcatttctgtcagctgctttgtctcagcatg

SEQ ID NO: 66 - DNA, 503 bp, Homo sapien
gtcgaccgtcagacagcaactcagagaataaccagagaacaaccagattgaaacaatggaggatctttgtgtggcaaacacactctttgccctcaatttattcaagcatctggcaaaagcaagccccacccagaacctcttcctctccccatggagcatctcgtccaccatggccatggtctacatgggctccaggggcagcaccgaagaccagatggccaaggtgcttcagtttaatgaagtgggagccaatgcagttacccccatgactccagagaactttaccagctgtgggttcatgcagcagatccagaagggtagttatcctgatgcgattttgcaggcacaagctgcagataaaatccattcatccttccgctctctcagctctgcaatcaatgcatccacagggaattatttactggaaagtgtcaataagctgtttggtgagaagtctgcgagcttccgggaagaatatattcgactctgtcagaaatattact

SEQ ID NO: 67 - DNA, 519 bp, Homo sapien
  misc_feature (1)...(519): n = A,T,C or G
cctttttttttttttttgaataaattttttttttatttttacaccataatccaattctagttatcttaattgaatttgaaaactttttcaattgcattaaatttacaaaaaagttctcccacattacactaaagcattcctcatgtttcacttccagtactcagatactgaatgagtaaaatcattttattggctctcttttaattaactccttcaaatgcacattgtttaaaaactgactaggtcaaaaatagttacncctgcaggttgacctattcagactttgccaaactcctccaagttcaatataaattgacgttttcagagtacaaagtcaattttacggaaacgctgttcctccttttccatggagccaatctgggtaattttttcattaaaattcttcttctgcctgtttgctgcggaactctttgagctgctgtagccgctcgatagtttcanaaatggtgcgttccccgtggaccttattgtcctcttgtgcggatatnaacagtgcca

SEQ ID NO: 68 - DNA, 495 bp, Homo sapien
gtcgactaaagctgaagagataaaagaggttgtggggctatgtcttaagacaaaagaacatttagaaaacctcaggaaatgatcagagtgggatagatgttactagaagaaacaaagaaattgaattcaattaggagttagaatcatttacaaagcaatggggaaagtaagcccctaaaaactattgtagcatatagtaaccagagccaaactctcataatatattccccaaggcaaaagaaaaatatttacaagattggcgttgttttatatgtttgcaaacttatttaataagtctggctttgtagatttcatatctgagtctgcattcaatcaaaatgtcttggctaaacttcatgaaaaaaccccagcctcataaattagtagttggaaaaaggaggcatatttagagctttttcagataattgtatttctttgatacattagactggacacacagtagtttgtttaaggttaattgcaatattgcaatga

SEQ ID NO: 69 - DNA, 525 bp, Homo sapien
  misc_feature (1)...(525): n = A,T,C or G
gtcgacgccaccatgttcgaggcgcgcctggtccagggctccatcctcaagaaggtgttggaggcactcaaggacctcatcaacgaggcctgctgggatattagctccagcggtgtaaacctgcagagcatggactcgtcccacgtctctttggtgcagctcaccctgcggtctgagggcttcgacacctaccgctgcgaccgcaacctggccatgggcgtgaacctcaccagtatgtccaaaatactaaaatgcgccggcaatgaagatatcattacactaagggccgaagataacgcggataccttggcgctagtatttgaagcaccaaaccaggagaaagtttcagactatgaaatgaagttgatggatttagatgttgaacaacttggaattccagaacaggagtacagctgtgtagtaaagatgccttctggtgaatttgcacgtatatgccgagatctcagccatattggagatgctgntgtaatttcctgtgcaaaagacggagtgaaattttctgca

SEQ ID NO: 70 - DNA, 511 bp, Homo sapien
gtcgacattttatatataatactactaatggcatagattaacaaaatattttacatgtaggaaaggacataagattacttttaaagaatagtatgaaatacacaatattcaaatgtgtttgcaatgcctaccaaatttcaaatgtgcctggatcatgtataaattaaggaaagaaaaaaggatcatgtataaattaaggaaagaaaaaatgtaagtatacaacctacacggtaaaaacaaaaaccaaacacctggttaaaaatatctatttaagctcgagtgtataaccttaaacaatttgtgtatcactagaaaaatggatttattagtaaaatttagggcagagattttattttggacaccactgcctttgtagaaaaatccaaagtggcataaaaagaaaaataaaatattaaaagaaaaaatatatattatcattcccatgttcccatcctgttactagcattgctgttctggtgcatcaatcctgagtactctaacttttgattta

SEQ ID NO: 71 - DNA, 464 bp, Homo sapien
cctttttttttttttttggaagagcttcttgcactgttataagaaagaacatgtgggagattgcaaacaaagcaacataaagagtatacagcctgtaggagtctgactaaagtaaaaaaaactcatgtctttgtttagtgagtatctgtatactaagttaatgcaatgccaattagattcaaattaaatcaagtacaagcaaatgtactgaaagtattaggaatgcatcatctactttgctaaataatttgcactccgcattctgcaattacatgagcatgccattggtataatattggttatataacatttaacatgttagtttttaaaagaatgtagatacattcatagagatcagtatttttacagatgtttttactataaaaggaaccatgtataacattgatttttaccttcagttttgataataggctgaagactgccttcaatcactttaatttttg

SEQ ID NO: 72 - DNA, 234 bp, Homo sapien
  misc_feature (1)...(234): n = A,T,C or G
aataaaanntgaacaaaaggaaaaggtggatataaagtggaacctgtgggaaagaggcaagggctgcaggacagaagagactgggaactgcaggggccctgggactcaggaggagatgctgattcagctcataggtgacccagtcctggccccggctgttcccaagagaaggctgtaagtacccagggaggtggtaagcaggatggaggaaaaatcagaggactgggggtcgac

SEQ ID NO: 73 - DNA, 143 bp, Homo sapien
gtcgactaaataagtcaattcctggaatttgaaagagcaaataaagacctgagaaccttccagaaaaaaaaaaaaaaaaaaaaaaaaaaaaaaaaaaaaaaaaaaaaaaaaaaaaaaaaaaaaaaaaaaaaaaaaaaaagggg

SEQ ID NO: 74 - DNA, 533 bp, Homo sapien
  misc_feature (1)...(533): n = A,T,C or G
gtcgacataatctaggcatgaagagcaaaaatatcccttccggagtctttgaagctgaaaatataaaacaaataaaaaataaaaaaataaaaacccacaaaaatgttgaaccaaacctccctgctaatctccatgcccacgttctttcccaccctgttcccagtcttctgacaaactgtgtacatagcggactcctcctttctcctccgaggtggttttaaaggctttttggtgtatagaagtttgtccatttgtaaaactccggattgcgttcctccccgccttccgccccttcccttccctaaagtgatgggctttctcttttctctttttagtttacccggtttctttttaagtaatgtggaagaaaatggtttattttgtattgnggtattgaatattgngttcctttttatgaggcaaacctgattgtaaacttcatgtaactatagactggaaaaaaatgagccgngccaaaagtctncccttctgtttcttcagcacattgacccatnncacacacatacacacca

SEQ ID NO: 75 - DNA, 485 bp, Homo sapien
gtcgaccttccctaggctgtttctgctgggcgctccgcgaagatgcagctcaagccgatggagatcaaccccgagatgctgaacaaagtgctgtcccggctgggggtcgccggccagtggcgcttcgtggacgtgctggggctggaagaggagtctctgggctcggtgccagcgcctgcctgcgcgctgctgctgctgtttcccctcacggcccagcatgagaacttcaggaaaaagcagattgaagagctgaagggacaagaagttagtcctaaagtgtacttcatgaagcagaccattgggaattcctgtggcacaatcggacttattcacgcagtggccaataatcaagacaaactgggatttgaggatggatcagttctgaaacagtttctttctgaaacagagaaaatgtcccctgaagacagagcaaaatgctttgaaaagaatgaggccatacaggcagcccatgatgccgtggcaca

SEQ ID NO: 76 - DNA, 417 bp, Homo sapien
cacgctggttttgcatcttcaggagacgctcgtagccctcgcgcttctcctcggccaattcgcggaagaagtggctcacgccttccagagccacatcatcgcggtcgaaatagaagcccagagagaggtaggtgtaggaggcctgcaggtacaaattgaccaggctgttgacggctgcctccacgtcggtggaataattctgacgaatctgggagctcatggttggttggcaagaaggagctaaccacaaaaacggtgctggcaggtcccagaagcaggagatggccgagaagatggtcccggaggttgcaagcggagaggaaatcggagggcggtcggaggctggaagagagtccccggatctgttccgtccaaacactgttgaagcaagagacagacccgcgggaccgcgtcgac

SEQ ID NO: 77 - DNA, 547 bp, Homo sapien
  misc_feature (1)...(547): n = A,T,C or G
gtcgaccttttattaagaatatattttatcaggcattttgataacaaactgttactctaagtataggtgatttacccagtgtattttaaaaagtaaatgaatcccactgtagtttttcttgaaggaaaaatcatttctccagttgctgaggggtactaaaagcttcatacacattagcagcaaagtctttcacttgctccattgtcaacagatcctgaacaaaatgactaggtgtttcactgcaaactgaatggatctgtccgtttactattggaattatcttagctaaaggcaggctgacactggaaagactattcatagagttaccatgttgcaggtcctgttcagtaggtcgaaagaactcagccatattgtctagaagtctactaaaacctcggtttaaacaggtattcaaaactgtactaaaatctgggctttccaacatgtctctagtttcattgagaagtttaatagtggtaatgtctcgaggagaangtccacaggcctgcactgctaatggagtttcttcatctggcatcatataatg

SEQ ID NO: 78 - DNA, 499 bp, Homo sapien
  misc_feature (1)...(499): n = A,T,C or G
cctttttttttttttttttttttnnaaaaaaaatctttttttatttcaaagattgcttcttatattgaagctcatattaaagcaacagtacaatgttcataaaatataagtgtgatgccgtaacattttcttacatgtcagaatactgatatttatatgtatactaaaataagaactttaaaattgtacaaatagatacattaaaaatgacatagaaatagggcgtctntcactgaaacaagacagttatatctggcacgtattagtttaagatgaaagtagaagcaaaaagatttacaagaatcagcagtaacaagattgatgctcaagagacataattgtacattgnattgtacatacattgtatgggtttaagctggctgaatnttatatatttcaagtttaaaaatgcactacatatagagtgtccagagtttaaggcgaaattacagctcanaactgntgncctttctaattttggggaagcttntttgacaac

SEQ ID NO: 79 - DNA, 370 bp, Homo sapien
  misc_feature (1)...(370): n = A,T,C or G
ccttttttttttttttttttttttaaggagcaatgacatttcctagaagttactttaagaatttccctagagggtcgggtatcatctcanccagatctttctcatccttcaaggccctgtttggtacagcttgctaggaagctgttccagactgcagcagccctctctggggtctctctaccacttcccaggcactcanaacttgtgcctcannanactgttttgtggcactgncccattctctgattctccatgtgagctggttttatcccatccagcatggctgtgaaatcctaaaggttcaaaccccagccactcttcacctatatttcccccaaatggctagcacgggaaagggcccaaaggtagg

SEQ ID NO: 80 - DNA, 428 bp, Homo sapien
gtcgacaaaaagggaaggaaggagagacagataactctcagtcatttaaaaaactacaataaaatattatgaattatcaattagatcaaagttcctcacagctatatttatataggtaaaaaaaaattaaataggctaaatgcccaaaaatttaagactggcaaaatatacttggctaaatactgtgcgtctctattaaataccatgtttcagaagaattattaatgacatgagaatatgctcaaaatacatattgatatgtgcaaatacatattgcaaagtaagattatagaatgatcctagttcaaaaatgtcacatatatatgtatttaaaaaaaaaggcagttaagatttacaacaaaatgttagtggtgggaccttctggtaggaatacagatttttttttattcagaagttttttgatgtcg

SEQ ID NO: 81 - DNA, 533 bp, Homo sapien
  misc_feature (1)...(533): n = A,T,C or G
cctttttttttttttttatttttaaaatttttttattttgaaataattataaattatcagaaagttgcaaacaaagcccagtcaggtcccatgtaccagtttcactgccaccatctttaaaggaggattagacgaatctgactgctaaaagtggcccagggattctggagaaaatccaacaggtttgctatcaggaaagcaatttcacttacaattcaggtttgactgcaagtgaaagtggttgaaacaagtgagaagntgattgcttcctcatataatagtctaaatgtaggtgtccaagcctggaatagaggtcctggtcctctaagttctcaggaacacaggcttcttttagccactccacatctctagggtgttgtcctcatggtccaaaatggngactggaattccagccatcacatntgctttccaggcagcaaaatggaagaaggggcacanaagaacagagatgacaataggtataaacaagctctctttttaaaggagattcccaggagctgctacatgacact

SEQ ID NO: 82 - DNA, 493 bp, Homo sapien
gtcgacccgcgaagatgcagctcaagccgatggagatcaaccccgagatgctgaacaaagtgctgtcccggctgggggtcgccggccagtggcgcttcgtggacgtgctggggctggaagaggagtctctgggctcggtgccagcgcctgcctgcgcgctgctgctgctgtttcccctcacggcccagcatgagaacttcaggaaaaagcagattgaagagctgaagggacaagaagttagtcctaaagtgtacttcatgaagcagaccattgggaattcctgtggcacaatcggacttattcacgcagtggccaataatcaagacaaactgggatttgaggatggatcagttctgaaacagtttctttctgaaacagagaaaatgtcccctgaagacagagcaaaatgctttgaaaagaatgaggccatacaggcagcccatgatgccgtggcacaggaaggccaatgtcggggtagatgacaaggtgaatt

SEQ ID NO: 83 - DNA, 501 bp, Homo sapien
  misc_feature (1)...(501): n = A,T,C or G
cctttttttttttttttgtaataaagacactgcttttatttagtttgatatgtttctttacagaatgcagaaaacacatcttaaaatcatatagaaggaaataaaaacacatcagtggttggtgaacacttgaatgtgagattggctctccatctcacagagtccaacggccatcaccagcccagcgctcaggggagcaggctgcctgcaaaggcattgttgctgttgttattctgttcactgccccatcgcctccagttgctatggcaacaggccattctgggccagccacgtctctgcatggcagtgcccaatggtggagttgctaggggcgacggagctgtttggaaggcctttcaaagccctcacctggaacattgggaattgtttattttttgatgaggncatcagaaataatcttcaccaggtcagatcccacttgtgctcctgtctctggggcaccaggggaaactctgacttggaggcatgagcccagtcacc

SEQ ID NO: 84 - DNA, 454 bp, Homo sapien
  misc_feature (1)...(454): n = A,T,C or G
ccttttttttttttttttttttttatgctaataaaacatcataatttaaggactacactgcattttttaattccataaattataatcctttaacatatatgaaagtttcatattcttaaagngctttaaaatatatttaatttttttaacaagtggaaaagaatgtttcttaaaagacatttaattttttagtggaaattaatattaccaaaaacattctgtgcataacaatttgaataacaatttttttatcttcaagaaatgggatttttatataaaatacacatgtagcactgaatgccaaagtgatgggtatccatggtcanaattcaaaattagattcgctattaaacctgtctggtttgtgtcctgagtgaanaatgatctcgagctggggagggaggtgcattgggtaatcagtgcttttgaaggtgaatttccttgctgngaaata

SEQ ID NO: 85 - DNA, 509 bp, Homo sapien
  misc_feature (1)...(509): n = A,T,C or G
gtcgaccgctctcagctctcggcgcacggcccagcttccttcaaaatgtctactgttcacgaaatcctgtgcaagctcagcttggagggtgatcactctacacccccaagtgcatatgggtctgtcaaagcctatactaactttgatgctgagcgggatgctttgaacattgaaacagccatcaagaccaaaggtgtggatgaggtcaccattgtcaacattttgaccaaccgcagcaatgcacagagacaggatattgccttcgcctaccagagaaggaccaaaaaggaacttgcatcagcactgaagtcagccttatctggccacctggagacggtgattttgggcctattgaagacacctgctcagtatgacgcttctgagctaaaagcttccatgaaggggctgggaaccgacgaggactctctcattgagatcatctgctccagaaccaaccaggagctgcaggaaattaacagagtctacaangaaatgtacaagactgatct

SEQ ID NO: 86 - DNA, 520 bp, Homo sapien
gtcgacgggcgccagggtctttgtggattgcatgttgacattgaccgtgagattcggcttcaaaccaatactgcctttggaatatgacagaatcaatagcccagagagcttagtcaaagacgatatcacggtctaccttaaccaaggcactttcttaagcagaaaatattgttgaggttacctttgctgctaaagatccaatcttctaacgccacaacagcatagcaaatcctaggataattcacctcctcatttgacaaatcagagctgtaattcactttaacaaattacgcatttctatcacgttcactaacagcttatgataagtctgtgtagtcttccttttctccagttctgttacccaatttagattagtaaagcgtacacaactggaaagactgctgtaataacacagccttgttatttttaagtcctattttgatattaatttctgattagttagtaaataacacctggattctatggaggacctcggtcttcatccaagtggcctgagtat

SEQ ID NO: 87 - DNA, 171 bp, Homo sapien
  misc_feature (1)...(171): n = A,T,C or G
gtcgacgagtacagtatcagctgagctgaccttactctgaggactaactcttttgctggaagcggtttctgatttacagctcttggtttctcccagacatgttggtgggagagattttggtttttaaggggttgttagatggagtaaannttctttaagnnttaatttttt

SEQ ID NO: 88 - DNA, 508 bp, Homo sapien
  misc_feature (1)...(508): n = A,T,C or G
cctttttttttttttttttttttttgnagtaaaaaatctttatttccaaaatgatttgttagccaaaagaactataaaccacctaacaagactttggtaagaaagagacttgatgcttcttataaattccccattgcaaacaaaaaataacaatccaacaagagtcatgttacccattcttagccattaacctggttttaagtctccaaaatcaggattttaaaatgtacccaactgggaccaaatacaaacatgagacactagggnggcttgtccttgattaggaatcaccagcttaaggaactttatcatgggctgagagttagatagatagcttanaacaacattgcaaaagngggtgcttctacatgaggacttttttccccccaagtagaaaaataattaaatcttgngtttctttatattgngctttttttgggagaaagcaattcatttaaggatttaaaacatgttggatacaaaggtagttcanagatgtaataatggt

SEQ ID NO: 89 - DNA, 508 bp, Homo sapien
gtcgacgggataaatagaaagcagaatgaattaatggaaaagaactcggctgttaggccattctctaaattctagtttagccaaaagtttatgtgtggtttggggcttcatttatttatctcatgagtaaaatggaataatacctaacaggcaggctctggaagttggaaatcacatacacacacacacacacacagacacacacacacacgatcaatcatgtagctcatattagatgttcaataaataacagctactacagatgcctatcagttgagtaagtagttcattaaattgagctcccaaaggtctcttctcttcacatccatatccgtttctgcagcaatcaaatagatacatgattgtttttctgtaagaaattactgcaaagagaatctttttctcctactaactgttccttctacctggtataggagataaatgtacgtttcttaattagctgactttttagtatgtcatttctgaaggaaaaataaattaaccttaa

SEQ ID NO: 90 - DNA, 531 bp, Homo sapien
gtcgacacgagtcccgcgttctctccttgaatccactcgccagcccgccgccctctgccgccgcaccctgcacacccgcccctctcctgtgccaggaacttgctactaccagcaccatgccctaccaatatccagcactgaccccggagcagaagaaggagctgtctgacatcgctcaccgcatcgtggcacctggcaagggcatcctggctgcagatgagtccactgggagcattgccaagcggctgcagtccattggcaccgagaacaccgaggagaaccggcgcttctaccgccagctgctgctgacagctgacgaccgcgtgaacccctgcattgggggtgtcatcctcttccatgagacactctaccagaaggcggatgatgggcgtcccttcccccaagttatcaaatccaagggcggtgttgtgggcatcaaggtagacaagggcgtggtccccctggcagggacaaatggcgagactaccacccaagggttggatgggctgtctgagcgctgtgcccagtaca

SEQ ID NO: 91 - DNA, 426 bp, Homo sapien
gtcgacaattgaggcctacaagagaggggagcctaggagcttggattgaccttctagtcaaccacctgacttcagcacaccattacaatcgggagactaaaccaacaaccagaggatctaaaatgtcacattcagattttcaggaagaaaatcttcattacagtggagcacaaatgttccatacaagacatcattgaggagccatgctgtccccttctaacctgaaacacattctttcccatcctggttgggcttctgtacctccttattaatttatgaacctgaagttgcttgaagtgttttgggcttaataaatggggtgaaagtataggtagcagtaacacctacatgaaacaatacaccttggatcttttaatctaaattacttttcttttttaagtctacttttaaaataaatacttctgt

SEQ ID NO: 92 - DNA, 223 bp, Homo sapien
gtcgacttttaaagcaattgactaggagaaactatttgtagcttatataacaaggactatatataaataaaaaactatttctatgaaaatcttaaaattacacacagtccgatgaaaataatcatatattaaaaaggcaaaccagaaaaataaatacagatgaccaaaatccatgtgacatatttggcctaattagtaattagaaaaaaaaaaaaaaaaaaaa

SEQ ID NO: 93 - DNA, 486 bp, Homo sapien
  misc_feature (1)...(486): n = A,T,C or G
cctttttttttttttttttttttttttttttctcaaatatccaattttattttatcattctcgcattgggggatgcgatctgcagctaggatcggaattcccaggcctatanatttttaaaccacaccacaggggtaaaccttaaaagaagngaaacctaacactatatatatttccatttctaaatacagtatattacanaagtttaaatatnccacctntgngtacttacaactntaaaaagatncaatanctctaccaattataaataatgtancatttcatattaaagacattatcgtncaatggaanaataggaaccctntaacgtatcactatcaaggttagngtctatatctacttganataaaatactgaaaattcagngtatgaagccaaatcctgatttaacaagttattggtagtataagtgataagtgttanctgatgaagggaaggcaaatgtggtaatttatatctctgaca

SEQ ID NO: 94 - DNA, 214 bp, Homo sapien
  misc_feature (1)...(214): n = A,T,C or G
cctttttttttttttttttttttttttttttttttngcaacacaagtcaatctttattgaaaactgcagtattaatacataacaattcttgttacaataaacgtgcttttganatttttaaatctgagctcatctcatcagattgcataaaaaattaaaatagtntcaattgacacctaactgaactggctcaggatggaaattccattccttg

SEQ ID NO: 95 - DNA, 463 bp, Homo sapien
gtcgaccagaattcagagcgaatggtcacagttggtcgctgggcaaagggaatgagtgcagactatgaagaaattttggatgtacctaaaccgcaaaaacccaaaacaaaaatacctaaagttgttaatttttgataacagctagcactatcatgagttactacctcattgttactttctaaaccaggcccgcttcacgagttagagttgagctcccctgtagccaggactatgctgtagatatcagtatgatctgggtgtggccaaaaacaattttctttattctgtctatcaaatagtacttctaccactgtttggagaaaattgaagaaaagaataagatgattaaatgaattctctaaaagaacatattttaagagacagaacttagacataaccaagtagttgtatacctgattgtaacaatcatcttttataaaagcaaaattatgcataaatgtaa

SEQ ID NO: 96 - DNA, 606 bp, Homo sapien
  misc_feature (1)...(606): n = A,T,C or G
gtcgactttaaaagtgcctcggcatcctgtattacatgtcatagaattgtaaagtcaacatcaattactagtaatcattctgcactcactgggtgcatagcatggttagaggggctagagatggacagtcatcaactggcggatatagcggtacatatgatccttagccaccagggcacaagcttaccagtagacaatacagacagagcttttgttgagctgtaactgagctatggaatagcttctttgatgtacctctttgccttaaattgctttttagttctaagattgtagaatgatcctttcaaattgtaatcttttctaacagagatattttaatatacttgctttcttaaaaaacaaaaaaactactgtcagtattaatactgagccagactggcatctacagatttcagatctatcattttattgattcttaagcttgtattaaaaactaggcaatatcatcatggatacataggagaagacacatttacaatcattcattgggccttttatctgtctatccatccatcatcatttgaggcctaatatatgccaagtactcacatggtatgcattgngacataaaaaagactgtctata

SEQ ID NO: 97 - DNA, 530 bp, Homo sapien
  misc_feature (1)...(530): n = A,T,C or G
cctttttttttttttttgtagattttttgctatgttactcaggctggtcttggactcctgggctcaagcgatcctcccaccttggcttcccaaagtgccaggattataggcatgagccaccatgctcggcctgctccttttcttgaaacacctcctctgtggtttagattccaggagactggaatggtctgccctggtgggctgctgagtcagggacctgaggtgtttgttcactggggaggcgggttcagatcaggaatgtaaggatgatggaaagaagggagtcactctggtttggtgggactggggagcaatcttgatcacggccacttacagcttctgccattgtccttcaccactatctcagcatctcggtccctcacgatgtccctccagtcaattgtgtccatgtgacaaagcttatcgttcttctcaatataaacaccccctgacagaatctcggtgagctgagtcaagcggagctggcgcanagcgtggctggagttggtgttatagttcaacatgacgaa

SEQ ID NO: 98 - DNA, 479 bp, Homo sapien
  misc_feature (1)...(479): n = A,T,C or G
gtcgacggttagtttctgcgacttgtgttgggactgctgataggaagatgtcttcaggaaatgctaaaattgggcaccctgcccccaacttcaaagccacagctgttatgccagatggtcagtttaaagatatcagcctgtctgactacaaaggaaaatatgttgtgttcttcttttaccctcttgacttcacctttgtgtgccccacggagatcattgctttcagtgatagggcagaagaatttaagaaactcaactgccaagtgattggtgcttctgtggattctcacttctgtcatctagcatgggtcaatacacctaagaaacaaggaggactgggacccatgaacattcctttggtatcagacccgaagcgcaccattgctcangattatggggtcttaaaggctgatgaaggcatctcgttcagggggcctttttatcattgatgataagggtattcttcggcagatcactgt

SEQ ID NO: 99 - DNA, 502 bp, Homo sapien
  misc_feature (1)...(502): n = A,T,C or G
cctttttttttttttttgtaagtttaaatttattttttaaaaatgcttgtcttcctcactagacaatcaactctatgagggcagagactatgtcaccactgtcccaccagcccctggcacacagtaggtactcaataaatatatgttggaaggatggatggaggtaatggatggaaagatggatggaaggatgaatggagggatggatgtgacccagctgaagtgtgagtaggaacattctcttattatgggtggaggaaagagagaggagattgagaaaataagataaaatacattgatgagcatcatttttggtgttcgaaaagtaggattgaattaggactaataaatctagagaattttacctctttcaatgcccaagccacacttttctatcactttgaaaccgaaaaagtaaatactttcccaacatttgctttgctggtaggaaatgctttaataaaaatgcaatctctangttgccatggcatcattaaaagaa

SEQ ID NO: 100 - DNA, 537 bp, Homo sapien
gtcgaccctttccataaatccttgatgattgacaacacccatttttccttttgccgaccccaagagttttgggagttgtagttaatcatcaagagaatttggggcttccaagttgttcgggccaaggacctgagacctgaagggttgactttacccatttgggtgggagtgttgagcatctgtccccctttagatctctgaagccacaaataggatgcttgggaagactcctagctgtcctttttcctctccacacagtgctcaaggccagcttatagtcatatatatcacccagacataaaggaaaagacacattttttaggaaatgtttttaataaaagaaaattacaaaaaaaaattttaaagacccctaaccctttgtgtgctctccattctgctccttccccatcgttgcccccatttctgaggtgcactgggaggctccccttctatttggggcttgatgacttttctttttgtagctggggctttgatgttcctttccagtgtcatttctcatccacataccctgacctg

SEQ ID NO: 101 - DNA, 611 bp, Homo sapien
  misc_feature (1)...(611): n = A,T,C or G
gtcgacctaaaatgaagtgtttgaaatcagaaatctatttctaatgtctcatagctttaaaactatttttgtccttatactcatacttgttattttattttattcatcctatatagccatttgactgaaatgtagaaaataatttattaaattgagaaaatatgcaggcattgaacaatctttcaagtattttgaataaaaattcaaattattatagattgcctggaattgttaagactgtcagaaggtcagctcattgatagctaagtagtatacactctgaaaaacagaatgtagaaatgggttttataaaagctgacctctagagtaaaggaggacccagcatgtgtaattcttcctcttaatactttaagaccactaatttgaggacttatggtttctcaccactgcactcttgcagctttcaagaaagtacttaagttttaaatgcccaggtgatttctaagactcttgaatagaattggttgggttcttctgatattgcattttcatgagaaaaaatttcagtggtacattaatttttatttttccttttgcttatagacttcgcatatcatttaaagtgatggttcgagcttnctctggatact

SEQ ID NO: 102 - DNA, 498 bp, Homo sapien
  misc_feature (1)...(498): n = A,T,C or G
cctttttttttttttttttaacgcatatttgtttttatttataggtaactaccacatgaattataaagacaacaaaggatgtcagaatgaacatggataggtgtatgcatactacggctaaggagaaacaatgttcctacatattatgggtagtgagaacattatctgtataacagggaactgtgattatttaaaaatatgcagaacttatttcatctgtgctttanaaataactgtatacagtgttataagttgaaaagaactcaaaataactaataccaaatatacacctatgtattanaattcaaaaaagctgctttctgtgaagtcaatcagctatattaaaaaatgacacaaatccaaaacaagatgcatgttatatataaagggacattgtaagtttccttgctgcattaaacccatggtttaatccatgaaatttccttttaattatcatttagacagaagcatgcaaatagtctcaggatctacttaaga

SEQ ID NO: 103 - DNA, 446 bp, Homo sapien
gtcgactcttggtgtttttgtatttccacctcacccccagcacatagcccagtctcttgcacaaattaagtacttaatgtgtgttgagctaaattgaataaaggattattagcattagcatattttgtgccttggttgtataagctggttgtttgttttgttacctttgcaaatatttatgattatcacccccccacatactaaattgtttttaaaagttttgcctttccttcagatactaccccaggcaatttgctgtagataatgtgattgcttccaatgacataattatcccaaactctctgccccggatatactttgccaaacgaaatttgaattctctgaataaattggtcatgtcctaaaaaaaaaaaaaaaaaaaaaaaaggggcggccgctcgagtctagagggccccgttttaaaccccgctgatcagcctcgactg

SEQ ID NO: 104 - DNA, 286 bp, Homo sapien
  misc_feature (1)...(286): n = A,T,C or G
gtcgaccttcgttatccgcgatgcgtntcctggcagctacattcctgctcctggcgctcagcaccgctgcccaggccgaaccggtgcagttcaaggactgcggttctgtggatggagttataaaggaagtgaatgtgagcccatgccccacccaaccctgccagctgagcaaaggacagtcttacagcgtcaatgtcaccttcaccagcaatattcagtctaaaagcagcaaggccgtggtgcatggcatcctgatgggcgtcccanttccctttcccattcctga

SEQ ID NO: 105 - DNA, 406 bp, Homo sapien
  misc_feature (1)...(406): n = A,T,C or G
gtcgacgcgtagcagagtggtcgttgtctttctaggtctcagccggtcgtcgcgacgttcgcccgctcgctctgaggctcctgaagccgaaaccagctagactttcctccttcccgcctgcctgtagcggcgttgttgccactccgccaccatgttcgaggcgcgcctggtccagggctccatcctcaagaaggtgttggaggcactcaaggacctcatcaacgaggcctgctgggatattagctccagcggtgtaaacctgcagagcatggactcgtcccacgtctctttggtgcagctcaccctgcggtctgagggcttcgacacctaccgctgcgaccgcaacctggccatgggcgtgaacctcaccagtatgtncaaaatactaaaatgcgccggcaatgaa

SEQ ID NO: 106 - DNA, 258 bp, Homo sapien
  misc_feature (1)...(258): n = A,T,C or G
gtcgacgattttttttgtacattttggctgcagtattggtggtagaatatactataatatggatcatctctacttctgtatttatttatttattactagacctcaaccacagtcttctttttccccttccacctctctttgcctgtaggatgtactgtatgtagtcatgcactttgtattaatatattanaaatctacagatctgttttgnactttttatactgttggatacttataatcaaaacttttactagggta

SEQ ID NO: 107 - DNA, 369 bp, Homo sapien
  misc_feature (1)...(369): n = A,T,C or G
gtcgacgtaaaatagaaacagaaggggactttatcaacctgattaactttctcaacatgttaaccctacagttaacattataatcaatggtgaatcattgagtactttccttctaagatcagaaacagttcaaagtccactctcaccatttctattcaacattgtactggaatcccagccagtgcagtaataccaataataaaaaattaaagtcataaagattgaaaaggatgaagtaaagctatttcaattntatttagaagtatttagaaaccccaaagaatctacaaaaaactaatagaaataagtgaatatatgaaggtcttactatacaagatcaacatatcaaaagcagtggtatttaagaaa

SEQ ID NO: 108 - DNA, 289 bp, Homo sapien
  misc_feature (1)...(289): n = A,T,C or G
gtcgacattgcatccttgaaatcctgggctcaggtgatcctcccgcctgagcctcctgagtatctgggactacagatgcgtgccaccaagcctggctaattttgtctcatgtcttctaaaaattattttgtgaagccccttcacaaaaaaccttaagggaaatctgatggtgctcaggaatctaactctccctaaaccatcctctttaactgcttctaaaatatctctgttggcctttcttancctttttctgtttccattcagtgctccaagcgctttttgtttctaa

SEQ ID NO: 109 - DNA, 444 bp, Homo sapien
  misc_feature (1)...(444): n = A,T,C or G
gtcgacctggcgttggcaccgctgaggaatgggcctgggcggggagggacatctctacaccgttcccatccgggaacagggcaacatctacaagcccaacaacaaggccatggcagacgagctgagcgagaagcaagtgtacgacgcgcacaccaaggagatcgacctggtcaaccgcgaccctaaacacctcaacgatgacgtggtcaagattgactttgaagatgtgattgcagaaccagaagggacacacagttttgacggcatttggaaggccagcttcaccaccttcactgtgacnaaatactggttttaccgcttgctgtctgccctctttggcatcccgatggcactcatctggggcatttaacttcgccattctctctttcctgcacatntgggcagttgtaaccatgcattaagagcttcctgattgagattcag

SEQ ID NO: 110 - DNA, 196 bp, Homo sapien
  misc_feature (1)...(196): n = A,T,C or G
ccttttttttttttttcattaaataanccatcatcacattagtacaatacaattttatattttttaaatatactatatatgttaaggataaggggtgaagttttcttcctttgtaatacctgttcaagagtttaatggattaggagattagngttaaccttgaggataaaagtncaaatttgtctcattaggacac

SEQ ID NO: 111 - DNA, 544 bp, Homo sapien
gtcgacctcagccggtcgtcgcgacgttcgcccgctcgctctgaggctcctgaagccgaaaccagctagactttcctccttcccgcctgcctgtagcggcgttgttgccactccgccaccatgttcgaggcgcgcctggtccagggctccatcctcaagaaggtgttggaggcactcaaggacctcatcaacgaggcctgctgggatattagctccagcggtgtaaacctgcagagcatggactcgtcccacgtctctttggtgcagctcaccctgcggtctgagggcttcgacacctaccgctgcgaccgcaacctggccatgggcgtgaacctcaccagtatgtccaaaatactaaaatgcgccggcaatgaagatatcattacactaagggccgaagataacgcggataccttggcgctagtatttgaagcaccaaaccaggagaaagtttcagactatgaaatgaagttgatggatttagatgttgaacaacttggaattccagaacaggagtactgctgtgtagtaaagatgccttctg

SEQ ID NO: 112 - DNA, 378 bp, Homo sapien
  misc_feature (1)...(378): n = A,T,C or G
gtcgacacggcttccgcacggtcatccgccccttctacctgaccaactcctcaggtgtggactagacggcgtggcccaagggtggtgagaaccggagaaccccaggacgccctcactgcaggctcccctcctcggcttccttcctctctgcaatgaccttcaacaaccggccaccagatgtcgccctactcacctgagcgctcagcttcaagaaattactggaaggcttccactagggtccaccaggagttctcccaccacctcaccagtttccaggtggtaagcaccaggacgccctcgaggttgctctgggatccccccacagcccctggncagtctgcccttgncactggtctgaaggtcattaaaattacattg

SEQ ID NO: 113 - DNA, 530 bp, Homo sapien
gtcgacgtcgttgtctttctaggtctcagccggtcgtcgcgacgttcgcccgctcgctctgaggctcctgaagccgaaaccagctagactttcctccttcccgcctgcctgtagcggcgttgttgccactccgccaccatgttcgaggcgcgcctggtccagggctccatcctcaagaaggtgttggaggcactcaaggacctcatcaacgaggcctgctgggatattagctccagcggtgtaaacctgcagagcatggactcgtcccacgtctctttggtgcagctcaccctgcggtctgagggcttcgacacctaccgctgcgaccgcaacctggccatgggcgtgaacctcaccagtatgtccaaaatactaaaatgcgccggcaatgaagatatcattacactaagggccgaagataacgcggataccttggcgctagtatttgaagcaccaaaccaggagaaagtttcagactatgaaatgaagttgatggatttagatgttgaacaacttggaattccagaaca

SEQ ID NO: 114 - DNA, 178 bp, Homo sapien
gtcgacatttcttcctaatattctataatctccaactcctgaaaacccctctctcaactaatactttgctgttgaaatgttgtgaaatgttaagtgtctggaaattttttttttctaagaaaaactattaaagtacttcctagtagggcaaaaaaaaaaaaaaaaaaaaaaaaaaaaa

SEQ ID NO: 115 - DNA, 211 bp, Homo sapien
  misc_feature (1)...(211): n = A,T,C or G
ccttttttttttttttttnggntcaatcttttatttggaacaaaggaaaaaaggactgacaccagtttagcctttgagtgtgcaaagctctgccctccctcccacccctnagccccaaatccaanatttcatagccctaacacccacccaagcagnttccctcacacatgccctttgntttcttcctctcttctatggttccttaggnaaa

SEQ ID NO: 116 - DNA, 439 bp, Homo sapien
gtcgacctgtcactcactacatgaataagcaaatattgtcttcaaaagaatgcacaagaaccacaattaagatgtcatattattttgaaagtacaaaatatactaaaagagtgtgtgtgtattcacgcagttactcgcttccatttttatgacctttcaactataggtaataactcttagagaaattaatttaatattagaatttctattatgaatcatgtgaaagcatgacattcgttcacaatagcactattttaaataaattataagctttaaggtacgaagtatttaatagatctaatcaaatatgttgattcatggctataataaagcaggagcaattataaaatcttcaatcaattgaacttttacaaaaaccacttgagaatttcatgagcactttaaaatctgaactttcaaagcttgctattaaatcatt

SEQ ID NO: 117 - DNA, 357 bp, Homo sapien
gtcgactccaaattgactttgcagcagggtggcagggtcaggagagtctggtcctgcctagctcagatttcatggcacctgcacttgaagcaagtcacttctttatcacaggtgtcttgaaacattagcttcttttaccaacctgagaaaattaggatgacctggcaaataagatcttgaataggccaaaagcaagtatcttgctgtgtgtagtctcttggttaaagtgaagaaacagtactgttcacacctttcttcactgagattccagtgtacatgagaacatatatttattgcatgattttctagatacacagtctatgcattattcatatacatttattttagcctaaagtg

SEQ ID NO: 118 - DNA, 431 bp, Homo sapien
cctccctgaggaaattaggaacctgttggcagatgttgaaacatttgtagcagatatactgaaaggagaaaatttatccaagaaagcaaaggaaaagagagaatcccttattaagaagataaaagatgtaaagtctatctatcttcaggaatttcaagacaaaggtgatgcagaagatggggaagaatatgatgacccttttgctgggcctccagacactatttcattagcctcagaacgatatgataaagacgatgaagccccctctgatggagcccagtttcctccaattgcagcacaagaccttccttttgttctaaaggctggctaccttgaaaaacgcagaaaagatcacagctttctgggatttgaatggcagaaaacggtggtgtgctctcagtaaaacggtattctattattatggaagtgat

SEQ ID NO: 119 - DNA, 131 bp, Homo sapien
cccctcgcccgtcacgcaccgcacgttcgtggggaacctggcgctaaaccattcgtagacgacctgcttctgggtcggggtttcgtacgtagcagagcagctccctcgctgcgatctattgaaaggtcgac

SEQ ID NO: 120 - DNA, 409 bp, Homo sapien
gtcgacgtaaaagccacacagaaatcaaaagataagaatatagtttcagctaccaaaaagcagcctcagaataaaagtgcatttcagaagacaggacccagctccttgaagtctcctggccgtaccccactgtccatcgtgagcctaccccagtcttctaccaaaacacaaactgcaccgaagtcagcacagactgtcgctaagagccagcattcaactaaagggcctcccagaagtggcaaaaccccagcttcaatcaggaaaccaccctcatctgttaaggatgcagatagtggagataaaaaacctactgcaaagaaaaaggaagatgatgaccattattttgtcatgactggaagtaagaaacctagaaaataaatacatactcattataaaaaaaaaaaaaaag

SEQ ID NO: 121 - DNA, 131 bp, Homo sapien; identical to SEQ ID NO: 119

SEQ ID NO: 122 - DNA, 130 bp, Homo sapien
gtcgacctttcaatagatcgcagcgagggagctgctctgctacgtacgaaaccccgacccagaagcaggtcgtctacgaatggtttagcgccaggttccccacgaacgtgcggtgcgtgacgggcgaggg

SEQ ID NO: 123 - DNA, 424 bp, Homo sapien
  misc_feature (1)...(424): n = A,T,C or G
gtcgacgagatgtggagtggctaaaagaagcctgtgttcctgagaacttagaggaccaggacctctattccaggcttggacacctacatttagactattatatgaggaagcaatcaacttctcacttgtttcaaccactttcacttgcagtcaaacctgaattgtaagtgaaattgctttcctgatagcaaacctgttggattttctccagaatccctgggccacttttagcagtcagattcgtctaatcctcctttaaagatggtggcagtgaaactggtacatgggacctgactgggctttgtttgcaactttctgataatttataattatttcaaaataaaaaaattttaaaaataaaaaaaaaaaaaaagggcggccgctcggagtctagagggcccgtttaaacccgntgatcagcctc

SEQ ID NO: 124 - DNA, 548 bp, Homo sapien
  misc_feature (1)...(548): n = A,T,C or G
cctttttttttttttttctctagtaatgactttattcatgaatctataatggaattcaaaatagcaaagaacatgaaaatgttcanattaatatttattaaccaaatgcatcanaaaatacatctattttcacatatcaaaagtgcctaaaatgcatgtganaatataaatattctccactttgnggaacttcaagataatgaaaaattgcttaatacactttgccacaaaaactcattacactgcaaatncagaanaaataaaataactcattacattgcagatncaaaagaaatcaaatgtaactggcaaaataaccatttcatggctaatctttnggnaaagngctattttcacactgaaaaaaagaanttagaaaagattaaaaattttaaattctgaaccatcattctnaaagtctgaagcgttttctttagtattcactatgttcatcacattcatgtgtncccaacatgagactaaacactatctcaaaatcttaaaaaatctttccatncacanattatttcctggaagntaaaaattat

SEQ ID NO: 125 - DNA, 562 bp, Homo sapien
  misc_feature (1)...(562): n = A,T,C or G
gtcgacgctcctaacaaagaagatatcttgaaaatttcagaggatgagcgcatggagctcagtaagagctttcgagtatactgtattatccttgtaaaacccaaagatgtgagtctttgggctgcagtaaaggagacttggaccaaacactgtgacaaagcagagttcttcagttctgaaaatgttaaagtgtttgagtcaattaatatggacacaaatgacatgtggttaatgatgagaaaagcttacaaatacgcctttgataagtatagagaccaatacaactggttcttccttgcacgccccactacgtttgctatcattgaaaacctaaagtattttttgttaaaaaaggatccatcacagcctttctatctaggccacactataaaatctggagaccttgaatatgtgggtatggaaggaggaattgtcttaagtgtagaatcaatgaaaagacttaacagccttctcaatatcccagaaaagtgtcctgaacagggagggatgatttggaagatatctgaagataaacagctagcagnttgcctgaaatatgctg

SEQ ID NO: 126 - DNA, 131 bp, Homo sapien; identical to SEQ ID NO: 119

SEQ ID NO: 127 - DNA, 512 bp, Homo sapien
  misc_feature (1)...(512): n = A,T,C or G
gtcgacgtccggcttcggagcgggagtgttcgttgtgccagcgactaaaaagagaattaaatatgggtgatgttgagaaaggcaagaagatttttattatgaagtgttcccagtgccacaccgttgaaaagggaggcaagcacaagactgggccaaatctccatggtctctttgggcggaagacaggtcaggcccctggatactcttacacagccgccaataagaacaaaggcatcatctggggagaggatacactgatggagtatttggagaatcccaagaagtacatccctggaacaaaaatgatctttgtcggcattaagaagaaggaagaaagggcagacttaatagcttatctcaaaaaagctactaatgagtaataattggccactgccttatttattacaaaacagaaatgtctcatgacttttttatgtgtaccatcctttaatagatctcatacaccagantttcagatcatgaatgactgacagaatattttgttgggcagt

SEQ ID NO: 128 - DNA, 483 bp, Homo sapien
  misc_feature (1)...(483): n = A,T,C or G
gtcgacgtttttgtgatactgacacatcccccctttcagaacaccctctgcccttggattctgtgcacaggaagctagttgctcccctgaatacactctttcttccttgtaatacagcctctgattttgagcccaagaataaagactacagttctcagactccttcgcaaataaattttgtgactaaactctagtcaacagtaagtgtcatgtagcagctcctgggaatctcctttaaaaagagagcttgtttatacctattgtcatctctgttcttctgtgccccttcttccattttgctgcctggaaagcagatgtgatggctggaattccagtcaccattttggaccatgaggacaacaccctanagatgtggagtggctaaaagaagcctgtgttcctgagaacttanaggaccangacctctattccaggcttgnacacctanatttanactattatatgaggaagcaatcaacttct

SEQ ID NO: 129 - DNA, 326 bp, Homo sapien
gtcgaccttttatctgtctatccatccatcatcatttgaaggcctaatatatgccaagtactcacatggtatgcattgagacataaaaaagactgtctataacctcaataagtattaaaaatcccattattacccataaggttcatcttatttcatttttagggaataaaattacatgtctatgaaatttcaattttaagcactattgtttttcatgaccataatttatttttaaaaataaattaaaggttaattatatgcatgtatgtatttctaataattaaaaatgtgttcaatccctgaaaaaaaaaaaaaaaaaaaaaaaa

SEQ ID NO: 130 - DNA, 276 bp, Homo sapien
  misc_feature (1)...(276): n = A,T,C or G
gtcgacggacaccagctgcggaanttgcggctttggcagattgaaatcatggcaggtccagaaagtgatgcgcaataccagttcactggtattaaaaaatatttcaactcttatactctcacaggtagaatgaactgtgtactggccacatatggaagcattgcattgattgtcttatatttcaagttaaggtccaaaaaaactccagctgtgaaagcaacataaatggattttaaactgtctacggttcttaacctcatctgttaagttcccatg

SEQ ID NO: 131 - DNA, 482 bp, Homo sapien
  misc_feature (1)...(482): n = A,T,C or G
ccttttttttttttttttaaattttaaggttatttttatttacaacttttgaaaaatgtacatttttttttacatgggttacttgtgcaaagttagatttggaagtgataaatgcataaaaggngacaatagaacattanacaaaacatttacaagccttgtcccatactgctacttaaaggtactatatatctaaaagtataaatatccaaaaaaagatcgcanacattggctttaaggttctcanatgctgaaagggaanaaattaaagcatgcagcaataactcaggatttgagtggaaaatagttngccacanatatgctatgctcccttccttgaattcattaaaactctaaaataaagatggacaattgagtttattcacttagggcagcactgatcctttaaaaagattaaaggagctccaactttccctagctnaaaaactcacnatngtttccattcctctgctcccacacct

SEQ ID NO: 132 - DNA, 428 bp, Homo sapien
  misc_feature (1)...(428): n = A,T,C or G
cctttttttttttttttgtctaaaaggcaaaaaactacaaacagcccaagtcctgagctccccaagacctggatcctccactgtccccctgaaacccggcaggaggcgggatggggagcacaanaggtgggttcttaaaaaagtcacccctggatgggaaagctcttcatcttctgccgccttcctntgcctcccgctgctgccgaggagagagatgganaggaccggggctatgccggcaaactcaacttcttcccctttaggactttggngatatagaggtanaanaaatcgcagtanaggactgtctggaccaggcctgccacaatggcnatgaggtcgaagaanccctcgaaanggtaagcgccananccagttgaanagatanagcgtggcggtaaacgcctagcgcaaacaagtagnggctg

SEQ ID NO: 133 - DNA, 537 bp, Homo sapien
gtcgaccccaaacccactccaccttactaccagacaaccttagccaaaccatttacccaaataaagtataggcgatagaaattgaaacctggcgcaatagatatagtaccgcaagggaaagatgaaaaattataaccaagcataatatagcaaggactaacccctataccttctgcataatgaattaactagaaataactttgcaaggagagccaaagctaagacccccgaaaccagacgagctacctaagaacagctaaaagagcacacccgtctatgtagcaaaatagtgggaagatttataggtagaggcgacaaacctaccgagcctggtgatagctggttgtccaagatagaatcttagttcaactttaaatttgcccacagaaccctctaaatccccttgtaaatttaactgttagtccaaagaggaacagctctttggacactaggaaaaaaccttgtagagagagtaaaaaatttaacacccatagtaggcctaaaagcagccaccaattaagaaagcgttcaagctca

SEQ ID NO: 134 - DNA, 535 bp, Homo sapien
  misc_feature (1)...(535): n = A,T,C or G
gtcgactcctctcacatggtggctttaggaagatccttggccaggagggtgatgccagctatcttgcttctgaaatatctacctgggatggagtgatagtaacaccttcagaaaaggcttatgagaagccaccagagaagaaggaaggagaggaagaagaggagaatacagaagaaccacctcaaggagaggaagaagaaagcatggaaactcaggagtgacattcccttcactccttttcctacccaagggggaagactggagcctaagctgcctgctactgggctttacatggtgacagacatttccgtgggatagggaagatagcaggaagaaaagtaaactccatagaagtgtcattccactgggttttgatattggcttagctgccagtctcccatttgtgacctatgccatccatctataatggaggataccaacatttcttcctaatattctataatctccaactcctgaaaaacccctctctcaactaatactttgctgttgaaatgttgngaaatgttaagtgtct

SEQ ID NO: 135 - DNA, 114 bp, Homo sapien
  misc_feature (1)...(114): n = A,T,C or G
gtcgacctcagcgtcattcagaannnggaaaagaatcaatgtaactcaagaaaggatgaaaataccctttcttcccatccacgtgtttccatctcaatcctcacagggtcctgg

SEQ ID NO: 136 - DNA, 354 bp, Homo sapien
agaagcgagatgacgaagggaacgtcatcgtttggaaagcgtcgcaataagacgcacacgttgtgccgccgctgtggctctaaggcctaccaccttcagaagtcgacctgtggcaaatgtggctaccctgccaagcgcaagagaaagtataactggagtgccaaggctaaaagacgaaataccaccggaactggtcgaatgaggcacctaaaaattgtataccgcagattcaggcatggattccgtgaaggaacaacacctaaacccaagagggcagctgttgcagcatccagttcatcttaagaatgtcaacgattagtcatgcaataaatgttctggttttaaaaaataaaa

SEQ ID NO: 137 - DNA, 347 bp, Homo sapien
  misc_feature (1)...(347): n = A,T,C or G
gtcgacggcgagattacgaggcgaggctcgcgcgcccgcccccgccctggcccccagtgcccacccggtcggcccggcacagccatgatcaaggcgatcctaatcttcaacaaccacgggaagccgcggctctccaagttctaccagccctacagtgaagatacacaacagcaaatcatcagggagactttccatttggtatctaagagagatgaaaatgtttgtaatttcctagaaggaggattattaattggaggatctgacaacaaactgatttatagacattatgcaacgttatattttgtcttctgtgnnggattcttnanaaagtgaacttggcattttag

SEQ ID NO: 138 - DNA, 434 bp, Homo sapien
  misc_feature (1)...(434): n = A,T,C or G
cctttttttttttttttggttaaatgacttactgtgtaattttatttcatattacacaaatgttaatcaaatgctgagtagacatgcagatgacaagcagtatatgacaaactctgaanaaatagttacatgtagagtttctcanatttttagtgtatctaanaattaactgaagagtttgttaagaatgcaggcttaaaggccaatccacagattataatttcatacaaacaggatggagcctaanaacctgtaaattattaaacaactgattaaaaatagagaggtttctatgaagttaggnntgtccttatttcttatttgaactggacaagtagaaggataataggtaggaccaagtgagcattatcagaatcaaagtagaggcaataacaagccaaggtgttttancctanctaaagaagctcgtcgac

SEQ ID NO: 139 - DNA, 553 bp, Homo sapien
gtcgacctgactataacagtgcctactatgttaacattagatgaacaagtgaattagaggatttttaaatgtgtatccatcagtgtatggacacactccctctaacttcttcaaaaaacaaaaattcctggtagagctaagtggtttttagaagtttggttttggtaactgatttctacgagataattgaacactttttaaaatagttgatcattatgtcaaacagccctcaacagtaaacttaaattaggtagaattatagtaagctggaagagaaaatgttcccaaagagcattagtccctttctggcaccttattacagatgaataaattgagactcacagaaattaaatgacttagccccagttatccaactaactccttaatgtgaggccatgattaggaataggcttctagtattcagtcccatattattttgactgtgtaataccacgtgccactttgattttaaagtcaaatctcggcttgaactgtatggggaaaaaaaaaatctccagctggctctgctgaatccccagaggggccctccact

SEQ ID NO: 140 - DNA, 450 bp, Homo sapien
  misc_feature (1)...(450): n = A,T,C or G
gtcgacgccggtgagttgggtgccggtggagtcgtgttggtcctcagaatccccgcgtagccgctgcctcctcctaccctcgccatgtttcttacccggtctgagtacgacaggggcgtgaatactttttctcccgaaggaagattatttcaagtggaatatgccattgaggctatcaagcttggttctacagccattgggatccagacatcagagggtgtgtgcctagctgtggagaagagaattacttccccactgatggagcccagcagcattgagaaaattgtagagattgatgctcacataggttgtgccatgagtgggctaattgctgatgctaagactttaattgataaagccagagtggagacacagaaccactggttcacctacaatgagacaatgaacagnggagagtgtgacccaagctgngtccaatctgnctttgca

SEQ ID NO: 141 - DNA, 140 bp, Homo sapien
acacacccctccctcacacagggctcgaccgccgctggcagttccagggctaaggatttcctgcacttacttgtggagaaggagttcatagctgggctcctggaggggagatagagcttctctttcgttcccgggtcgac

SEQ ID NO: 142 - DNA, 591 bp, Homo sapien
  misc_feature (1)...(591): n = A,T,C or G
gtcgacctggacttgcagtgtaaacagagacgctgcaaattgcttgtggacggtgtaggccgctgcaggccaccatgaaccggcttccggatgactacgacccctacgcggttgaagagcctagcgacgaggagccggctttgagcagctctgaggatgaagtggatgtgcttttacatggaactcctgaccaaaaacgaaaactcatcagagaatgtcttaccggagaaagtgaatcatctagtgaagatgaatttgaaaaggagatggaagctgaattaaattctaccatgaaaacaatggaggacaagttatcctctctgggaactggatcttcctcaggaaatggaaaagttgcaacagctccgacaaggtactacgatgatatatattttgattctgattccgaggatgaagacagagcagtacaggtgaccaagaaaaaaaagaagaaacaacacaagattccaacaaatgacgaattactgtatgatcctgaaaaagataacagagatcaggcctgggttgatgcacagngaaaggggttaccatggtttgggancacaggagatcacgtcaacaacagcctgt

SEQ ID NO: 143 - DNA, 538 bp, Homo sapien
  misc_feature (1)...(538): n = A,T,C or G
gtcgacaaataagaagacaccttcagcatcttaaactagaataaataaaagaagggtggcctcctagaatttaagtcaggagggaggtggtgggcaatggatgacaagctctactttgaagaggttgaatttcagctgaccactactaaagcagtacaagcttttcctttcagcaagtgtcttcccagaaatgtgatagcaatttttaggaagaatttggcaaacataatgtttagcagatttgcaacaaatgctataagctcaaatttttttttttttttttttnggcagcacactcagccctccaaggggaagtggattatttttcttgcaagtgcattancangggaggtattaaggacagcaacattccttcctgtataaaaaaataaataaataaaagaagaaaggattattgaggccctctctgctgnatgtaatgtacttcangatgttggtanaaaagatatcaacctanaataagnttcacaanaatacatttggtttcacngaaagtttaaagtcaatctggacattc

SEQ ID NO: 144 - DNA, 401 bp, Homo sapien
gtcgacctgttccctttttgggcctgtctccccatgtatatgttgaggggttggacttcagggcctgtgagaggccttccaacttagactttctccccaggagcataaattcagtgaatctacgtgactctcagtgatggcatcattgcctaatatccacccagcttctgcttgaaaacttccagagactggttcacatgggggtataaaagcccaggccccttgccccaacttgggacaactatgaagagtttccagctccacagctccctgaggggctggccgaggcctttgtggggtttgcctcacaacccaatttatccctctggccaattctgcttcaatcactccctgccaggtgttgaccttgaatgtactcccccaataaacctcctgcaagc

SEQ ID NO: 145 - DNA, 367 bp, Homo sapien
ccttttttttttttttttagttagaaattacaagtttatttttatattttgaaaaaggcataatagaaaacaaaaataaacaaccaggcatatcaatatttgtgacatacacatacacacaaaaatgaatataggaaataacacgaagaaaaagcatagtatgttttgaaaccaacgtggggcatgaacagatttttgatgaaatacaactaaaggttttaagtgtctatgtaatgttcgagatattacgatcactcttatcctactagcaaaaattagcaaactaggctttaaaacatgattcctgttgttttagcaggatttattttggtaatgatcctgcttccttataaacaactacgtcgac

SEQ ID NO: 146 - DNA, 395 bp, Homo sapien
  misc_feature (1)...(395): n = A,T,C or G
gtcgacaagaaagcccccttaatgtttttaactgatgatatttttttaagcttaccaatataagtatttttaaaggttctatttttcaaagtcataacaatgattgttcttgttttctctcatagaatagactgccatcggataaagagtggtccctagcttctatttttccaagtaaataagtagaacatgttcttgggattataccattaaatgttaattttcttgaagaagaaagattgttgtctgccaagattttatgttagcgctcggattgaggcagaaaacggaagcaccaggtttaacactgggatgacttgggttgtgttcctggaggtttgaagngggccttccccgccttttgagggggaaaactgactgntttgaacacatat

SEQ ID NO: 147 - DNA, 455 bp, Homo sapien
  misc_feature (1)...(455): n = A,T,C or G
gtcgactaaaaactggaacggtgaaggtgacagcagtcggttggagcgagcatcccccaaagttcacaatgtggccgaggactttgattgcacattgttgtttttttaatagtcattccaaatatgagatgcgttgttacaggaagtcccttgccatcctaaaagccaccccacttctctctaaggagaatggcccagtcctctcccaagtccacacaggggaggtgatagcattgctttcgtgtaaattatgtaatgcaaaatttttttaatcttcgccttaatacttttttattttgttttattttgaatgatgagccttcgtgcccccccttcccccttttttgtcccccaacttgagatgtatgaaggcttttggtctccctgggagtgggtggangcagccagggcttacctgtacactggacttgagaccagttgaaataaaagtgcac

SEQ ID NO: 148 - DNA, 518 bp, Homo sapien
  misc_feature (1)...(518): n = A,T,C or G
gtcgacctcacgccttcgccgtagcatctttcgcagcggaccgaagagaagaaaagtaggccagagccgaactctcttcctgccaagatgtctattggtgtgccgattaaagtactgcatgaggccgagggccacattgtgacatgtgagacgaacaccggtgaggtatatcgggggaagctcattgaagcagaggacaacatgaactgccagatgtccaacatcacagtcacatacagagatggccgagtggcacagctggagcaggtatacatccgtggcagcaaaatccgctttctgattttgcctgacatgctgaagaacgcacccatgttaaagagcatgaaaaataaaaaccaaggctcaggggctggccgaggaaaagctgctattctcaaggcccaagtggccgcaagaggaagaggacgtggaatgggacgtggaaacatctttcaaaagcgaagggataattttctaagttgaacagaactttgtccttttttctttcangttatctg

SEQ ID NO: 149 - DNA, 442 bp, Homo sapien
  misc_feature (1)...(442): n = A,T,C or G
ccttttttttttttttttcttttcataaaatttttactttatgaattaaatacattgagaaacagngaaaatatatttacagtcatttgaagngggcactactaacatatttaatttaaaaaaatctttgctgtttctttgcctgtttctttcaaagagaattttaaatatgactttagcttttaaaaaatacaatanggaaataattacattcttaatatgaaaacattttacaacgtatcaccatggtcaattaattctgaatatcacttaaaagttgatgttaaaatgtaaagngaatatttcctttcttgttanaaaatcaaaaagattatctcattaaaaacaccttnggncctaagacttatgatctgaanatgnccttttgaaaagnatcttccatggctacaactaaaaaanacccggtaacacttgtgcacgg

SEQ ID NO: 150 - DNA, 341 bp, Homo sapien
  misc_feature (1)...(341): n = A,T,C or G
gtnnacctattattaccccatgatacagtttagaaaacaaattcatgcactaagtaaatggaccaaatcgtaagtcactgccttttgctccagagttggctgctttgattactcctacacttaactagtcaactttaaagaaaaaaatttttttttctgtgaaggaaattaagtgcctattttcanagagctaaaagcaatcaaggcatctactgtgttattttcctatccatgtngactcatgtttaaggttgactaggaagacataatcattggctgctaataacaaatngatttcttttnataaaaaatttaaaagagtntntaatgctttattttat

SEQ ID NO: 151 - DNA, 459 bp, Homo sapien
  misc_feature (1)...(459): n = A,T,C or G
gtcgaccaggtcttgaccctggtcaacaagagaataggcctttaccgtcactttgacgagaccgtcaataggtacaagcaatcccgggacatctccaccctcaacagtggcaagaagagcctggagactgaacacaaggccttgaccagtgagattgcactgctgcagtccaggctgaagacagagggctctgatctgtgcgacagagtgagcgaaatgcagaagctggatgcacaggtcaaggagctggtgctgaagtcggcggtggaggctgagcgcctggtggctggcaagctcaagaaagacacgtacattgagaatgagaagctcatctcaggaaagcgccaggagctggtcaccaagatcgaccacatcctggatgccctgtagcccctgcccgcatcctncagggggcccagggtgcctgcactttgctgtgggnangcagattgggtggta

SEQ ID NO: 152 - DNA, 242 bp, Homo sapien
gtcgacccaaggtcacaggagcattgcgtcgctgatggggttgaagtttggtttggttcttgtttcagcccaatatgtagagaacatttgaaacagtctgcacctttgatacggtattgcatttccaaagccaccaatccattttgtggattttatgtgtctgtggcttaataatcatagtaacaacaataatacctttttctccattttgcttgcaggaaacataccttaagttttttttg

SEQ ID NO: 153 - DNA, 57 bp, Homo sapien
  misc_feature (1)...(57): n = A,T,C or G
ccttttttttttttttttttttccacatcactcaggttttatngaatttataaaatt

SEQ ID NO: 154 - DNA, 437 bp, Homo sapien
  misc_feature (1)...(437): n = A,T,C or G
cctttttttttttttttggtaatncagttttaatttattttcatcactttttcttcataatccagatattttaaaatgcaaagaaaattaactttcaatgatatgttcagggactggcactaaaaaaaattttcagactgcaaatgagttatacaaatgaaaatatcaaatggagatccagttatcaaaatgaaagcactcaacatattaaaagttcacaagtatttgtattgagcacattacaaaagtcagcttgctaactgttgtgattttaaagaactattgcanaagtctgaanaaaatanatttattagttaacttataaagagattaaagaggctgaaacaagtnttaaaaanaaatttgngcctttattanaatgttaggcgtcnacgcggccgctcnngtctanagggcccgtttaaacccgctgatca

SEQ ID NO: 155 - DNA, 518 bp, Homo sapien
gtcgacgtgagccacagtcacgccactgcattctatcctgggcaacagatggagaccttgtctcaaaaaaaaaaaattcctgacatcgctatgtattcccaactttatcatttgtctgcctgtttagttttgacttatgttttttttttttcccccctgtggacatgtagttgacggaaatcgtgaaggaactttaatattttatttaaatttcccaaaactaatcatgccttatgtgactaatcttcagtgataatatttcatctactgatatattttcttgaggtgtgtaattttcagtataccttaatcatttggtataaaaaagagagaggtttttgatatatgaatgctgttcttgtaaaaatcaatcttgacactttattttaaactttttattggtaatgacagtgggttttgtacatcatgattttcaatttaggatatctgtctaatttgttttttcagagtaactatattggaattcaataaaaatattcaaaatttttcttaaaaaa

SEQ ID NO: 156 - DNA, 600 bp, Homo sapien
gtcgacgtttatttaagttcatgtttcactgtttgcactttgcattgaacaatgggtttattcgctgatgtaaacggttcgagtgaagaattaatgcagtaagtatgacaacacatacacacttgcctctccccatctccagaagaggggagcagagtccgagcttatctaaatatgaatgtggccacaaagctgtggaaggtgacaaagcttaaacacctttgccctggctctgcattgtcacctagagagcaagaggtctatagaaacatcatgtcacatgaaacgattctctgctttttggttctgaacttgaagtccctaaactgcaaaatctaagagttgggtggttattaaaatgcttttaaaaagttaactgtggcaccaattctaatgtaatccaacttgtgactgtttttttttgttttgttttgtttttgtgtgtgtgtgtgtgtggcactgggaaaagtggaaacaaacatgtattgaaatacatattggaaataaaaatggtttgagcgtcagtgatattctcccagaatgtacttatcttacctcgcatgtactgtagtcactcagtatttgtatatgttgctagaa

SEQ ID NO: 157 - DNA, 542 bp, Homo sapien
gtcgacggctgggaagtcagttcgttctctcctctcctctcttcttgtttgaacatggtgcggactaaagcagacagtgttccaggcacttacagaaaagtggtggctgctcgagcccccagaaaggtgcttggttcttccacctctgccactaattcgacatcagtttcatcgaggaaagctgaaaataaatatgcaggagggaaccccgtttgcgtgcgcccaactcccaagtggcaaaaaggaattggagaattctttaggttgtcccctaaagattctgaaaaagagaatcagattcctgaagaggcaggaagcagtggcttaggaaaagcaaagagaaaagcatgtcctttgcaacctgatcacacaaatgatgaaaaagaatagaactttctcattcatctttgaataacgtctccttgtttaccctggtattctagaatgtaaatttacataaatgtgtttgttccaattagctttgttgaacaggcatttaattaaaaaatttaggtttaaatttagatgttcaaaagtagttg

SEQ ID NO: 158 - DNA, 526 bp, Homo sapien
cacctcaggctgtggctctttgggcttcttcctaatgcagaagaagttgcccagcagcaaaatcagggaggaggtgagcacctcggcccccgccaggatgaacacgtacatgtagacgtgggtcgcatccaggagtttgcctcccgaagggggcccgacgagcacggccaccgcctccatcagcagcaccaggccaatggcactggagaacttgtaggagatgccaaagaagatgcagaagaccacgaggccgccgtagtcgcccgccgtagagcccgccaggtccgcgaggccgttgaagaacatggagaagctgaagaggtagacggagtagggccgcaccttcccaagccccgccacgaagcccgcggccggccgcgcgaagatgtcaatgaagcccaggatggtgagcaggaaggcggccttggtgtcgggcacgcccaggtccttggcgtagctcaccacgaacacgggcgggacgaagagccccagcaccatgaccgaggcggccacggcgtaaagcaca

SEQ ID NO: 159 - DNA, 306 bp, Homo sapien
  misc_feature (1)...(306): n = A,T,C or G
ccttttttttttttttttttttttttnggatgtatnngaaattttttctatatanatcatgtgtgacttccataaagaaaaataaacacctatncacagtttacctaatatgtgtaatgttaatgaaaagaatcaaagaaagatgttcgttcattaactctctaaatnaaattgtttttccatttttaccaacttgataccttaatcaagncactcttgttcttccttaagtgcaaatgaattttttgtttgggttgggggacaacacaaaatacaaacctgggttggattcactgaaaggcccaa

SEQ ID NO: 160 - DNA, 528 bp, Homo sapien
ctgaagagcggcttgctcttcacatcctcaggactcaggggctggtccctgagcacgtggaaacaaggactttgcacagcaccttccagcccaacatttcccagggaaaacttcagatgtgggtggatgttttccccaagagtttggggccaccaggccctcctttcaacatcacaccccggaaagccaagaaatactacctgcgtgtgatcatctggaacaccaaggacgttatcttggacgagaaaagcatcacaggagaggaaatgagtgacatctacgtcaaaggctggattcctggcaatgaagaaaacaaacagaaaacagatgtccattacagatctttggatggtgaagggaattttaactggcgatttgttttcccgtttgactaccttccagccgaacaactctgtatcgttgcgaaaaaagagcatttctggagtattgaccaaacggaatttcgaatcccacccaggctgatcattcagatatgggacaatgacaagttttctctggatgactact

SEQ ID NO: 161 - DNA, 527 bp, Homo sapien
  misc_feature (1)...(527): n = A,T,C or G
ccttttttttttttttttggtcttacaactctattgtaaactatactagactatagagggacttctacatctttcaagatgtgtttaataaaggtctgtttataataacttttgaggcatgaatctagcaaatagtactttatacaatgtcccttgtcattaccaactcataaatattaagtgtttttcagtgacttatgtttggatgtggtagtgctgatcagggccatgtgctgatgtcctggagagcaaaatcaatccaaagnggngctgctatttgtgacagaacatgtttatttactcagccccggagacaaaaggaaaattgatatgggggagcgggaaataggagaactattaaatgtagtgaagaaatttcacaggtctaaaggaactattaaaaggaaggataaagtagattctatactataaaacagaatcctacctctgataaaagacaaatcagcctgaatttttgaataatcaataggattcaaaatgactattttcaattgcaatctcattct

SEQ ID NO: 162 - DNA, 77 bp, Homo sapien
  misc_feature (1)...(77): n = A,T,C or G
ccttttttttttttttttttttnnttttttttttttttttttttagggaaanaaatctgggttccttttatttttga

SEQ ID NO: 163 - DNA, 645 bp, Homo sapien
gtcgacaaacaatgaatagtttttcattgtaccatgaaatatccagaacatacttatatgtaaagtattatttatttgaatctacaaaaaacaacaaataatttttaaatataaggattttcctagatattgcacgggagaatatacaaatagcaaaattgaggccaagggccaagagaatatccgaactttaatttcaggaattgaatgggtttgctagaatgtgatatttgaagcatcacataaaaatgatgggacaataaattttgccataaagtcaaatttagctggaaatcctggatttttttctgttaaatctggcaaccctagtctgctagccaggatccacaagtccttgttccactgtgccttggtttctcctttatttctaagtggaaaaagtattagccaccatcttacctcacagtgatgttgtgaggacatgtggaagcactttaagttttttcatcataacataaattattttcaagtgtaacttattaacctatttattatttatgtatttatttaagcatcaaatatttgtgcaagaatttggaaaaatagaagatgaatcattgattgaatagttataaagatgttatagtaaatttattttattttagatattaaatgatgttttat

SEQ ID NO: 164 - DNA, 434 bp, Homo sapien
  misc_feature (1)...(434): n = A,T,C or G
gtcgaccggacgcggcggcattaaacggttgcaggcgtagcagagtggtcgttgtctttctaggtctcagccggtcgtcgcgacgttcgcccgctcgctctgaggctcctgaagccgaaaccagctagactttcctccttcccgcctgcctgtagcggcgttgttgccactccgccaccatgttcgaggcgcgcctggtccagggctccatcctcaagaaggtgttggaggcactcaaggacctcatcaacgaggcctgctgggatattagctccagcggtgtaaacctgcagagcatggactcgtcccacgtctctttggtgcagctcaccctgcggtctgagggcttngacacctaccgctgcgaccgcaacctggccatgggcgtgaacctcaccagtatgtncaaaatactaaaatgcgccngcaatgaa

SEQ ID NO: 165 - DNA, 388 bp, Homo sapien
  misc_feature (1)...(388): n = A,T,C or G
gtcgaccattcatatatatatgcatatatatgtgaagctccatatttctgttgctttaaagaagtaaaaccttccatttaaataagatgacatgcntaanataacaaagcttccttgatttccttttcctgtgtaattnaatagatttgttgactagtgcttgggcacattataaatcagngttatttgctcttggagccattttttaaaaaaaattttggcagtgagcagttgaatttatcttgaatttatcatgtgtgtgtatttctgaagcagctacatagcagaacattttaagagattctgttagcccacatgttcatgttggttgctgctgaatggtaaatattaaataaaattaccagattaatcttaaaaaaaaaaaaaa

SEQ ID NO: 166 - DNA, 443 bp, Homo sapien
  misc_feature (1)...(443): n = A,T,C or G
gtcgaccttgctttcttaaaaaacaaaaaaactactgtcagtattaatactgagccagactggcatctacagatttcagatctatcattttattgattcttaagcttgtattaaaaactaggcaatatcatcatggatacataggagaagacacatttacaatcattcattgggccttttatctgtctatccatccatcatcatttgaaggcctaatatatgccaagtactcacatggtatgcattgagacataaaaaagactgtctataacctcaataagtattaaaaatcccattattacccataaggntcatcttatttcatttttagggaataaaattacatgtctatgaaatttcaattttaagcactattgnttttcatgaccataatttatttttaaaaataaattaaaggttaattataaaaaaaaaaaaaaaag

SEQ ID NO: 167 - DNA, 608 bp, Homo sapien
  misc_feature (1)...(608): n = A,T,C or G
gtcgactgcgcctctccgaacgcaacatgaaggtgctccttgccgccgccctcatcgcggggtccgtcttcttcctgctgctgccgggaccttctgcggccgatgagaagaagaaggggcccaaagtcaccgtcaaggtgtattttgacctacgaattggagatgaagatgtaggccgggtgatctttggtctcttcggaaagactgttccaaaaacagtggataattttgtggccttagctacaggagagaaaggatttggctacaaaaacagcaaattccatcgtgtaatcaaggacttcatgatccagggcggagacttcaccaggggagatggcacaggaggaaagagcatctacggtgagcgcttccccgatgagaacttcaaactgaagcactacgggcctggctgggtgagcatggccaacgcaggcaaagacaccaacggctcccagttcttcatcacgacagtcaagacagcctggctagatggcaagcatgtggtgtttggcaaagttctagagggcatggangtggtgcggaangtggagagcaccaagacagacagccgggataaacccntgaangatgtgatcatcgcagactgc

SEQ ID NO: 168 - DNA, 569 bp, Homo sapien
  misc_feature (1)...(569): n = A,T,C or G
gtcgacgcggncggccggacagactgacgtgtgagctgcatcgcgggaggcgcatggnggggatggcgctggcgcgggcctggaagcagatgtcctggttctactaccagtacctgctggtcacggcgctctacatgctggagccctgggagcggacggtgttcaattccatgctggtttccattgtggggatggcactatacacaggatacgtcttcatgccccagcacatcatggcgatattgcactactttgaaatcgtacaatgaccaagatgcgaccaggatcagaggttncttggggaagacccaccctacgaagttggaatgagaccatcagatgtgataagaaactcttctagatgtcaacataaccaaccttataaagactaaaattcatgagtagaacaggaaaatcatcctgactcatgtgttgtgttctttatttttaattttncaaagaggctcttgtatagcagttttttgtctattttaacattgtaagtcattttgtnctttganatcantattttcttaacctttgtgactgtttcaatattacccccgnga

SEQ ID NO: 169 - DNA, 216 bp, Homo sapien
gtcgaccgggaacccatctataaagtaaggcacactcgtaatggttgaattgtgttctggttaatttcctaaaggacttcacagttgcacttatgaaaatgattttatattgaaatgatatttgcataagaaaaagcatgtgattaattgcatattgcttgagtgttcatctgtgaatgtgaaaaataagctgtttttttttattagatatttgca

SEQ ID NO: 170 - DNA, 284 bp, Homo sapien
  misc_feature (1)...(284): n = A,T,C or G
cctttttttttttttttgaaatggancttctgaatcgaaaagtttttcactttaaatgttggatgagtgctaccaaaacactnngcatcttagggcaagtgtcgctgagcacctgcttccccatattctcagcannatcatttcagttcttagcaatctggcaggcaaaaggaaagtctgattttgntngaattngcattttcctgattaccancaaactantttaagcttaatgggcacntnntatttctattctctgaactgcccatttttctaccattcag

SEQ ID NO: 171 - DNA, 541 bp, Homo sapien
cagacagcactgtgttggcgtacaggtctttgcggatgtccacgtcacacttcatgatggagttgaaggtagtttcgtggatgccacaggactccatgcccaggaaggaaggctggaagagtgcctcagggcagcggaaccgctcattgccaatggtgatgacctggccgtcaggcagctcgtagctcttctccagggaggagctggaagcagccgtggccatctcttgctcgaagtccagggcgacgtagcacagcttctccttaatgtcacgcacgatttcccgctcggccgtggtggtgaagctgtagccgcgctcggtgaggatcttcatgaggtagtcagtcaggtcccggccagccaggtccagacgcaggatggcatgggggagggcatacccctcgtagatgggcacagtgtgggtgaccccgtcaccggagtccatcacgatgccagtggtacggccagaggcgtacagggatagcacagcctggatagcaacgtacatggctggggtgttgaaggtctcaaacatgatctg

SEQ ID NO: 172 - DNA, 573 bp, Homo sapien
gtcgactttcaacaaatcctgaagtctttctgtgaagtgaccagttctgaactttgaagataaataattgctgtaaattccttttgattttctttttccaggttcatggtccttggtaatttcattcatggaaaaaaatcttattataataacaacaaagatttgtatatttttgactttatatttcctgagctctcctgactttgtgaaaaagggtggatgaaaatgcattccgaatctgtgagggcccaaaacagaatttaggggtgggtgaaagcacttgtgctttagctttttcatattaaatatatattatatttaaacattcatggcatagatgatgatttacagacaatttaaaagttcaagtctgtactgttacagtttgagaattgtagataacatcatacataagtcatttagtaacagcctttgtgaaatgaacttgtttactattggagataaccacacttaataaagaagagacagtgaaagtaccatcataattaacctaaatttttgttatagcagagtttcttgtttaaaaaaaaataaaatcatctgaaaagcaaa

SEQ ID NO: 173 - DNA, 545 bp, Homo sapien
gtcgacctgggctggacgtggttttgtctgctgcgcccgctcttcgcgctctcgtttcattttctgcagcgcgccagcaggatggcccacaagcagatctactactcggacaagtacttcgacgaacactacgagtaccggcatgttatgttacccagagaactttccaaacaagtacctaaaactcatctgatgtctgaagaggagtggaggagacttggtgtccaacagagtctaggctgggttcattacatgattcatgagccagaaccacatattcttctctttagacgacctcttccaaaagatcaacaaaaatgaagtttatctggggatcgtcaaatctttttcaaatttaatgtatatgtgtatataaggtagtattcagtgaatacttgagaaatgtacaaatctttcatccatacctgtgcatgagctgtattcttcacagcaacagagctcagttaaatgcaactgcaagtaggttactgtaagatgtttaagataaaagttcttccagtcagtttttctcttaagtgcctgtt

SEQ ID NO: 174 - DNA, 469 bp, Homo sapien
gtcgacaaagaatcacagctttctctccatgttttattaacacacagaaaaatactttgaaaaatataccatttctcaaaaatgaaatgtatgatttgctacaaatggccatatggaaaatatgatacctgcttatttttgactcagggtgcattcaatttttatactaactgaaaattacatgattgcgttttgttttaaaagtgaaaaaaagtaataactgcttttagccttgtaatattgaatgcgtcaattggctccccttgtagaatgttgaatggctatcactggtgacagatgttctgtacatcgcagtaatactgcttatataattgtgataattttccgcttcttatttgtcatttttagtgatttaaaaatcccttgatgactccctgaaaaatgactgatgtttttcctatattaagtaatttctgctggtaaagtgtaagtcttttaataatttctt

SEQ ID NO: 175 - DNA, 108 bp, Homo sapien
  misc_feature (1)...(108): n = A,T,C or G
ccttttttttttttttttngaaattnaagtaacttnatnnaaattcaaaaacaatncttaaaactgnntttagagtcaagacccttttgtattataaaaatcacaagt

SEQ ID NO: 176 - DNA, 426 bp, Homo sapien
gtcgactgtttagaagttatacacagagagaaggggaaaagaaactccatcaatcaagctaaaggcagcaaaggaaaatttgaaaagaagcaacgagactgtttaacaaagaacatcaaataagatgatggaactagaagaaaaacaccaatgtccttaattatataaaaacatcaatgtccttaattatataaatttttaaccctcaattgggttaaaaaatcagatttgtactaagagatgtatctttaaaagcaaaagaaagaataaaaagatcaacaagtaaaacaaagtaggagtcagaattaatattagacaaaataaaggtgaaaaatactaaatgcaagaaataatattttagatgacaaaaatgtatgagccataaaaaagtcatgagtttttataaacctaaaatatagcgtcgac

SEQ ID NO: 177 - DNA, 538 bp, Homo sapien
  misc_feature (1)...(538): n = A,T,C or G
cctttttttttttttttttttttttttggangnattngaaattttttctatatanatcatgtgtgacttccataaagaaaaataaacacctatacacagtttacctaatatgtgtaatgttaatgaaaagaatcaaagaaagatgttcgttcattaactctntaaatcaaattgtttttccatttttaccaacttgataccttaatcaagtcactcttgttcttccttaagtgcaaatgaattttttgtttgggttgggggacaacacaaaatacaaacctgggttggattcactgaaaggcccaanaaagggccttantctaggaagtanagngtganatgatacacccacaggctggngcattctggnccacacaaanacgtgctgntccccgccctactgntnaaaacagntctgttttgctnanatgctgctgntgcaacctgcaggtccatganaagaacaactccctggttgtttacancccgngagtgttttgngaatttgcacctacatttcccatgtgatatggactca

SEQ ID NO: 178 - DNA, 566 bp, Homo sapien
gtcgacttggaagcaggtttatttattatatacttgcaattgaatataagatacagacatatatatgtgttatgtatttctagaaatgcacataacatatatttgcctattgtttaatgttttttccagatatttattacagaagggcatggagggatacctacttattcttcattatgagaacaattaaaggcatttattagataggaaattaacagatcatctgcttctataactttattagctacattaaataggcagtgagcaataatttaaaaactcaccattatataaaataataaataacaaagtaaaagttaatgttataaaaataaactgatagtaaggaaaatctaaatgggcatgatcccattttagaagaccaaatgattaatagggttgtcatgttataatagacaattgtctaattatttctgtgtttttatttagtgggtagcagaagttgttcagaagagcagaaatatgtagaaaacatctctaaatttttggcaatttgaaatagcaattctgaggcacacagctcatctacaaaaatcttttgcaga

SEQ ID NO: 179 - DNA, 277 bp, Homo sapien
  misc_feature (1)...(277): n = A,T,C or G
gncgacgggaaaggaatattatggcannaagctgagcaagcaattctggtggaaagtcaaacctgtcagtgctccacaccagggctgtggtcctcccagacatgcataggaatggccacaggtttacactgccttcccagcaattataagcacaccagattcagggagactgaccaccaagggatagtgtaaaaggacattttctcagttgggtccatcagcagtttttcttcctgcatttattgnngaaaactatngtttcatttcttcttttata

SEQ ID NO: 180 - DNA, 349 bp, Homo sapien
  misc_feature (1)...(349): n = A,T,C or G
ccttttttttttttttttttttttttttttttttttttttttagnataaggaaaagctacaaacctcaaggntgttttatttaaaccaaataatntgagcaagacatatntacattaaaaacaaatgaacacattaaaatttcactattttacaatctaaattctagcaacatatacaaatactgagngactacagtacatgccgnggtaananaagtacattntggganaatatnactgacnctcaaaccatttttatttccaatatgtatttcaatacatgtttgtttccacttttcccagngccacacacacncncacacaaaaacaaaacaaaacaaaaaaaaac

SEQ ID NO: 181 - DNA, 435 bp, Homo sapien
  misc_feature (1)...(435): n = A,T,C or G
ccttttttttttttttttgacatttacaggtatttatttgagtaagagctcataaaatatatttttataatatgcacaagaaaaaatacatttgaatgaataaaaaataaaatgacaggaggtgacagaatttagtgtttataaatgaggtcataaagaactttaataattcanagaanaagttcaaagtgtatttaaaagttgagaccctgctttacaatattttataattttaaaaaaaggcgtttaaaggtgataggtgacttaataattttccactttcaaaatgggtttctagacactgttatgaagctgctatgtactaataatactttgcttgccaaagtgtttgggttttgttgttgtttgtttgtttgtttgtttttggttcatgaacaacagtgtctagaaacccactttcaaaatggggtcgac

SEQ ID NO: 182 - DNA, 328 bp, Homo sapien
gtcgaccattgtatctttttcttttctatccctttacatttactctttcagaatccttatgttttactgttttcagaaaacttagtttttaaaatattctgctaatcatttttcatataagtttacattaaataagtcttttaaagtttattataattaaataaagtttattttcacatgtgttttcatatctactgtctcagaactttctccttgccctatttttcctattttatcccctttttgcatcttttgagttgactttttatgattttatttttctctctttactagtttggatattatctaccccactaatattctttca

SEQ ID NO: 183 - DNA, 491 bp, Homo sapien
  misc_feature (1)...(491): n = A,T,C or G
ccttttttttttttttttttttttttttttttacaaacctcaaggttgttttatttaaaccaaataatctgagcaagacatatatacattaaaaacaaatgaacacattaaaatttcactattttacaatctaaattctagcaacatatacaaatactgagtgactacagtacatgccgaggtaagataagtacattctggganaatatcactgacgctcaaaccatttttatttccaatatgtatttcaatacatgtttgtttccacttttcccagngccacacacacacacacaaaaacaaaacaaaacaaaaaaaaacagtcacaagttggattacattanaattggngccacagttgactttaaaagcattttaataaccacccaactcttanattttgcagtttagggacttcaagttcanaaccaaaaagcanagaatcgtttcatgtgacatgatgtttctatagacctcttgctctctaggtc

SEQ ID NO: 184 - DNA, 478 bp, Homo sapien
gtcgacggctgctgttggttgggggccgtcccgctcctaaggcaggaagatggtggccgcaaagaagacgaaaaagtcgctggagtcgatcaactctaggctccaactcgttatgaaaagtgggaagtacgtcctggggtacaagcagactctgaagatgatcagacaaggcaaagcgaaattggtcattctcgctaacaactgcccagctttgaggaaatctgaaatagagtactatgctatgttggctaaaactggtgtccatcactacagtggcaataatattgaactgggcacagcatgcggaaaatactacagagtgtgcacactggctatcattgatccaggtgactctgacatcattagaagcatgccagaacagactggtgaaaagtaaaccttttcacctacaaaatttcacctgcaaaccttaaacctgcaaaattttcctttaataaaatttgcttgttttaaaaaa

SEQ ID NO: 185 - DNA, 596 bp, Homo sapien
  misc_feature (1)...(596): n = A,T,C or G
gtcgacggacgaggagtgcggcactgatgagtactgcgctagtcccacccgcggaggggacgcgggcgtgcaaatctgtctcgcctgcaggaagcgccgaaaacgctgcatgcgtcacgctatgtgctgccccgggaattactgcaaaaatggaatatgtgtgtcttctgatcaaaatcatttccgaggagaaattgaggaaaccatcactgaaagctttggtaatgatcatagcaccttggatgggtattccagaagaaccaccttgtcttcaaaaatgtatcacaccaaaggacaagaaggttctgtttgtctccggtcatcagactgtgcctcaggattgtgttgtgctagacacttctggtccaagatctgtaaacctgtcctgaaagaaggtcaagtgtgtaccaagcataggagaaaaggctctcatggactagaaatattccagcgttgttactgtggagaaggtctgtcttgccggatacagaaagatcaccatcaagccagtaattcttctaggcttcacacttgncagagacactaaaccagctatccaaatgcagtgaactccttttatataatagatgctatga

SEQ ID NO: 186 - DNA, 314 bp, Homo sapien
  misc_feature (1)...(314): n = A,T,C or G
gtcgactgcctatttaatgtagctaataaagttatagaagcagatgatctgttaatttcctatctaataaatgcctttaattgttctcataatgaagaataagtaggtatccctccatgcccttctgtaataaatatctggaaaaaacattaaacaataggcaaatatatgttatgtgcatttctagaaatacataacacatatatatgtctgtatcttatattcaattgcaagtatataataaataaacctgcttccaaacaacaaaaaaaaaaaaaaaaaaaaaaaannaaaaaaaaaaaaaaaaaaaaaaa

SEQ ID NO: 187 - DNA, 331 bp, Homo sapien
  misc_feature (1)...(331): n = A,T,C or G
cctttttttttttttttattcctcagngcttttgattttaattcttttggcatatctaaatgtcagaaagtgaatatatacatacagaattcaaaacaccttcctaaaatggttattattggccantcattnacatctttattttgaaagtctgaattgncaaatagttctaaagtgcattcttgcagctaataaatagcagcatttgtttataaaaccttaagaaattcagaccagggctgganaagtcacaataaaaaatcagacatgatctanatatagtcttccttaatcatctaagacaaacacttgtgtgaattagtttataagg

SEQ ID NO: 188 - DNA, 567 bp, Homo sapien
gtcgacgctgaagaaggaaaagaaatgtgtgaaactcataggagttcccgctgacgctgaggccttaagtgaaagaagtggaaacacccctaactctcccaggttagctgctgaatcaaagcttcaaacagaagttaaagaaggaaaagaaacttcaagcaaattggaaaaagaaacttgtaagaaattacaccctattctatatgtgtcttctaaatctactccagagacccagtgccctcaacagtaaagacttgtctttaataagagtacggtgccacttgcctcaaaagttactatggtgcttaagattgtcttgatctgacatatatcaccttctgggttatttactcattgtgccaggacctggcattttcatgtgcctttgaccaagtgttcagaatttgcttgactctaacctggagagcttcttaagtgatgccccttcatggagcttctatgacagtgaataaactattaattgaaggaaaatgttataattaatgtatctatttgctgcattgtatatggattaaatgataaaaaacaagtaatctaccctcagag

SEQ ID NO: 189 - DNA, 130 bp, Homo sapien
  misc_feature (1)...(130): n = A,T,C or G
cctttttttttttttttttttttttttttttttttttttttttttatcncctaagnanattttaatataaattttgaacagttataaaaaanaaananggcctttgggtcaataacanaacataacaaaa

SEQ ID NO: 190 - DNA, 426 bp, Homo sapien
gtcgaccaacttcccacatatatttactaagatgattaagacttacattttctgcacaggtctgcaaaaacaaaaattataaactagtccatccaagaaccaaagtttgtataaacaggttgctataagcttggtgaaatgaaaatggaacatttcaatcaaacatttcctatataacaattattatatttacaatttggtttctgcaatatttttcttatgtccacccttttaaaaattattatttgaagtaatttatttacaggaaatgttaatgagatgtattttcttatagagatatttcttacagaaagctttgtagcagaatatatttgcagctattgactttgtaatttaggaaaaatgtataataagataaaatctattaaatttttctcctctaaaaactgaaaaaaaaaaaaaaag

SEQ ID NO: 191 - DNA, 550 bp, Homo sapien
  misc_feature (1)...(550): n = A,T,C or G
cctttttttttttttttttttttttttttttttagttngggatatgacctttattgaacttatccaccanagnggaaataatgtctgtacaaaaccaaatgtttgttactataacttctgcatcacaattaaaatccaaacagttttttaaaaacagtcaactcaatcaaaacccactacttcanaatcaatagcttntttgaagccacagtaacacttaaatatggttaanactcgaatgcanaaatttggttggttggaaagctaattaaacttccaacttgctcaaatagaattacaaaaaggcaaaattgtgtttttcacananatacagnccactggaatcaccaacactggacagctgttanagtatttanagtcctganataacaaggaatccaggcntcctttaaacagtcttctgttgncctttcttcccaatcananatttgtggatgtgtggaatgacaccnccaccagcaattgtagccttgatganngaatccaattcttcatctccacgaatagcaagttgcaagtgacgaggggt

SEQ ID NO: 192 - DNA, 299 bp, Homo sapien
  misc_feature (1)...(299): n = A,T,C or G
cctttttttttttttttgaaattnnaaattttattacaaaaactttttattgctataagaaaaatatgtattaattctacaaaataacattcagattatgttctaattcaattattcaatacaatttattctcttgtaaataagagaaacttatttagaatataaaattataacctaatgacaaagctctagtaaattgngaactacacctctacaccgggcttaaatgcatcctgattaatgatttcttcatacatgtcacttattttatccaaaaaaggatttgagttctcgtcgac

SEQ ID NO: 193 - DNA, 536 bp, Homo sapiens
  misc_feature (1)...(536): n = A,T,C or G
ttttttttttttttttttattctnncaatttttatttctcttacatgctcaaagaagccaagcaaatccaggtatacatgtatatgttttaattttacaggagagagaaagaggtataaggcaagaattaactacattttcatttcactatttctttatgagctctattttgctgctaagttcaagtttcaaaaaaattattaattcctctgctatgttatcttgtcccaattcacaaaataacagggatttccccatgtgactcaaaagcaagaatcttactcctaaataacataaacagcaatatgtgtgactactgtcattcattaacttcgatggtgaagttcattaaactgaccattaaaagaacatttgaacaattccaaaagggagcaaggataaatctccaaatcacccaatagacaaggaacccagagatgacatacagngtgctcacttccacccactgccactgagaacactgattgctctcttcaaacacagagcgaagaatgggcctcatgtcacatggggca

SEQ ID NO: 194 - DNA, 566 bp, Homo sapien
  misc_feature (1)...(566): n = A,T,C or G
gtcgactgcactattacccagggcagatattatgagaaactgtttcttctctaagggtttatggcagactttgcttttttaacatgtgagaaatgaattttttattttgtgatttatgtgatttcttttgctgagtgaaggaaaggagaaattgttgctattgtcagcatcttaaaggtatttccagtcaaggcaaggctaagtgctttgtgatagtattaagcaagtcatgttttgaatggattacctgtagtgactcattggaatgatataattatacaagtaatgccaaaaaccaagtcaaagcctaattaaccaaagcactcatttaaaaatcatcatgtttggacctatctggacctctcagcactgtaaaatagttttggttttgtggcatatgaatagctgtttaacaaatcaaagttagctntttgcttctcagcttttttgggcaatacaagttaagttcttaatggggagacattatcatggcatgacttaagggaacattggtttgtgaaggaaaaacagattatctaaagccatctctatgtttctgttcagat

SEQ ID NO: 195 - DNA, 217 bp, Homo sapien
gtcgacataaataaatggaagaaatatcatgttcatgggcttcaaaagtcaacagtaaagatgccattttttcctaaattgatctacaggttcagtgcaattccttccgaatctcaccagggtttttggtagacataaacaagtttattctaaaatttgtatggaaaggcacaggtcctggaataactaaagcaaccttacaaaaaaaaaaaaaaag

SEQ ID NO: 196 - DNA, 391 bp, Homo sapien
  misc_feature (1)...(391): n = A,T,C or G
gtcgacggacagacttaggagttttgtttagagcagttaacatctgaagtgtctaatgcattaacttttgtaaggtactgaatacttaatatgtgggaaacccttttgcgtggtccttaggcttacaatgtgcactgaatcgtttcatgtaagaatccaaagtggacaccattaacaggtctttgaaatatgcatgtactttatattttctatatttgtaactttgcatgttcttgttttgttatataaaaaaattgtaaatgtttaatatctgactgaaattaaacgagcgaagatgagcaccaaaaaaaaaaaaaaaaaaaaaaaaaaaaaannnaaaaaaaaaaannaaaaaaaaaaaaaaaaaaaaaaaaaaaaaaaaaaaaaaaaa

SEQ ID NO: 197 - DNA, 445 bp, Homo sapien
gtcgactggatctttatgtcaatgtgtacatagtacaagcttttttactggaattgaggtttaaaaccacacactgcccttttggtggtgtgcctgttgggccaaaaattgggtgataatgtagtgtcactttctcagctcaatgcagtttctactttttcttatgggaaaatttttcataaaacctttttgcaccaaaacccaggggtgttttttgcaatatccttgttatcctcgtagtgtgccaagtcagaggctttctcttgcccttttcctgctgtgttctcaggcctcccaagggctgtttgactcaacagtctacatccttcgttgtgttttggagaatgtgggggtgggggtcagagttcaaggtgtctgttcccttttcctgtgaactctttctagtccctatttggggagggtggctggaaacagatttttgctg

SEQ ID NO: 198 - DNA, 463 bp, Homo sapien
  misc_feature (1)...(463): n = A,T,C or G
gtcgacgtcagtattaatactgagccagactggcatctacagatttcagatctatcattttattgattcttaagcttgtattaaaaactaggcaatatcatcatggatacataggagaagacacatttacaatcattcattgggccttttatctgtctatccatccatcatcatttgaaggcctaatatatgccaagtactcacatggtatgcattgagacataaaaaagactgtctataacctcaataagtattaaaaatcccattattacccataaggttcatcttatttcatttttagggaataaaattacatgtctatgaaatttcaattttaagcactattgtttttcatgaccataatttatttttaaaaataaattaaaggttaattatatgcatgtatgtatttctaataattaaaaatgtgttcaatccctganaaaaaaaaaaaaaaaaaaaa

SEQ ID NO: 199 - DNA, 129 bp, Homo sapien
  misc_feature (1)...(129): n = A,T,C or G
gtcgaccggcgggcagctgcagcttctgctgctgaggccgggattgctacgactgggactgaagactcagacgatgccctgctgaagatgaccatcagccancaagagtttggccgnactgggcttcct

SEQ ID NO: 200 - DNA, 523 bp, Homo sapien
  misc_feature (1)...(523): n = A,T,C or G
cctttttttttttttttttttttttnaaatctttatttaaaagtccatgctaataatgngtttacatttttacagttacattatgatagaaactgttggattttttaaatatctaaaacaatggcccactgaanaaaggaacaattaactctttaattaattccttaggataaatacccanaaatttaacagctagggcagacttntaatacaataccgaaagtccttccaaaaaccaagnggttgccaacttatgtcccttagcattataacattcttgagccaatagtgtaaaaatacgctgacaattttataggcaaacattactcaaggtatcttactttccacttattactaaagtaattaacccctaaacagatgctcctcaacagngggactacatcctggtaaacctatcataagttgaaactatcaagttgaaatgcatttagtaccctgataaacctatcataaagttgaaaatttgtaaattgaaccagtgtaaatcagaggccatnttact

SEQ ID NO: 201 - DNA, 532 bp, Homo sapien
  misc_feature (1)...(532): n = A,T,C or G
cctttttttttttttttacacttgagcttagccaaaaggctgagaagcgatttttttttaaaagctgttctttaccatggtttaaacgctaaaatgcatagctataaaaacaaaacactgagctaatctgattacatccagcttttgcactcaatagcccttgaccctccagtcataagcaagcctgtcattcgcccagccctgctatacattctcattatagtttcgtttcaaatccagtgttacagaaacaaaacaccaagccctcaatcatgctatgcgtatctttatgtgtgcatgtcttatgtatgtttaaaataaacatttttaaatgttttaggccaggcttggnggctcattcagttttagtttgctttttttttgccattctttgttattttgngaataagtaaaacatttaaatacttaagtcacatctgtataaaaagtatattcataggaaggaatttaacaattttaataaaacttattagcatatcaatgagtttcaagatacacctgaaactaaatt

SEQ ID NO: 202 - DNA, 114 bp, Homo sapien
ctccttggtgtggtcttctctgagtgaatgtcacaaggccggtgacaggagggggtggaggtgaggggacaaagtagaggccgagggtcagtgcctttggagaaagtccagaga

SEQ ID NO: 203 - DNA, 304 bp, Homo sapien
  misc_feature (1)...(304): n = A,T,C or G
gtcgacctttttttcccaacttcttgctttctattggattgttagggatttctgtttttcactttatttctctctgcttatttgaaagctatacagcatggttttctttctttagggatcactcttccactttactttttaaagatggataaattttatacatttaaaaaatttaatctgtatttgtatcttcttcctgagtggaccttagcatgttataaatgctcactgaataattctcattgttaattagagtttggttttattnttttaaanncaatgtacttacttattcttagngtaa

SEQ ID NO: 204 - DNA, 581 bp, Homo sapiens
  misc_feature (1)...(581): n = A,T,C or G
cngcgttgtgaggtgagcnntttcagaagcgcgatcccaggacacgtcgggaagcaagcatccntttagctgcttggaaagaggaccaaagacggctaaaanntcatttggaaatatctctaaatatttgttaccatgtataagctgctaaagagaaattgggcccaacaaaactaattgaataattgaggcagatttgtgtgtatcatcaaattctatccagaagttgaagaatctgaatttaaagattgtgtgcatttaataagaggatgacctttcagtttaatttcactatagaagaccatctggaaaatgaattaacacccattagagatggagctttgaccctggattcctcaaaagagctgtcagtctcagaaagtcaaaaaggagaagagagggacagaaaatgttctgcagaacaatttgacttgcctcaggatcacttgtgggaacataagtcaatggaaaatgcagctccctctcaagacacagacagtccactcagtgcagccagcagttcaaggaacttggagccacatggaaaacagccctccttgagagctgccaaagagcatgct

SEQ ID NO: 205 - DNA, 409 bp, Homo sapiens
  misc_feature (1)...(409): n = A,T,C or G
gccctgaagaacagtgcctggatgtggtgacccactggatccaggaaggtgaagaagggcgtccaaaggatgaccgccacctccgtggctgtggctaccttcccggctgcccgggctccaatggtttccacaacaacgacaccttccacttcctgaaatgctgcaacaccaccaaatgcaacgagggcccaatcctggagcttgaaaatctgccgcagaatggccgccagtgttacagctgcaaggggaacagcacccatggatgctcctctgaagagactttcctcattgactgccggggccccatgaatcaatgtctggtagccacgcgngcgacgtcacagagacncggaaaaaccaaagctatatnggtaaagaggctgtgcaacccgctctcaatgtgccaaca

SEQ ID NO: 206 - DNA, 561 bp, Homo sapiens
  misc_feature (1)...(561): n = A,T,C or G
gtntcatgggaaaggacatgtctctcgaagaaaggttataaaccctgagatatgagggtttttttgagacatccgagcctgtttcgttccgggntgggancaggaataaccctgacttctgagctttcataaccccaggatcctccagaaaatttgcggcgcgctgagggaaaaccttgctgaagctgtacattggaatgcgtttacagtcattgtaatggaagcaaaatacatgaaggaaaaactgttatttgtatccctgcttattgcacctgacgactagttgcagatggttttgtttacctaagaaaacttgtgatataaatgaaaaaaacacctgttttcctagagtcattggttacaaatatgcttcgtctaagagctatttgtccattctcctggagagtgtttcaatttcgacccatcagttgtgaaccactaattattcagatgaataagtgtacagatgaggagcaaatgtttggttttattgaaagaaacaaagccatactttcagaaaagcaagtgggatgtgcatttgatatgctttggaagcttcaa

SEQ ID NO: 207 - DNA, 461 bp, Homo sapiens
  misc_feature (1)...(461): n = A,T,C or G
ggtntttccagccaatgtgacctttaaaacctatgaaggtntnatgcacagttcgtgtcaacaggaaatgatggatgtcaagcaattcattgataaactcctacctccaattgattgacgtcactaagaggccttgtgtagaagtacaccagcatcattgtagtagagtgtaaaccttttcccatgcccagtcttcaaatttctaatgttttgcagtgttaaaatgttttgcaaatacatgccgataacacagatcaaataatatctcctcatgagaaatttatgatcttttaagtttctatacatgtattcttataagacgacccaggatctactatattagaatagatgaagcaggtagcttcttttttctcaaatgtaattcagcaaaataatacagtactgccaccagattttttattacatcatttgaaaattagcagtatgcttaatgaaaattt

SEQ ID NO: 208 - DNA, 296 bp, Homo sapiens
  misc_feature (1)...(296): n = A,T,C or G
gatgaacatccatccnaattncgaagagcctatattataccctcttcaagaatttgcatggcatcaatatctacaggagaaaaaaagggaactcaaaaatgaaacctgggaatattcttcctctgtgatttcttttgttaatggtcagtttctgggtgatgcattggatctgcagaaatgggcccacgaggtgtgggatatagttgacattaaaccctctgcactttatgacgcactcactgaggatttttccgctaagttcttaagagacaccaagcatgatttcgtgtttttgg

SEQ ID NO: 209 - DNA, 282 bp, Homo sapiens
  misc_feature (1)...(282): n = A,T,C or G
gcataataaatgctttgagcttcttgactatcatatacctaaagaaagtgcatcagagaatnatattcctgacttttnnctgactggcaaaaagcnagctttatcttgtcttataggatgcttagtttgccactncacttcaaaccaatgggacagtcntanatggngngacagtgttnaancncaccaaaaggntncntttccntggggccancnctgtcntnancctcnctaanctatttgnanaattttaancncnngttaantaaaaaaaaaaaaaaa

SEQ ID NO: 210 - DNA, 1445 bp, Homo sapiens
ggcgttgtgaggtgagctttttcagaagcgcgatcccaggacacgtcgggaagcaagcatccccagagctgcttggaaagaggaccaaagacgtctaaaaagtcatttggaaatatctctaaatatttgttaccatgtataagctgctaaagagaaattgggcccaacaaaactaattgaataattgaggcagatttgtgtgtatcatcaaattctatccagaagttgaagaatctgaatttaaagattgtgtgcatttaataagaggatgacctttcagtttaatttcactatagaagaccatctggaaaatgaattaacacccattagagatggagctttgaccctggattcctcaaaagagctgtcagtctcagaaagtcaaaaaggagaagagagggacagaaaatgttctgcagaacaatttgacttgcctcaggatcacttgtgggaacataagtcaatggaaaatgcagctccctctcaagacacagacagtccactcagtgcagccagcagttcaaggaacttggagccacatggaaaacagccctccttgagagctgccaaagagcatgctatgcctaaagatttaaagaagatgttagaaaataaagtcatagaaacattaccaggtttccagcatgttaagttatcagtagtgaaaaccatcttgttgaaagagaacttccctggagaaaacatagtttcaaaaagcttttcttctcactctgatctgattacaggtgtttatgagggaggcttaaaaatctgggaatgtacctttgacctcctggcttatttcacaaaggccaaagtgaaatttgctgggaaaaaagtcttggatcttggttgtggatcaggtttactaggtataactgcattcaagggagggtccaaagaaattcactttcaagattataacagtatggtgattgatgaagtaaccttacctaatgtagtagctaactccactttggaagatgaagaaaatgatgtaaatgagccagatgtgaaaagatgcaggaaaccaaaagtaacacaactatataaatgccgatttttttctggtgagtggtctgagttttgtaagcttgtactaagtagtgaaaaactttttgtaaaatatgatctcattctcacctcagaaaccatttacaacccagattattatagtaatttgcaccagactttccttagactgttaagtaaaaatggacgtgtacttttggccagcaaagcacattattttggtgtaggtggaggtgttcatctctttcagaagtttgtagaagaaagagatgtttttaagaccagaatactcaaaataattgatgaaggattgaagaggttcataattgaaataacttttaagtttcctggttaattaacattcactgagtatccaaaatgaaataaacagaaggaccaaaaaaaaaaaaaaa

SEQ ID NO: 211 - DNA, 414 bp, Homo sapiens
aaaaagggaaggaaggagagacagataactctcagtcatttaaaaaactacaataaaatattatgaattatcaattagatcaaagttcctcacagctatatttatataggtaaaaaaaaattaaataggctaaatgcccaaaaatttaagactggcaaaatatacttggctaaatactgtgcgtctctattaaataccatgtttcagaagaattattaatgacatgagaatatgctcaaaatacatattgatatgtgcaaatacatattgcaaagtaagattatagaatgatcctagttcaaaaatgtcacatatatatgtatttaaaaaaaaaggcagttaagatttacaacaaaatgttagtggtgggaccttctggtaggaatacagatttttttttattcagaagttttt

SEQ ID NO: 212 - DNA, 720 bp, Homo sapiens
gtcgacgtaaaatagaaacagaaggggactttatcaacctgattaactttctcaacatgttaaccctacagttaacattataatcaatggtgaatcattgagtactttccttctaagatcagaaacagttcaaagtccactctcaccatttctattcaacattgtactggaatcccagccagtgcagtaataccaataataaaaaattaaagtcataaagattgaaaaggatgaagtaaagctatttcaattctatttagaagtatttagaaaccccaaagaatctacaaaaaactaatagaaataagtgaatatatgaaggtcttactatacaagatcaacatatcaaaagcagtggtatttaagaaaaggttggagactatttataataaacagtggttgaattttgttaatgctttttctgtattttttgaaatgatcttattatttttctctttgctaaaaatgtgagtaaccttgagttgactttctgtgtaaatcaaccttgtgtcccaggaaaaaactccaattgatcatgatgtgttatcctttttatacattgctgtattcaatatgctaatatatttattttttgtgtctatttcatgagggatatcagtatgtaattgttttttcttgttatatctttgttggttttattaatcaacattatgctaacttcatacaatatattggaacatgctccctccttttattttc

SEQ ID NO: 213 - DNA, 1114 bp, Homo sapiens
gctcctaacaaagaagatatcttgaaaatttcagaggatgagcgcatggagctcagtaagagctttcgagtatactgtattatccttgtaaaacccaaagatgtgagtctttgggctgcagtaaaggagacttggaccaaacactgtgacaaagcagagttcttcagttctgaaaatgttaaagtgtttgagtcaattaatatggacacaaatgacatgtggttaatgatgagaaaagcttacaaatacgcctttgataagtatagagaccaatacaactggttcttccttgcacgccccactacgtttgctatcattgaaaacctaaagtattttttgttaaaaaaggatccatcacagcctttctatctaggccacactataaaatctggagaccttgaatatgtgggtatggaaggaggaattgtcttaagtgtagaatcaatgaaaagacttaacagccttctcaatatcccagaaaagtgtcctgaacagggagggatgatttggaagatatctgaagataaacagctagcagtttgcctgaaatatgctggagtatttgcagaaaatgcagaagatgctgatggaaaagatgtatttaataccaaatctgttgggctttctattaaagaggcaatgacttatcaccccaaccaggtagtagaaggctgttgttcagatatggctgttacttttaatggactgactccaaatcagatgcatgtgatgatgtatggggtataccgccttagggcatttgggcatattttcaatgatgcattggttttcttacctccaaatggttctgacaatgactgagaagtggtagaaaagcgtgaatatgatctttgtataggacgtgtgttgtcattatttgtagtagtaactacatatccaatacagctgtatgtttctttttcttttctaatttggtggcactggtataaccacacattaaagtcagtagtacatttttaaatgagggtggtttttttctttaaaacacatgaacattgtaaatgtgttggaaagaagtgttttaagaataataattttgcaaataaactattaataaatattatatgtgataaattctaaaaaaaaaaaaaaa

SEQ ID NO: 214 - DNA, 1495 bp, Homo sapiens
gtaacggatggtgcgccaacgtgagaggaaacccgtgcgcggctgcgctttcctgtccccaagccgttctagacgcggatgaagtgcaaaacaaacttctccatagaggagttgttgcaaagttccagtttataccaaacagtaatcagattccattggaagctaaagattttgagagccttttgtactatatgcaactaacttgatttcaagcttgggaacttttaaaaaaaacattaaagcaaaatgaaaaatgctttctgaaagcagctcctttttgaaaggtgtgatgcttggaagccattttctgtgctttgatccactaatgctaaggacacattaggattggtcatggaaatagaatgcaccaccatgagcatcatcacctacaagctcctaacaaagaagatatcttgaaaatttcagaggatgagcgcatggagctcagtaagagctttcgagtatactgtattatccttgtaaaacccaaagatgtgagtctttgggctgcagtaaaggagacttggaccaaacactgtgacaaagcagagttcttcagttctgaaaatgttaaagagtttgagtcaattaatatggacacaaatgacatgtggttaatgatgagaaaagcttacaaatacgcctttgataagtatagagaccaatacaactggttcttccttgcacgccccactacgtttgctatcattgaaaacctaaagtattttttgttaaaaaaggatccatcacagcctttctatctaggccacactataaaatctggagaccttgaatatgtgggtatggaaggaggaattgtcttaagtgtagaatcaatgaaaagacttaacagccttctcaatatcccagaaaagtgtcctgaacagggagggatgatttggaagatatccgaagataaacagctagcagtttgcctgaaatatgctggagtatttgcagaaaatgcagaagatgctgatggaaaagatgtatttaataccaaatctgttgggctttctattaaagaggcaatgacttatcaccccaaccaggtagtagaaggctgttgttcagatatggctgttacttttaatggactgactccaaatcagatgcatgtgatgatgtatggggtataccgccttagggcatttgggcatattttcaatgatgcattggttttcttacctccaaatggttctgacaatgactgagaagtggtagaaaagcgtgaatatgatctttgtataggacgtgtgttgtcattatttgtagtagtaactacatatccaatacagctgtatgtttctttttcttttctaatttggtggcactggtataaccacccattaaagtcagtagtacatttttaaatgagggtggtttttttctttaaaacacatgaacattgtaaatgtgttggaaaaaagtgttttaagaataataattttgcaaataaactattaataaatattatatgtgataaattctaacc

SEQ ID NO: 215 - DNA, 838 bp, Homo sapiens
ggctgggaagtcagttcgttctctcctctcctctcttcttgtttgaacatggtgcggactaaagcagacagtgttccaggcacttacagaaaagtggtggctgctcgagcccccagaaaggtgcttggttcttccacctctgccactaattcgacatcagtttcatcgaggaaagctgaaaataaatatgcaggagggaaccccgtttgcgtgcgcccaactcccaagtggcaaaaaggaattggagaattctttaggttgtcccctaaagattctgaaaaagagaatcagattcctgaagaggcaggaagcagtggcttaggaaaagcaaagagaaaagcatgtcctttgcaacctgatcacacaaatgatgaaaaagaatagaactttctcattcatctttgaataacgtctccttgtttaccctggtattctagaatgtaaatttacataaatgtgtttgttccaattagctttgttgaacaggcatttaattaaaaaatttaggtttaaatttagatgttcaaaagtagttgtgaaatttgagaatttgtaagactaattatggtaacttagcttagtattcaatataatgcattgtttggtttcttttaccaaattaagtgtctagttcttgctaaaatcaagtcattgcattgtgttctaattacaagtatgttgtatttgagatttgcttagattgttgtactgctgccatttttattggtgtttgattattggaatggtgccatattgtcactccttctacttgctttaaaaagcagagttagatttttgcacattaaaaaattcagtattaattaaaaaaaaaaaaaaa

SEQ ID NO: 216 - DNA, 938 bp, Homo sapiens
cacctcaggctgtggctctttgggcttcttcctaatgcagaagaagttgcccagcagcaaaatcagggaggaggtgagcacctcggcccccgccaggatgaacacgtacatgtagacgtgggtcgcatccaggagtttgcctcccgaagggggcccgacgagcacggccaccgcctccatcagcagcaccaggccaatggcactggagaacttgtaggagatgccaaagaagatgcagaagaccacgaggccgccgtagtcgcccgccgtagagcccgccaggtccgcgaggccgttgaagaacatggagaagctgaagaggtagacggagtagggccgcaccttcccaagccccgccacgaagcccgcggccggccgcgcgaagatgtcaatgaagcccaggatggtgagcaggaaggcggccttggtgtcgggcacgcccaggtccttggcgtagctcaccacgaacacgggcgggacgaagagccccagcaccatgaccgaggcggccacggcgtaaagcacaaagccgcggtcccggaagacgctcaggtctagcaggcgccgggagggtcgcggcggccccgagcccggctgggccgtgaccaccaggggcctcatgagtgcggcacacacgcagcagttgagcagcaggccgcccaggatgaggaagccgccccgccagccgtagcggtcctgcagcagctgccccagcgggctcagggcacacaggaagacagggctacctgctgccgccagcccgttggccatggggcgccgcttgctgaagtagcggttcagcatgatgagcgagggctggaagttgagtgccaaacccaaccccgtgatgaccccagtggtgaggtagacctggatgatgctccggcaaaaggacgcagccaccatgcccagcgacgcaaagagaccccccacaagca

SEQ ID NO: 217 - DNA, 1982 bp, Homo sapiens
ggcgagaggcgggctgaggcggcccagcggcggcaggtgaggcggaaccaaccctcctggccatgggaggggccgtggtggacgagggccccacaggcgtcaaggcccctgacggcggctggggctgggccgtgctcttcggctgtttcgtcatcactggcttctcctacgccttccccaaggccgtcagtgtcttcttcaaggagctcatacaggagtttgggatcggctacagcgacacagcctggatctcctccatcctgctggccatgctctacgggacaggtccgctctgcagtgtgtgcgtgaaccgctttggctgccggcccgtcatgcttgtggggggtctctttgcgtcgctgggcatggtggctgcgtccttttgccggagcatcatccaggtctacctcaccactggggtcatcacggggttgggtttggcactcaacttccagccctcgctcatcatgctgaaccgctacttcagcaagcggcgccccatggccaacgggctggcggcagcaggtagccctgtcttcctgtgtgccctgagcccgctggggcagctgctgcaggaccgctacggctggcggggcggcttcctcatcctgggcggcctgctgctcaactgctgcgtgtgtgccgcactcatgaggcccctggtggtcacggcccagccgggctcggggccgccgcgaccctcccggcgcctgctagacctgagcgtcttccgggaccgcggctttgtgctttacgccgtggccgcctcggtcatggtgctggggctcttcgtcccgcccgtgttcgtggtgagctacgccaaggacctgggcgtgcccgacaccaaggccgccttcctgctcaccatcctgggcttcattgacatcttcgcgcggccggccgcgggcttcgtggcggggcttgggaaggtgcggccctactccgtctacctcttcagcttctccatgttcttcaacggcctcgcggacctggcgggctctacggcgggcgactacggcggcctcgtggtcttctgcatcttctttggcatctcctacggcatggtgggggccctgcagttcgaggtgctcatggccatcgtgggcacccacaagttctccagtgccattggcctggtgctgctgatggaggcggtggccgtgctcgtcgggcccccttcgggaggcaaactcctggatgcgacccacgtctacatgtacgtgttcatcctggcgggggccgaggtgctcacctcctccctgattttgctgctgggcaacttcttctgcattaggaagaagcccaaagagccacagcctgaggtggcggccgcggaggaggagaagctccacaagcctcctgcagactcgggggtggacttgcgggaggtggagcatttcctgaaggctgagcctgagaaaaacggggaggtggttcacaccccggaaacaagtgtctgagtggctgggcggggccggcaggcacagggaggaggtacagaagccggcaacgcttgctatttattttacaaactggactggctcaggcagggccacggctgggctccagctgccggcccagcggatcgtcgcccgatcagtgttttgagggggaaggtggcggggtgggaaccgtgtcattccagagtggatctgcggtgaagccaagccgcaaggttacaaggcatcctcaccaggggccccgcctgctgctcccaggtggcctgcggccactgctatgctcaaggacctggaaacccatgcttcgagacaacgtgactttaatgggagggtgggtgggccgcagacaggctggcagggcaggtgctgcgtggggccctctccagcccgtcctaccctgggctcacatggggcctgtgcccacccctcttgagtgtcttggggacagctctttccacccctggaagatggaaataaacctgcgtgtgggtggagtgttctcgtgccgaattcaaaaagctt

SEQ ID NO: 218 - DNA, 592 bp, Homo sapiens
aggtctcatgggaaaggtcatgtctctcgaagaaaggttataaaccctgagatatgagggttgggcgagacatccgagcctgtttcgttccgtgttgggaccaggaataaccctgacttctgagctttcataaccccaggatcctccagaaaatttgcggcgcgctgagggaaaaccttgctgaagctgtacattggaatgcgtttacagtcattgtaatggaagcaaaatacatgaaggaaaaactgttatttgtatccctgcttattgcacctgacgactagttgcagatggttttgtttacctaagaaaacttgtgatataaatgaaaaaaacacctgttttcctagagtcattggttacaaatatgcttcgtctaagagctatttgtccattctcctggagagtgtttcaatttcgacccatcagttgtgaaccactaattattcagatgaataagtgtacagatgaggagcaaatgtttggttttattgaaagaaacaaagccatactttcagaaaagcaagtgggatgtgcatttgatatgctttggaagcttcaaaagcagaagaccagcctgttaaaaaatgct

SEQ ID NO: 219 - DNA, 650 bp, Homo sapiens
ctgctacccatccctttatgaagaggttttgggagaggagcaagagggagtctgagcaccagccgcagccggggccaaagtttgtggggtcagggccccatccagcagctgccctgccccatgtgacatgaggcccattcttcgctctgtgtttgaagagagcaatcagtgttctcagtggcagtgggtggaagtgagcacactgtatgtcatctctgggttccttgtctattgggtgatttggagatttatccttgctcccttttggaattgttcaaatgttcttttaatggtcagtttaatgaacttcaccatcgaagttaatgaatgacagtagtcacacatattgctgtttatgttatttaggagtaagattcttgcttttgagtcacatggggaaatccctgttattttgtgaattgggacaagataacatagcagaggaattaataatttttttgaaacttgaacttagcagcaaaatagagctcataaagaaatagtgaaatgaaaatgtagttaattcttgccttatacctctttctctctcctgtaaaattaaaacatatacatgtatacctggatttgcttggcttctttgagcatgtaagagaaataaaaattgaaagaataaaaaaaaaaaaaaaaaa

SEQ ID NO: 220 - DNA, 782 bp, Homo sapiens
ggtgaatccagccaatgtgacctttaaaacctatgaaggtatgatgcacagttcgtgtcaacaggaaatgatggatgtcaagcaattcattgataaactcctacctccaattgattgacgtcactaagaggccttgtgtagaagtacaccagcatcattgtagtagagtgtaaaccttttcccatgcccagtcttcaaatttctaatgttttgcagtgttaaaatgttttgcaaatacatgccgataacacagatcaaataatatctcctcatgagaaatttatgatcttttaagtttctatacatgtattcttataagacgacccaggatctactatattagaatagatgaagcaggtagcttcttttttctcaaatgtaattcagcaaaataatacagtactgccaccagattttttattacatcatttgaaaattagcagtatgcttaatgaaaatttgttcaggtataaatgagcagttaagatataaacaatttatgcatgctgtgacttagtctatggatttattccaaaattgcttagtcaccatgcagtgtctgtatttttatatatgtgttcatatatacataatgattataatacataataagaatgaggtggtattacattattcctaataatagggataatgctgtttattgtcaagaaaaagtaaaatcgttctcttcaattaatggcccttttattttgggaccaggcttttattctccctgatattatttctatttaatactcttttctctcaaaaaaaaaaaaaaa

SEQ ID NO: 221 - DNA, 2417 bp, Homo sapiens
cttccttccgcttgcgctgtgagctgaggcggtgtatgtgcggcaataacatgtcaaccccgctgcccgccatcgtgcccgccgcccggaaggccaccgctgcggtgattttcctgcatggattgggagatactgggcacggatgggcagaagcctttgcaggtatcagaagttcacatatcaaatatatctgcccgcatgcgcctgttaggcctgttacattaaatatgaacgtggctatgccttcatggtttgatattattgggctttcaccagattcacaggaggatgaatctgggattaaacaggcagcagaaaatataaaagctttgattgatcaagaagtgaagaatggcattccttctaacagaattattttgggagggttttctcagggaggagctttatctttatatactgcccttaccacacagcagaaactggcaggtgtcactgcactcagttgctggcttccacttcgggcttcctttccacagggtcctatcggtggtgctaatagagatatttctattctccagtgccacggggattgtgaccctttggttcccctgatgtttggttctcttacggtggaaaaactaaaaacattggtgaatccagccaatgtgacctttaaaacctatgaaggtatgatgcacagttcgtgtcaacaggaaatgatggatgtcaagcaattcattgataaactcctacctccaattgattgacgtcactaagaggccttgtgtagaagtacaccagcatcattgtagtagagtgtaaaccttttcccatgcccagtcttcaaatttctaatgttttgcagtgttaaaatgttttgcaaatacatgccgataacacagatcaaataatatctcctcatgagaaatttatgatcttttaagtttctatacatgtattcttataagacgacccaggatctactatattagaatagatgaagcaggtagcttcttttttctcaaatgtaattcagcaaaataatacagtactgccaccagattttttattacatcatttgaaaattagcagtatgcttaatgaaaatttgttcaggtataaatgagcagttaagatataaacaatttatgcatgctgtgacttagtctatggatttattccaaaattgcttagtcaccatgcagtgtctgtatttttatatatgtgttcatatatacataatgattataatacataataagaatgaggtggtattacattattcctaataatagggataatgctgtttattgtcaagaaaaagtaaaatcgttctcttcaattaatggcccttttattttgggaccaggcttttattttccctgatattatttctatttaatactcttttctctcaagaaaaaaaaaaaagtttgttttttctttattgtccttcatagcaggccaagtattgcctctctgcaatagacagctactgtcaatacatgctgtaatttgacattctgggtcacagatataaggtatttaaaatctatttatgctttatagagaaaccagacattaaaacttcatgcactacttatttcgaattactgtaccttatccaaatttacacctagctattaggatcttcaacccaggtaacaggaataattctgtggtttcatttttctgtaaacaactgaaagaataattagatcatattctagtatgttctgaaatatctttaagactgatcttaaaaactaacttctaagatgatttcatcttctcatagtatagagtttactttgtacacgttgaaaccaactactgtagaagatgaggaatctattgtaattttttgctttattttcatctgccagtggacttatttgaattttcactttagtcaaattattttttgtattagtttttgatgcagacataaaaatagcaatcattttaaattgtcaaaatttccagattactggtaaaaattatttgaaaacaaacttatgggtaataaaggctagtcagaaccctataccataaagtgtagttaccatacagattaatatgtagcaaaaatgtatgcttgatatttctcaactgtgttaatttttctgctgtattccagctgaccaaaacaatattaagaatgcatctttataaatgggtgctaattgataatggaaataatttagtaatggactatacaggatgttaataatgaagccatatgtttatgtctggatttaaaaattttaaacaatcatttactatgtcatttttctttaccttgaagaacataaactgttatttcacttctacaaatcagcaagatattatttatggcaagaaatattccattgaaatattgtgctgtaacatgggaaagtgtaaatgtttttcatggtttctatcaatgtgaaataaaatttaattctgaaaaa

SEQ ID NO: 222 - DNA, 1466 bp, Homo sapiens
ggtggtggggctcttcagctgccccaactttcagattgcgaagagcgccgctgagaatctgaagaataatcatccatccaaatttgaagatcctatattagttcctcttcaagaatttgcatggcatcaatatctacaggagaaaaaaagggaactcaaaaatgaaacctgggaatattcttcctctgtgatttcttttgttaatggtcagtttctgggtgatgcattggatctgcagaaatgggcccacgaggtgtgggatatagttgacattaaaccctctgcactttatgacgcactcactgaggatttttccgctaagttcttaagagacaccaagcatgatttcgtgtttttggacatttgtattgattcttctccaattggaagattgatttttgagctatactgtgatgtgtgtcccaaaacatgtaaaaattttcaggtcttgtgcacaggaaaagcagggttttctcaacgtggcataagactacattacaaaaattccatttttcatcgaatagtacagaatggctggatacaaggaggggatatagtgtatggaaaaggagataatggagagtcgatttatggtccaacatttgaaggacaggctcccatgcagatggaactgttgggaatggcatcaaaattatccaggatacagggacaatcatgacaaagagggaataggaacagagtcagaaatttaaggaagaaagccacatgcttcaatatgcaagattttcaacatgcaagagggagctttttgaaactagaaaatctactttctttctaaagacacatcttctaaacatttaggaaaactaatgtcaccctatataacaaagagagtttctctgaaagaaaataatgtttattcaggaatagggtattgctgtaggcatacatgtgccataggaaacgatgtgcatattcaggaaggtaaaggcagacaaagggttttaaaggaaaattggggaagattacgtaattgttttgaaatgattatccttggctatagcgatcagtaacaagagtccaaggttggactggacaggtgtccctgcagaagtattaatatttcctgcataaggtcgcaatggcctttatgcaaggttgtggcttttgtagttctttgtgattgttttgctatcaggcatacaagtgtgagagttctctgttcatagctttccttggctctatttgtcagcattttttaaacatgactacattttgattctgaccactattacactaattttatattagaatgaacaatagaagtttcaaggtgattataagaataaagagaataaagagcagagtaacatcagcactgatagtgaatgtaccctagaaagacatgctcataggatacagttgacccttgagcaacatggatttgaaatgtacgagtccacttaaagaaacttacagtcagtttctttaaaaaaaaaaaaaaa